(19) United States
(12) Patent Application Publication
(10) Pub. No.: US 2019/0166840 A1
(43) Pub. Date: Jun. 6, 2019

(54) SUBSTITUTED PYRAZOLINYL DERIVATIVES, PROCESSES FOR THEIR PREPARATION AND THEIR USE AS HERBICIDES AND/OR PLANT GROWTH REGULATORS

(71) Applicant: BAYER CROPSCIENCE AKTIENGESELLSCHAFT, Monheim am Rhein (DE)

(72) Inventors: Jana FRANKE, Mainz (DE); Hendrik HELMKE, Liederbach (DE); Jens FRACKENPOHL, Frankfurt (DE); Anu Bheemaiah MACHETTIRA, Frankfurt am Main (DE); Hansjoerg DIETRICH, Liederbach am Taunus (DE); Christopher Hugh ROSINGER, Hofheim (DE); Dirk SCHMUTZLER, Hattersheim (DE); Eimar GATZWEILER, Bad Nauheim (DE); Peter LUEMMEN, Idstein (DE)

(21) Appl. No.: 16/324,482

(22) PCT Filed: Aug. 4, 2017
(86) PCT No.: PCT/EP2017/069802

§ 371 Date: Feb. 8, 2019

(30) Foreign Application Priority Data
Aug. 11, 2016 (EP) 16183780.2

(51) Int. Cl. A01N 43/56; C07D 401/14; C07D 403/04

(52) U.S. Cl. CPC A01N 43/56; C07D 403/04; C07D 401/14

(57) ABSTRACT

Primarily, the present invention relates to certain substituted pyrazolinylpyrrolones and pyrazolinylhydantoins of the formula (I) defined below or salts thereof and to their use as herbicides, in particular for controlling broad-leaved weeds and/or weed grasses in crops of useful plants and/or as plant growth regulators for influencing the growth of crops of useful plants. Furthermore, the present invention relates to herbicidal and/or plant growth-regulating compositions comprising one or more compounds of formula (I) and to processes for preparing the compounds of the formula (I).

The invention relates to the technical field of crop protection agents, in particular that of herbicides for the selective control of broad-leaved weeds and weed grasses in crops of useful plants.

Primarily, the present invention relates to certain substituted pyrazolinylpyrrolones and pyrazolinylhydantoins of the formula (I) defined below or salts thereof and to their use as herbicides, in particular for controlling broad-leaved weeds and/or weed grasses in crops of useful plants and/or as plant growth regulators for influencing the growth of crops of useful plants. Furthermore, the present invention relates to herbicidal and/or plant growth-regulating compositions comprising one or more compounds of formula (I) and to processes for preparing the compounds of the formula (I).

In their application, crop protection agents known to date for the selective control of harmful plants in crops of useful plants or active compounds for controlling unwanted vegetation sometimes have disadvantages, be it (a) that they have no or else insufficient herbicidal activity against particular harmful plants, (b) that the spectrum of harmful plants which can be controlled with an active compound is not wide enough, (c) that their selectivity in crops of useful plants is too low and/or (d) that they have a toxicologically unfavourable profile. Furthermore, some active compounds which can be used as plant growth regulators for a number of useful plants cause unwanted reduced harvest yields in other useful plants or are not compatible with the crop plant, or only within a narrow application rate range. Some of the known active compounds cannot be produced economically on an industrial scale owing to precursors and reagents which are difficult to obtain, or they have only insufficient chemical stabilities. In the case of other active compounds, the activity is too highly dependent on environmental conditions, such as weather and soil conditions.

The herbicidal activity of these known compounds, in particular at low application rates, and/or their compatibility with crop plants remain deserving of improvement.

It is known that certain substituted pyrazolinylureas can be used as insecticidally active compounds (cf. U.S. Pat. No. 4,863,947). It is further known that substituted pyrazolinylcarboxylic esters can be used in mixtures with herbicidally active compounds (cf. WO 2005/092103, EP1410715). The action of certain substituted pyrazolines for increasing the stress defence in plants with regard to abiotic stress is described in EP 2289310. It is also known that certain substituted spirocyclic pyrazolines can be used as pharmaceutically active compounds, for example as agonists of α7-nicotinergic acetylcholine receptors (cf. WO2008/000469). Furthermore, certain substituted 3-aminopyrazolines have been described as pharmaceutically active compounds for inhibiting D-amino acid oxidases (cf. WO2007/093829).

Various documents describe substituted pyrrolones and hydantoin having herbicidal properties. WO2016/071359 and WO2016/071360 disclose pyrrolones carrying heterocyclic substituents at the nitrogen including, for example, isoxazolines which are optionally substituted further. Furthermore, substituted pyrrolones and their herbicidal properties are described in CH633678, EP0297378, EP0334133, EP0339390 and EP0286816. Additionally, WO2016/071361, WO2016/071362, WO2016/071363 and WO2016/071364 describe substituted hydantoins which also carry heterocyclic substituents at the nitrogen, for example isoxazolines which are optionally substituted further.

For the reasons stated above, there is still a need for potent herbicides and/or plant growth regulators for the selective use in plant crops or the use on non-crop land, where these active ingredients preferably should have further advantageous properties in application, for example with respect to their compatibility with crop plants.

It is the primary object of the present invention to provide compounds having herbicidal activity (herbicides) which are highly effective against economically important harmful plants even at relatively low application rates and can be used selectively in crop plants, preferably with good activity against harmful plants, and at the same time preferably have good compatibility with crop plants. Preferably, these herbicidal compounds should be particularly effective and efficient against a broad spectrum of weed grasses and preferably also have good activity against a large number of weeds.

Surprisingly, it has now been found that certain substituted pyrazolinylpyrrolones and pyrazolinylhydantoins of the formula (I), defined below, or their salts achieve this object and are particularly suitable as herbicides.

Primarily, the present invention thus provides pyrazolinylpyrrolones and pyrazolinylhydantoins of the general formula (I) or salts thereof

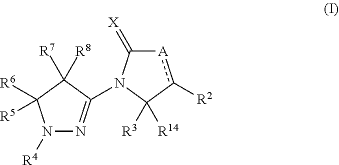

in which
X represents oxygen or sulfur,
A represents the grouping C—R¹ or the grouping N—R⁹ (N=nitrogen) where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents hydrogen, halogen, hydroxy, (C₁-C₈)-alkyl, (C₁-C₈)-haloalkyl, (C₁-C₈)-hydroxyalkyl, (C₁-C₈)-alkoxy, (C₁-C₈)-alkoxyalkyl, (C₁-C₈)-alkoxy-(C₁-C₈)-alkyloxy, (C₁-C₈)-alkylthio, (C₁-C₈)-haloalkoxy, (C₁-C₈)-haloalkylthio, (C₃-C₁₀)-cycloalkyl, aryl, heteroaryl, (C₃-C₈)-cycloalkyl-(C₁-C₈)-alkyl, heterocyclyl, (C₂-C₈)-alkenyl, (C₂-C₈)-alkenyloxy, (C₂-C₈)-alkynyl, (C₂-C₈)-alkynyloxy, NR¹⁰R¹¹, aryl-(C₁-C₈)-alkyl, heteroaryl-(C₁-C₈)-alkyl or heterocyclyl-(C₁-C₈)-alkyl,
R² represents hydrogen, halogen, hydroxy, (C₁-C₈)-alkyl, (C₁-C₈)-haloalkyl, (C₁-C₈)-hydroxyalkyl, hydroxycarbonyl-(C₁-C₈)-alkyl, (C₁-C₈)-alkoxy, (C₁-C₈)-alkoxyalkyl, (C₁-C₈)-alkoxy-(C₁-C₈)-alkyloxy, (C₁-C₈)-alkylthio, (C₁-C₈)-haloalkoxy, (C₁-C₈)-haloalkylthio, (C₃-C₁₀)-cycloalkyl, aryl, heteroaryl, (C₃-C₅)-cycloalkyl-(C₁-C₈)-alkyl, heterocyclyl, (C₂-C₈)-alkenyl, (C₂-C₈)-alkenyloxy, (C₂-C₈)-alkynyl, (C₂-C₈)-alkynyloxy, NR¹⁰R¹¹, aryl-(C₁-C₈)-alkyl, heteroaryl-(C₁-C₈)-alkyl, heterocyclyl-(C₁-C₈)-alkyl, (C₁-C₈)-cyanoalkyl, C(O)R¹², C(O)OR¹², C(O)NR¹⁰R¹¹, SO₂R¹³, (C₁-C₈)-alkoxycarbonyl-(C₁-C₈)-alkyl, (C₂-C₈)-alkenyloxycarbonyl-(C₁-C₈)-alkyl, aryl-(C₁-C₈)-alkoxycarbonyl-(C₁-C₈)-alkyl, heteroaryl-(C₁-C₈)-alkoxycarbonyl-(C₁-C₈)-alkyl, aryloxycarbonyl-(C₁-C₈)-alkyl, arylcarbonyl-(C₁-C₈)-alkyl, (C₁-C₈)-alkylcarbonyl-(C₁-C₈)-alkyl, heteroarylcarbonyl-(C₁-C₈)-alkyl or heterocyclylcarbonyl-(C₁-C₈)-alkyl,
   or where R¹ and R² together with the two carbon atoms to which they are attached form a fully saturated or partially saturated ring which has 3 to 7 members in total, which is optionally interrupted by one to three heteroatoms from the group consisting of N, O and S and which is optionally substituted further,
R³ represents hydroxy, hydrothio, halogen, NR¹⁰R¹¹, (C₁-C₈)-alkoxy, (C₃-C₁₀)-cycloalkyl-(C₁-C₈)-alkoxy, aryl-(C₁-C₈)-alkoxy, (C₁-C₈)-alkoxy-(C₁-C₈)-alkoxy, arylcarbonyloxy, (C₁-C₈)-alkylcarbonyloxy, aryl-(C₁-C₈)-alkylcarbonyloxy, heteroarylcarbonyloxy, (C₃-C₁₀)-cycloalkylcarbonyloxy, heterocyclylcarbonyloxy, (C₁-C₈)-haloalkyl-carbonyloxy, (C₂-C₈)-alkenylcarbonyloxy, OC(O)OR¹², OC(O)SR¹², OC(S)OR¹², OC(S)SR¹², OC(O)NR¹⁰R¹¹, OC(S)NR¹⁰R¹¹, OSO₂R³, OSO₂OR¹² or OCHO,
R⁴ represents hydrogen, (C₁-C₈)-alkyl, (C₁-C₈)-haloalkyl, (C₃-C₁₀)-cycloalkyl, (C₃-C₁₀)-cycloalkyl-(C₁-C₈)-alkyl, aryl, heteroaryl, heterocyclyl, aryl-(C₁-C₈)-alkyl, heteroaryl-(C₁-C₈)-alkyl, heterocyclyl-(C₁-C₈)-alkyl, (C₂-C₈)-alkenyl, (C₂-C₈)-alkynyl, (C₂-C₈)-haloalkenyl, (C₂-C₈)-haloalkynyl, (C₄-C₁₀)-cycloalkenyl, aryl-(C₂-C₈)-alkenyl, heteroaryl-(C₂-C₈)-alkenyl, heterocyclyl-(C₂-C₈)-alkenyl, arylcarbonyl-(C₁-C₈)-alkyl, (C₁-C₈)-alkylcarbonyl-(C₁-C₈)-alkyl, heteroarylcarbonyl-(C₁-C₈)-alkyl, (C₃-C₁₀)-cycloalkylcarbonyl-(C₁-C₈)-alkyl, aryl-(C₁-C₈)-alkoxycarbonyl-(C₁-C₈)-alkyl, (C₁-C₈)-alkoxycarbonyl-(C₁-C₈)-alkyl, (C₁-C₈)-alkoxy-(C₁-C₈)-alkyl, (C₁-C₈)-haloalkoxy-(C₁-C₈)-alkyl, C(O)R¹², SO₂R¹³, CHO, R¹⁰R¹¹N—(C₁-C₈)-alkyl or cyano-(C₁-C₈)-alkyl,
R⁵ represents hydrogen, (C₁-C₈)-alkyl, (C₁-C₈)-haloalkyl, (C₃-C₁₀)-cycloalkyl, (C₃-C₁₀)-cycloalkyl-(C₁-C₈)-alkyl, aryl, heteroaryl, heterocyclyl, aryl-(C₁-C₈)-alkyl, heteroaryl-(C₁-C₈)-alkyl, heterocyclyl-(C₁-C₈)-alkyl, cyano, C(O)OR¹², C(O)NR¹⁰R¹¹, R¹²O(O)C—(C₁-C₈)-alkyl, R¹²O—(C₁-C₈)-alkyl, (C₁-C₈)-alkylcarbonyloxy-(C₁-C₈)-alkyl, (C₃-C₁₀)-cycloalkylcarbonyloxy-(C₁-C₈)-alkyl, arylcarbonyloxy-(C₁-C₈)-alkyl, heteroarylcarbonyloxy-(C₁-C₈)-alkyl or heterocyclylcarbonyloxy-(C₁-C₈)-alkyl,
R⁶ represents hydrogen, (C₁-C₈)-alkyl, (C₁-C₈)-haloalkyl, (C₃-C₁₀)-cycloalkyl, (C₃-C₁₀)-cycloalkyl-(C₁-C₈)-alkyl, aryl, heteroaryl, heterocyclyl, aryl-(C₁-C₈)-alkyl, heteroaryl-(C₁-C₈)-alkyl or heterocyclyl-(C₁-C₈)-alkyl,
R⁷ and R⁸ are identical or different and independently of one another represent hydrogen, halogen or (C₁-C₈)-alkyl,
R⁹ represents hydrogen, hydroxy, (C₁-C₈)-alkyl, (C₁-C₈)-haloalkyl, (C₁-C₈)-hydroxyalkyl, hydroxycarbonyl-(C₁-C₈)-alkyl, (C₁-C₈)-alkoxy, (C₁-C₈)-alkoxy-(C₁-C₈)-alkyl, (C₁-C₈)-alkoxyalkyloxy, (C₁-C₈)-haloalkoxy, (C₃-C₁₀)-cycloalkyl, aryl, heteroaryl, (C₃-C₁₀)-cycloalkyl-(C₁-C₈)-alkyl, heterocyclyl, (C₂-C₈)-alkenyl, (C₂-C₈)-alkenyloxy, (C₂-C₈)-alkynyl, (C₂-C₈)-alkynyloxy, NR¹⁰R¹¹, aryl-(C₁-C₈)-alkyl, heteroaryl-(C₁-C₈)-alkyl, heterocyclyl-(C₁-C₈)-alkyl, (C₁-C₈)-cyanoalkyl, C(O)R¹², C(O)OR¹², C(O)NR¹⁰R¹¹, SO₂R¹³, (C₁-C₈)-alkoxycarbonyl-(C₁-C₈)-alkyl, (C₂-C₈)-alkenyloxycarbonyl-(C₁-C₈)-alkyl, aryl-(C₁-C₈)-alkoxycarbonyl-(C₁-C₈)-alkyl, heteroaryl-(C₁-C₈)-alkoxycarbonyl-(C₁-C₈)-alkyl, aryloxycarbonyl-(C₁-C₈)-alkyl, arylcarbonyl-(C₁-C₈)-alkyl, (C₁-C₈)-alkylcarbonyl-(C₁-C₈)-alkyl, heteroarylcarbonyl-(C₁-C₈)-alkyl or heterocyclylcarbonyl-(C₁-C₈)-alkyl,
   or where R² and R⁹ together with the nitrogen atom or carbon atom to which they are respectively attached form a fully saturated or partially saturated ring which has 3 to 7 members in total, which is optionally interrupted by one to three heteroatoms from the group consisting of N, O and S and which is optionally substituted further,
R¹⁰ and R¹¹ are identical or different and independently of one another represent hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₂-C₈)-haloalkynyl, (C₃-C₁₀)-cycloalkyl, (C₄-C₁₀)-cycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₁₀)-cycloalkyl-(C₁-C₈)-alkyl, (C₄-C₁₀)-cycloalkenyl-(C₁-C₈)-alkyl, C(O)R¹², SO₂R¹³, heterocyclyl, (C₁-C₈)-alkoxycarbonyl-(C₁-C₈)-alkyl, (C₁-C₈)-alkoxycarbonyl, aryl-(C₁-C₈)-alkoxycarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl or heterocyclyl-(C₁-C₈)-alkyl,
R¹² represents (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₂-C₈)-haloalkynyl, (C₃-C₁₀)-cycloalkyl, (C₄-C₁₀)-cycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₁₀)-cycloalkyl-(C₁-C₈)-alkyl, (C₄-C₁₀)-cycloalkenyl-(C₁-C₈)-alkyl, (C₁-C₈)-alkoxycarbonyl-(C₁-C₈)-alkyl, (C₂-C₈)-alkenyloxycarbonyl-(C₁-C₈)-alkyl, aryl-(C₁-C₈)-alkoxycarbonyl-(C₁-C₈)-alkyl, hydroxycarbonyl-(C₁-C₈)-alkyl, heterocyclyl or heterocyclyl-(C₁-C₈)-alkyl,
R¹³ represents (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₂-C₈)-haloalkynyl, (C₃-C₁₀)-cycloalkyl, (C₄-C₁₀)-cycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₁₀)-cycloalkyl-(C₁-C₈)-alkyl, (C₄-C₁₀)-cycloalkenyl-(C₁-C₈)-alkyl or NR¹⁰R¹¹,
R¹⁴ represents hydrogen or (C₁-C₈)-alkyl,
   or where R³ and R¹⁴ together with the carbon atom to which they are attached form a carbonyl group,

where the cyclic structural elements (in particular the structural elements aryl, cycloalkyl, cycloalkenyl, heteroaryl and heterocyclyl) of the radicals mentioned respectively in R¹, R², R³, R⁴, R⁵, R⁶, R⁹, R¹⁰, R¹¹, R¹² and R¹³ and the rings formed by R¹ and R² or R² and R⁹, having 3 to 7 members in total, are in each case unsubstituted or substituted by one or more radicals selected from the group consisting of halogen, nitro, hydroxy, cyano, NR¹⁰R¹¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxy-carbonyl, (C₁-C₄)-haloalkoxy-carbonyl, (C₁-C₄)-alkylcarboxy, (C₃-C₆)-cycloalkyl, (C₃-C₆)-cycloalkyl-(C₁-C₆)-alkyl, (C₁-C₄)-alkoxy-carbonyl-(C₁-C₄)-alkyl, hydroxycarbonyl, hydroxycarbonyl-(C₁-C₄)-alkyl, R¹⁰R¹¹N-carbonyl, and where the structural elements cycloalkyl, cycloalkenyl and heterocyclyl have n oxo groups, where n=0, 1 or 2.

Here, in connection with the compounds of the formula (I) with regard to the radicals mentioned in R¹, R², R³, R⁴, R⁵, R⁶, R⁹, R¹⁰, R¹¹, R¹² and R¹³, the structural elements aryl, heteroaryl and heterocyclyl have the following meaning:

“Aryl” represents a mono-, bis- or polycyclic aromatic system having 6 to 14, preferably 6 to 10, ring carbon atoms, particularly preferably phenyl.

“Heteroaryl” represents a fully unsaturated aromatic 5- to 7-membered, preferably 5- or 6-membered, heterocyclic structural element having 1, 2 or 3 identical or different heteroatoms from the group consisting of N, O and S in the ring, where, however, two oxygen atoms are not directly adjacent to one another.

“Heterocyclyl” represents a 3- to 9-membered, preferably 3- to 6-membered, saturated or partially saturated heterocyclic structural element having at least one carbon ring atom and 1, 2 or 3 identical or different heteroatoms from the group consisting of N, O and S in the ring, where, however, two oxygen atoms are not directly adjacent to one another.

The compounds of the formula (I) according to the invention and/or their salts have excellent herbicidal activity against a broad spectrum of economically important mono- and dicotyledonous annual harmful plants. The compounds according to the invention also have good control over perennial harmful plants which are difficult to control and produce shoots from rhizomes, root stocks or other perennial organs. The compounds according to the invention have a broader spectrum of activity against weeds, i.e. the compounds according to the invention and/or their salts can be used to control effectively a relatively large number of different weeds.

The compounds of the formula (I) according to the invention and/or their salts have proven to be very effective in controlling harmful plants such as Alopecurus myosuroides, Avena fatua, Cyperus esculentus, Echinochloa crus-galli, Lolium multiflorum, Setaria viridis, Abutilon theophrasti, Amaranthus retroflexus, Polygonum convolvulus (=Fallopia convolvulus), Stellaria media, Viola tricolor, and Veronica persica, where the preferred and particularly preferred compounds according to the invention exhibited in the biological tests an 80% to 100% herbicidal action against one, several or all of the specified harmful plants, and in so doing simultaneously more acceptable and mostly very slight damage to the useful plant, in particular in oilseed rape, soya beans, cotton and cereals (and here in particular maize, barley, wheat, rye, oats, triticale, millet, rice).

By addition of a suitable inorganic or organic acid onto a basic group, such as, for example, amino or alkylamino, the compounds of the formula (I) are able to form salts. Suitable acidic groups present, such as, for example, carboxylic acid groups, are able to form inner salts with groups which for their part can be protonated, such as amino groups.

The compounds of the formula (I) may preferably be present in the form of agriculturally usable salts, where the type of salt is otherwise generally immaterial. In general, suitable salts are the salts of those cations or the acid addition salts of those acids whose cations and anions, respectively, have no adverse effect on the herbicidal activity of the compounds of the formula (I).

The compounds of the general formula (I) can form salts by addition of a suitable inorganic or organic acid, for example mineral acids, for example HCl, HBr, H₂SO₄, H₃PO₄ or HNO₃, or organic acids, for example carboxylic acids such as formic acid, acetic acid, propionic acid, oxalic acid, lactic acid or salicylic acid or sulfonic acids, for example p-toluenesulfonic acid, onto a basic group, for example amino, alkylamino, dialkylamino, piperidino, morpholino or pyridino. In such a case, these salts will comprise the conjugate base of the acid as the anion. Suitable substituents in deprotonated form, for example sulfonic acids, particular sulfonamides or carboxylic acids, are capable of forming internal salts with groups, such as amino groups, which are themselves protonatable. Salts may also be formed by action of a base on compounds of the general formula (I). Examples of suitable bases are organic amines such as trialkylamines, morpholine, piperidine and pyridine, and the hydroxides, carbonates and hydrogencarbonates of ammonium, alkali metals or alkaline earth metals, especially sodium hydroxide, potassium hydroxide, sodium carbonate, potassium carbonate, sodium hydrogencarbonate and potassium hydrogencarbonate. These salts are compounds in which the acidic hydrogen is replaced by an agriculturally suitable cation, for example metal salts, especially alkali metal salts or alkaline earth metal salts, in particular sodium and potassium salts, or else ammonium salts, salts with organic amines or quaternary ammonium salts, for example with cations of the formula [NR^{a}R^{b}R^{C}R^{d}]⁺ in which R^{a} to R^{d} are each independently an organic radical, especially alkyl, aryl, arylalkyl or alkylaryl. Also suitable are alkylsulfonium and alkylsulfoxonium salts, such as (C₁-C₄)-trialkylsulfonium and (C₁-C₄)-trialkylsulfoxonium salts.

The substituted pyrazolinylpyrrolones and pyrazolinylhydantoins of the formula (I) according to the invention can, depending on external conditions such as pH, solvent and temperature, be present in various tautomeric structures, all of which are embraced by the formula (I). The compound of the formula (I) thus also includes tautomers, even if formally the formula (I) correctly describes only one of the respective tautomers which are in equilibrium with one another or which can be converted into one another.

The compounds of the formula (I) also include all physical forms in which they may be present as a pure substance or, if appropriate, as a mixture with other substances, in particular also polymorphic crystal forms of the compounds of the formula (I) or salts thereof or solvent adducts (for example hydrates).

The compounds of the formula (I) according to the invention and salts thereof are also referred to hereinafter as “compounds of the general formula (I)” or “compounds of the formula (I)”.

Preferred compounds of the general formula (I) according to the invention are those

in which
X represents oxygen or sulfur, preferably oxygen,
A represents the grouping C—R¹ or the grouping N—R⁹ (N=nitrogen), where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents hydrogen, halogen, hydroxy, (C₁-C₇)-alkyl, (C₁-C₇)-haloalkyl, (C₁-C₇)-hydroxyalkyl, (C₁-C₇)-alkoxy, (C₁-C₇)-alkoxyalkyl, (C₁-C₇)-alkoxy-(C₁-C₇)-alkyloxy, (C₁-C₇)-haloalkoxy, (C₃-C₁₀)-cycloalkyl, (C₂-C₇)-alkenyl, (C₂-C₇)-alkenyloxy, (C₂-C₇)-alkynyl or (C₂-C₇)-alkynyloxy,
R² represents hydrogen, halogen, hydroxy, (C₁-C₇)-alkyl, (C₁-C₇)-haloalkyl, (C₁-C₇)-hydroxyalkyl, (C₁-C₇)-alkoxy, (C₁-C₇)-alkoxyalkyl, (C₁-C₇)-alkoxy-(C₁-C₇)-alkyloxy, (C₁-C₇)-haloalkoxy, (C₃-C₁₀)-cycloalkyl, (C₂-C₇)-alkenyl, (C₂-C₇)-alkenyloxy, (C₂-C₇)-alkynyl, (C₂-C₇)-alkynyloxy or NR¹⁰R¹¹,
   or where R¹ and R² together with the two carbon atoms to which they are attached form a fully saturated or partially saturated ring which has 3 to 7 members in total, which is optionally interrupted by one to three heteroatoms from the group consisting of N, O and S and which is optionally substituted further,
R³ represents hydroxy, hydrothio, halogen, NR¹⁰R¹¹, (C₁-C₇)-alkoxy, (C₃-C₁₀)-cycloalkyl-(C₁-C₇)-alkoxy, aryl-(C₁-C₇)-alkoxy, (C₁-C₇)-alkoxy-(C₁-C₇)-alkoxy, arylcarbonyloxy, (C₁-C₇)-alkylcarbonyloxy, aryl-(C₁-C₇)-alkylcarbonyloxy, heteroarylcarbonyloxy, (C₃-C₁₀)-cycloalkylcarbonyloxy, heterocyclylcarbonyloxy, (C₁-C₇)-haloalkylcarbonyloxy, (C₂-C₇)-alkenylcarbonyloxy, OC(O)OR¹², OC(O)SR¹², OC(S)OR¹², OC(S)SR¹², OC(O)NR¹⁰R¹¹, OC(S)NR¹⁰R¹¹, OSO₂R¹³, OSO₂OR¹² or OCHO,
R⁴ represents hydrogen, (C₁-C₇)-alkyl, (C₁-C₇)-haloalkyl, (C₃-C₁₀)-cycloalkyl, (C₃-C₁₀)-cycloalkyl-(C₁-C₇)-alkyl, aryl, heteroaryl, heterocyclyl, aryl-(C₁-C₇)-alkyl, heteroaryl-(C₁-C₇)-alkyl, heterocyclyl-(C₁-C₇)-alkyl, (C₂-C₇)-alkenyl, (C₂-C₇)-alkynyl, (C₂-C₇)-haloalkenyl, (C₂-C₇)-haloalkynyl, (C₄-C₅)-cycloalkenyl, aryl-(C₂-C₇)-alkenyl, heteroaryl-(C₂-C₇)-alkenyl, heterocyclyl-(C₂-C₇)-alkenyl, arylcarbonyl-(C₁-C₇)-alkyl, (C₁-C₇)-alkylcarbonyl-(C₁-C₇)-alkyl, heteroarylcarbonyl-(C₁-C₇)-alkyl, (C₃-C₁₀)-cycloalkylcarbonyl-(C₁-C₇)-alkyl, aryl-(C₁-C₇)-alkoxycarbonyl-(C₁-C₇)-alkyl, (C₁-C₇)-alkoxycarbonyl-(C₁-C₇)-alkyl, (C₁-C₇)-alkoxy-(C₁-C₇)-alkyl, (C₁-C₇)-haloalkoxy-(C₁-C₇)-alkyl, C(O)R¹², SO₂R¹³, CHO, R¹⁰R¹¹N—(C₁-C₈)-alkyl or cyano-(C₁-C₈)-alkyl,
R⁵ represents hydrogen, (C₁-C₇)-alkyl, (C₁-C₇)-haloalkyl, (C₃-C₁₀)-cycloalkyl, (C₃-C₁₀)-cycloalkyl-(C₁-C₇)-alkyl, aryl, heteroaryl, heterocyclyl, aryl-(C₁-C₇)-alkyl, heteroaryl-(C₁-C₇)-alkyl, heterocyclyl-(C₁-C₇)-alkyl, cyano, C(O)OR¹², C(O)NR¹⁰R¹¹, R¹²O(O)C—(C₁-C₇)-alkyl, R¹²O—(C₁-C₇)-alkyl, (C₁-C₇)-alkylcarbonyloxy-(C₁-C₇)-alkyl, (C₃-C₁₀)-cycloalkylcarbonyloxy-(C₁-C₇)-alkyl, arylcarbonyloxy-(C₁-C₇)-alkyl, heteroarylcarbonyloxy-(C₁-C₇)-alkyl or heterocyclylcarbonyloxy-(C₁-C₇)-alkyl,
R⁶ represents hydrogen, (C₁-C₇)-alkyl, (C₁-C₇)-haloalkyl, (C₃-C₁₀)-cycloalkyl, (C₃-C₁₀)-cycloalkyl-(C₁-C₇)-alkyl, aryl, heteroaryl, heterocyclyl, aryl-(C₁-C₇)-alkyl, heteroaryl-(C₁-C₇)-alkyl or heterocyclyl-(C₁-C₇)-alkyl,
R⁷ and R⁸ are identical or different and independently of one another represent hydrogen, halogen or (C₁-C₇)-alkyl,
R⁹ represents hydrogen, hydroxy, (C₁-C₇)-alkyl, (C₁-C₇)-haloalkyl, (C₁-C₇)-alkoxy, (C₁-C₇)-alkoxy-(C₁-C₇)-alkyl, (C₃-C₁₀)-cycloalkyl, (C₂-C₇)-alkenyl, (C₂-C₇)-alkynyl or (C₁-C₇)-cyanoalkyl,
   or where R² and R⁹ together with the nitrogen atom or carbon atom to which they are respectively attached form a fully saturated or partially saturated ring which has 3 to 7 members in total, which is optionally interrupted by one to three heteroatoms from the group consisting of N, O and S and which is optionally substituted further,
R¹⁰ and R¹¹ are identical or different and independently of one another represent hydrogen, (C₁-C₇)-alkyl, (C₂-C₇)-alkenyl, (C₂-C₇)-alkynyl, (C₁-C₇)-cyanoalkyl, (C₁-C₈)-haloalkyl, (C₂-C₇)-haloalkenyl, (C₂-C₇)-haloalkynyl, (C₃-C₁₀)-cycloalkyl, (C₄-C₈)-cycloalkenyl, (C₁-C₇)-alkoxy-(C₁-C₇)-alkyl, (C₁-C₇)-haloalkoxy-(C₁-C₇)-alkyl, (C₁-C₇)-alkylthio-(C₁-C₇)-alkyl, (C₁-C₇)-haloalkylthio-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, (C₃-C₁₀)-cycloalkyl-(C₁-C₇)-alkyl, (C₄-C₅)-cycloalkenyl-(C₁-C₇)-alkyl, C(O)R¹², SO₂R¹³, heterocyclyl, (C₁-C₇)-alkoxycarbonyl-(C₁-C₇)-alkyl, (C₂-C₇)-alkoxycarbonyl, aryl-(C₁-C₇)-alkoxycarbonyl-(C₁-C₇)-alkyl, aryl-(C₁-C₇)-alkoxycarbonyl, heteroaryl-(C₁-C₇)-alkoxycarbonyl, (C₂-C₇)-alkenyloxycarbonyl, (C₂-C₇)-alkynyloxycarbonyl or heterocyclyl-(C₁-C₇)-alkyl,
R¹² represents (C₁-C₇)-alkyl, (C₂-C₇)-alkenyl, (C₂-C₇)-alkynyl, (C₁-C₇)-cyanoalkyl, (C₁-C₈)-haloalkyl, (C₂-C₇)-haloalkenyl, (C₂-C₇)-haloalkynyl, (C₃-C₁₀)-cycloalkyl, (C₄-C₅)-cycloalkenyl, (C₁-C₇)-alkoxy-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, (C₃-C₁₀)-cycloalkyl-(C₁-C₇)-alkyl, (C₄-C₅)-cycloalkenyl-(C₁-C₇)-alkyl, (C₁-C₇)-alkoxycarbonyl-(C₁-C₇)-alkyl, (C₂-C₇)-alkenyloxycarbonyl-(C₁-C₇)-alkyl, aryl-(C₁-C₇)-alkoxycarbonyl-(C₁-C₇)-alkyl, hydroxycarbonyl-(C₁-C₇)-alkyl, heterocyclyl or heterocyclyl-(C₁-C₇)-alkyl,
R¹³ represents (C₁-C₇)-alkyl, (C₂-C₇)-alkenyl, (C₂-C₇)-alkynyl, (C₁-C₇)-cyanoalkyl, (C₁-C₈)-haloalkyl, (C₂-C₇)-haloalkenyl, (C₂-C₇)-haloalkynyl, (C₃-C₁₀)-cycloalkyl, (C₄-C₅)-cycloalkenyl, (C₁-C₇)-alkoxy-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, heterocyclyl-(C₁-C₇)-alkyl, (C₃-C₁₀)-cycloalkyl-(C₁-C₇)-alkyl, (C₄-C₈)-cycloalkenyl-(C₁-C₇)-alkyl or NR¹⁰R¹¹,
R¹⁴ represents hydrogen or (C₁-C₄)-alkyl,
   or where R³ and R¹⁴ together with the carbon atom to which they are attached form a carbonyl group,

where the cyclic structural elements (in particular the structural elements aryl, cycloalkyl, cycloalkenyl, heteroaryl and heterocyclyl) of the radicals mentioned respectively in R¹, R², R³, R⁴, R, R⁶, R⁹, R¹⁰, R¹¹, R¹² and R¹³ and the rings formed by R¹ and R² or R² and R⁹, having 3 to 7 members in total, are in each case unsubstituted or substituted by one or more radicals selected from the group consisting of halogen, nitro, hydroxy, cyano, NR¹⁰R¹¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxy-carbonyl, (C₁-C₄)-haloalkoxy-carbonyl, (C₁-C₄)-alkylcarboxy, (C₃-C₆)-cycloalkyl, (C₃-C₆)-cycloalkyl-(C₁-C₆)-alkyl, (C₁-C₄)-alkoxy-carbonyl-(C₁-C₄)-alkyl, hydroxycarbonyl, hydroxycarbonyl-(C₁-C₄)-alkyl, R¹⁰R¹¹N-carbonyl, and where the structural elements cycloalkyl, cycloalkenyl and heterocyclyl have n oxo groups, where n=0, 1 or 2.

More preferred compounds of the general formula (I) according to the invention are those in which
X represents oxygen,
A represents the grouping C—R¹ or the grouping N—R⁹ (N=nitrogen), where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents hydrogen, halogen, hydroxy, (C₁-C₆)-alkyl, (C₁-C₆)-haloalkyl, (C₁-C₆)-hydroxyalkyl, (C₁-C₆)-alkoxy, (C₁-C₆)-alkoxyalkyl, (C₁-C₆)-alkoxy-(C₁-C₆)-alkyloxy, (C₁-C₆)-haloalkoxy, (C₃-C₁₀)-cycloalkyl, (C₂-C₆)-alkenyl, (C₂-C₆)-alkenyloxy, (C₂-C₆)-alkynyl or (C₂-C₆)-alkynyloxy,
R² represents hydrogen, halogen, hydroxy, (C₁-C₆)-alkyl, (C₁-C₆)-haloalkyl, (C₁-C₆)-hydroxyalkyl, (C₁-C₆)-alkoxy, (C₁-C₆)-alkoxyalkyl, (C₁-C₆)-alkoxy-(C₁-C₆)-alkyloxy, (C₁-C₆)-haloalkoxy, (C₃-C₁₀)-cycloalkyl, (C₂-C₆)-alkenyl, (C₂-C₆)-alkenyloxy, (C₂-C₆)-alkynyl, (C₂-C₆)-alkynyloxy or NR¹⁰R¹¹,
   or where R¹ and R² together with the two carbon atoms to which they are attached form a fully saturated or partially saturated ring which has 3 to 7 members in total, which is optionally interrupted by one to three heteroatoms from the group consisting of N, O and S and which is optionally substituted further,
R³ represents hydroxy, hydrothio, halogen, (C₁-C₆)-alkoxy, (C₃-C₁₀)-cycloalkyl-(C₁-C₆)-alkoxy, aryl-(C₁-C₆)-alkoxy, (C₁-C₆)-alkoxy-(C₁-C₆)-alkoxy, arylcarbonyloxy, (C₁-C₆)-alkylcarbonyloxy, aryl-(C₁-C₆)-alkylcarbonyloxy, heteroarylcarbonyloxy, (C₃-C₁₀)-cycloalkylcarbonyloxy, heterocyclylcarbonyloxy, (C₁-C₆)-haloalkyl-carbonyloxy, (C₂-C₆)-alkenylcarbonyloxy, OC(O)OR¹² or OSO₂R¹³,
R⁴ represents hydrogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₆)-alkyl, heteroaryl-(C₁-C₆)-alkyl, heterocyclyl-(C₁-C₆)-alkyl, (C₂-C₆)-alkenyl, (C₂-C₆)-alkynyl, (C₂-C₆)-haloalkenyl, (C₂-C₆)-haloalkynyl, (C₄-C₈)-cycloalkenyl, aryl-(C₂-C₆)-alkenyl, heteroaryl-(C₂-C₆)-alkenyl, heterocyclyl-(C₂-C₆)-alkenyl, arylcarbonyl-(C₁-C₆)-alkyl, (C₁-C₆)-alkylcarbonyl-(C₁-C₆)-alkyl, heteroarylcarbonyl-(C₁-C₆)-alkyl, (C₃-C₁₀)-cycloalkylcarbonyl-(C₁-C₆)-alkyl, aryl-(C₁-C₆)-alkoxycarbonyl-(C₁-C₆)-alkyl, (C₁-C₆)-alkoxycarbonyl-(C₁-C₆)-alkyl, (C₁-C₆)-alkoxy-(C₁-C₆)-alkyl, (C₁-C₆)-haloalkoxy-(C₁-C₆)-alkyl, C(O)R¹², SO₂R¹³, CHO, R¹⁰R¹¹N—(C₁-C₈)-alkyl or cyano-(C₁-C₈)-alkyl,
R⁵ represents hydrogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₆)-alkyl, heteroaryl-(C₁-C₆)-alkyl, heterocyclyl-(C₁-C₆)-alkyl, cyano, C(O)OR¹², C(O)NR¹⁰R¹¹, R¹²O(O)C—(C₁-C₆)-alkyl, R¹²O—(C₁-C₆)-alkyl, (C₁-C₆)-alkylcarbonyloxy-(C₁-C₆)-alkyl, (C₃-C₁₀)-cycloalkylcarbonyloxy-(C₁-C₆)-alkyl, arylcarbonyloxy-(C₁-C₆)-alkyl, heteroarylcarbonyloxy-(C₁-C₆)-alkyl or heterocyclylcarbonyloxy-(C₁-C₆)-alkyl,
R⁶ represents hydrogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₆)-alkyl, heteroaryl-(C₁-C₆)-alkyl or heterocyclyl-(C₁-C₆)-alkyl,
R⁷ and R⁸ are identical or different and independently of one another represent hydrogen, halogen or (C₁-C₆)-alkyl,
R⁹ represents hydrogen, hydroxy, (C₁-C₆)-alkyl, (C₁-C₆)-haloalkyl, (C₁-C₆)-alkoxy, (C₁-C₆)-alkoxy-(C₁-C₆)-alkyl, (C₃-C₁₀)-cycloalkyl, (C₂-C₆)-alkenyl, (C₂-C₆)-alkynyl or (C₁-C₆)-cyanoalkyl,
   or where R² and R⁹ together with the nitrogen atom or carbon atom to which they are respectively attached form a fully saturated or partially saturated ring which has 3 to 7 members in total, which is optionally interrupted by one to three heteroatoms from the group consisting of N, O and S and which is optionally substituted further,
R¹⁰ and R¹¹ are identical or different and independently of one another represent hydrogen, (C₁-C₆)-alkyl, (C₂-C₆)-alkenyl, (C₂-C₆)-alkynyl, (C₁-C₆)-cyanoalkyl, (C₁-C₇)-haloalkyl, (C₂-C₆)-haloalkenyl, (C₂-C₆)-haloalkynyl, (C₃-C₁₀)-cycloalkyl, (C₄-C₇)-cycloalkenyl, (C₁-C₆)-alkoxy-(C₁-C₆)-alkyl, (C₁-C₆)-haloalkoxy-(C₁-C₆)-alkyl, (C₁-C₆)-alkylthio-(C₁-C₆)-alkyl, (C₁-C₆)-haloalkylthio-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, (C₃-C₁₀)-cycloalkyl-(C₁-C₆)-alkyl, (C₄-C₇)-cycloalkenyl-(C₁-C₆)-alkyl, C(O)R¹², SO₂R¹³, heterocyclyl, (C₁-C₆)-alkoxycarbonyl-(C₁-C₆)-alkyl, (C₁-C₆)-alkoxycarbonyl, aryl-(C₁-C₆)-alkoxycarbonyl-(C₁-C₆)-alkyl, aryl-(C₁-C₆)-alkoxycarbonyl, heteroaryl-(C₁-C₆)-alkoxycarbonyl, (C₂-C₆)-alkenyloxycarbonyl, (C₂-C₆)-alkynyloxycarbonyl or heterocyclyl-(C₁-C₆)-alkyl,
R¹² represents (C₁-C₆)-alkyl, (C₂-C₆)-alkenyl, (C₂-C₆)-alkynyl, (C₁-C₆)-cyanoalkyl, (C₁-C₇)-haloalkyl, (C₂-C₆)-haloalkenyl, (C₂-C₆)-haloalkynyl, (C₃-C₁₀)-cycloalkyl, (C₄-C₇)-cycloalkenyl, (C₁-C₆)-alkoxy-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, (C₃-C₁₀)-cycloalkyl-(C₁-C₆)-alkyl, (C₄-C₇)-cycloalkenyl-(C₁-C₆)-alkyl, (C₁-C₆)-alkoxycarbonyl-(C₁-C₆)-alkyl, (C₂-C₆)-alkenyloxycarbonyl-(C₁-C₆)-alkyl, aryl-(C₁-C₆)-alkoxycarbonyl-(C₁-C₆)-alkyl, hydroxycarbonyl-(C₁-C₆)-alkyl, heterocyclyl or heterocyclyl-(C₁-C₆)-alkyl,
R¹³ represents (C₁-C₆)-alkyl, (C₂-C₆)-alkenyl, (C₂-C₆)-alkynyl, (C₁-C₆)-cyanoalkyl, (C₁-C₇)-haloalkyl, (C₂-C₆)-haloalkenyl, (C₂-C₆)-haloalkynyl, (C₃-C₁₀)-cycloalkyl, (C₄-C₇)-cycloalkenyl, (C₁-C₆)-alkoxy-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, heterocyclyl-(C₁-C₆)-alkyl, (C₃-C₁₀)-cycloalkyl-(C₁-C₆)-alkyl, (C₄-C₇)-cycloalkenyl-(C₁-C₆)-alkyl or NR¹⁰R¹¹,
R¹⁴ represents hydrogen,
   or where R³ and R¹⁴ together with the carbon atom to which they are attached form a carbonyl group,

where the cyclic structural elements (in particular the structural elements aryl, cycloalkyl, cycloalkenyl, heteroaryl and heterocyclyl) of the radicals mentioned respectively in R¹, R², R³, R⁴, R⁵, R⁶, R⁹, R¹⁰, R¹¹, R¹² and R¹³ and the rings formed by R¹ and R² or R² and R⁹, having 3 to 7 members in total, are in each case unsubstituted or substituted by one or more radicals selected from the group consisting of halogen, nitro, hydroxy, cyano, NR¹⁰R¹¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxy-carbonyl, (C₁-C₄)-haloalkoxy-carbonyl, (C₁-C₄)-alkylcarboxy, (C₃-C₆)-cycloalkyl, (C₃-C₆)-cycloalkyl-(C₁-C₆)-alkyl, (C₁-C₄)-alkoxy-carbonyl-(C₁—C₄)-alkyl, hydroxycarbonyl, hydroxycarbonyl-(C₁-C₄)-alkyl, R¹⁰R¹¹N-carbonyl, and where the structural elements cycloalkyl, cycloalkenyl and heterocyclyl have n oxo groups, where n=0, 1 or 2.

The invention more preferably provides compounds of the general formula (I) in which
X represents oxygen,
A represents the grouping C—R¹ or the grouping N—R⁹ (N=nitrogen), where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents hydrogen, fluorine, chlorine, bromine, iodine, hydroxy, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 3-methylcyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, trifluoromethyl, pentafluoroethyl, 1,1,2,2-tetrafluoroethyl, heptafluoropropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, difluoro-tert-butyl, chloromethyl, bromomethyl, hydroxymethyl, hydroxyethyl, hydroxy-n-propyl, methoxy, ethoxy, n-propyloxy, isopropyloxy, n-butyloxy, tert-butyloxy, methoxymethyl, ethoxymethyl, n-propyloxymethyl, isopropyloxymethyl, methoxyethyl, ethoxyethyl, n-propyloxyethyl, isopropyloxyethyl, methoxymethoxy, methoxyethoxy, methoxy-n-propyloxy, methoxy-n-butyloxy, ethoxymethoxy, ethoxyethoxy, ethoxy-n-propyloxy, ethoxy-n-butyloxy, n-propyloxymethoxy, isopropyloxymethoxy, trifluoromethoxy, difluoromethoxy, pentafluoroethoxy, 2,2,1,1-tetrafluoroethoxy, 2,2,2-trifluoroethoxy, 2,2-difluoroethoxy, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl, prop-2-en-1-yloxy, but-3-en-1-yloxy, pent-4-en-1-yloxy, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, prop-2-yn-1-yloxy, but-3-yn-1-yloxy or but-2-yn-1-yloxy,
R² represents hydrogen, fluorine, chlorine, bromine, iodine, hydroxy, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 3-methylcyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, trifluoromethyl, pentafluoroethyl, 1,1,2,2-tetrafluoroethyl, heptafluoropropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, difluoro-tert-butyl, chloromethyl, bromomethyl, hydroxymethyl, hydroxyethyl, hydroxy-n-propyl, methoxy, ethoxy, n-propyloxy, isopropyloxy, n-butyloxy, tert-butyloxy, methoxymethyl, ethoxymethyl, n-propyloxymethyl, isopropyloxymethyl, n-butyloxymethyl, methoxyethyl, ethoxyethyl, n-propyloxyethyl, isopropyloxyethyl, methoxy-n-propyl, methoxy-n-butyl, methoxymethoxy, methoxyethoxy, methoxy-n-propyloxy, methoxy-n-butyloxy, ethoxymethoxy, ethoxyethoxy, ethoxy-n-propyloxy, ethoxy-n-butyloxy, n-propyloxymethoxy, isopropyloxymethoxy, trifluoromethoxy, difluoromethoxy, pentafluoroethoxy, 2,2,1,1-tetrafluoroethoxy, 2,2,2-trifluoroethoxy, 2,2-difluoroethoxy, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl, prop-2-en-1-yloxy, but-3-en-1-yloxy, pent-4-en-1-yloxy, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, prop-2-yn-1-yloxy, but-3-yn-1-yloxy, but-2-yn-1-yloxy or NR¹⁰R¹¹,
   or where R¹ and R² together with the two carbon atoms to which they are attached form a fully saturated or partially saturated ring which has 3 to 7 members in total, which is optionally interrupted by one to three heteroatoms from the group consisting of N, O and S and which is optionally substituted further,
R³ represents hydroxy, hydrothio, fluorine, chlorine, bromine, iodine, methoxy, ethoxy, n-propyloxy, 1-methylethoxy, n-butyloxy, 1-methylpropyloxy, 2-methylpropyloxy, 1,1-dimethylethoxy, n-pentyloxy, 1-methylbutyloxy, 2-methylbutyloxy, 3-methylbutyloxy, 1,1-dimethylpropyloxy, 1,2-dimethylpropyloxy, 2,2-dimethylpropyloxy, 1-ethylpropyloxy, n-hexyloxy, 1-methylpentyloxy, 2-methylpentyloxy, 3-methylpentyloxy, 4-methylpentyloxy, 1,1-dimethylbutyloxy, 1,2-dimethylbutyloxy, 1,3-dimethylbutyloxy, 2,2-dimethylbutyloxy, 2,3-dimethylbutyloxy, 3,3-dimethylbutyloxy, 1-ethylbutyloxy, 2-ethylbutyloxy, 1,1,2-trimethylpropyloxy, 1,2,2-trimethylpropyloxy, 1-ethyl-1-methylpropyloxy, 1-ethyl-2-methylpropyloxy, cyclopropylmethoxy, cyclobutylmethoxy, cyclopentylmethoxy, cyclohexylmethoxy, aryl-(C₁-C₆)-alkoxy, methoxymethoxy, methoxyethoxy, methoxy-n-propyloxy, methoxy-n-butyloxy, ethoxymethoxy, ethoxyethoxy, ethoxy-n-propyloxy, ethoxy-n-butyloxy, n-propyloxymethoxy, isopropyloxymethoxy, arylcarbonyloxy, (C₁-C₆)-alkylcarbonyloxy, aryl-(C₁-C₆)-alkylcarbonyloxy, heteroarylcarbonyloxy, (C₃-C₁₀)-cycloalkylcarbonyloxy, heterocyclylcarbonyloxy, (C₁-C₆)-haloalkylcarbonyloxy, (C₂-C₆)-alkenylcarbonyloxy, OC(O)OR¹² or OSO₂R¹³,
R⁴ represents hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 3-methylcyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, cyclopropylmethyl, cyclobutylmethyl, cyclopentylmethyl, cyclohexylmethyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, 3,3,3-trifluoroprop-1-yl, 3,3,3-trifluoroprop-2-yl, difluoro-tert-butyl, optionally substituted phenyl, optionally substituted heteroaryl, optionally substituted heterocyclyl, aryl-(C₁-C₆)-alkyl, heteroaryl-(C₁-C₆)-alkyl, heterocyclyl-(C₁-C₆)-alkyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, (C₂-C₆)-haloalkenyl, (C₂-C₆)-haloalkynyl, (C₃-C₅)-halocycloalkyl, (C₄-C₅)-cycloalkenyl, (C₄-C₅)-halocycloalkenyl, aryl-(C₂-C₆)-alkenyl, heteroaryl-(C₂-C₆)-alkenyl, heterocyclyl-(C₂-C₆)-alkenyl, arylcarbonyl-(C₁-C₆)-alkyl, (C₁-C₆)-alkylcarbonyl-(C₁-C₆)-alkyl, heteroarylcarbonyl-(C₁-C₆)-alkyl, (C₃-C₁₀)-cycloalkylcarbonyl-(C₁-C₆)-alkyl, aryl-(C₁-C₆)-alkoxycarbonyl-(C₁-C₆)-alkyl, (C₁-C₆)-alkoxycarbonyl-(C₁-C₆)-alkyl, methoxymethyl, ethoxymethyl, n-propyloxymethyl, isopropyloxymethyl, n-butyloxymethyl, methoxyethyl, ethoxyethyl, n-propyloxyethyl, isopropyloxyethyl, methoxy-n-propyl, methoxy-n-butyl, trifluoromethoxymethyl, trifluoromethoxyethyl, trifluoromethoxy-n-propyl, difluoromethoxymethyl, difluoromethoxyethyl, difluoromethoxy-n-propyl, 2,2-difluoroethoxymethyl, 2,2-difluoroethoxyethyl, 2,2-difluoroethoxy-n-propyl, 2,2,2-trifluoroethoxymethyl, 2,2,2-trifluoroethoxyethyl, 2,2,2-trifluoroethoxy-n-propyl, pentafluoroethoxymethyl, pentafluoroethoxyethyl, pentafluoroethoxy-n-propyl, C(O)R¹², SO₂R¹³, CHO, cyanomethyl, cyanoethyl or cyano-n-propyl,
R⁵ represents hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, pentafluoroethyl, 1,1,2,2-tetrafluoroethyl, heptafluoro-n-propyl, Heptafluoroisopropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, 3,3,3-trifluoroprop-1-yl, 3,3,3-trifluoroprop-2-yl, difluoro-tert-butyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 3-methylcyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, cyclopropylmethyl, cyclobutylmethyl, cyclopentylmethyl, cyclohexylmethyl, optionally substituted phenyl, optionally substituted heteroaryl, optionally substituted heterocyclyl, aryl-(C₁-C₆)-alkyl, heteroaryl-(C₁-C₆)-alkyl, heterocyclyl-(C₁-C₆)-alkyl, cyano, C(O)OR¹², C(O)NR¹⁰R¹¹, R¹²O(O)C—(C₁-C₆)-alkyl, R¹²O—(C₁-C₆)-alkyl, methylcarbonyloxymethyl, ethylcarbonyloxymethyl, n-propylcarbonyloxymethyl, 1-methylethylcarbonyloxymethyl or 1,1-dimethylethylcarbonyloxymethyl,
R⁶ represents hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, 3,3,3-trifluoroprop-1-yl, 3,3,3-trifluoroprop-2-yl, difluoro-tert-butyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 3-methylcyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, cyclopropylmethyl, cyclobutylmethyl, cyclopentylmethyl, cyclohexylmethyl, optionally substituted phenyl, optionally substituted heteroaryl, optionally substituted heterocyclyl, aryl-(C₁-C₆)-alkyl, heteroaryl-(C₁-C₆)-alkyl or heterocyclyl-(C₁-C₆)-alkyl,
R⁷ and R⁸ are identical or different and independently of one another represent hydrogen, fluorine, chlorine, bromine, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl,
R⁹ represents hydrogen, hydroxy, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, 2,2-difluoroethyl, 2,2,2-trifluoroethyl, 3,3,3-trifluoroprop-1-yl, 3,3,3-trifluoroprop-2-yl, difluoromethyl, methoxy, ethoxy, n-propyloxy, n-butyloxy, cyanomethyl, cyanoethyl, cyano-n-propyl, cyclopropyl, cyclobutyl, cyclopentyl, cyclohexyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl, prop-2-yn-1-yl, but-2-yn-1-yl, but-3-yn-1-yl, pent-2-yn-1-yl, pent-3-yn-1-yl, pent-4-yn-1yl, methoxymethyl, methoxyethyl, ethoxymethyl, methoxy-n-propyl, n-propyloxymethyl or ethoxyethyl,
   or where R² and R⁹ together with the nitrogen atom or carbon atom to which they are respectively attached form a fully saturated or partially saturated ring which has 3 to 7 members in total, which is optionally interrupted by one to three heteroatoms from the group consisting of N, O and S and which is optionally substituted further,
R¹⁰ and R¹¹ are identical or different and independently of one another represent hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, (C₁-C₆)-haloalkyl, (C₂-C₆)-haloalkenyl, (C₂-C₆)-haloalkynyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 3-methylcyclobutyl, 1-cyanocyclobutyl, 2-cyanocyclobutyl, 3-cyanocyclobutyl, 3-methoxycyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, 4-methoxycyclohexyl, cyclopropylmethyl, cyclobutylmethyl, cyclopentylmethyl, cyclohexylmethyl, (C₃-C₆)-halocycloalkyl, (C₄-C₆)-cycloalkenyl, (C₄-C₆)-halocycloalkenyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, trifluoromethoxymethyl, trifluoromethoxyethyl, trifluoromethoxy-n-propyl, difluoromethoxymethyl, difluoromethoxyethyl, difluoromethoxy-n-propyl, 2,2-difluoroethoxymethyl, 2,2-difluoroethoxyethyl, 2,2-difluoroethoxy-n-propyl, 2,2,2-trifluoroethoxymethyl, 2,2,2-trifluoroethoxyethyl, 2,2,2-trifluoroethoxy-n-propyl, pentafluoroethoxymethyl, pentafluoroethoxyethyl, pentafluoroethoxy-n-propyl, methylthiomethyl, methylthioethyl, ethylthioethyl, methylthio-n-propyl, ethylthio-n-propyl, trifluoromethylthiomethyl, trifluoromethylthioethyl, trifluoromethylthio-n-propyl, optionally substituted phenyl, aryl-(C₁-C₅)-alkyl, optionally substituted heteroaryl, heteroaryl-(C₁-C₅)-alkyl, (C₄-C₆)-cycloalkenyl-(C₁-C₅)-alkyl, C(O)R¹², SO₂R¹³, optionally substituted heterocyclyl, (C₁-C₅)-alkoxycarbonyl-(C₁-C₅)-alkyl, (C₁-C₅)-alkoxycarbonyl, aryl-(C₁-C₅)-alkoxycarbonyl-(C₁-C₅)-alkyl, aryl-(C₁-C₅)-alkoxycarbonyl, heteroaryl-(C₁-C₅)-alkoxycarbonyl, (C₂-C₆)-alkenyloxycarbonyl, (C₂-C₆)-alkynyloxycarbonyl or heterocyclyl-(C₁-C₅)-alkyl,
R¹² represents methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 2-cyanocyclopropyl, 1-methylcyclobutyl, 2-methylcyclobutyl, 3-methylcyclobutyl, 2-cyanocyclobutyl, 3-cyanocyclobutyl, 3-methoxycyclobutyl, 1-allylcyclopropyl, 1-ethylcyclopropyl, 1-methylcyclohexyl, 2-methylcyclohexyl, 3-methylcyclohexyl, 2-methoxycyclohexyl, 3-methoxycyclohexyl, 4-methoxycyclohexyl, cyclopropylmethyl, cyclobutylmethyl, cyclopentylmethyl, cyclohexylmethyl, trifluoromethyl, pentafluoroethyl, 1,1,2,2-tetrafluoroethyl, heptafluoropropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, 3,3-difluoro-n-propyl, 3,3,3-trifluoro-n-propyl, 4,4-difluoro-n-butyl, 4,4,4-trifluoro-n-butyl, (C₂-C₆)-haloalkenyl, (C₂-C₆)-haloalkynyl, (C₃-C₆)-halocycloalkyl, (C₄-C₆)-cycloalkenyl, (C₄-C₆)-halocycloalkenyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, optionally substituted phenyl, aryl-(C₁-C₆)-alkyl, optionally substituted heteroaryl, heteroaryl-(C₁-C₆)-alkyl, (C₄-C₆)-cycloalkenyl-(C₁-C₆)-alkyl, (C₁-C₆)-alkoxycarbonyl-(C₁-C₆)-alkyl, (C₂-C₆)-alkenyloxycarbonyl-(C₁-C₆)-alkyl, aryl-(C₁-C₆)-alkoxycarbonyl-(C₁-C₆)-alkyl, hydroxycarbonyl-(C₁-C₆)-alkyl, optionally substituted heterocyclyl or heterocyclyl-(C₁-C₆)-alkyl,
R¹³ represents methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 2-cyanocyclopropyl, 1-methylcyclobutyl, 2-methylcyclobutyl, 3-methylcyclobutyl, 2-cyanocyclobutyl, 3-cyanocyclobutyl, 3-methoxycyclobutyl, 1-allylcyclopropyl, 1-ethylcyclopropyl, 1-methylcyclohexyl, 2-methylcyclohexyl, 3-methylcyclohexyl, 2-methoxycyclohexyl, 3-methoxycyclohexyl, 4-methoxycyclohexyl, cyclopropylmethyl, cyclobutylmethyl, cyclopentylmethyl, cyclohexylmethyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, 3,3,3-trifluoro-n-propyl, difluoro-tert-butyl, (C₂-C₆)-haloalkenyl, (C₂-C₆)-haloalkynyl, (C₃-C₆)-halocycloalkyl, (C₄-C₆)-cycloalkenyl, (C₄-C₆)-halocycloalkenyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, optionally substituted phenyl, aryl-(C₁-C₆)-alkyl, optionally substituted heteroaryl, heteroaryl-(C₁-C₆)-alkyl, heterocyclyl-(C₁-C₆)-alkyl, (C₄-C₆)-cycloalkenyl-(C₁-C₆)-alkyl or NR¹⁰R¹¹,
R¹⁴ represents hydrogen,
   or R³ and R¹⁴ with the atom to which they are attached form a carbonyl group.

The invention furthermore preferably provides compounds of the general formula (I) in which
X represents oxygen,
A represents the grouping C—R¹ or the grouping N—R⁹ (N=nitrogen) where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents hydrogen, chlorine, bromine, iodine, methyl, ethyl, n-propyl, 1-methylethyl, 1,1-dimethylethyl, trifluoromethyl, pentafluoroethyl, difluoromethyl, chloromethyl, methoxy, ethoxy, trifluoromethoxy, difluoromethoxy, 2,2,2-trifluoroethoxy, 2,2-difluoroethoxy, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, ethynyl, 1-propynyl, 2-propynyl, 1-butynyl, 2-butynyl, 3-butynyl, prop-2-yn-1-yloxy, but-3-yn-1-yloxy, but-2-yn-1-yloxy, prop-2-en-1-yloxy, but-3-en-1-yloxy, pent-4-en-1-yloxy, cyclopropyl, cyclobutyl, cyclopentyl or cyclohexyl,
R² represents hydrogen, chlorine, bromine, iodine, methyl, ethyl, n-propyl, 1-methylethyl, 1,1-dimethylethyl, trifluoromethyl, pentafluoroethyl, difluoromethyl, chloromethyl, methoxy, ethoxy, trifluoromethoxy, difluoromethoxy, 2,2,2-trifluoroethoxy, 2,2-difluoroethoxy, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, ethynyl, 1-propynyl, 2-propynyl, 1-butynyl, 2-butynyl, 3-butynyl, prop-2-yn-1-yloxy, but-3-yn-1-yloxy, but-2-yn-1-yloxy, prop-2-en-1-yloxy, but-3-en-1-yloxy, pent-4-en-1-yloxy, cyclopropyl, cyclobutyl, cyclopentyl, cyclohexyl, dimethylamino, methylamino, amino, ethoxyethylamino, methoxyethylamino, cyclopropylamino, cyclobutylamino, 2,2-dimethylprop-1-ylamino, prop-2-yn-1-ylamino, prop-2-en-1-ylamino, cyclopropylmethylamino or 2-methylprop-2-en-1-ylamino,
   or where R¹ and R² together with the two carbon atoms to which they are attached form a fully saturated or partially saturated ring which has 3 to 7 members in total, which is optionally interrupted by one to three heteroatoms from the group consisting of N, O and S and which is optionally substituted further,
R³ represents hydroxy, hydrothio, chlorine, bromine, methylcarbonyloxy, ethylcarbonyloxy, n-propylcarbonyloxy, 1-methylethylcarbonyloxy, n-butylcarbonyloxy, 1-methylpropylcarbonyloxy, 2-methylpropylcarbonyloxy, 1,1-dimethylethylcarbonyloxy, n-pentylcarbonyloxy, 1-methylbutylcarbonyloxy, 2-methylbutylcarbonyloxy, 3-methylbutylcarbonyloxy, 1,1-dimethylpropylcarbonyloxy, 1,2-dimethylpropylcarbonyloxy, 2,2-dimethylpropylcarbonyloxy, 1-ethylpropylcarbonyloxy, n-hexylcarbonyloxy, 1-methylpentylcarbonyloxy, 2-methylpentylcarbonyloxy, 3-methylpentylcarbonyloxy, 4-methylpentylcarbonyloxy, 1,1-dimethylbutylcarbonyloxy, 1,2-dimethylbutylcarbonyloxy, 1,3-dimethylbutylcarbonyloxy, 2,2-dimethylbutylcarbonyloxy, 2,3-dimethylbutylcarbonyloxy, 3,3-dimethylbutylcarbonyloxy, 1-ethylbutylcarbonyloxy, 2-ethylbutylcarbonyloxy, 1,1,2-trimethylpropylcarbonyloxy, 1,2,2-trimethylpropylcarbonyloxy, 1-ethyl-1-methylpropylcarbonyloxy, 1-ethyl-2-methylpropylcarbonyloxy, methoxy, ethoxy, n-propyloxy, n-butyloxy, benzyloxy, cyclopropylmethoxy, cyclobutylmethoxy, cyclopentylmethoxy, cyclohexylmethoxy, methoxymethoxy, methoxyethoxy, methoxy-n-propyloxy, methoxy-n-butyloxy, ethoxymethoxy, ethoxyethoxy, phenylcarbonyloxy, benzylcarbonyloxy, heteroarylcarbonyloxy, cyclopropylcarbonyloxy, cyclobutylcarbonyloxy, cyclopentylcarbonyloxy, cyclohexylcarbonyloxy, heterocyclylcarbonyloxy, trifluoromethylcarbonyloxy, difluoromethylcarbonyloxy, methoxycarbonyloxy, ethoxycarbonyloxy, n-propyloxycarbonyloxy, n-butyloxycarbonyloxy, 1,1-dimethylethyloxycarbonyloxy, 2,2-dimethylpropyloxycarbonyloxy, methylsulfonyloxy, ethylsulfonyloxy, n-propylsulfonyloxy, 1-methylethylsulfonyloxy or cyclopropylsulfonyloxy,
R⁴ represents hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, cyclopropyl, cyclobutyl, cyclopentyl, cyclohexyl, spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl, bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, adamantan-2-yl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 3-methylcyclobutyl, 1-cyanocyclobutyl, 2-cyanocyclobutyl, 3-cyanocyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, cyclopropylmethyl, cyclobutylmethyl, cyclopentylmethyl, cyclohexylmethyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, 3,3,3-trifluoroprop-1-yl, 3,3,3-trifluoroprop-2-yl, difluoro-tert-butyl, optionally substituted phenyl, optionally substituted heteroaryl, optionally substituted heterocyclyl, aryl-(C₁-C₆)-alkyl, heteroaryl-(C₁-C₆)-alkyl, heterocyclyl-(C₁-C₆)-alkyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, (C₂-C₆)-haloalkenyl, (C₂-C₆)-haloalkynyl, (C₃-C₅)-halocycloalkyl, (C₄-C₅)-cycloalkenyl, (C₄-C₅)-halocycloalkenyl, aryl-(C₂-C₆)-alkenyl, heteroaryl-(C₂-C₆)-alkenyl, heterocyclyl-(C₂-C₆)-alkenyl, arylcarbonyl-(C₁-C₆)-alkyl, (C₁-C₆)-alkylcarbonyl-(C₁-C₆)-alkyl, heteroarylcarbonyl-(C₁-C₆)-alkyl, (C₃-C₁₀)-cycloalkylcarbonyl-(C₁-C₆)-alkyl, aryl-(C₁-C₆)-alkoxycarbonyl-(C₁-C₆)-alkyl, (C₁-C₆)-alkoxycarbonyl-(C₁-C₆)-alkyl, methoxymethyl, ethoxymethyl, n-propyloxymethyl, isopropyloxymethyl, n-butyloxymethyl, methoxyethyl, ethoxyethyl, n-propyloxyethyl, isopropyloxyethyl, methoxy-n-propyl, methoxy-n-butyl, trifluoromethoxymethyl, trifluoromethoxyethyl, trifluoromethoxy-n-propyl, difluoromethoxymethyl, difluoromethoxyethyl, difluoromethoxy-n-propyl, 2,2-difluoroethoxymethyl, 2,2-difluoroethoxyethyl, 2,2-difluoroethoxy-n-propyl, 2,2,2-trifluoroethoxymethyl, 2,2,2-trifluoroethoxyethyl, 2,2,2-trifluoroethoxy-n-propyl, pentafluoroethoxymethyl, pentafluoroethoxyethyl, pentafluoroethoxy-n-propyl, C(O)R¹², SO₂R¹³, CHO, cyanomethyl, cyanoethyl or cyano-n-propyl,
R⁵ represents hydrogen, methyl, ethyl, n-propyl, 1-methylethyl, n-butyl, 1-methylpropyl, 2-methylpropyl, 1,1-dimethylethyl, n-pentyl, cyclopropyl, cyclobutyl, cyclopentyl, cyclohexyl, cyclopropylmethyl, cyclobutylmethyl, cyclopentylmethyl, cyclohexylmethyl, optionally substituted phenyl, optionally substituted heteroaryl, optionally substituted heterocyclyl, benzyl, 4-chlorophenylmethyl, 3-chlorophenylmethyl, 2-chlorophenylmethyl, 4-fluorophenylmethyl, 3-fluorophenylmethyl, 2-fluorophenylmethyl, heteroaryl-(C₁-C₆)-alkyl, heterocyclyl-(C₁-C₆)-alkyl, C(O)OR¹², C(O)NR¹⁰R¹¹, R¹²O(O)C—(C₁-C₆)-alkyl, R¹²O—(C₁-C₆)-alkyl, methylcarbonyloxymethyl, ethylcarbonyloxymethyl, n-propylcarbonyloxymethyl, 1-methylethylcarbonyloxymethyl or 1,1-dimethylethylcarbonyloxymethyl,
R⁶ represents hydrogen, methyl, ethyl, n-propyl, 1-methylethyl, n-butyl, 1-methylpropyl, 2-methylpropyl, 1,1-dimethylethyl, n-pentyl, cyclopropyl, cyclobutyl, cyclopentyl, cyclohexyl, cyclopropylmethyl, cyclobutylmethyl, cyclopentylmethyl, cyclohexylmethyl, optionally substituted phenyl, optionally substituted heteroaryl, optionally substituted heterocyclyl, benzyl, 4-chlorophenylmethyl, 3-chlorophenylmethyl, 2-chlorophenylmethyl, 4-fluorophenylmethyl, 3-fluorophenylmethyl, 2-fluorophenylmethyl, heteroaryl-(C₁-C₆)-alkyl or heterocyclyl-(C₁-C₆)-alkyl,
R⁷ and R⁸ are identical or different and independently of one another represent hydrogen, chlorine, bromine, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl or 1,1-dimethylpropyl,
R⁹ represents hydrogen, hydroxy, methyl, ethyl, n-propyl, 1-methylethyl, n-butyl, 1-methylpropyl, 2-methylpropyl, 1,1-dimethylethyl, n-pentyl, difluoromethyl, 2,2-difluoroethyl, 2,2,2-trifluoroethyl, methoxy, ethoxy, cyanomethyl, cyanoethyl, cyano-n-propyl, cyclopropyl, cyclobutyl, cyclopentyl, cyclohexyl, prop-2-en-1-yl, 1-methylethenyl, 1-butenyl, 2-butenyl, 3-butenyl, prop-2-yn-1-yl, but-2-yn-1-yl, but-3-yn-1-yl, pent-2-yn-1-yl, pent-3-yn-1-yl, pent-4-yn-1yl, methoxymethyl, methoxyethyl, ethoxymethyl, methoxy-n-propyl, n-propyloxymethyl or ethoxyethyl,
   or where R² and R⁹ together with the nitrogen atom or carbon atom to which they are respectively attached form a fully saturated or partially saturated ring which has 3 to 7 members in total, which is optionally interrupted by one to three heteroatoms from the group consisting of N, O and S and which is optionally substituted further,
R¹⁰ and R¹¹ are identical or different and independently of one another represent hydrogen, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, (C₁-C₆)-haloalkyl, (C₂-C₆)-haloalkenyl, (C₂-C₆)-haloalkynyl, cyclopropyl, cyclobutyl, cyclopentyl, cyclohexyl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 1-cyanocyclopropyl, 2-cyanocyclopropyl, 1-methylcyclobutyl, 2-methylcyclobutyl, 3-methylcyclobutyl, 1-cyanocyclobutyl, 2-cyanocyclobutyl, 3-cyanocyclobutyl, 3-methoxycyclobutyl, 1-allylcyclopropyl, 1-vinylcyclobutyl, 1-vinylcyclopropyl, 1-ethylcyclopropyl, 1-methylcyclohexyl, 2-methylcyclohexyl, 3-methylcyclohexyl, 1-methoxycyclohexyl, 2-methoxycyclohexyl, 3-methoxycyclohexyl, 4-methoxycyclohexyl, cyclopropylmethyl, cyclobutylmethyl, cyclopentylmethyl, cyclohexylmethyl, (C₃-C₆)-halocycloalkyl, (C₄-C₆)-cycloalkenyl, (C₄-C₆)-halocycloalkenyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, trifluoromethoxymethyl, trifluoromethoxyethyl, trifluoromethoxy-n-propyl, difluoromethoxymethyl, difluoromethoxyethyl, difluoromethoxy-n-propyl, 2,2-difluoroethoxymethyl, 2,2-difluoroethoxyethyl, 2,2-difluoroethoxy-n-propyl, 2,2,2-trifluoroethoxymethyl, 2,2,2-trifluoroethoxyethyl, 2,2,2-trifluoroethoxy-n-propyl, pentafluoroethoxymethyl, pentafluoroethoxyethyl, pentafluoroethoxy-n-propyl, methylthiomethyl, methylthioethyl, ethylthioethyl, methylthio-n-propyl, ethylthio-n-propyl, trifluoromethylthiomethyl, trifluoromethylthioethyl, trifluoromethylthio-n-propyl, optionally substituted phenyl, aryl-(C₁-C₅)-alkyl, optionally substituted heteroaryl, heteroaryl-(C₁-C₅)-alkyl, (C₄-C₆)-cycloalkenyl-(C₁-C₅)-alkyl, C(O)R¹², SO₂R¹³, optionally substituted heterocyclyl, (C₁-C₅)-alkoxycarbonyl-(C₁-C₅)-alkyl, (C₁-C₅)-alkoxycarbonyl, aryl-(C₁-C₅)-alkoxycarbonyl-(C₁-C₅)-alkyl, aryl-(C₁-C₅)-alkoxycarbonyl, heteroaryl-(C₁-C₅)-alkoxycarbonyl, (C₂-C₆)-alkenyloxycarbonyl, (C₂-C₆)-alkynyloxycarbonyl or heterocyclyl-(C₁-C₅)-alkyl,
R¹² represents methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, cyclopropyl, cyclobutyl, cyclopentyl, cyclohexyl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 2-cyanocyclopropyl, 1-methylcyclobutyl, 2-methylcyclobutyl, 3-methylcyclobutyl, 2-cyanocyclobutyl, 3-cyanocyclobutyl, 3-methoxycyclobutyl, 1-allylcyclopropyl, 1-ethylcyclopropyl, 1-methylcyclohexyl, 2-methylcyclohexyl, 3-methylcyclohexyl, 2-methoxycyclohexyl, 3-methoxycyclohexyl, 4-methoxycyclohexyl, cyclopropylmethyl, cyclobutylmethyl, cyclopentylmethyl, cyclohexylmethyl, trifluoromethyl, pentafluoroethyl, 1,1,2,2-tetrafluoroethyl, heptafluoropropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, 3,3-difluoro-n-propyl, 3,3,3-trifluoro-n-propyl, 4,4-difluoro-n-butyl, 4,4,4-trifluoro-n-butyl, (C₂-C₆)-haloalkenyl, (C₂-C₆)-haloalkynyl, (C₃-C₆)-halocycloalkyl, (C₄-C₆)-cycloalkenyl, (C₄-C₆)-halocycloalkenyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, optionally substituted phenyl, aryl-(C₁-C₆)-alkyl, optionally substituted heteroaryl, heteroaryl-(C₁-C₆)-alkyl, (C₄-C₆)-cycloalkenyl-(C₁-C₆)-alkyl, (C₁-C₆)-alkoxycarbonyl-(C₁-C₆)-alkyl, (C₂-C₆)-alkenyloxycarbonyl-(C₁-C₆)-alkyl, aryl-(C₁-C₆)-alkoxycarbonyl-(C₁-C₆)-alkyl, hydroxycarbonyl-(C₁-C₆)-alkyl, optionally substituted heterocyclyl or heterocyclyl-(C₁-C₆)-alkyl,
R¹³ represents methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, cyclopropylmethyl, cyclobutylmethyl, cyclopentylmethyl, cyclohexylmethyl, cyanomethyl, cyanoethyl, cyano-n-propyl, cyano-n-butyl, ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl, cyclopropyl, cyclobutyl, cyclopentyl, cyclohexyl, 1-methylcyclopropyl, 2-methylcyclopropyl, 2,2-dimethylcyclopropyl, 2,3-dimethylcyclopropyl, 1,1′-bi(cyclopropyl)-1-yl, 1,1′-bi(cyclopropyl)-2-yl, 2′-methyl-1,1′-bi(cyclopropyl)-2-yl, 2-cyanocyclopropyl, 1-methylcyclobutyl, 2-methylcyclobutyl, 3-methylcyclobutyl, 2-cyanocyclobutyl, 3-cyanocyclobutyl, 3-methoxycyclobutyl, 1-allylcyclopropyl, 1-ethylcyclopropyl, 1-methylcyclohexyl, 2-methylcyclohexyl, 3-methylcyclohexyl, 2-methoxycyclohexyl, 3-methoxycyclohexyl, 4-methoxycyclohexyl, cyclopropylmethyl, cyclobutylmethyl, cyclopentylmethyl, cyclohexylmethyl, trifluoromethyl, pentafluoroethyl, 1,1,2,2-tetrafluoroethyl, heptafluoro-n-propyl, heptafluoroisopropyl, nonafluorobutyl, chlorodifluoromethyl, bromodifluoromethyl, dichlorofluoromethyl, iododifluoromethyl, bromofluoromethyl, 1-fluoroethyl, 2-fluoroethyl, fluoromethyl, difluoromethyl, 2,2-difluoroethyl, 2,2,2-trifluoroethyl, 3,3,3-trifluoro-n-propyl, difluoro-tert-butyl, (C₂-C₆)-haloalkenyl, (C₂-C₆)-haloalkynyl, (C₃-C₆)-halocycloalkyl, (C₄-C₆)-cycloalkenyl, (C₄-C₆)-halocycloalkenyl, methoxymethyl, ethoxymethyl, ethoxyethyl, methoxyethyl, methoxy-n-propyl, ethoxy-n-propyl, methoxybutyl, methoxyisopropyl, isopropoxymethyl, isopropoxyethyl, optionally substituted phenyl, aryl-(C₁-C₆)-alkyl, optionally substituted heteroaryl, heteroaryl-(C₁-C₆)-alkyl, heterocyclyl-(C₁-C₆)-alkyl, (C₄-C₆)-cycloalkenyl-(C₁-C₆)-alkyl or NR¹⁰R¹¹,
R¹⁴ represents hydrogen,
   or where R³ and R¹⁴ together with the carbon atom to which they are attached form a carbonyl group.

The abovementioned general or preferred radical definitions apply both to the end products of the general formula (I) and, correspondingly, to the starting materials or the intermediates required in each case for the preparation. These radical definitions can be combined with one another as desired, i.e. including combinations between the given preferred ranges.

Primarily for reasons of higher herbicidal activity, better selectivity and/or better producibility, compounds of the abovementioned formula (I) according to the invention or their salts or their use according to the invention are of particular interest in which individual radicals have one of the preferred meanings already specified or specified below, or in particular those in which one or more of the preferred meanings already specified or specified below occur in combination.

With regard to the compounds according to the invention, the terms used above and further below will be elucidated. These are familiar to the person skilled in the art and especially have the definitions elucidated hereinafter:

Unless defined differently below, names of chemical groups are generally to be understood such that attachment to the skeleton or the remainder of the molecule is via the structural element mentioned last, i.e. for example in the case of (C₂-C₈)-alkenyloxy via the oxygen atom and in the case of heterocyclyl-(C₁-C₈)-alkyl or R¹²O(O)C—(C₁-C₈)-alkyl in each case via the carbon atom of the alkyl group.

According to the invention, “alkylsulfonyl”—alone or as part of a chemical group—refers to straight-chain or branched alkylsulfonyl, preferably having 1 to 8 or 1 to 6 carbon atoms, for example (but not limited to) (C₁-C₆)-alkylsulfonyl such as methylsulfonyl, ethylsulfonyl, propylsulfonyl, 1-methylethylsulfonyl, butylsulfonyl, 1-methylpropylsulfonyl, 2-methylpropylsulfonyl, 1,1-dimethylethylsulfonyl, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl.

“Alkylthio”—alone or as part of a chemical group—denotes an alkyl radical attached via a sulfur atom and refers to straight-chain or branched S-alkyl, preferably having 1 to 8 or 1 to 6 carbon atoms, such as (C₁-C₁₀)—, (C₁-C₆)- or (C₁-C₄)-alkylthio, for example (but not limited to) (C₁-C₆)-alkylthio such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio.

According to the invention, alkenylthio denotes an alkenyl radical attached via a sulfur atom, alkynylthio denotes an alkynyl radical attached via a sulfur atom, cycloalkylthio denotes a cycloalkyl radical attached via a sulfur atom, and cycloalkenylthio denotes a cycloalkenyl radical attached via a sulfur atom.

“Alkoxy” denotes an alkyl radical attached via an oxygen atom, for example (but not limited to) (C₁-C₆)-alkoxy such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy. Alkenyloxy denotes an alkenyl radical attached via an oxygen atom, and alkynyloxy denotes an alkynyl radical attached via an oxygen atom, such as (C₂-C₁₀)—, (C₂-C₆)- or (C₂-C₄)-alkenoxy and (C₃-C₁₀)—, (C₃-C₆)- or (C₃-C₄)-alkynoxy.

“Cycloalkyloxy” denotes a cycloalkyl radical attached via an oxygen atom and cycloalkenyloxy denotes a cycloalkenyl radical attached via an oxygen atom.

According to the invention, “alkylcarbonyl” (alkyl-C(═O)—), unless defined differently elsewhere, represents alkyl radicals attached to the skeleton via —C(═O)—, such as (C₁-C₁₀)—, (C₁-C₆)- or (C₁-C₄)-alkylcarbonyl. Here, the number of the carbon atoms refers to the alkyl radical in the alkylcarbonyl group.

Analogously, “alkenylcarbonyl” and “alkynylcarbonyl”, unless defined differently elsewhere, in accordance with the invention, respectively represent alkenyl and alkynyl radicals attached to the skeleton via —C(═O)—, such as (C₂-C₁₀)—, (C₂-C₆)- or (C₂-C₄)-alkenylcarbonyl and (C₂-C₁₀)—, (C₂-C₆)- and (C₂-C₄)-alkynylcarbonyl. Here, the number of the carbon atoms refers to the alkenyl or alkynyl radical in the alkenylcarbonyl or alkynylcarbonyl group.

Alkoxycarbonyl (alkyl-O—C(═O)—), unless defined differently elsewhere: alkyl radicals attached to the skeleton via —O—C(═O)—, such as (C₁-C₁₀)—, (C₁-C₆)- or (C₁-C₄)-alkoxycarbonyl. Here, the number of the carbon atoms refers to the alkyl radical in the alkoxycarbonyl group. Analogously, “alkenyloxycarbonyl” and “alkynyloxycarbonyl”, unless defined differently elsewhere, in accordance with the invention, respectively represent alkenyl and alkynyl radicals attached to the skeleton via —O—C(═O)—, such as (C₂-C₁₀)—, (C₂-C₆)- or (C₂-C₄)-alkenyloxycarbonyl and (C₃-C₁₀)—, (C₃-C₆)- and (C₃-C₄)-alkynyloxycarbonyl. Here, the number of the carbon atoms refers to the alkenyl or alkynyl radical in the alkenylcarbonyl or alkynylcarbonyl group.

According to the invention, the term “alkylcarbonyloxy” (alkyl-C(═O)—O—), unless defined differently elsewhere, represents alkyl radicals attached to the skeleton via the oxygen of a carbonyloxy group (—C(═O)—O—), such as (C₁-C₁₀)—, (C₁-C₆)- or (C₁-C₄)-alkylcarbonyloxy. Here, the number of the carbon atoms refers to the alkyl radical in the alkylcarbonyloxy group.

Analogously, “alkenylcarbonyloxy” and “alkynylcarbonyloxy” are defined in accordance with the invention respectively as alkenyl and alkynyl radicals attached to the skeleton via the oxygen of (—C(═O)—O—), such as (C₂-C₁₀)—, (C₂-C₆)- or (C₂-C₄)-alkenylcarbonyloxy or (C₂-C₁₀)—, (C₂-C₆)- or (C₂-C₄)-alkynylcarbonyloxy. Here, the number of the carbon atoms refers to the alkenyl or alkynyl radical in the alkenyl- or alkynylcarbonyloxy group respectively.

In short forms such as C(O)R¹², C(O)OR¹², OC(O)NR¹⁰R¹¹ or C(O)NR¹⁰R¹¹, the short form O shown in brackets represents an oxygen atom attached to the adjacent carbon atom via a double bond.

In short forms such as OC(S)OR¹², OC(S)SR¹³, OC(S)NR¹⁰R¹¹, the short form S shown in brackets represents a sulfur atom attached to the adjacent carbon atom via a double bond.

The term “aryl” means a mono-, bi- or polycyclic aromatic system having preferably 6 to 14, in particular 6 to 10, carbon ring atoms, for example phenyl, naphthyl, anthryl, phenanthrenyl and the like, preferably phenyl.

The term aryl also includes polycyclic systems, such as tetrahydronaphthyl, indenyl, indanyl, fluorenyl, biphenylyl, where the point of attachment is on the aromatic system. Preferred aryl substituents here are, for example, hydrogen, halogen, alkyl, cycloalkyl, cycloalkylalkyl, cycloalkenyl, halocycloalkyl, alkenyl, alkynyl, aryl, arylalkyl, arylalkenyl, heteroaryl, heteroarylalkyl, heterocyclyl, heterocyclylalkyl, alkoxyalkyl, alkylthio, haloalkylthio, haloalkyl, alkoxy, haloalkoxy, cycloalkoxy, cycloalkylalkoxy, aryloxy, heteroraryloxy, alkoxyalkoxy, alkynylalkoxy, alkenyloxy, bis-alkylaminoalkoxy, tris-[alkyl]silyl, bis-[alkyl]arylsilyl, bis-[alkyl]alkylsilyl, tris-[alkyl]silylalkynyl, arylalkynyl, heteroarylalkynyl, alkylalkynyl, cycloalkylalkynyl, haloalkylalkynyl, heterocyclyl-N-alkoxy, nitro, cyano, amino, alkylamino, bis-alkylamino, alkylcarbonylamino, cycloalkylcarbonylamino, arylcarbonylamino, alkoxycarbonylamino, alkoxycarbonylalkylamino, arylalkoxycarbonylalkylamino, hydroxycarbonyl, alkoxycarbonyl, aminocarbonyl, alkylaminocarbonyl, cycloalkylaminocarbonyl, bis-alkylaminocarbonyl, heteroarylalkoxy, arylalkoxy.

A heterocyclic radical (heterocyclyl) comprises at least one heterocyclic ring (=carbocyclic ring in which at least one carbon atom is replaced by a heteroatom, preferably by at least one heteroatom from the group consisting of N, O, S), which is saturated or partially saturated and may be unsubstituted or substituted, where the point of attachment is located at a ring atom. Here, the heterocyclyl radical or the heterocyclic ring may be fused to other carbocyclic or heterocyclic rings or aryl- or heteroaryl rings. In the case of heterocyclyl, polycyclic systems are also included, for example 8-azabicyclo[3.2.1]octanyl, 8-azabicyclo[2.2.2]octanyl or 1-azabicyclo[2.2.1]heptyl. Heterocyclyl also includes spirocyclic systems, such as, for example, 1-oxa-5-aza-spiro[2.3]hexyl. Unless defined otherwise, the heterocyclic ring contains 3 to 9 ring atoms, in particular 3 to 6 ring atoms, and one or more, preferably 1 to 4, in particular 1, 2 or 3 heteroatoms in the heterocyclic ring, preferably from the group N, O and S, where, however, two oxygen atoms are not directly adjacent to one another, for example having one heteroatom from the group consisting of N, O and S 1- or 2- or 3-pyrrolidinyl, 3,4-dihydro-2H-pyrrol-2- or -3-yl, 2,3-dihydro-1H-pyrrol-1- or -2- or -3- or -4- or -5-yl; 2,5-dihydro-1H-pyrrol-1- or -2- or -3-yl, 1- or 2- or 3- or 4-piperidinyl; 2,3,4,5-tetrahydropyridin-2- or -3- or -4- or -5-yl or -6-yl; 1,2,3,6-tetrahydropyridin-1- or -2- or -3- or -4- or -5- or -6-yl; 1,2,3,4-tetrahydropyridin-1- or -2- or -3- or -4- or -5- or -6-yl; 1,4-dihydropyridin-1- or -2- or -3- or -4-yl; 2,3-dihydropyridin-2- or -3- or -4- or -5- or -6-yl; 2,5-dihydropyridin-2- or -3- or -4- or -5- or -6-yl, 1- or 2- or 3- or 4-azepanyl; 2,3,4,5-tetrahydro-1H-azepin-1- or -2- or -3- or -4- or -5- or -6- or -7-yl; 2,3,4,7-tetrahydro-1H-azepin-1- or -2- or -3- or -4- or -5- or -6- or -7-yl; 2,3,6,7-tetrahydro-1H-azepin-1- or -2- or -3- or -4-yl; 3,4,5,6-tetrahydro-2H-azepin-2- or -3- or -4- or -5- or -6- or -7-yl; 4,5-dihydro-1H-azepin-1- or -2- or -3- or -4-yl; 2,5-dihydro-1H-azepin-1- or -2- or -3- or -4- or -5- or -6- or -7-yl; 2,7-dihydro-1H-azepin-1- or -2- or -3- or -4-yl; 2,3-dihydro-1H-azepin-1- or -2- or -3- or -4- or -5- or -6- or -7-yl; 3,4-dihydro-2H-azepin-2- or -3- or -4- or -5- or -6- or -7-yl; 3,6-dihydro-2H-azepin-2- or -3- or -4- or -5- or -6- or -7-yl; 5,6-dihydro-2H-azepin-2- or -3- or -4- or -5- or -6- or -7-yl; 4,5-dihydro-3H-azepin-2- or -3- or -4- or -5- or -6- or -7-yl; 1H-azepin-1- or -2- or -3- or -4- or -5- or -6- or -7-yl; 2H-azepin-2- or -3- or -4- or -5- or -6- or -7-yl; 3H-azepin-2- or -3- or -4- or -5- or -6- or -7-yl; 4H-azepin-2- or -3- or -4- or -5- or -6- or -7-yl, 2- or 3-oxolanyl (=2- or 3-tetrahydrofuranyl); 2,3-dihydrofuran-2- or -3- or -4- or -5-yl; 2,5-dihydrofuran-2- or -3-yl, 2- or 3- or 4-oxanyl (=2- or 3- or 4-tetrahydropyranyl); 3,4-dihydro-2H-pyran-2- or -3- or -4- or -5- or -6-yl; 3,6-dihydro-2H-pyran-2- or -3- or -4- or -5- or -6-yl; 2H-pyran-2- or -3- or -4- or -5- or -6-yl; 4H-pyran-2- or -3- or -4-yl, 2- or -3- or -4-oxepanyl; 2,3,4,5-tetrahydrooxepin-2- or -3- or -4- or -5- or -6- or -7-yl; 2,3,4,7-tetrahydrooxepin-2- or -3- or -4- or -5- or -6- or -7-yl; 2,3,6,7-tetrahydrooxepin-2- or -3- or -4-yl; 2,3-dihydrooxepin-2- or -3- or -4- or -5- or -6- or -7-yl; 4,5-dihydrooxepin-2- or -3- or -4-yl; 2,5-dihydrooxepin-2- or -3- or -4- or -5- or -6- or -7-yl; oxepin-2- or -3- or -4- or -5- or -6- or -7-yl; 2- or 3-tetrahydrothiophenyl; 2,3-dihydrothiophen-2- or -3- or -4- or -5-yl; 2,5-dihydrothiophen-2- or -3-yl; tetrahydro-2H-thiopyran-2- or -3- or -4-yl; 3,4-dihydro-2H-thiopyran-2- or -3- or -4- or -5- or -6-yl; 3,6-dihydro-2H-thiopyran-2- or -3- or -4- or -5- or -6-yl; 2H-thiopyran-2- or -3- or -4- or -5- or -6-yl; 4H-thiopyran-2- or -3- or -4-yl. Preferred 3-membered and 4-membered heterocycles are, for example, 1- or 2-aziridinyl, oxiranyl, thiiranyl, 1- or 2- or 3-azetidinyl, 2- or 3-oxetanyl, 2- or 3-thietanyl, 1,3-dioxetan-2-yl. Further examples of “heterocyclyl” are a partially or fully hydrogenated heterocyclic radical having two heteroatoms from the group consisting of N, O and S, for example 1- or 2- or 3- or 4-pyrazolidinyl; 4,5-dihydro-3H-pyrazol-3- or -4- or -5-yl; 4,5-dihydro-1H-pyrazol-1- or -3- or -4- or -5-yl; 2,3-dihydro-1H-pyrazol-1- or -2- or -3- or -4- or -5-yl; 1- or 2- or 3- or 4-imidazolidinyl; 2,3-dihydro-1H-imidazol-1- or -2- or -3- or -4-yl; 2,5-dihydro-1H-imidazol-1- or -2- or -4- or -5-yl; 4,5-dihydro-1H-imidazol-1- or -2- or -4- or -5-yl; hexahydropyridazin-1- or -2- or -3- or -4-yl; 1,2,3,4-tetrahydropyridazin-1- or -2- or -3- or -4- or -5- or -6-yl; 1,2,3,6-tetrahydropyridazin-1- or -2- or -3- or -4- or -5- or -6-yl; 1,4,5,6-tetrahydropyridazin-1- or -3- or -4- or -5- or -6-yl; 3,4,5,6-tetrahydropyridazin-3- or -4- or -5-yl; 4,5-dihydropyridazin-3- or -4-yl; 3,4-dihydropyridazin-3- or -4- or -5- or -6-yl; 3,6-dihydropyridazin-3- or -4-yl; 1,6-dihydropyridazin-1- or -3- or -4- or -5- or -6-yl; hexahydropyrimidin-1- or -2- or -3- or -4-yl; 1,4,5,6-tetrahydropyrimidin-1- or -2- or -4- or -5- or -6-yl; 1,2,5,6-tetrahydropyrimidin-1- or -2- or -4- or -5- or -6-yl; 1,2,3,4-tetrahydropyrimidin-1- or -2- or -3- or -4- or -5- or -6-yl; 1,6-dihydropyrimidin-1- or -2- or -4- or -5- or -6-yl; 1,2-dihydropyrimidin-1- or 2- or 4- or 5- or 6-yl; 2,5-dihydropyrimidin-2- or 4- or 5-yl; 4,5-dihydropyrimidin-4- or 5- or -6-yl; 1,4-dihydropyrimidin-1- or -2- or -4- or -5- or -6-yl; 1- or 2- or 3-piperazinyl; 1,2,3,6-tetrahydropyrazin-1- or -2- or -3- or -5- or -6-yl; 1,2,3,4-tetrahydropyrazin-1- or -2- or -3- or -4- or -5- or -6-yl; 1,2-dihydropyrazin-1- or -2- or -3- or -5- or -6-yl; 1,4-dihydropyrazin-1- or -2- or -3-yl; 2,3-dihydropyrazin-2- or -3- or -5- or -6-yl; 2,5-dihydropyrazin-2- or -3-yl; 1,3-dioxolan-2- or -4- or -5-yl; 1,3-dioxol-2- or -4-yl; 1,3-dioxan-2- or -4- or -5-yl; 4H-1,3-dioxin-2- or -4- or -5- or -6-yl; 1,4-dioxan-2- or -3- or -5- or -6-yl; 2,3-dihydro-1,4-dioxin-2- or -3- or -5- or -6-yl; 1,4-dioxin-2- or -3-yl; 1,2-dithiolan-3- or -4-yl; 3H-1,2-dithiol-3- or -4- or -5-yl; 1,3-dithiolan-2- or -4-yl; 1,3-dithiol-2- or -4-yl; 1,2-dithian-3- or -4-yl; 3,4-dihydro-1,2-dithiin-3- or -4- or -5- or -6-yl; 3,6-dihydro-1,2-dithiin-3- or -4-yl; 1,2-dithiin-3- or -4-yl; 1,3-dithian-2- or -4- or -5-yl; 4H-1,3-dithiin-2- or -4- or -5- or -6-yl; isoxazolidin-2- or -3- or -4- or -5-yl; 2,3-dihydroisoxazol-2- or -3- or -4- or -5-yl; 2,5-dihydroisoxazol-2- or -3- or -4- or -5-yl; 4,5-dihydroisoxazol-3- or -4- or -5-yl; 1,3-oxazolidin-2- or -3- or -4- or -5-yl; 2,3-dihydro-1,3-oxazol-2- or -3- or -4- or -5-yl; 2,5-dihydro-1,3-oxazol-2- or -4- or -5-yl; 4,5-dihydro-1,3-oxazol-2- or -4- or -5-yl; 1,2-oxazinan-2- or -3- or -4- or -5- or -6-yl; 3,4-dihydro-2H-1,2-oxazin-2- or -3- or -4- or -5- or -6-yl; 3,6-dihydro-2H-1,2-oxazin-2- or -3- or -4- or -5- or -6-yl; 5,6-dihydro-2H-1,2-oxazin-2- or -3- or -4- or -5- or -6-yl; 5,6-dihydro-4H-1,2-oxazin-3- or -4- or -5- or -6-yl; 2H-1,2-oxazin-2- or -3- or -4- or -5- or -6-yl; 6H-1,2-oxazin-3- or -4- or -5- or -6-yl; 4H-1,2-oxazin-3- or -4- or -5- or -6-yl; 1,3-oxazinan-2- or -3- or -4- or -5- or -6-yl; 3,4-dihydro-2H-1,3-oxazin-2- or -3- or -4- or -5- or -6-yl; 3,6-dihydro-2H-1,3-oxazin-2- or -3- or -4- or -5- or -6-yl; 5,6-dihydro-2H-1,3-oxazin-2- or -4- or -5- or -6-yl; 5,6-dihydro-4H-1,3-oxazin-2- or -4- or -5- or -6-yl; 2H-1,3-oxazin-2- or -4- or -5- or -6-yl; 6H-1,3-oxazin-2- or -4- or -5- or -6-yl; 4H-1,3-oxazin-2- or -4- or -5- or -6-yl; morpholin-2- or -3- or -4-yl; 3,4-dihydro-2H-1,4-oxazin-2- or -3- or -4- or -5- or -6-yl; 3,6-dihydro-2H-1,4-oxazin-2- or -3- or -5- or -6-yl; 2H-1,4-oxazin-2- or -3- or -5- or -6-yl; 4H-1,4-oxazin-2- or -3-yl; 1,2-oxazepan-2- or -3- or -4- or -5- or -6- or -7-yl; 2,3,4,5-tetrahydro-1,2-oxazepin-2- or -3- or -4- or -5- or -6- or -7-yl; 2,3,4,7-tetrahydro-1,2-oxazepin-2- or -3- or -4- or -5- or -6- or -7-yl; 2,3,6,7-tetrahydro-1,2-oxazepin-2- or -3- or -4- or -5- or -6- or -7-yl; 2,5,6,7-tetrahydro-1,2-oxazepin-2- or -3- or -4- or -5- or -6- or -7-yl; 4,5,6,7-tetrahydro-1,2-oxazepin-3- or -4- or -5- or -6- or -7-yl; 2,3-dihydro-1,2-oxazepin-2- or -3- or -4- or -5- or -6- or -7-yl; 2,5-dihydro-1,2-oxazepin-2- or -3- or -4- or -5- or -6- or -7-yl; 2,7-dihydro-1,2-oxazepin-2- or -3- or -4- or -5- or -6- or -7-yl; 4,5-dihydro-1,2-oxazepin-3- or -4- or -5- or -6- or -7-yl; 4,7-dihydro-1,2-oxazepin-3- or -4- or -5- or -6- or -7-yl; 6,7-dihydro-1,2-oxazepin-3- or -4- or -5- or -6- or -7-yl; 1,2-oxazepin-3- or -4- or -5- or -6- or -7-yl; 1,3-oxazepan-2- or -3- or -4- or -5- or -6- or -7-yl; 2,3,4,5-tetrahydro-1,3-oxazepin-2- or -3- or -4- or -5- or -6- or -7-yl; 2,3,4,7-tetrahydro-1,3-oxazepin-2- or -3- or -4- or -5- or -6- or -7-yl; 2,3,6,7-tetrahydro-1,3-oxazepin-2- or -3- or -4- or -5- or -6- or -7-yl; 2,5,6,7-tetrahydro-1,3-oxazepin-2- or -4- or -5- or -6- or -7-yl; 4,5,6,7-tetrahydro-1,3-oxazepin-2- or -4- or -5- or -6- or -7-yl; 2,3-dihydro-1,3-oxazepin-2- or -3- or -4- or -5- or -6- or -7-yl; 2,5-dihydro-1,3-oxazepin-2- or -4- or -5- or -6- or -7-yl; 2,7-dihydro-1,3-oxazepin-2- or -4- or -5- or -6- or -7-yl; 4,5-dihydro-1,3-oxazepin-2- or -4- or -5- or -6- or -7-yl; 4,7-dihydro-1,3-oxazepin-2- or -4- or -5- or -6- or -7-yl; 6,7-dihydro-1,3-oxazepin-2- or -4- or -5- or -6- or -7-yl; 1,3-oxazepin-2- or -4- or -5- or -6- or -7-yl; 1,4-oxazepan-2- or -3- or -5- or -6- or -7-yl; 2,3,4,5-tetrahydro-1,4-oxazepin-2- or -3- or -4- or -5- or -6- or -7-yl; 2,3,4,7-tetrahydro-1,4-oxazepin-2- or -3- or -4- or -5- or -6- or -7-yl; 2,3,6,7-tetrahydro-1,4-oxazepin-2- or -3- or -5- or -6- or -7-yl; 2,5,6,7-tetrahydro-1,4-oxazepin-2- or -3- or -5- or -6- or -7-yl; 4,5,6,7-tetrahydro-1,4-oxazepin-2- or -3- or -4- or -5- or -6- or -7-yl; 2,3-dihydro-1,4-oxazepin-2- or -3- or -5- or -6- or -7-yl; 2,5-dihydro-1,4-oxazepin-2- or -3- or -5- or -6- or -7-yl; 2,7-dihydro-1,4-oxazepin-2- or -3- or -5- or -6- or -7-yl; 4,5-dihydro-1,4-oxazepin-2- or -3- or -4- or -5- or -6- or -7-yl; 4,7-dihydro-1,4-oxazepin-2- or -3- or -4- or -5- or -6- or -7-yl; 6,7-dihydro-1,4-oxazepin-2- or -3- or -5- or -6- or -7-yl; 1,4-oxazepin-2- or -3- or -5- or -6- or -7-yl; isothiazolidin-2- or -3- or -4- or -5-yl; 2,3-dihydroisothiazol-2- or -3- or -4- or -5-yl; 2,5-dihydroisothiazol-2- or -3- or -4- or -5-yl; 4,5-dihydroisothiazol-3- or -4- or -5-yl; 1,3-thiazolidin-2- or -3- or -4- or -5-yl; 2,3-dihydro-1,3-thiazol-2- or -3- or -4- or -5-yl; 2,5-dihydro-1,3-thiazol-2- or -4- or -5-yl; 4,5-dihydro-1,3-thiazol-2- or -4- or -5-yl; 1,3-thiazinan-2- or -3- or -4- or -5- or -6-yl; 3,4-dihydro-2H-1,3-thiazin-2- or -3- or -4- or -5- or -6-yl; 3,6-dihydro-2H-1,3-thiazin-2- or -3- or -4- or -5- or -6-yl; 5,6-dihydro-2H-1,3-thiazin-2- or -4- or -5- or -6-yl; 5,6-dihydro-4H-1,3-thiazin-2- or -4- or -5- or -6-yl; 2H-1,3-thiazin-2- or -4- or -5- or -6-yl; 6H-1,3-thiazin-2- or -4- or -5- or -6-yl; 4H-1,3-thiazin-2- or -4- or -5- or -6-yl. Further examples of “heterocyclyl” are a partly or fully hydrogenated heterocyclic radical having 3 heteroatoms from the group of N, O and S, for example 1,4,2-dioxazolidin-2- or -3- or -5-yl; 1,4,2-dioxazol-3- or -5-yl; 1,4,2-dioxazinan-2- or -3- or -5- or -6-yl; 5,6-dihydro-1,4,2-dioxazin-3- or -5- or -6-yl; 1,4,2-dioxazin-3- or -5- or -6-yl; 1,4,2-dioxazepan-2- or -3- or -5- or -6- or -7-yl; 6,7-dihydro-5H-1,4,2-dioxazepin-3- or -5- or -6- or -7-yl; 2,3-dihydro-7H-1,4,2-dioxazepin-2- or -3- or -5- or -6- or -7-yl; 2,3-dihydro-5H-1,4,2-dioxazepin-2- or -3- or -5- or -6- or -7-yl; 5H-1,4,2-dioxazepin-3- or -5- or -6- or -7-yl; 7H-1,4,2-dioxazepin-3- or -5- or -6- or -7-yl. Structural examples of heterocycles are also given below (where the arrow indicates the position of the bond to the remainder of the molecule):

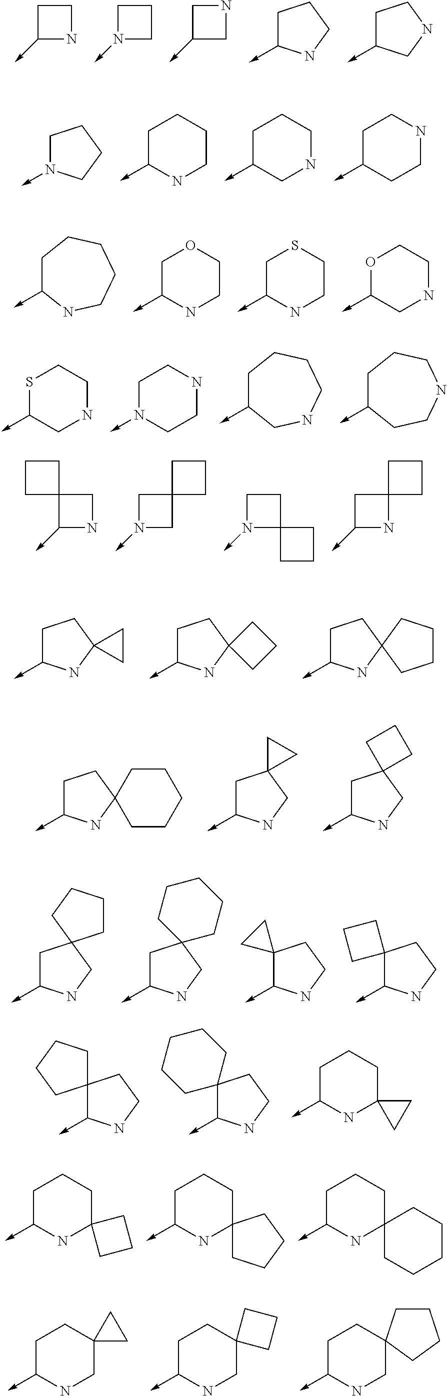

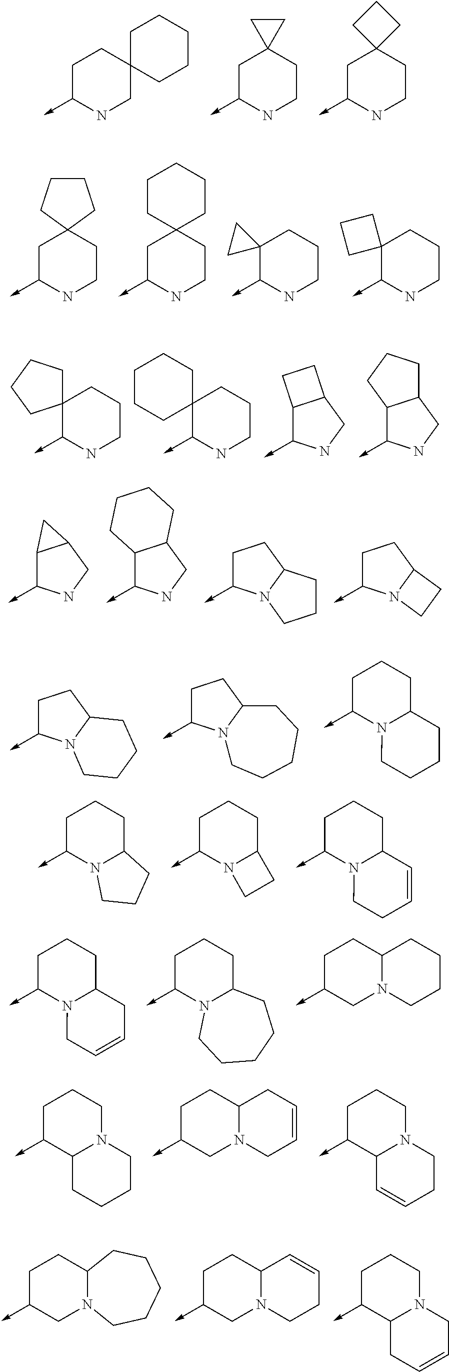

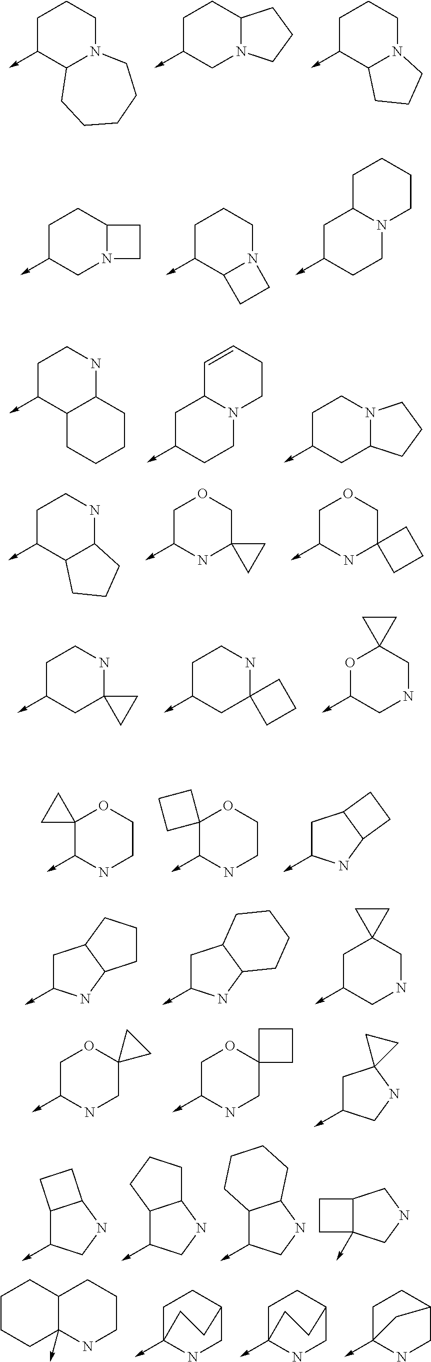

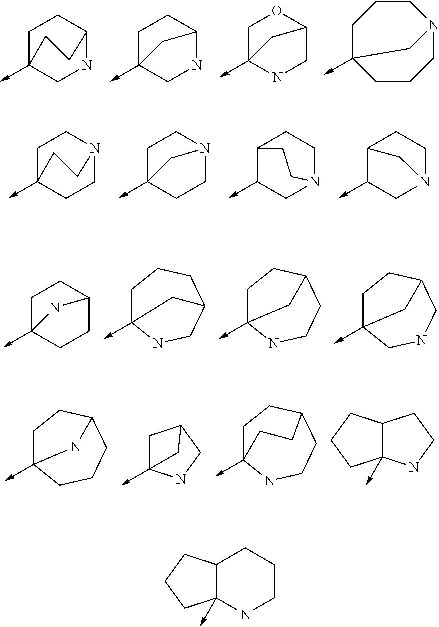

The heterocycles listed above are preferably substituted, for example, by hydrogen, halogen, alkyl, haloalkyl, hydroxyl, alkoxy, cycloalkoxy, aryloxy, alkoxyalkyl, alkoxyalkoxy, cycloalkyl, halocycloalkyl, aryl, arylalkyl, heteroaryl, heterocyclyl, alkenyl, alkylcarbonyl, cycloalkylcarbonyl, arylcarbonyl, heteroarylcarbonyl, alkoxycarbonyl, hydroxycarbonyl, cycloalkoxycarbonyl, cycloalkylalkoxycarbonyl, alkoxycarbonylalkyl, arylalkoxycarbonyl, arylalkoxycarbonylalkyl, alkynyl, alkynylalkyl, alkylalkynyl, trisalkylsilylalkynyl, nitro, amino, cyano, haloalkoxy, haloalkylthio, alkylthio, hydrothio, hydroxyalkyl, oxo, heteroarylalkoxy, arylalkoxy, heterocyclylalkoxy, heterocyclylalkylthio, heterocyclyloxy, heterocyclylthio, heteroaryloxy, bisalkylamino, alkylamino, cycloalkylamino, hydroxycarbonylalkylamino, alkoxycarbonylalkylamino, arylalkoxycarbonylalkylamino, alkoxycarbonylalkyl(alkyl)amino, aminocarbonyl, alkylaminocarbonyl, bisalkylaminocarbonyl, cycloalkylaminocarbonyl, hydroxycarbonylalkylaminocarbonyl, alkoxycarbonylalkylaminocarbonyl, arylalkoxycarbonylalkylaminocarbonyl.

When a base structure is substituted “by one or more” radicals or structural elements from a list of radicals (=group) or a generically defined group of radicals, this in each case includes simultaneous substitution by a plurality of identical and/or structurally different radicals.

If the heterocycle is a partly or fully saturated nitrogen-containing heterocycle, this may be joined to the rest of the molecule either via carbon or via the nitrogen.

Suitable substituents for a substituted heterocyclic radical are the substituents specified further down, and additionally also oxo and thioxo. The oxo group as a substituent on a ring carbon atom is then, for example, a carbonyl group in the heterocyclic ring. As a result, lactones and lactams are preferably also included. The oxo group may also occur on the ring heteroatoms, which may exist in different oxidation states, for example in the case of N and S, and in that case form, for example, the divalent —N(O)—, —S(O)— (also SO for short) and —S(O)₂— (also SO₂ for short) groups in the heterocyclic ring. In the case of —N(O)—and —S(O)— groups, both enantiomers in each case are included.

According to the invention, the expression “heteroaryl” refers to heteroaromatic compounds, i.e. fully unsaturated aromatic heterocyclic compounds, preferably 5- to 7-membered rings having 1 to 4, preferably 1 or 2, identical or different heteroatoms, preferably O, S or N. Inventive heteroaryls are, for example, 1H-pyrrol-1-yl; 1H-pyrrol-2-yl; 1H-pyrrol-3-yl; furan-2-yl; furan-3-yl; thien-2-yl; thien-3-yl, 1H-imidazol-1-yl; 1H-imidazol-2-yl; 1H-imidazol-4-yl; 1H-imidazol-5-yl; 1H-pyrazol-1-yl; 1H-pyrazol-3-yl; 1H-pyrazol-4-yl; 1H-pyrazol-5-yl, 1H-1,2,3-triazol-1-yl, 1H-1,2,3-triazol-4-yl, 1H-1,2,3-triazol-5-yl, 2H-1,2,3-triazol-2-yl, 2H-1,2,3-triazol-4-yl, 1H-1,2,4-triazol-1-yl, 1H-1,2,4-triazol-3-yl, 4H-1,2,4-triazol-4-yl, 1,2,4-oxadiazol-3-yl, 1,2,4-oxadiazol-5-yl, 1,3,4-oxadiazol-2-yl, 1,2,3-oxadiazol-4-yl, 1,2,3-oxadiazol-5-yl, 1,2,5-oxadiazol-3-yl, azepinyl, pyridin-2-yl, pyridin-3-yl, pyridin-4-yl, pyrazin-2-yl, pyrazin-3-yl, pyrimidin-2-yl, pyrimidin-4-yl, pyrimidin-5-yl, pyridazin-3-yl, pyridazin-4-yl, 1,3,5-triazin-2-yl, 1,2,4-triazin-3-yl, 1,2,4-triazin-5-yl, 1,2,4-triazin-6-yl, 1,2,3-triazin-4-yl, 1,2,3-triazin-5-yl, 1,2,4-, 1,3,2-, 1,3,6- and 1,2,6-oxazinyl, isoxazol-3-yl, isoxazol-4-yl, isoxazol-5-yl, 1,3-oxazol-2-yl, 1,3-oxazol-4-yl, 1,3-oxazol-5-yl, isothiazol-3-yl, isothiazol-4-yl, isothiazol-5-yl, 1,3-thiazol-2-yl, 1,3-thiazol-4-yl, 1,3-thiazol-5-yl, oxepinyl, thiepinyl, 1,2,4-triazolonyl and 1,2,4-diazepinyl, 2H-1,2,3,4-tetrazol-5-yl, 1H-1,2,3,4-tetrazol-5-yl, 1,2,3,4-oxatriazol-5-yl, 1,2,3,4-thiatriazol-5-yl, 1,2,3,5-oxatriazol-4-yl, 1,2,3,5-thiatriazol-4-yl. The heteroaryl groups according to the invention may also be substituted by one or more identical or different radicals. If two adjacent carbon atoms are part of a further aromatic ring, the systems are fused heteroaromatic systems, such as benzofused or polyannulated heteroaromatics. Preferred examples are quinolines (e.g. quinolin-2-yl, quinolin-3-yl, quinolin-4-yl, quinolin-5-yl, quinolin-6-yl, quinolin-7-yl, quinolin-8-yl); isoquinolines (e.g. isoquinolin-1-yl, isoquinolin-3-yl, isoquinolin-4-yl, isoquinolin-5-yl, isoquinolin-6-yl, isoquinolin-7-yl, isoquinolin-8-yl); quinoxaline; quinazoline; cinnoline; 1,5-naphthyridine; 1,6-naphthyridine; 1,7-naphthyridine; 1,8-naphthyridine; 2,6-naphthyridine; 2,7-naphthyridine; phthalazine; pyridopyrazines; pyridopyrimidines; pyridopyridazines; pteridines; pyrimidopyrimidines. Examples of heteroaryl are also 5- or 6-membered benzofused rings from the group of 1H-indol-1-yl, 1H-indol-2-yl, 1H-indol-3-yl, 1H-indol-4-yl, 1H-indol-5-yl, 1H-indol-6-yl, 1H-indol-7-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, 2H-indazol-7-yl, 2H-isoindol-2-yl, 2H-isoindol-1-yl, 2H-isoindol-3-yl, 2H-isoindol-4-yl, 2H-isoindol-5-yl, 2H-isoindol-6-yl; 2H-isoindol-7-yl, 1H-benzimidazol-1-yl, 1H-benzimidazol-2-yl, 1H-benzimidazol-4-yl, 1H-benzimidazol-5-yl, 1H-benzimidazol-6-yl, 1H-benzimidazol-7-yl, 1,3-benzoxazol-2-yl, 1,3-benzoxazol-4-yl, 1,3-benzoxazol-5-yl, 1,3-benzoxazol-6-yl, 1,3-benzoxazol-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,2-benzisoxazol-3-yl, 1,2-benzisoxazol-4-yl, 1,2-benzisoxazol-5-yl, 1,2-benzisoxazol-6-yl, 1,2-benzisoxazol-7-yl, 1,2-benzisothiazol-3-yl, 1,2-benzisothiazol-4-yl, 1,2-benzisothiazol-5-yl, 1,2-benzisothiazol-6-yl, 1,2-benzisothiazol-7-yl.

The term “halogen” denotes, for example, fluorine, chlorine, bromine or iodine. If the term is used for a radical, “halogen” denotes, for example, a fluorine, chlorine, bromine or iodine atom.

According to the invention, “alkyl” denotes a straight-chain or branched open-chain, saturated hydrocarbon radical which is optionally mono- or polysubstituted, and in the latter case is referred to as “substituted alkyl”. Preferred substituents are halogen atoms, alkoxy, haloalkoxy, cyano, alkylthio, haloalkylthio, amino or nitro groups, particular preference being given to methoxy, methyl, fluoroalkyl, cyano, nitro, fluorine, chlorine, bromine or iodine. The prefix “bis” also includes the combination of different alkyl radicals, e.g. methyl(ethyl) or ethyl(methyl).

“Haloalkyl”, “-alkenyl” and “-alkynyl” respectively denote alkyl, alkenyl and alkynyl partly or fully substituted by identical or different halogen atoms, for example monohaloalkyl such as CH₂CH₂C₁, CH₂CH₂Br, CHClCH₃, CH₂C₁, CH₂F; perhaloalkyl such as CCl₃, CClF₂, CFCl₂, CF₂CClF₂, CF₂CClFCF₃; polyhaloalkyl such as CH₂CHFCl, CF₂CCIFH, CF₂CBrFH, CH₂CF₃; the term perhaloalkyl also encompasses the term perfluoroalkyl.

Partly fluorinated alkyl denotes a straight-chain or branched, saturated hydrocarbon which is mono- or polysubstituted by fluorine, where the fluorine atoms in question may be present as substituents on one or more different carbon atoms of the straight-chain or branched hydrocarbon chain, for example CHFCH₃, CH₂CH₂F, CH₂CH₂CF₃, CHF₂, CH₂F, CHFCF₂CF₃.

Partly fluorinated haloalkyl denotes a straight-chain or branched, saturated hydrocarbon which is substituted by different halogen atoms with at least one fluorine atom, where any other halogen atoms optionally present are selected from the group consisting of fluorine, chlorine or bromine, iodine. The corresponding halogen atoms may be present as substituents on one or more different carbon atoms of the straight-chain or branched hydrocarbon chain. Partly fluorinated haloalkyl also includes full substitution of the straight or branched chain by halogen including at least one fluorine atom. Haloalkoxy is, for example, OCF₃, OCHF₂, OCH₂F, OCF₂CF₃, OCH₂CF₃ and OCH₂CH₂C₁; the situation is equivalent for haloalkenyl and other halogen-substituted radicals.

The expression “(C₁-C₄)-alkyl” mentioned here by way of example is a brief notation for straight-chain or branched alkyl having one to 4 carbon atoms according to the range stated for carbon atoms, i.e. encompasses the methyl, ethyl, 1-propyl, 2-propyl, 1-butyl, 2-butyl, 2-methylpropyl or tert-butyl radicals. General alkyl radicals with a larger specified range of carbon atoms, e.g. “(C₁-C₆)-alkyl”, correspondingly also encompass straight-chain or branched alkyl radicals with a greater number of carbon atoms, i.e. according to the example also the alkyl radicals having 5 and 6 carbon atoms.

Unless stated specifically, preference is given to the lower carbon skeletons, for example having from 1 to 6 carbon atoms, or having from 2 to 6 carbon atoms in the case of unsaturated groups, in the case of the hydrocarbyl radicals such as alkyl, alkenyl and alkynyl radicals, including in composite radicals. Alkyl radicals, including in composite radicals such as alkoxy, haloalkyl, etc., are, for example, methyl, ethyl, n-propyl or i-propyl, n-, i-, t- or 2-butyl, pentyls, hexyls such as n-hexyl, i-hexyl and 1,3-dimethylbutyl, heptyls such as n-heptyl, 1-methylhexyl and 1,4-dimethylpentyl; alkenyl and alkynyl radicals are defined as the possible unsaturated radicals corresponding to the alkyl radicals, where at least one double bond or triple bond is present. Preference is given to radicals having one double bond or triple bond.

The term “alkenyl” also includes, in particular, straight-chain or branched open-chain hydrocarbon radicals having more than one double bond, such as 1,3-butadienyl and 1,4-pentadienyl, but also allenyl or cumulenyl radicals having one or more cumulated double bonds, for example allenyl (1,2-propadienyl), 1,2-butadienyl and 1,2,3-pentatrienyl. Alkenyl denotes, for example, vinyl which may optionally be substituted by further alkyl radicals, for example (but not limited thereto) (C₂-C₆)-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “alkynyl” also includes, in particular, straight-chain or branched open-chain hydrocarbon radicals having more than one triple bond, or else having one or more triple bonds and one or more double bonds, for example 1,3-butatrienyl or 3-penten-1-yn-1-yl. (C₂-C₆)-Alkynyl is, for example, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl.

The term “cycloalkyl” refers to a carbocyclic saturated ring system having preferably 3-8 ring carbon atoms, for example cyclopropyl, cyclobutyl, cyclopentyl or cyclohexyl, which optionally has further substitution, preferably by hydrogen, alkyl, alkoxy, cyano, nitro, alkylthio, haloalkylthio, halogen, alkenyl, alkynyl, haloalkyl, amino, alkylamino, bisalkylamino, alkoxycarbonyl, hydroxycarbonyl, arylalkoxycarbonyl, aminocarbonyl, alkylaminocarbonyl, cycloalkylaminocarbonyl. In the case of cycloalkyl, cyclic systems with substituents are included, also including substituents with a double bond on the cycloalkyl radical, for example an alkylidene group such as methylidene. In the case of cycloalkyl, polycyclic aliphatic systems are also included, for example bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl and adamantan-2-yl, but also systems such as 1,1′-bi(cyclopropyl)-1-yl, 1,1′-bi(cyclopropyl)-2-yl, for example. The term “(C₃-C₇)-cycloalkyl” is a brief notation for cycloalkyl having three to 7 carbon atoms, corresponding to the range specified for carbon atoms.

In the case of cycloalkyl, spirocyclic aliphatic systems are also included, for example spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl.

“Cycloalkenyl” denotes a carbocyclic, nonaromatic, partly unsaturated ring system having preferably 4-8 carbon atoms, e.g. 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl, also including substituents with a double bond on the cycloalkenyl radical, for example an alkylidene group such as methylidene. In the case of cycloalkenyl, the explanations for substituted cycloalkyl apply correspondingly.

The term “alkylidene”, also, for example, in the form (C₁-C₁₀)-alkylidene, means the radical of a straight-chain or branched open-chain hydrocarbon radical which is attached via a double bond. Possible bonding sites for alkylidene are naturally only positions on the base structure where two hydrogen atoms can be replaced by the double bond; radicals are, for example, ═CH₂, ═CH—CH₃, ═C(CH₃)—CH₃, ═C(CH₃)—C₂H₅ or ═C(C₂H₅)—C₂H₅. Cycloalkylidene denotes a carbocyclic radical attached via a double bond.

If the compounds can form, through a hydrogen shift, tautomers whose structure is not formally covered by the formula (I), these tautomers are nevertheless covered by the definition of the inventive compounds of the formula (I), unless a particular tautomer is under consideration. For example, many carbonyl compounds may be present both in the keto form and in the enol form, both forms being encompassed by the definition of the compound of the formula (I).

Depending on the nature of the substituents and the manner in which they are attached, the compounds of the general formula (I) may be present as stereoisomers. The formula (I) embraces all possible stereoisomers defined by the specific three-dimensional form thereof, such as enantiomers, diastereomers, Z and E isomers. If, for example, one or more alkenyl groups are present, diastereomers (Z and E isomers) may occur. If, for example, one or more asymmetric carbon atoms are present, enantiomers and diastereomers may occur. Stereoisomers can be obtained from the mixtures obtained in the preparation by customary separation methods. The chromatographic separation can be effected either on the analytical scale to find the enantiomeric excess or the diastereomeric excess, or else on the preparative scale to produce test specimens for biological testing. It is likewise possible to selectively prepare stereoisomers by using stereoselective reactions with use of optically active starting materials and/or auxiliaries. The invention thus also relates to all stereoisomers which are embraced by the general formula (I) but are not shown in their specific stereomeric form, and to mixtures thereof.

If the compounds are obtained as solids, the purification can also be carried out by recrystallization or digestion. If individual compounds (I) cannot be obtained in a satisfactory manner by the routes described above, they can be prepared by derivatization of other compounds (I).

Suitable isolation methods, purification methods and methods for separating stereoisomers of compounds of the formula (I) are methods generally known to the person skilled in the art from analogous cases, for example by physical processes such as crystallization, chromatographic methods, in particular column chromatography and HPLC (high pressure liquid chromatography), distillation, optionally under reduced pressure, extraction and other methods, any mixtures that remain can generally be separated by chromatographic separation, for example on chiral solid phases. Suitable for preparative amounts or on an industrial scale are processes such as crystallization, for example of diastereomeric salts which can be obtained from the diastereomer mixtures using optically active acids and, if appropriate, provided that acidic groups are present, using optically active bases.

In the context of the present invention, more preference is given to the following compounds of the formula (I) in which:
X represents oxygen,
A represents the grouping C—R¹ or the grouping N—R⁹ (N=nitrogen), where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents hydrogen, halogen, (C₁-C₄)-alkyl, (C₁-C₄)-haloalkyl, (C₁-C₄)-hydroxyalkyl, (C₁-C₄)-alkoxy, (C₁-C₄)-haloalkoxy or (C₃-C₈)-cycloalkyl,
R² represents hydrogen, halogen, hydroxy, (C₁-C₄)-alkyl, (C₁-C₄)-haloalkyl, (C₁-C₄)-hydroxyalkyl, (C₁-C₄)-alkoxy, (C₁-C₄)-haloalkoxy or (C₃-C₈)-cycloalkyl, (C₂-C₄)-alkenyl, (C₂-C₄)-alkynyl, (C₂-C₄)-alkynyloxy or NR¹⁰R¹¹,
   or where R¹ and R² together with the two carbon atoms to which they are attached form a fully saturated or partially saturated ring which has 3 to 7 members in total, which is optionally interrupted by one to three heteroatoms from the group consisting of N, O and S and which is optionally substituted further,
R³ represents hydroxy, halogen, (C₁-C₄)-alkoxy, (C₃-C₁₀)-cycloalkyl-(C₁-C₄)-alkoxy, aryl-(C₁-C₄)-alkoxy, arylcarbonyloxy, (C₁-C₄)-alkylcarbonyloxy, aryl-(C₁-C₄)-alkylcarbonyloxy, heteroarylcarbonyloxy, (C₃-C₈)-cycloalkylcarbonyloxy, heterocyclylcarbonyloxy, (C₁-C₄)-haloalkyl-carbonyloxy, (C₂-C₄)-alkenylcarbonyloxy, OC(O)OR¹² or OSO₂R¹³,
R⁴ represents hydrogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₆)-alkyl, heteroaryl-(C₁-C₆)-alkyl, heterocyclyl-(C₁-C₆)-alkyl, (C₂-C₆)-alkenyl, (C₂-C₆)-alkynyl, (C₂-C₆)-haloalkenyl, (C₂-C₆)-haloalkynyl, (C₄-C₈)-cycloalkenyl, aryl-(C₂-C₆)-alkenyl, heteroaryl-(C₂-C₆)-alkenyl, heterocyclyl-(C₂-C₆)-alkenyl, arylcarbonyl-(C₁-C₆)-alkyl, (C₁-C₆)-alkylcarbonyl-(C₁-C₆)-alkyl, heteroarylcarbonyl-(C₁-C₆)-alkyl, (C₃-C₁₀)-cycloalkylcarbonyl-(C₁-C₆)-alkyl, aryl-(C₁-C₆)-alkoxycarbonyl-(C₁-C₆)-alkyl, (C₁-C₆)-alkoxycarbonyl-(C₁-C₆)-alkyl, (C₁-C₆)-alkoxy-(C₁-C₆)-alkyl, (C₁-C₆)-haloalkoxy-(C₁-C₆)-alkyl, C(O)R¹², SO₂R¹³, CHO, R¹⁰R¹¹N—(C₁-C₈)-alkyl or cyano-(C₁-C₈)-alkyl,
R⁵ represents hydrogen, (C₁-C₄)-alkyl, (C₁-C₄)-haloalkyl, (C₃-C₅)-cycloalkyl, (C₃-C₅)-cycloalkyl-(C₁-C₄)-alkyl, aryl, heteroaryl, heterocyclyl, aryl-(C₁-C₄)-alkyl, heteroaryl-(C₁-C₄)-alkyl, heterocyclyl-(C₁-C₄)-alkyl, cyano, C(O)OR¹², C(O)NR¹⁰R¹¹, R¹²O(O)C—(C₁-C₄)-alkyl, R¹²O—(C₁-C₄)-alkyl, (C₁-C₄)-alkylcarbonyloxy-(C₁-C₄)-alkyl, (C₃-C₅)-cycloalkylcarbonyloxy-(C₁-C₄)-alkyl, arylcarbonyloxy-(C₁-C₄)-alkyl, heteroarylcarbonyloxy-(C₁-C₄)-alkyl or heterocyclylcarbonyloxy-(C₁-C₄)-alkyl,
R⁶ represents hydrogen, (C₁-C₄)-alkyl, (C₁-C₄)-haloalkyl, (C₃-C₅)-cycloalkyl or (C₃-C₅)-cycloalkyl-(C₁-C₄)-alkyl,
R⁷ and R⁸ are identical or different and independently of one another represent hydrogen, halogen or (C₁-C₄)-alkyl,
R⁹ represents hydrogen, hydroxy, (C₁-C₄)-alkyl, (C₁-C₄)-haloalkyl, (C₁-C₄)-alkoxy, (C₁-C₄)-alkoxy-(C₁-C₄)-alkyl, (C₃-C₈)-cycloalkyl, (C₂-C₄)-alkenyl or (C₂-C₄)-alkynyl,
   or where R² and R⁹ together with the nitrogen atom or carbon atom to which they are respectively attached form a fully saturated ring which has 3 to 7 members in total, which is optionally interrupted by one heteroatom from the group consisting of N, O and S and which is optionally substituted further,
R¹⁰ and R¹¹ are identical or different and independently of one another represent hydrogen, (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-haloalkoxy-(C₁-C₄)-alkyl, (C₁-C₄)-alkylthio-(C₁-C₄)-alkyl, (C₁-C₄)-haloalkylthio-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₈)-cycloalkyl-(C₁-C₄)-alkyl, (C₄-C₇)-cycloalkenyl-(C₁-C₄)-alkyl, C(O)R¹², SO₂R¹³, heterocyclyl, (C₁-C₄)-alkoxycarbonyl-(C₁-C₄)-alkyl, (C₁-C₄)-alkoxycarbonyl, aryl-(C₁-C₄)-alkoxycarbonyl-(C₁-C₄)-alkyl, aryl-(C₁-C₄)-alkoxycarbonyl, heteroaryl-(C₁-C₄)-alkoxycarbonyl, (C₂-C₄)-alkenyloxycarbonyl, (C₂-C₄)-alkynyloxycarbonyl or heterocyclyl-(C₁-C₄)-alkyl,
R¹² represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₅)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₅)-cycloalkyl-(C₁-C₄)-alkyl, (C₄-C₇)-cycloalkenyl-(C₁-C₄)-alkyl, (C₁-C₄)-alkoxycarbonyl-(C₁-C₄)-alkyl, (C₂-C₄)-alkenyloxycarbonyl-(C₁-C₄)-alkyl, aryl-(C₁-C₄)-alkoxycarbonyl-(C₁-C₄)-alkyl, hydroxycarbonyl-(C₁-C₄)-alkyl, heterocyclyl or heterocyclyl-(C₁-C₄)-alkyl,
R¹³ represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, heterocyclyl-(C₁-C₄)-alkyl, (C₃-C₅)-cycloalkyl-(C₁-C₄)-alkyl, (C₄-C₇)-cycloalkenyl-(C₁-C₄)-alkyl or NR¹⁰R¹¹,
R¹⁴ represents hydrogen,
   or where R³ and R¹⁴ together with the carbon atom to which they are attached form a carbonyl group,

where the cyclic structural elements (in particular the structural elements aryl, cycloalkyl, cycloalkenyl, heteroaryl and heterocyclyl) of the radicals mentioned respectively in R¹, R², R³, R⁴, R⁵, R⁶, R⁹, R¹⁰, R¹¹, R¹² and R¹³ and the rings formed by R¹ and R² or R² and R⁹, having 3 to 7 members in total, are in each case unsubstituted or substituted by one or more radicals selected from the group consisting of halogen, nitro, hydroxy, cyano, NR¹⁰R¹¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxy-carbonyl, (C₁-C₄)-haloalkoxy-carbonyl, (C₁-C₄)-alkylcarboxy, (C₃-C₆)-cycloalkyl, (C₃-C₆)-cycloalkyl-(C₁-C₆)-alkyl, (C₁-C₄)-alkoxy-carbonyl-(C₁—C₄)-alkyl, hydroxycarbonyl, hydroxycarbonyl-(C₁-C₄)-alkyl, R¹⁰R¹¹N-carbonyl, and where the structural elements cycloalkyl, cycloalkenyl and heterocyclyl have n oxo groups, where n=0, 1 or 2.

In the context of the present invention, more preference is given to the following compounds of the formula (I) in which:
X represents oxygen,
A represents the grouping C—R¹ or the grouping N—R⁹ (N=nitrogen), where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents hydrogen, halogen, (C₁-C₄)-alkyl, (C₁-C₄)-haloalkyl, (C₁-C₄)-alkoxy, (C₁-C₄)-haloalkoxy or (C₅-C₇)-cycloalkyl,
R² represents hydrogen, halogen, (C₁-C₄)-alkyl, (C₁-C₄)-haloalkyl, (C₁-C₄)-alkoxy, (C₁-C₄)-haloalkoxy or (C₅-C₇)-cycloalkyl,
R³ represents hydroxy, arylcarbonyloxy, (C₁-C₄)-alkylcarbonyloxy, aryl-(C₁-C₄)-alkylcarbonyloxy, (C₁-C₄)-haloalkyl-carbonyloxy, OC(O)OR¹² or OSO₂R¹³,
R⁴ represents hydrogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₆)-alkyl, heteroaryl-(C₁-C₆)-alkyl, heterocyclyl-(C₁-C₆)-alkyl, (C₂-C₆)-alkenyl, (C₂-C₆)-alkynyl, (C₂-C₆)-haloalkenyl, (C₂-C₆)-haloalkynyl, (C₄-C₈)-cycloalkenyl, aryl-(C₂-C₆)-alkenyl, heteroaryl-(C₂-C₆)-alkenyl, heterocyclyl-(C₂-C₆)-alkenyl, arylcarbonyl-(C₁-C₆)-alkyl, (C₁-C₆)-alkylcarbonyl-(C₁-C₆)-alkyl, heteroarylcarbonyl-(C₁-C₆)-alkyl, (C₃-C₁₀)-cycloalkylcarbonyl-(C₁-C₆)-alkyl, aryl-(C₁-C₆)-alkoxycarbonyl-(C₁-C₆)-alkyl, (C₁-C₆)-alkoxycarbonyl-(C₁-C₆)-alkyl, (C₁-C₆)-alkoxy-(C₁-C₆)-alkyl, (C₁-C₆)-haloalkoxy-(C₁-C₆)-alkyl, C(O)R¹², SO₂R¹³, CHO, R¹⁰R¹¹N—(C₁-C₈)-alkyl or cyano-(C₁-C₈)-alkyl,
R⁵ represents hydrogen, (C₁-C₄)-alkyl, (C₁-C₄)-haloalkyl, (C₅-C₈)-cycloalkyl, aryl, heteroaryl, heterocyclyl, C(O)OR¹², C(O)NR¹⁰R¹¹ or R¹²O(O)C—(C₁-C₄)-alkyl,
R⁶ represents hydrogen, (C₁-C₄)-alkyl or (C₁-C₄)-haloalkyl,
R⁷ and R⁸ are identical or different and independently of one another represent hydrogen, halogen or (C₁-C₄)-alkyl,
R⁹ represents hydrogen, hydroxy, (C₁-C₄)-alkyl, (C₁-C₄)-haloalkyl, (C₁-C₄)-alkoxy or (C₃-C₆)-cycloalkyl,
   or where R² and R⁹ together with the nitrogen atom or carbon atom to which they are respectively attached form a preferably fully saturated ring which has 3 to 7 members in total, which is optionally interrupted by one heteroatom from the group consisting of N and O and which is optionally substituted further,
R¹⁰ and R¹¹ are identical or different and independently of one another represent hydrogen, (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-haloalkoxy-(C₁-C₄)-alkyl, (C₁-C₄)-alkylthio-(C₁-C₄)-alkyl, (C₁-C₄)-haloalkylthio-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₅)-cycloalkyl-(C₁-C₄)-alkyl, (C₄-C₇)-cycloalkenyl-(C₁-C₄)-alkyl, C(O)R¹², SO₂R¹³, heterocyclyl, (C₁-C₄)-alkoxycarbonyl-(C₁-C₄)-alkyl, (C₁-C₄)-alkoxycarbonyl, aryl-(C₁-C₄)-alkoxycarbonyl-(C₁-C₄)-alkyl, aryl-(C₁-C₄)-alkoxycarbonyl, heteroaryl-(C₁-C₄)-alkoxycarbonyl, (C₂-C₄)-alkenyloxycarbonyl, (C₂-C₄)-alkynyloxycarbonyl or heterocyclyl-(C₁-C₄)-alkyl,
R¹² represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₅)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₅)-cycloalkyl-(C₁-C₄)-alkyl, (C₄-C₇)-cycloalkenyl-(C₁-C₄)-alkyl, (C₁-C₄)-alkoxycarbonyl-(C₁-C₄)-alkyl, (C₂-C₄)-alkenyloxycarbonyl-(C₁-C₄)-alkyl, aryl-(C₁-C₄)-alkoxycarbonyl-(C₁-C₄)-alkyl, hydroxycarbonyl-(C₁-C₄)-alkyl, heterocyclyl or heterocyclyl-(C₁-C₄)-alkyl,
R¹³ represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, heterocyclyl-(C₁-C₄)-alkyl, (C₃-C₈)-cycloalkyl-(C₁-C₄)-alkyl, (C₄-C₇)-cycloalkenyl-(C₁-C₄)-alkyl or NR¹⁰R¹¹,
R¹⁴ represents hydrogen,
   or where R³ and R¹⁴ together with the carbon atom to which they are attached form a carbonyl group,

where the cyclic structural elements (in particular the structural elements aryl, cycloalkyl, cycloalkenyl, heteroaryl and heterocyclyl) of the radicals mentioned respectively in R¹, R², R³, R⁴, R⁵, R⁶, R⁹, R¹⁰, R¹¹, R¹² and R¹³ and the ring optionally formed by R² and R⁹, having 3 to 7 members in total, are in each case unsubstituted or substituted by one or more radicals selected from the group consisting of halogen, nitro, hydroxy, cyano, NR¹⁰R¹¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxy-carbonyl, (C₁-C₄)-haloalkoxy-carbonyl, (C₁-C₄)-alkylcarboxy, (C₃-C₆)-cycloalkyl, (C₃-C₆)-cycloalkyl-(C₁-C₆)-alkyl, (C₁-C₄)-alkoxy-carbonyl-(C₁-C₄)-alkyl, hydroxycarbonyl, hydroxycarbonyl-(C₁-C₄)-alkyl, R¹⁰R¹¹N-carbonyl, and where the structural elements cycloalkyl, cycloalkenyl and heterocyclyl have n oxo groups, where n=0, 1 or 2.

In the context of the present invention, even more preference is given to the following compounds of the formula (I) in which:
X represents oxygen,
A represents the grouping C—R¹ or the grouping N—R⁹ (N=nitrogen), where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents hydrogen, halogen, (C₁-C₄)-alkyl, (C₁-C₄)-alkoxy or (C₅-C₇)-cycloalkyl,
R² represents hydrogen, halogen, (C₁-C₄)-alkyl, (C₁-C₄)-alkoxy or (C₅-C₇)-cycloalkyl,
R³ represents hydroxy, arylcarbonyloxy, (C₁-C₄)-alkylcarbonyloxy, (C₁-C₄)-haloalkyl-carbonyloxy, OC(O)O—(C₁-C₄)-alkyl, OSO₂—(C₁-C₄)-alkyl or OSO₂-aryl,
R⁴ represents hydrogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₆)-alkyl, heteroaryl-(C₁-C₆)-alkyl, heterocyclyl-(C₁-C₆)-alkyl, (C₂-C₆)-alkenyl, (C₂-C₆)-alkynyl, (C₂-C₆)-haloalkenyl, (C₂-C₆)-haloalkynyl, (C₄-C₈)-cycloalkenyl, aryl-(C₂-C₆)-alkenyl, heteroaryl-(C₂-C₆)-alkenyl, heterocyclyl-(C₂-C₆)-alkenyl, arylcarbonyl-(C₁-C₆)-alkyl, (C₁-C₆)-alkylcarbonyl-(C₁-C₆)-alkyl, heteroarylcarbonyl-(C₁-C₆)-alkyl, (C₃-C₁₀)-cycloalkylcarbonyl-(C₁-C₆)-alkyl, aryl-(C₁-C₆)-alkoxycarbonyl-(C₁-C₆)-alkyl, (C₁-C₆)-alkoxycarbonyl-(C₁-C₆)-alkyl, (C₁-C₆)-alkoxy-(C₁-C₆)-alkyl, (C₁-C₆)-haloalkoxy-(C₁-C₆)-alkyl, C(O)R¹², SO₂R¹³, CHO, R¹⁰R¹¹N—(C₁-C₈)-alkyl or cyano-(C₁-C₈)-alkyl,
R⁵ represents hydrogen, (C₁-C₃)-alkyl, (C₁-C₃)-haloalkyl, aryl, heteroaryl, C(O)O—(C₁-C₄)-alkyl or (C₁-C₄)-alkyl-O(O)C—(C₁-C₄)-alkyl,
R⁶ is hydrogen or (C₁-C₄)-alkyl,
R⁷ and R⁸ are identical or different and independently of one another represent hydrogen, bromine, chlorine or (C₁-C₃)-alkyl,
R⁹ represents hydrogen, hydroxy, (C₁-C₄)-alkyl, (C₁-C₄)-haloalkyl, (C₁-C₄)-alkoxy or (C₃-C₆)-cycloalkyl,
   or where R² and R⁹ together with the nitrogen atom or carbon atom to which they are respectively attached form a fully saturated ring which has 4 to 6 members in total, which is optionally interrupted by one heteroatom from the group consisting of N and O and which is optionally substituted further,
R¹⁰ and R¹¹ are identical or different and independently of one another represent hydrogen, (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-haloalkoxy-(C₁-C₄)-alkyl, (C₁-C₄)-alkylthio-(C₁-C₄)-alkyl, (C₁-C₄)-haloalkylthio-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₅)-cycloalkyl-(C₁-C₄)-alkyl, (C₄-C₇)-cycloalkenyl-(C₁-C₄)-alkyl, C(O)R¹², SO₂R¹³, heterocyclyl, (C₁-C₄)-alkoxycarbonyl-(C₁-C₄)-alkyl, (C₁-C₄)-alkoxycarbonyl, aryl-(C₁-C₄)-alkoxycarbonyl-(C₁-C₄)-alkyl, aryl-(C₁-C₄)-alkoxycarbonyl, heteroaryl-(C₁-C₄)-alkoxycarbonyl, (C₂-C₄)-alkenyloxycarbonyl, (C₂-C₄)-alkynyloxycarbonyl or heterocyclyl-(C₁-C₄)-alkyl,
R¹² represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₅)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₅)-cycloalkyl-(C₁-C₄)-alkyl, (C₄-C₇)-cycloalkenyl-(C₁-C₄)-alkyl, (C₁-C₄)-alkoxycarbonyl-(C₁-C₄)-alkyl, (C₂-C₄)-alkenyloxycarbonyl-(C₁-C₄)-alkyl, aryl-(C₁-C₄)-alkoxycarbonyl-(C₁-C₄)-alkyl, hydroxycarbonyl-(C₁-C₄)-alkyl, heterocyclyl or heterocyclyl-(C₁-C₄)-alkyl,
R¹³ represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, heterocyclyl-(C₁-C₄)-alkyl, (C₃-C₅)-cycloalkyl-(C₁-C₄)-alkyl, (C₄-C₇)-cycloalkenyl-(C₁-C₄)-alkyl or NR¹⁰R¹¹,
R¹⁴ represents hydrogen,
   or where R³ and R¹⁴ together with the carbon atom to which they are attached form a carbonyl group,

where the cyclic structural elements (in particular the structural elements aryl, cycloalkyl, cycloalkenyl, heteroaryl and heterocyclyl) of the radicals mentioned respectively in R¹, R², R³, R⁴, R⁵, R⁹, R¹⁰, R¹¹, R¹² and R¹² and the ring optionally formed by R² and R⁹, having 4 to 6 members in total, are in each case unsubstituted or substituted by one or more radicals selected from the group consisting of halogen, nitro, hydroxy, cyano, NR¹⁰R¹¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxy-carbonyl, (C₁-C₄)-haloalkoxy-carbonyl, (C₁-C₄)-alkylcarboxy, (C₃-C₆)-cycloalkyl, (C₃-C₆)-cycloalkyl-(C₁-C₆)-alkyl, (C₁-C₄)-alkoxy-carbonyl-(C₁-C₄)-alkyl, hydroxycarbonyl, hydroxycarbonyl-(C₁-C₄)-alkyl, R¹⁰R¹¹N-carbonyl, and where the structural elements cycloalkyl, cycloalkenyl and heterocyclyl have n oxo groups, where n=0, 1 or 2.

In the context of the present invention, even more preference is given to the following compounds of the formula (I) in which:
X represents oxygen,
A represents the grouping C—R¹ or the grouping N—R⁹ (N=nitrogen) where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents hydrogen, halogen, (C₁-C₃)-alkyl, (C₁-C₃)-alkoxy or (C₅-C₇)-cycloalkyl,
R² represents hydrogen, halogen, (C₁-C₃)-alkyl, (C₁-C₃)-alkoxy or (C₅-C₇)-cycloalkyl,
R³ represents hydroxy, arylcarbonyloxy, (C₁-C₄)-alkylcarbonyloxy, OC(O)O—(C₁-C₄)-alkyl or OSO₂—(C₁-C₄)-alkyl,
R⁴ represents hydrogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₆)-alkyl, heteroaryl-(C₁-C₆)-alkyl, heterocyclyl-(C₁-C₆)-alkyl, (C₂-C₆)-alkenyl, (C₂-C₆)-alkynyl, (C₂-C₆)-haloalkenyl, (C₂-C₆)-haloalkynyl, (C₄-C₈)-cycloalkenyl, aryl-(C₂-C₆)-alkenyl, heteroaryl-(C₂-C₆)-alkenyl, heterocyclyl-(C₂-C₆)-alkenyl, arylcarbonyl-(C₁-C₆)-alkyl, (C₁-C₆)-alkylcarbonyl-(C₁-C₆)-alkyl, heteroarylcarbonyl-(C₁-C₆)-alkyl, (C₃-C₁₀)-cycloalkylcarbonyl-(C₁-C₆)-alkyl, aryl-(C₁-C₆)-alkoxycarbonyl-(C₁-C₆)-alkyl, (C₁-C₆)-alkoxycarbonyl-(C₁-C₆)-alkyl, (C₁-C₆)-alkoxy-(C₁-C₆)-alkyl, (C₁-C₆)-haloalkoxy-(C₁-C₆)-alkyl, C(O)R¹², SO₂R¹³, CHO, R¹⁰R¹¹N—(C₁-C₈)-alkyl or cyano-(C₁-C₈)-alkyl,
R⁵ represents hydrogen, (C₁-C₃)-alkyl, (C₁-C₃)-haloalkyl, phenyl, N-heteroaryl, C(O)O—(C₁-C₄)-alkyl or (C₁-C₄)-alkyl-O(O)C—(C₁-C₄)-alkyl,
R⁶ represents hydrogen or (C₁-C₃)-alkyl,
R⁷ represents hydrogen, bromine, chlorine or (C₁-C₃)-alkyl,
R⁸ represents hydrogen, bromine or chlorine,
R⁹ represents (C₁-C₄)-alkyl or (C₁-C₄)-alkoxy,
   or where R² and R⁹ together with the nitrogen atom or carbon atom to which they are respectively attached form a fully saturated ring which has 5 or 6 members in total, which is optionally interrupted by one heteroatom from the group consisting of N and O and which is optionally substituted further,
R¹⁰ and R¹¹ are identical or different and independently of one another represent hydrogen, (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₅)-cycloalkyl, (C₄-C₇)-cycloalkenyl, (C₁-C₄)-alkoxy-(C₁-C₄)-alkyl, (C₁-C₄)-haloalkoxy-(C₁-C₄)-alkyl, (C₁-C₄)-alkylthio-(C₁-C₄)-alkyl, (C₁-C₄)-haloalkylthio-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₅)-cycloalkyl-(C₁-C₄)-alkyl, (C₄-C₇)-cycloalkenyl-(C₁-C₄)-alkyl, C(O)R¹², SO₂R¹³, heterocyclyl, (C₁-C₄)-alkoxycarbonyl-(C₁-C₄)-alkyl, (C₁-C₄)-alkoxycarbonyl, aryl-(C₁-C₄)-alkoxycarbonyl-(C₁-C₄)-alkyl, aryl-(C₁-C₄)-alkoxycarbonyl, heteroaryl-(C₁-C₄)-alkoxycarbonyl, (C₂-C₄)-alkenyloxycarbonyl, (C₂-C₄)-alkynyloxycarbonyl or heterocyclyl-(C₁-C₄)-alkyl,
R¹² represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₅)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₅)-cycloalkyl-(C₁-C₄)-alkyl, (C₄-C₇)-cycloalkenyl-(C₁-C₄)-alkyl, (C₁-C₄)-alkoxycarbonyl-(C₁-C₄)-alkyl, (C₂-C₄)-alkenyloxycarbonyl-(C₁-C₄)-alkyl, aryl-(C₁-C₄)-alkoxycarbonyl-(C₁-C₄)-alkyl, hydroxycarbonyl-(C₁-C₄)-alkyl, heterocyclyl or heterocyclyl-(C₁-C₄)-alkyl,
R¹³ represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, heterocyclyl-(C₁-C₄)-alkyl, (C₃-C₅)-cycloalkyl-(C₁-C₄)-alkyl, (C₄-C₇)-cycloalkenyl-(C₁-C₄)-alkyl or NR¹⁰R¹¹,
R¹⁴ represents hydrogen,
   or where R³ and R¹⁴ together with the carbon atom to which they are attached form a carbonyl group,

where the cyclic structural elements (in particular the structural elements aryl, cycloalkyl, cycloalkenyl, heteroaryl and heterocyclyl) of the radicals mentioned respectively in R⁴, R⁵, R¹⁰, R¹¹, R¹² and R¹³ and the ring optionally formed by R² and R⁹, having 5 or 6 members in total, are in each case unsubstituted or substituted by one or more radicals selected from the group consisting of halogen, nitro, hydroxy, cyano, NR¹⁰R¹¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxy-carbonyl, (C₁-C₄)-haloalkoxy-carbonyl, (C₁-C₄)-alkylcarboxy, (C₃-C₆)-cycloalkyl, (C₃-C₆)-cycloalkyl-(C₁-C₆)-alkyl, (C₁-C₄)-alkoxy-carbonyl-(C₁-C₄)-alkyl, hydroxycarbonyl, hydroxycarbonyl-(C₁-C₄)-alkyl, R¹⁰R¹¹N-carbonyl, and where the structural elements cycloalkyl, cycloalkenyl and heterocyclyl have n oxo groups, where n=0, 1 or 2.

In the context of the present invention, more preference is given to compounds of the formula (I) in which:

R¹ represents hydrogen, methyl, chlorine, bromine, methoxy or cyclohexyl,

and/or

R² represents hydrogen, methyl, chlorine, bromine, methoxy or cyclohexyl.

In the context of the present invention, preference is furthermore given to compounds of the formula (I) in which:

R⁹ represents methyl, ethyl, isopropyl or methoxy, where R² preferably simultaneously represents hydrogen or methyl,

or R² and R⁹ together with the nitrogen atom or carbon atom to which they are attached form a fully saturated ring which has a total of 5 members and which is optionally interrupted by a further heteroatom from the group consisting of N and O, preferably by O.

In the context of the present invention, R³ preferably represents hydroxy, OC(O)CH₃ (acetoxy), OC(O)CH₂CH₃ (propionyloxy), OC(O)OCH₃ (methylcarbonato), or OSO₂CH₃ (mesylate) and simultaneously R¹⁴ represents hydrogen,

or R³ and R¹⁴ together with the carbon atom to which they are attached form a carbonyl group.

In the context of the present invention, R⁴ particularly preferably represents the radicals listed in Table 1 below.

In the context of the present invention, preference is given to compounds of the formula (I) in which:

R⁵ represents hydrogen, methyl, trifluoromethyl, CO₂CH₃, CH₂CO₂CH₃, CH₂OC(O)CH₃, optionally substituted phenyl (and here preferably chlorophenyl or methoxyphenyl) or pyridinyl (and here preferably pyridin-2-yl), and R⁶ represents hydrogen or methyl.

In the context of the present invention, R⁵ and R⁶ each particularly preferably represents the radicals listed in Table 1 below.

In the context of the present invention, preference is given to compounds of the formula (I) in which:

R⁷ represents hydrogen, bromine, chlorine or methyl, and

R⁸ represents hydrogen, bromine or chlorine.

In the context of the present invention, preference is given to compounds of the formula (I) in which:

R⁵ represents hydrogen, methyl, trifluoromethyl, CO₂CH₃, CH₂CO₂CH₃, CH₂OC(O)CH₃, optionally substituted phenyl (and here preferably chlorophenyl or methoxyphenyl) or pyridinyl (and here preferably pyridin-2-yl), and R⁶ represents hydrogen or methyl, R⁷ represents hydrogen or methyl and R⁸ represents hydrogen.

In the context of the present invention, particular preference is given to compounds of the formula (I) in which:
X represents oxygen,
A represents the grouping C—R¹ or the grouping N—R⁹ (N=nitrogen), where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents hydrogen, methyl, chlorine, bromine, methoxy or cyclohexyl,
R² represents hydrogen, methyl, chlorine, bromine, methoxy or cyclohexyl,
R³ represents hydroxy, OC(O)CH₃ (acetoxy), OC(O)CH₂CH₃ (propionyloxy), OC(O)OCH₃ (methylcarbonato), or OSO₂CH₃ (mesylate) and simultaneously R¹⁴ represents hydrogen,
   or R³ and R¹⁴ together with the carbon atom to which they are attached form a carbonyl group,
R⁴ represents hydrogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₆)-alkyl, heteroaryl-(C₁-C₆)-alkyl, heterocyclyl-(C₁-C₆)-alkyl, (C₂-C₆)-alkenyl, (C₂-C₆)-alkynyl, (C₂-C₆)-haloalkenyl, (C₂-C₆)-haloalkynyl, (C₄-C₈)-cycloalkenyl, aryl-(C₂-C₆)-alkenyl, heteroaryl-(C₂-C₆)-alkenyl, heterocyclyl-(C₂-C₆)-alkenyl, arylcarbonyl-(C₁-C₆)-alkyl, (C₁-C₆)-alkylcarbonyl-(C₁-C₆)-alkyl, heteroarylcarbonyl-(C₁-C₆)-alkyl, (C₃-C₁₀)-cycloalkylcarbonyl-(C₁-C₆)-alkyl, aryl-(C₁-C₆)-alkoxycarbonyl-(C₁-C₆)-alkyl, (C₁-C₆)-alkoxycarbonyl-(C₁-C₆)-alkyl, (C₁-C₆)-alkoxy-(C₁-C₆)-alkyl, (C₁-C₆)-haloalkoxy-(C₁-C₆)-alkyl, C(O)R¹², SO₂R¹³, CHO, R¹⁰R¹¹N—(C₁-C₈)-alkyl or cyano-(C₁-C₈)-alkyl, where R⁴ represents in particular the radicals listed in Table 1 below,
R⁵ represents hydrogen, methyl, trifluoromethyl, CO₂CH₃, CH₂CO₂CH₃, CH₂OC(O)CH₃, optionally substituted phenyl (and here preferably chlorophenyl or methoxyphenyl) or pyridinyl (and here preferably pyridin-2-yl), and R⁶ represents hydrogen or methyl,
R⁷ represents hydrogen, bromine, chlorine or methyl,
R⁸ represents hydrogen, bromine or chlorine,
R⁹ represents methyl, ethyl, isopropyl or methoxy, where R² preferably simultaneously represents hydrogen or methyl,
   or R² and R⁹ together with the nitrogen atom or carbon atom to which they are attached form a fully saturated ring which has a total of 5 members and which is optionally interrupted by a further heteroatom from the group consisting of N and O, preferably by O,
R¹⁰ and R¹¹ are identical or different and independently of one another represent hydrogen, (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₅)-cycloalkyl, (C₄-C₇)-cycloalkenyl, (C₁-C₄)-alkoxy-(C₁-C₄)-alkyl, (C₁-C₄)-haloalkoxy-(C₁-C₄)-alkyl, (C₁-C₄)-alkylthio-(C₁-C₄)-alkyl, (C₁-C₄)-haloalkylthio-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₅)-cycloalkyl-(C₁-C₄)-alkyl, (C₄-C₇)-cycloalkenyl-(C₁-C₄)-alkyl, C(O)R¹², SO₂R¹³, heterocyclyl, (C₁-C₄)-alkoxycarbonyl-(C₁-C₄)-alkyl, (C₁-C₄)-alkoxycarbonyl, aryl-(C₁-C₄)-alkoxycarbonyl-(C₁-C₄)-alkyl, aryl-(C₁-C₄)-alkoxycarbonyl, heteroaryl-(C₁-C₄)-alkoxycarbonyl, (C₂-C₄)-alkenyloxycarbonyl, (C₂-C₄)-alkynyloxycarbonyl or heterocyclyl-(C₁-C₄)-alkyl,
R¹² represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₈)-cycloalkyl-(C₁-C₄)-alkyl, (C₄-C₇)-cycloalkenyl-(C₁-C₄)-alkyl, (C₁-C₄)-alkoxycarbonyl-(C₁-C₄)-alkyl, (C₂-C₄)-alkenyloxycarbonyl-(C₁-C₄)-alkyl, aryl-(C₁-C₄)-alkoxycarbonyl-(C₁-C₄)-alkyl, hydroxycarbonyl-(C₁-C₄)-alkyl, heterocyclyl or heterocyclyl-(C₁-C₄)-alkyl,
R¹³ represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, heterocyclyl-(C₁-C₄)-alkyl, (C₃-C₈)-cycloalkyl-(C₁-C₄)-alkyl, (C₄-C₇)-cycloalkenyl-(C₁-C₄)-alkyl or NR¹⁰R¹¹,

where the cyclic structural elements (in particular the structural elements aryl, cycloalkyl, cycloalkenyl, heteroaryl and heterocyclyl) of the radicals mentioned respectively in R⁴, R⁵, R¹⁰, R¹¹, R¹² and R¹³ and the ring optionally formed by R² and R⁹, having 5 members in total, are in each case unsubstituted or substituted by one or more radicals selected from the group consisting of halogen, nitro, hydroxy, cyano, NR¹⁰R¹¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxy-carbonyl, (C₁-C₄)-haloalkoxy-carbonyl, (C₁-C₄)-alkylcarboxy, (C₃-C₆)-cycloalkyl, (C₃-C₆)-cycloalkyl-(C₁-C₆)-alkyl, (C₁-C₄)-alkoxy-carbonyl-(C₁-C₄)-alkyl, hydroxycarbonyl, hydroxycarbonyl-(C₁-C₄)-alkyl, R¹⁰R¹¹N-carbonyl, and where the structural elements cycloalkyl, cycloalkenyl and heterocyclyl have n oxo groups, where n=0, 1 or 2.

In the context of the present invention, particular preference is given to compounds of the formula (I) in which:
X represents oxygen,
A represents the grouping C—R¹ or the grouping N—R⁹ (N=nitrogen), where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents methyl, chlorine, bromine, methoxy or cyclohexyl,
R² represents hydrogen, methyl, chlorine, bromine, methoxy or cyclohexyl,
R³ represents hydroxy, OC(O)CH₃ (acetoxy), OC(O)CH₂CH₃ (propionyloxy), OC(O)OCH₃ (methylcarbonato), or OSO₂CH₃ (mesylate) and simultaneously R¹⁴ represents hydrogen,
   or R³ and R¹⁴ together with the carbon atom to which they are attached form a carbonyl group,
R⁴ represents hydrogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₆)-alkyl, heteroaryl-(C₁-C₆)-alkyl, heterocyclyl-(C₁-C₆)-alkyl, (C₂-C₆)-alkenyl, (C₂-C₆)-alkynyl, (C₂-C₆)-haloalkenyl, (C₂-C₆)-haloalkynyl, (C₄-C₈)-cycloalkenyl, aryl-(C₂-C₆)-alkenyl, heteroaryl-(C₂-C₆)-alkenyl, heterocyclyl-(C₂-C₆)-alkenyl, arylcarbonyl-(C₁-C₆)-alkyl, (C₁-C₆)-alkylcarbonyl-(C₁-C₆)-alkyl, heteroarylcarbonyl-(C₁-C₆)-alkyl, (C₃-C₁₀)-cycloalkylcarbonyl-(C₁-C₆)-alkyl, aryl-(C₁-C₆)-alkoxycarbonyl-(C₁-C₆)-alkyl, (C₁-C₆)-alkoxycarbonyl-(C₁-C₆)-alkyl, (C₁-C₆)-alkoxy-(C₁-C₆)-alkyl, (C₁-C₆)-haloalkoxy-(C₁-C₆)-alkyl, C(O)R¹², SO₂R¹³, CHO, R¹⁰R¹¹N—(C₁-C₈)-alkyl or cyano-(C₁-C₈)-alkyl, where R⁴ represents in particular the radicals listed in Table 1 below,
R⁵ represents hydrogen, methyl, trifluoromethyl, CO₂CH₃, CH₂CO₂CH₃, CH₂OC(O)CH₃, optionally substituted phenyl (and here preferably chlorophenyl or methoxyphenyl) or pyridinyl (and here preferably pyridin-2-yl), and R⁶ represents hydrogen or methyl,
R⁷ represents hydrogen or methyl,
R⁸ represents hydrogen,
R⁹ represents methyl, ethyl, isopropyl or methoxy, where R² preferably simultaneously represents hydrogen or methyl,
   or R² and R⁹ together with the nitrogen atom or carbon atom to which they are attached form a fully saturated ring which has a total of 5 members and which is optionally interrupted by a further heteroatom from the group consisting of N and O, preferably by O,
R¹⁰ and R¹¹ are identical or different and independently of one another represent hydrogen, (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₅)-cycloalkyl, (C₄-C₇)-cycloalkenyl, (C₁-C₄)-alkoxy-(C₁-C₄)-alkyl, (C₁-C₄)-haloalkoxy-(C₁-C₄)-alkyl, (C₁-C₄)-alkylthio-(C₁-C₄)-alkyl, (C₁-C₄)-haloalkylthio-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₈)-cycloalkyl-(C₁-C₄)-alkyl, (C₄-C₇)-cycloalkenyl-(C₁-C₄)-alkyl, C(O)R¹², SO₂R¹³, heterocyclyl, (C₁-C₄)-alkoxycarbonyl-(C₁-C₄)-alkyl, (C₁-C₄)-alkoxycarbonyl, aryl-(C₁-C₄)-alkoxycarbonyl-(C₁-C₄)-alkyl, aryl-(C₁-C₄)-alkoxycarbonyl, heteroaryl-(C₁-C₄)-alkoxycarbonyl, (C₂-C₄)-alkenyloxycarbonyl, (C₂-C₄)-alkynyloxycarbonyl or heterocyclyl-(C₁-C₄)-alkyl,
R¹² represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₈)-cycloalkyl-(C₁-C₄)-alkyl, (C₄-C₇)-cycloalkenyl-(C₁-C₄)-alkyl, (C₁-C₄)-alkoxycarbonyl-(C₁-C₄)-alkyl, (C₂-C₄)-alkenyloxycarbonyl-(C₁-C₄)-alkyl, aryl-(C₁-C₄)-alkoxycarbonyl-(C₁-C₄)-alkyl, hydroxycarbonyl-(C₁-C₄)-alkyl, heterocyclyl or heterocyclyl-(C₁-C₄)-alkyl,
R¹³ represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, heterocyclyl-(C₁-C₄)-alkyl, (C₃-C₈)-cycloalkyl-(C₁-C₄)-alkyl, (C₄-C₇)-cycloalkenyl-(C₁-C₄)-alkyl or NR¹⁰R¹¹,

where the cyclic structural elements (in particular the structural elements aryl, cycloalkyl, cycloalkenyl, heteroaryl and heterocyclyl) of the radicals mentioned respectively in R⁴, R⁵, R¹⁰, R¹¹, R¹² and R¹³ and the ring optionally formed by R² and R⁹, having 5 members in total, are in each case unsubstituted or substituted by one or more radicals selected from the group consisting of halogen, nitro, hydroxy, cyano, NR¹⁰R¹¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxy-carbonyl, (C₁-C₄)-haloalkoxy-carbonyl, (C₁-C₄)-alkylcarboxy, (C₃-C₆)-cycloalkyl, (C₃-C₆)-cycloalkyl-(C₁-C₆)-alkyl, (C₁-C₄)-alkoxy-carbonyl-(C₁-C₄)-alkyl, hydroxycarbonyl, hydroxycarbonyl-(C₁-C₄)-alkyl, R¹⁰R¹¹N-carbonyl, and where the structural elements cycloalkyl, cycloalkenyl and heterocyclyl have n oxo groups, where n=0, 1 or 2.

Furthermore, particular preference in the context of the present invention is given to the combinations of the definitions of R⁴, R⁵, R⁶, R⁷ and R⁸ given in Table 1 below.

If in Table 1 for the radicals R⁴, R⁵ and R⁶ a structural element is defined by a structural formula containing a broken line, this broken line means that at this position R⁴, R⁵ or R⁶ is attached to the remainder of the molecule.

TABLE 1

| No. | R⁴ | R⁵ | R⁶ | R⁷ | R⁸ |
|---|---|---|---|---|---|
| 1 | H | CH₃ | CH₃ | H | H |
| 2 | CH₃ | CH₃ | CH₃ | H | H |
| 3 | Et | CH₃ | CH₃ | H | H |
| 4 | n-Pr | CH₃ | CH₃ | H | H |
| 5 | i-Pr | CH₃ | CH₃ | H | H |
| 6 | n-Bu | CH₃ | CH₃ | H | H |
| 7 | neopentyl | CH₃ | CH₃ | H | H |
| 8 | t-Bu | CH₃ | CH₃ | H | H |
| 9 | n-pentyl | CH₃ | CH₃ | H | H |
| 10 | n-hexyl | CH₃ | CH₃ | H | H |
| 11 | c-Pr | CH₃ | CH₃ | H | H |
| 12 | c-Bu | CH₃ | CH₃ | H | H |
| 13 | c-pentyl | CH₃ | CH₃ | H | H |
| 14 | c-hexyl | CH₃ | CH₃ | H | H |
| 15 | 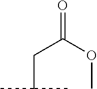 | CH₃ | CH₃ | H | H |
| 16 | 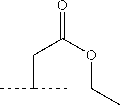 | CH₃ | CH₃ | H | H |
| 17 | 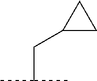 | CH₃ | CH₃ | H | H |
| 18 | 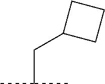 | CH₃ | CH₃ | H | H |
| 19 | 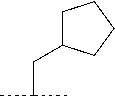 | CH₃ | CH₃ | H | H |
| 20 | 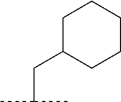 | CH₃ | CH₃ | H | H |
| 21 | phenyl | CH₃ | CH₃ | H | H |
| 22 | 2-fluorophenyl | CH₃ | CH₃ | H | H |
| 23 | 3-fluorophenyl | CH₃ | CH₃ | H | H |
| 24 | 4-fluorophenyl | CH₃ | CH₃ | H | H |
| 25 | 2,4-difluorophenyl | CH₃ | CH₃ | H | H |
| 26 | 2,5-difluorophenyl | CH₃ | CH₃ | H | H |
| 27 | 2,6-difluorophenyl | CH₃ | CH₃ | H | H |
| 28 | 2,3-difluorophenyl | CH₃ | CH₃ | H | H |
| 29 | 3,4-difluorophenyl | CH₃ | CH₃ | H | H |
| 30 | 3,5-difluorophenyl | CH₃ | CH₃ | H | H |
| 31 | 2,4,5-trifluorophenyl | CH₃ | CH₃ | H | H |
| 32 | 3,4,5-trifluorophenyl | CH₃ | CH₃ | H | H |
| 33 | 2-chlorophenyl | CH₃ | CH₃ | H | H |
| 34 | 3-chlorophenyl | CH₃ | CH₃ | H | H |
| 35 | 4-chlorophenyl | CH₃ | CH₃ | H | H |
| 36 | 2,4-dichlorophenyl | CH₃ | CH₃ | H | H |
| 37 | 2,5-dichlorophenyl | CH₃ | CH₃ | H | H |
| 38 | 2,6-dichlorophenyl | CH₃ | CH₃ | H | H |
| 39 | 2,3-dichlorophenyl | CH₃ | CH₃ | H | H |
| 40 | 3,4-dichlorophenyl | CH₃ | CH₃ | H | H |
| 41 | 3,5-dichlorophenyl | CH₃ | CH₃ | H | H |
| 42 | 2,4,5-trichlorophenyl | CH₃ | CH₃ | H | H |
| 43 | 3,4,5-trichlorophenyl | CH₃ | CH₃ | H | H |
| 44 | 2,4,6-trichlorophenyl | CH₃ | CH₃ | H | H |
| 45 | 2-bromophenyl | CH₃ | CH₃ | H | H |
| 46 | 3-bromophenyl | CH₃ | CH₃ | H | H |
| 47 | 4-bromophenyl | CH₃ | CH₃ | H | H |
| 48 | 2-iodophenyl | CH₃ | CH₃ | H | H |
| 49 | 3-iodophenyl | CH₃ | CH₃ | H | H |
| 50 | 4-iodophenyl | CH₃ | CH₃ | H | H |
| 51 | 2-bromo-4-fluorophenyl | CH₃ | CH₃ | H | H |
| 52 | 2-bromo-4-chlorophenyl | CH₃ | CH₃ | H | H |
| 53 | 3-bromo-4-fluorophenyl | CH₃ | CH₃ | H | H |
| 54 | 3-bromo-4-chlorophenyl | CH₃ | CH₃ | H | H |
| 55 | 3-bromo-5-fluorophenyl | CH₃ | CH₃ | H | H |
| 56 | 3-bromo-5-chlorophenyl | CH₃ | CH₃ | H | H |
| 57 | 2-fluoro-4-bromophenyl | CH₃ | CH₃ | H | H |
| 58 | 2-chloro-4-bromophenyl | CH₃ | CH₃ | H | H |
| 59 | 3-fluoro-4-bromophenyl | CH₃ | CH₃ | H | H |
| 60 | 3-chloro-4-bromophenyl | CH₃ | CH₃ | H | H |
| 61 | 2-chloro-4-fluorophenyl | CH₃ | CH₃ | H | H |
| 62 | 3-chloro-4-fluorophenyl | CH₃ | CH₃ | H | H |
| 63 | 2-fluoro-3-chlorophenyl | CH₃ | CH₃ | H | H |
| 64 | 2-fluoro-4-chlorophenyl | CH₃ | CH₃ | H | H |
| 65 | 2-fluoro-5-chlorophenyl | CH₃ | CH₃ | H | H |
| 66 | 3-fluoro-4-chlorophenyl | CH₃ | CH₃ | H | H |
| 67 | 3-fluoro-5-chlorophenyl | CH₃ | CH₃ | H | H |
| 68 | 2-fluoro-6-chlorophenyl | CH₃ | CH₃ | H | H |
| 69 | 2-methylphenyl | CH₃ | CH₃ | H | H |
| 70 | 3-methylphenyl | CH₃ | CH₃ | H | H |
| 71 | 4-methylphenyl | CH₃ | CH₃ | H | H |
| 72 | 2,4-dimethylphenyl | CH₃ | CH₃ | H | H |
| 73 | 2,5-dimethylphenyl | CH₃ | CH₃ | H | H |
| 74 | 2,6-dimethylphenyl | CH₃ | CH₃ | H | H |
| 75 | 2,3-dimethylphenyl | CH₃ | CH₃ | H | H |
| 76 | 3,4-dimethylphenyl | CH₃ | CH₃ | H | H |
| 77 | 3,5-dimethylphenyl | CH₃ | CH₃ | H | H |
| 78 | 2,4,5-trimethylphenyl | CH₃ | CH₃ | H | H |
| 79 | 3,4,5-trimethylphenyl | CH₃ | CH₃ | H | H |
| 80 | 2,4,6-trimethylphenyl | CH₃ | CH₃ | H | H |
| 81 | 2-methoxyphenyl | CH₃ | CH₃ | H | H |
| 82 | 3-methoxyphenyl | CH₃ | CH₃ | H | H |
| 83 | 4-methoxyphenyl | CH₃ | CH₃ | H | H |
| 84 | 2,4-dimethoxyphenyl | CH₃ | CH₃ | H | H |
| 85 | 2,5-dimethoxyphenyl | CH₃ | CH₃ | H | H |
| 86 | 2,6-dimethoxyphenyl | CH₃ | CH₃ | H | H |
| 87 | 2,3-dimethoxyphenyl | CH₃ | CH₃ | H | H |
| 88 | 3,4-dimethoxyphenyl | CH₃ | CH₃ | H | H |
| 89 | 3,5-dimethoxyphenyl | CH₃ | CH₃ | H | H |
| 90 | 2,4,5-trimethoxyphenyl | CH₃ | CH₃ | H | H |
| 91 | 3,4,5-trimethoxyphenyl | CH₃ | CH₃ | H | H |
| 92 | 2,4,6-trimethoxyphenyl | CH₃ | CH₃ | H | H |
| 93 | 2-trifluoromethoxyphenyl | CH₃ | CH₃ | H | H |
| 94 | 3-trifluoromethoxyphenyl | CH₃ | CH₃ | H | H |
| 95 | 4-trifluoromethoxyphenyl | CH₃ | CH₃ | H | H |
| 96 | 2-difluoromethoxyphenyl | CH₃ | CH₃ | H | H |
| 97 | 3-difluoromethoxyphenyl | CH₃ | CH₃ | H | H |
| 98 | 4-difluoromethoxyphenyl | CH₃ | CH₃ | H | H |
| 99 | 2-trifluoromethylphenyl | CH₃ | CH₃ | H | H |
| 100 | 3-trifluoromethylphenyl | CH₃ | CH₃ | H | H |
| 101 | 4-trifluoromethylphenyl | CH₃ | CH₃ | H | H |
| 102 | 2-difluoromethylphenyl | CH₃ | CH₃ | H | H |
| 103 | 3-difluoromethylphenyl | CH₃ | CH₃ | H | H |
| 104 | 4-difluoromethylphenyl | CH₃ | CH₃ | H | H |
| 105 | 3,5-bis(trifluoromethyl)phenyl | CH₃ | CH₃ | H | H |
| 106 | 3-trifluoromethyl-5-fluorophenyl | CH₃ | CH₃ | H | H |
| 107 | 3-trifluoromethyl-5-chlorophenyl | CH₃ | CH₃ | H | H |
| 106 | 3-methyl-5-fluorophenyl | CH₃ | CH₃ | H | H |
| 107 | 3-methyl-5-chlorophenyl | CH₃ | CH₃ | H | H |
| 108 | 3-methoxy-5-fluorophenyl | CH₃ | CH₃ | H | H |
| 109 | 3-methoxy-5-chlorophenyl | CH₃ | CH₃ | H | H |
| 110 | 3-trifluoromethoxy-5-chlorophenyl | CH₃ | CH₃ | H | H |
| 111 | 2-ethoxyphenyl | CH₃ | CH₃ | H | H |
| 112 | 3-ethoxyphenyl | CH₃ | CH₃ | H | H |
| 113 | 4-ethoxyphenyl | CH₃ | CH₃ | H | H |
| 114 | 2-methylthiophenyl | CH₃ | CH₃ | H | H |
| 115 | 3-methylthiophenyl | CH₃ | CH₃ | H | H |
| 116 | 4-methylthiophenyl | CH₃ | CH₃ | H | H |
| 117 | 2-trifluoromethylthiophenyl | CH₃ | CH₃ | H | H |
| 118 | 3-trifluoromethylthiophenyl | CH₃ | CH₃ | H | H |
| 119 | 4-trifluoromethylthiophenyl | CH₃ | CH₃ | H | H |
| 120 | 2-cyanophenyl | CH₃ | CH₃ | H | H |
| 121 | 3-cyanophenyl | CH₃ | CH₃ | H | H |
| 122 | 4-cyanophenyl | CH₃ | CH₃ | H | H |
| 123 | 2-ethoxyphenyl | CH₃ | CH₃ | H | H |
| 124 | 3-ethylphenyl | CH₃ | CH₃ | H | H |
| 125 | 4-ethylphenyl | CH₃ | CH₃ | H | H |
| 126 | 2-methoxycarbonylphenyl | CH₃ | CH₃ | H | H |
| 127 | 3-methoxycarbonylphenyl | CH₃ | CH₃ | H | H |
| 128 | 4-methoxycarbonylphenyl | CH₃ | CH₃ | H | H |
| 129 | 2-ethoxycarbonylphenyl | CH₃ | CH₃ | H | H |
| 130 | 3-ethoxycarbonylphenyl | CH₃ | CH₃ | H | H |
| 131 | 4-ethoxycarbonylphenyl | CH₃ | CH₃ | H | H |
| 132 | pyridin-2-yl | CH₃ | CH₃ | H | H |
| 133 | pyridin-3-yl | CH₃ | CH₃ | H | H |
| 134 | pyridin-4-yl | CH₃ | CH₃ | H | H |
| 135 | pyrazin-2-yl | CH₃ | CH₃ | H | H |
| 136 | pyridazin-3-yl | CH₃ | CH₃ | H | H |
| 137 | pyridazin-4-yl | CH₃ | CH₃ | H | H |
| 138 | pyrimidin-2-yl | CH₃ | CH₃ | H | H |
| 139 | pyrimidin-5-yl | CH₃ | CH₃ | H | H |
| 140 | pyrimidin-4-yl | CH₃ | CH₃ | H | H |
| 141 | pyridazin-3-ylmethyl | CH₃ | CH₃ | H | H |
| 142 | pyridazin-4-ylmethyl | CH₃ | CH₃ | H | H |
| 143 | pyrimidin-2-ylmethyl | CH₃ | CH₃ | H | H |
| 144 | pyrimidin-5-ylmethyl | CH₃ | CH₃ | H | H |
| 145 | pyrimidin-4-ylmethyl | CH₃ | CH₃ | H | H |
| 146 | pyrazin-2-ylmethyl | CH₃ | CH₃ | H | H |
| 147 | 3-chloropyrazin-2-yl | CH₃ | CH₃ | H | H |
| 148 | 3-bromopyrazin-2-yl | CH₃ | CH₃ | H | H |
| 149 | 3-methoxypyrazin-2-yl | CH₃ | CH₃ | H | H |
| 150 | 3-ethoxypyrazin-2-yl | CH₃ | CH₃ | H | H |
| 151 | 3-trifluoromethylpyrazin-2-yl | CH₃ | CH₃ | H | H |
| 152 | 3-cyanopyrazin-2-yl | CH₃ | CH₃ | H | H |
| 153 | naphth-2-yl | CH₃ | CH₃ | H | H |
| 154 | naphth-1-yl | CH₃ | CH₃ | H | H |
| 155 | quinolin-4-yl | CH₃ | CH₃ | H | H |
| 156 | quinolin-6-yl | CH₃ | CH₃ | H | H |
| 157 | quinolin-8-yl | CH₃ | CH₃ | H | H |
| 158 | quinolin-2-yl | CH₃ | CH₃ | H | H |
| 159 | quinoxalin-2-yl | CH₃ | CH₃ | H | H |
| 160 | 2-naphthylmethyl | CH₃ | CH₃ | H | H |
| 161 | 1-naphthylmethyl | CH₃ | CH₃ | H | H |
| 162 | quinolin-4-ylmethyl | CH₃ | CH₃ | H | H |
| 163 | quinolin-6-ylmethyl | CH₃ | CH₃ | H | H |
| 164 | quinolin-8-ylmethyl | CH₃ | CH₃ | H | H |
| 165 | quinolin-2-ylmethyl | CH₃ | CH₃ | H | H |
| 166 | quinoxalin-2-ylmethyl | CH₃ | CH₃ | H | H |
| 167 | pyrazin-2-ylmethyl | CH₃ | CH₃ | H | H |
| 168 | 4-chloropyridin-2-yl | CH₃ | CH₃ | H | H |
| 169 | 3-chloropyridin-4-yl | CH₃ | CH₃ | H | H |
| 170 | 2-chloropyridin-3-yl | CH₃ | CH₃ | H | H |
| 171 | 2-chloropyridin-4-yl | CH₃ | CH₃ | H | H |
| 172 | 2-chloropyridin-5-yl | CH₃ | CH₃ | H | H |
| 173 | 2,6-dichloropyridin-4-yl | CH₃ | CH₃ | H | H |
| 174 | 3-chloropyridin-5-yl | CH₃ | CH₃ | H | H |
| 175 | 3,5-dichloropyridin-2-yl | CH₃ | CH₃ | H | H |
| 176 | 3-chloro-5-trifluoromethylpyridin-2- | CH₃ | CH₃ | H | H |
| | yl | | | | |
| 177 | (4-chloropyridin-2-yl)methyl | CH₃ | CH₃ | H | H |
| 178 | (3-chloropyridin-4-yl)methyl | CH₃ | CH₃ | H | H |
| 179 | (2-chloropyridin-3-yl)methyl | CH₃ | CH₃ | H | H |
| 180 | (2-chloropyridin-4-yl)methyl | CH₃ | CH₃ | H | H |
| 181 | (2-chloropyridin-5-yl)methyl | CH₃ | CH₃ | H | H |
| 182 | (2,6-dichloropyridin-4-yl)methyl | CH₃ | CH₃ | H | H |
| 183 | (3-chloropyridin-5-yl)methyl | CH₃ | CH₃ | H | H |
| 184 | (3,5-dichloropyridin-2-yl)methyl | CH₃ | CH₃ | H | H |
| 185 | thiophen-2-yl | CH₃ | CH₃ | H | H |
| 186 | thiophen-3-yl | CH₃ | CH₃ | H | H |
| 187 | 5-methylthiophen-2-yl | CH₃ | CH₃ | H | H |
| 188 | 5-ethylthiophen-2-yl | CH₃ | CH₃ | H | H |
| 189 | 5-chlorothiophen-2-yl | CH₃ | CH₃ | H | H |
| 190 | 5-bromothiophen-2-yl | CH₃ | CH₃ | H | H |
| 191 | 4-methylthiophen-2-yl | CH₃ | CH₃ | H | H |
| 192 | 3-methylthiophen-2-yl | CH₃ | CH₃ | H | H |
| 193 | 5-fluorothiophen-3-yl | CH₃ | CH₃ | H | H |
| 194 | 3,5-dimethylthiophen-2-yl | CH₃ | CH₃ | H | H |
| 195 | 3-ethylthiophen-2-yl | CH₃ | CH₃ | H | H |
| 196 | 4,5-dimethylthiophen-2-yl | CH₃ | CH₃ | H | H |
| 197 | 3,4-dimethylthiophen-2-yl | CH₃ | CH₃ | H | H |
| 198 | 4-chlorothiophen-2-yl | CH₃ | CH₃ | H | H |
| 199 | furan-2-yl | CH₃ | CH₃ | H | H |
| 200 | 5-methylfuran-2-yl | CH₃ | CH₃ | H | H |
| 201 | 5-ethylfuran-2-yl | CH₃ | CH₃ | H | H |
| 202 | 5-methoxycarbonylfuran-2-yl | CH₃ | CH₃ | H | H |
| 203 | 5-chlorofuran-2-yl | CH₃ | CH₃ | H | H |
| 204 | 5-bromofuran-2-yl | CH₃ | CH₃ | H | H |
| 205 | 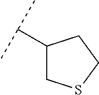 | CH₃ | CH₃ | H | H |
| 206 | 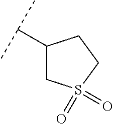 | CH₃ | CH₃ | H | H |
| 207 | 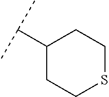 | CH₃ | CH₃ | H | H |
| 208 | 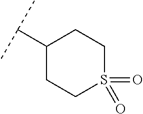 | CH₃ | CH₃ | H | H |
| 209 | 1-(4-methylphenyl)ethyl | CH₃ | CH₃ | H | H |
| 210 | 1-(3-methylphenyl)ethyl | CH₃ | CH₃ | H | H |
| 211 | 1-(2-methylphenyl)ethyl | CH₃ | CH₃ | H | H |
| 212 | 1-(4-chlorophenyl)ethyl | CH₃ | CH₃ | H | H |
| 213 | 1-(3-chlorophenyl)ethyl | CH₃ | CH₃ | H | H |
| 214 | 1-(2-chlorophenyl)ethyl | CH₃ | CH₃ | H | H |
| 215 | 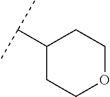 | CH₃ | CH₃ | H | H |
| 216 | 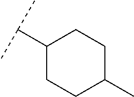 | CH₃ | CH₃ | H | H |
| 217 | benzyl | CH₃ | CH₃ | H | H |
| 218 | (4-fluorophenyl)methyl | CH₃ | CH₃ | H | H |
| 219 | (3-fluorophenyl)methyl | CH₃ | CH₃ | H | H |
| 220 | (2-fluorophenyl)methyl | CH₃ | CH₃ | H | H |
| 221 | (2,4-difluorophenyl)methyl | CH₃ | CH₃ | H | H |
| 222 | (3,5-difluorophenyl)methyl | CH₃ | CH₃ | H | H |
| 223 | (2,5-difluorophenyl)methyl | CH₃ | CH₃ | H | H |
| 224 | (2,6-difluorophenyl)methyl | CH₃ | CH₃ | H | H |
| 225 | (2,4,5-trifluorophenyl)methyl | CH₃ | CH₃ | H | H |
| 226 | (2,4,6-trifluorophenyl)methyl | CH₃ | CH₃ | H | H |
| 227 | (4-chlorophenyl)methyl | CH₃ | CH₃ | H | H |
| 228 | (3-chlorophenyl)methyl | CH₃ | CH₃ | H | H |
| 229 | (2-chlorophenyl)methyl | CH₃ | CH₃ | H | H |
| 230 | (2,4-dichlorophenyl)methyl | CH₃ | CH₃ | H | H |
| 231 | (3,5-dichlorophenyl)methyl | CH₃ | CH₃ | H | H |
| 232 | (2,5-dichlorophenyl)methyl | CH₃ | CH₃ | H | H |
| 233 | (2,6-dichlorophenyl)methyl | CH₃ | CH₃ | H | H |
| 234 | (2,4,5-trichlorophenyl)methyl | CH₃ | CH₃ | H | H |
| 235 | (2,4,6-trichlorophenyl)methyl | CH₃ | CH₃ | H | H |
| 236 | (4-bromophenyl)methyl | CH₃ | CH₃ | H | H |
| 237 | (3-bromophenyl)methyl | CH₃ | CH₃ | H | H |
| 238 | (2-bromophenyl)methyl | CH₃ | CH₃ | H | H |
| 239 | (4-iodophenyl)methyl | CH₃ | CH₃ | H | H |
| 240 | (3-iodophenyl)methyl | CH₃ | CH₃ | H | H |
| 241 | (2-iodophenyl)methyl | CH₃ | CH₃ | H | H |
| 242 | (3-chloro-5-trifluoromethylpyridin-2- | CH₃ | CH₃ | H | H |
| | yl)methyl | | | | |
| 243 | (2-bromo-4-fluorophenyl)methyl | CH₃ | CH₃ | H | H |
| 244 | (2-bromo-4-chlorophenyl)methyl | CH₃ | CH₃ | H | H |
| 245 | (3-bromo-4-fluorophenyl)methyl | CH₃ | CH₃ | H | H |
| 246 | (3-bromo-4-chlorophenyl)methyl | CH₃ | CH₃ | H | H |
| 247 | (3-bromo-5-fluorophenyl)methyl | CH₃ | CH₃ | H | H |
| 248 | (3-bromo-5-chlorophenyl)methyl | CH₃ | CH₃ | H | H |
| 249 | (2-fluoro-4-bromophenyl)methyl | CH₃ | CH₃ | H | H |
| 250 | (2-chloro-4-bromophenyl)methyl | CH₃ | CH₃ | H | H |
| 251 | (3-fluoro-4-bromophenyl)methyl | CH₃ | CH₃ | H | H |
| 252 | (3-chloro-4-bromophenyl)methyl | CH₃ | CH₃ | H | H |
| 253 | (2-chloro-4-fluorophenyl)methyl | CH₃ | CH₃ | H | H |
| 254 | (3-chloro-4-fluorophenyl)methyl | CH₃ | CH₃ | H | H |
| 255 | (2-fluoro-3-chlorophenyl)methyl | CH₃ | CH₃ | H | H |
| 256 | (2-fluoro-4-chlorophenyl)methyl | CH₃ | CH₃ | H | H |
| 257 | (2-fluoro-5-chlorophenyl)methyl | CH₃ | CH₃ | H | H |
| 258 | (3-fluoro-4-chlorophenyl)methyl | CH₃ | CH₃ | H | H |
| 259 | (3-fluoro-5-chlorophenyl)methyl | CH₃ | CH₃ | H | H |
| 260 | (2-fluoro-6-chlorophenyl)methyl | CH₃ | CH₃ | H | H |
| 261 | phenylethyl | CH₃ | CH₃ | H | H |
| 262 | 3-trifluoromethyl-4-chlorophenyl | CH₃ | CH₃ | H | H |
| 263 | 3-chloro-4-trifluoromethylphenyl | CH₃ | CH₃ | H | H |
| 264 | 2-chloro-4-trifluoromethylphenyl | CH₃ | CH₃ | H | H |
| 265 | 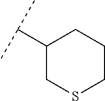 | CH₃ | CH₃ | H | H |
| 266 | 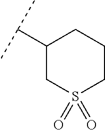 | CH₃ | CH₃ | H | H |
| 267 | (3,5-dichloropyridin-2-yl)methyl | CH₃ | CH₃ | H | H |
| 268 | (4-trifluoromethylphenyl)methyl | CH₃ | CH₃ | H | H |
| 269 | (3-trifluoromethylphenyl)methyl | CH₃ | CH₃ | H | H |
| 270 | (2-trifluoromethylphenyl)methyl | CH₃ | CH₃ | H | H |
| 271 | (4-trifluoromethoxyphenyl)methyl | CH₃ | CH₃ | H | H |
| 272 | (3-trifluoromethoxyphenyl)methyl | CH₃ | CH₃ | H | H |
| 273 | (2-trifluoromethoxyphenyl)methyl | CH₃ | CH₃ | H | H |
| 274 | (4-methoxyphenyl)methyl | CH₃ | CH₃ | H | H |
| 275 | (3-methoxyphenyl)methyl | CH₃ | CH₃ | H | H |
| 276 | (2-methoxyphenyl)methyl | CH₃ | CH₃ | H | H |
| 277 | (4-methylphenyl)methyl | CH₃ | CH₃ | H | H |
| 278 | (3-methylphenyl)methyl | CH₃ | CH₃ | H | H |
| 279 | (2-methylphenyl)methyl | CH₃ | CH₃ | H | H |
| 280 | (4-cyanophenyl)methyl | CH₃ | CH₃ | H | H |
| 281 | (3-cyanophenyl)methyl | CH₃ | CH₃ | H | H |
| 282 | (2-cyanophenyl)methyl | CH₃ | CH₃ | H | H |
| 283 | (2,4-diethylphenyl)methyl | CH₃ | CH₃ | H | H |
| 284 | (3,5-diethylphenyl)methyl | CH₃ | CH₃ | H | H |
| 285 | (3,4-dimethylphenyl)methyl | CH₃ | CH₃ | H | H |
| 286 | (3,5-dimethoxyphenyl)methyl | CH₃ | CH₃ | H | H |
| 287 | 1-phenyleth-1-yl | CH₃ | CH₃ | H | H |
| 288 | 1-methylprop-1-yl | CH₃ | CH₃ | H | H |
| 289 | 2-methylprop-1-yl | CH₃ | CH₃ | H | H |
| 290 | 1-ethylprop-1-yl | CH₃ | CH₃ | H | H |
| 291 | 1-methylbut-1-yl | CH₃ | CH₃ | H | H |
| 292 | 2-methylbut-1-yl | CH₃ | CH₃ | H | H |
| 293 | 3-methylbut-1-yl | CH₃ | CH₃ | H | H |
| 294 | 2,2-difluoroethyl | CH₃ | CH₃ | H | H |
| 295 | 2,2,2-trifluoroethyl | CH₃ | CH₃ | H | H |
| 296 | 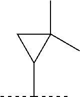 | CH₃ | CH₃ | H | H |
| 297 | 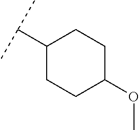 | CH₃ | CH₃ | H | H |
| 298 | 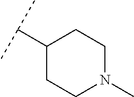 | CH₃ | CH₃ | H | H |
| 299 | 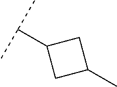 | CH₃ | CH₃ | H | H |
| 300 | 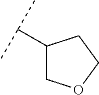 | CH₃ | CH₃ | H | H |
| 301 | H | CH₃ | H | H | H |
| 302 | CH₃ | CH₃ | H | H | H |
| 303 | Et | CH₃ | H | H | H |
| 304 | n-Pr | CH₃ | H | H | H |
| 305 | i-Pr | CH₃ | H | H | H |
| 306 | n-Bu | CH₃ | H | H | H |
| 307 | neopentyl | CH₃ | H | H | H |
| 308 | t-Bu | CH₃ | H | H | H |
| 309 | n-pentyl | CH₃ | H | H | H |
| 310 | n-hexyl | CH₃ | H | H | H |
| 311 | c-Pr | CH₃ | H | H | H |
| 312 | c-Bu | CH₃ | H | H | H |
| 313 | c-pentyl | CH₃ | H | H | H |
| 314 | c-hexyl | CH₃ | H | H | H |
| 315 | 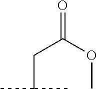 | CH₃ | H | H | H |
| 316 | 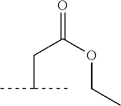 | CH₃ | H | H | H |
| 317 | 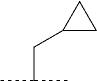 | CH₃ | H | H | H |
| 318 | 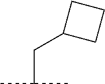 | CH₃ | H | H | H |
| 319 | 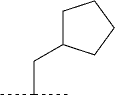 | CH₃ | H | H | H |
| 320 | 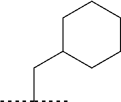 | CH₃ | H | H | H |
| 321 | phenyl | CH₃ | H | H | H |
| 322 | 2-fluorophenyl | CH₃ | H | H | H |
| 323 | 3-fluorophenyl | CH₃ | H | H | H |
| 324 | 4-fluorophenyl | CH₃ | H | H | H |
| 325 | 2,4-difluorophenyl | CH₃ | H | H | H |
| 326 | 2,5-difluorophenyl | CH₃ | H | H | H |
| 327 | 2,6-difluorophenyl | CH₃ | H | H | H |
| 328 | 2,3-difluorophenyl | CH₃ | H | H | H |
| 329 | 3,4-difluorophenyl | CH₃ | H | H | H |
| 330 | 3,5-difluorophenyl | CH₃ | H | H | H |
| 331 | 2,4,5-trifluorophenyl | CH₃ | H | H | H |
| 332 | 3,4,5-trifluorophenyl | CH₃ | H | H | H |
| 333 | 2-chlorophenyl | CH₃ | H | H | H |
| 334 | 3-chlorophenyl | CH₃ | H | H | H |
| 335 | 4-chlorophenyl | CH₃ | H | H | H |
| 336 | 2,4-dichlorophenyl | CH₃ | H | H | H |
| 337 | 2,5-dichlorophenyl | CH₃ | H | H | H |
| 338 | 2,6-dichlorophenyl | CH₃ | H | H | H |
| 339 | 2,3-dichlorophenyl | CH₃ | H | H | H |
| 340 | 3,4-dichlorophenyl | CH₃ | H | H | H |
| 341 | 3,5-dichlorophenyl | CH₃ | H | H | H |
| 342 | 2,4,5-trichlorophenyl | CH₃ | H | H | H |
| 343 | 3,4,5-trichlorophenyl | CH₃ | H | H | H |
| 344 | 2,4,6-trichlorophenyl | CH₃ | H | H | H |
| 345 | 2-bromophenyl | CH₃ | H | H | H |
| 346 | 3-bromophenyl | CH₃ | H | H | H |
| 347 | 4-bromophenyl | CH₃ | H | H | H |
| 348 | 2-iodophenyl | CH₃ | H | H | H |
| 349 | 3-iodophenyl | CH₃ | H | H | H |
| 350 | 4-iodophenyl | CH₃ | H | H | H |
| 351 | 2-bromo-4-fluorophenyl | CH₃ | H | H | H |
| 352 | 2-bromo-4-chlorophenyl | CH₃ | H | H | H |
| 353 | 3-bromo-4-fluorophenyl | CH₃ | H | H | H |
| 354 | 3-bromo-4-chlorophenyl | CH₃ | H | H | H |
| 355 | 3-bromo-5-fluorophenyl | CH₃ | H | H | H |
| 356 | 3-bromo-5-chlorophenyl | CH₃ | H | H | H |
| 357 | 2-fluoro-4-bromophenyl | CH₃ | H | H | H |
| 358 | 2-chloro-4-bromophenyl | CH₃ | H | H | H |
| 359 | 3-fluoro-4-bromophenyl | CH₃ | H | H | H |
| 360 | 3-chloro-4-bromophenyl | CH₃ | H | H | H |
| 361 | 2-chloro-4-fluorophenyl | CH₃ | H | H | H |
| 362 | 3-chloro-4-fluorophenyl | CH₃ | H | H | H |
| 363 | 2-fluoro-3-chlorophenyl | CH₃ | H | H | H |
| 364 | 2-fluoro-4-chlorophenyl | CH₃ | H | H | H |
| 365 | 2-fluoro-5-chlorophenyl | CH₃ | H | H | H |
| 366 | 3-fluoro-4-chlorophenyl | CH₃ | H | H | H |
| 367 | 3-fluoro-5-chlorophenyl | CH₃ | H | H | H |
| 368 | 2-fluoro-6-chlorophenyl | CH₃ | H | H | H |
| 369 | 2-methylphenyl | CH₃ | H | H | H |
| 370 | 3-methylphenyl | CH₃ | H | H | H |
| 371 | 4-methylphenyl | CH₃ | H | H | H |
| 372 | 2,4-dimethylphenyl | CH₃ | H | H | H |
| 373 | 2,5-dimethylphenyl | CH₃ | H | H | H |
| 374 | 2,6-dimethylphenyl | CH₃ | H | H | H |
| 375 | 2,3-dimethylphenyl | CH₃ | H | H | H |
| 376 | 3,4-dimethylphenyl | CH₃ | H | H | H |
| 377 | 3,5-dimethylphenyl | CH₃ | H | H | H |
| 378 | 2,4,5-trimethylphenyl | CH₃ | H | H | H |
| 379 | 3,4,5-trimethylphenyl | CH₃ | H | H | H |
| 380 | 2,4,6-trimethylphenyl | CH₃ | H | H | H |
| 381 | 2-methoxyphenyl | CH₃ | H | H | H |
| 382 | 3-methoxyphenyl | CH₃ | H | H | H |
| 383 | 4-methoxyphenyl | CH₃ | H | H | H |
| 384 | 2,4-dimethoxyphenyl | CH₃ | H | H | H |
| 385 | 2,5-dimethoxyphenyl | CH₃ | H | H | H |
| 386 | 2,6-dimethoxyphenyl | CH₃ | H | H | H |
| 387 | 2,3-dimethoxyphenyl | CH₃ | H | H | H |
| 388 | 3,4-dimethoxyphenyl | CH₃ | H | H | H |
| 389 | 3,5-dimethoxyphenyl | CH₃ | H | H | H |
| 390 | 2,4,5-trimethoxyphenyl | CH₃ | H | H | H |
| 391 | 3,4,5-trimethoxyphenyl | CH₃ | H | H | H |
| 392 | 2,4,6-trimethoxyphenyl | CH₃ | H | H | H |
| 393 | 2-trifluoromethoxyphenyl | CH₃ | H | H | H |
| 394 | 3-trifluoromethoxyphenyl | CH₃ | H | H | H |
| 395 | 4-trifluoromethoxyphenyl | CH₃ | H | H | H |
| 396 | 2-difluoromethoxyphenyl | CH₃ | H | H | H |
| 397 | 3-difluoromethoxyphenyl | CH₃ | H | H | H |
| 398 | 4-difluoromethoxyphenyl | CH₃ | H | H | H |
| 399 | 2-trifluoromethylphenyl | CH₃ | H | H | H |
| 400 | 3-trifluoromethylphenyl | CH₃ | H | H | H |
| 401 | 4-trifluoromethylphenyl | CH₃ | H | H | H |
| 402 | 2-difluoromethylphenyl | CH₃ | H | H | H |
| 403 | 3-difluoromethylphenyl | CH₃ | H | H | H |
| 404 | 4-difluoromethylphenyl | CH₃ | H | H | H |
| 405 | 3,5-bis(trifluoromethyl)phenyl | CH₃ | H | H | H |
| 406 | 3-trifluoromethyl-5-fluorophenyl | CH₃ | H | H | H |
| 407 | 3-trifluoromethyl-5-chlorophenyl | CH₃ | H | H | H |
| 406 | 3-methyl-5-fluorophenyl | CH₃ | H | H | H |
| 407 | 3-methyl-5-chlorophenyl | CH₃ | H | H | H |
| 408 | 3-methoxy-5-fluorophenyl | CH₃ | H | H | H |
| 409 | 3-methoxy-5-chlorophenyl | CH₃ | H | H | H |
| 410 | 3-trifluoromethoxy-5-chlorophenyl | CH₃ | H | H | H |
| 411 | 2-ethoxyphenyl | CH₃ | H | H | H |
| 412 | 3-ethoxyphenyl | CH₃ | H | H | H |
| 413 | 4-ethoxyphenyl | CH₃ | H | H | H |
| 414 | 2-methylthiophenyl | CH₃ | H | H | H |
| 415 | 3-methylthiophenyl | CH₃ | H | H | H |
| 416 | 4-methylthiophenyl | CH₃ | H | H | H |
| 417 | 2-trifluoromethylthiophenyl | CH₃ | H | H | H |
| 418 | 3-trifluoromethylthiophenyl | CH₃ | H | H | H |
| 419 | 4-trifluoromethylthiophenyl | CH₃ | H | H | H |
| 420 | 2-cyanophenyl | CH₃ | H | H | H |
| 421 | 3-cyanophenyl | CH₃ | H | H | H |
| 422 | 4-cyanophenyl | CH₃ | H | H | H |
| 423 | 2-ethoxyphenyl | CH₃ | H | H | H |
| 424 | 3-ethylphenyl | CH₃ | H | H | H |
| 425 | 4-ethylphenyl | CH₃ | H | H | H |
| 426 | 2-methoxycarbonylphenyl | CH₃ | H | H | H |
| 427 | 3-methoxycarbonylphenyl | CH₃ | H | H | H |
| 428 | 4-methoxycarbonylphenyl | CH₃ | H | H | H |
| 429 | 2-ethoxycarbonylphenyl | CH₃ | H | H | H |
| 430 | 3-ethoxycarbonylphenyl | CH₃ | H | H | H |
| 431 | 4-ethoxycarbonylphenyl | CH₃ | H | H | H |
| 432 | pyridin-2-yl | CH₃ | H | H | H |
| 433 | pyridin-3-yl | CH₃ | H | H | H |
| 434 | pyridin-4-yl | CH₃ | H | H | H |
| 435 | pyrazin-2-yl | CH₃ | H | H | H |
| 436 | pyridazin-3-yl | CH₃ | H | H | H |
| 437 | pyridazin-4-yl | CH₃ | H | H | H |
| 438 | pyrimidin-2-yl | CH₃ | H | H | H |
| 439 | pyrimidin-5-yl | CH₃ | H | H | H |
| 440 | pyrimidin-4-yl | CH₃ | H | H | H |
| 441 | pyridazin-3-ylmethyl | CH₃ | H | H | H |
| 442 | pyridazin-4-ylmethyl | CH₃ | H | H | H |
| 443 | pyrimidin-2-ylmethyl | CH₃ | H | H | H |
| 444 | pyrimidin-5-ylmethyl | CH₃ | H | H | H |
| 445 | pyrimidin-4-ylmethyl | CH₃ | H | H | H |
| 446 | pyrazin-2-ylmethyl | CH₃ | H | H | H |
| 447 | 3-chloropyrazin-2-yl | CH₃ | H | H | H |
| 448 | 3-bromopyrazin-2-yl | CH₃ | H | H | H |
| 449 | 3-methoxypyrazin-2-yl | CH₃ | H | H | H |
| 450 | 3-ethoxypyrazin-2-yl | CH₃ | H | H | H |
| 451 | 3-trifluoromethylpyrazin-2-yl | CH₃ | H | H | H |
| 452 | 3-cyanopyrazin-2-yl | CH₃ | H | H | H |
| 453 | naphth-2-yl | CH₃ | H | H | H |
| 454 | naphth-1-yl | CH₃ | H | H | H |
| 455 | quinolin-4-yl | CH₃ | H | H | H |
| 456 | quinolin-6-yl | CH₃ | H | H | H |
| 457 | quinolin-8-yl | CH₃ | H | H | H |
| 458 | quinolin-2-yl | CH₃ | H | H | H |
| 459 | quinoxalin-2-yl | CH₃ | H | H | H |
| 460 | 2-naphthylmethyl | CH₃ | H | H | H |
| 461 | 1-naphthylmethyl | CH₃ | H | H | H |
| 462 | quinolin-4-ylmethyl | CH₃ | H | H | H |
| 463 | quinolin-6-ylmethyl | CH₃ | H | H | H |
| 464 | quinolin-8-ylmethyl | CH₃ | H | H | H |
| 465 | quinolin-2-ylmethyl | CH₃ | H | H | H |
| 466 | quinoxalin-2-ylmethyl | CH₃ | H | H | H |
| 467 | pyrazin-2-ylmethyl | CH₃ | H | H | H |
| 468 | 4-chloropyridin-2-yl | CH₃ | H | H | H |
| 469 | 3-chloropyridin-4-yl | CH₃ | H | H | H |
| 470 | 2-chloropyridin-3-yl | CH₃ | H | H | H |
| 471 | 2-chloropyridin-4-yl | CH₃ | H | H | H |
| 472 | 2-chloropyridin-5-yl | CH₃ | H | H | H |
| 473 | 2,6-dichloropyridin-4-yl | CH₃ | H | H | H |
| 474 | 3-chloropyridin-5-yl | CH₃ | H | H | H |
| 475 | 3,5-dichloropyridin-2-yl | CH₃ | H | H | H |
| 476 | 3-chloro-5-trifluoromethylpyridin-2- | CH₃ | H | H | H |
| | yl | | | | |
| 477 | (4-chloropyridin-2-yl)methyl | CH₃ | H | H | H |
| 478 | (3-chloropyridin-4-yl)methyl | CH₃ | H | H | H |
| 479 | (2-chloropyridin-3-yl)methyl | CH₃ | H | H | H |
| 480 | (2-chloropyridin-4-yl)methyl | CH₃ | H | H | H |
| 481 | (2-chloropyridin-5-yl)methyl | CH₃ | H | H | H |
| 482 | (2,6-dichloropyridin-4-yl)methyl | CH₃ | H | H | H |
| 483 | (3-chloropyridin-5-yl)methyl | CH₃ | H | H | H |
| 484 | (3,5-dichloropyridin-2-yl)methyl | CH₃ | H | H | H |
| 485 | thiophen-2-yl | CH₃ | H | H | H |
| 486 | thiophen-3-yl | CH₃ | H | H | H |
| 487 | 5-methylthiophen-2-yl | CH₃ | H | H | H |
| 488 | 5-ethylthiophen-2-yl | CH₃ | H | H | H |
| 489 | 5-chlorothiophen-2-yl | CH₃ | H | H | H |
| 490 | 5-bromothiophen-2-yl | CH₃ | H | H | H |
| 491 | 4-methylthiophen-2-yl | CH₃ | H | H | H |
| 492 | 3-methylthiophen-2-yl | CH₃ | H | H | H |
| 493 | 5-fluorothiophen-3-yl | CH₃ | H | H | H |
| 494 | 3,5-dimethylthiophen-2-yl | CH₃ | H | H | H |
| 495 | 3-ethylthiophen-2-yl | CH₃ | H | H | H |
| 496 | 4,5-dimethylthiophen-2-yl | CH₃ | H | H | H |
| 497 | 3,4-dimethylthiophen-2-yl | CH₃ | H | H | H |
| 498 | 4-chlorothiophen-2-yl | CH₃ | H | H | H |
| 499 | furan-2-yl | CH₃ | H | H | H |
| 500 | 5-methylfuran-2-yl | CH₃ | H | H | H |
| 501 | 5-ethylfuran-2-yl | CH₃ | H | H | H |
| 502 | 5-methoxycarbonylfuran-2-yl | CH₃ | H | H | H |
| 503 | 5-chlorofuran-2-yl | CH₃ | H | H | H |
| 504 | 5-bromofuran-2-yl | CH₃ | H | H | H |
| 505 | 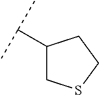 | CH₃ | H | H | H |
| 506 | 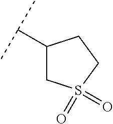 | CH₃ | H | H | H |
| 507 | 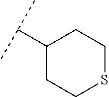 | CH₃ | H | H | H |
| 508 | 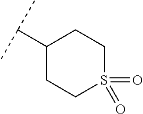 | CH₃ | H | H | H |
| 509 | 1-(4-methylphenyl)ethyl | CH₃ | H | H | H |
| 510 | 1-(3-methylphenyl)ethyl | CH₃ | H | H | H |
| 511 | 1-(2-methylphenyl)ethyl | CH₃ | H | H | H |
| 512 | 1-(4-chlorophenyl)ethyl | CH₃ | H | H | H |
| 513 | 1-(3-chlorophenyl)ethyl | CH₃ | H | H | H |
| 514 | 1-(2-chlorophenyl)ethyl | CH₃ | H | H | H |
| 515 | 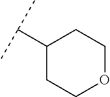 | CH₃ | H | H | H |
| 516 | 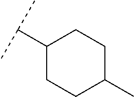 | CH₃ | H | H | H |
| 517 | benzyl | CH₃ | H | H | H |
| 518 | (4-fluorophenyl)methyl | CH₃ | H | H | H |
| 519 | (3-fluorophenyl)methyl | CH₃ | H | H | H |
| 520 | (2-fluorophenyl)methyl | CH₃ | H | H | H |
| 521 | (2,4-difluorophenyl)methyl | CH₃ | H | H | H |
| 522 | (3,5-difluorophenyl)methyl | CH₃ | H | H | H |
| 523 | (2,5-difluorophenyl)methyl | CH₃ | H | H | H |
| 524 | (2,6-difluorophenyl)methyl | CH₃ | H | H | H |
| 525 | (2,4,5-trifluorophenyl)methyl | CH₃ | H | H | H |
| 526 | (2,4,6-trifluorophenyl)methyl | CH₃ | H | H | H |
| 527 | (4-chlorophenyl)methyl | CH₃ | H | H | H |
| 528 | (3-chlorophenyl)methyl | CH₃ | H | H | H |
| 529 | (2-chlorophenyl)methyl | CH₃ | H | H | H |
| 530 | (2,4-dichlorophenyl)methyl | CH₃ | H | H | H |
| 531 | (3,5-dichlorophenyl)methyl | CH₃ | H | H | H |
| 532 | (2,5-dichlorophenyl)methyl | CH₃ | H | H | H |
| 533 | (2,6-dichlorophenyl)methyl | CH₃ | H | H | H |
| 534 | (2,4,5-trichlorophenyl)methyl | CH₃ | H | H | H |
| 535 | (2,4,6-trichlorophenyl)methyl | CH₃ | H | H | H |
| 536 | (4-bromophenyl)methyl | CH₃ | H | H | H |
| 537 | (3-bromophenyl)methyl | CH₃ | H | H | H |
| 538 | (2-bromophenyl)methyl | CH₃ | H | H | H |
| 539 | (4-iodophenyl)methyl | CH₃ | H | H | H |
| 540 | (3-iodophenyl)methyl | CH₃ | H | H | H |
| 541 | (2-iodophenyl)methyl | CH₃ | H | H | H |
| 542 | (3-chloro-5-trifluoromethylpyridin-2- | CH₃ | H | H | H |
| | yl)methyl | | | | |
| 543 | (2-bromo-4-fluorophenyl)methyl | CH₃ | H | H | H |
| 544 | (2-bromo-4-chlorophenyl)methyl | CH₃ | H | H | H |
| 545 | (3-bromo-4-fluorophenyl)methyl | CH₃ | H | H | H |
| 546 | (3-bromo-4-chlorophenyl)methyl | CH₃ | H | H | H |
| 547 | (3-bromo-5-fluorophenyl)methyl | CH₃ | H | H | H |
| 548 | (3-bromo-5-chlorophenyl)methyl | CH₃ | H | H | H |
| 549 | (2-fluoro-4-bromophenyl)methyl | CH₃ | H | H | H |
| 550 | (2-chloro-4-bromophenyl)methyl | CH₃ | H | H | H |
| 551 | (3-fluoro-4-bromophenyl)methyl | CH₃ | H | H | H |
| 552 | (3-chloro-4-bromophenyl)methyl | CH₃ | H | H | H |
| 553 | (2-chloro-4-fluorophenyl)methyl | CH₃ | H | H | H |
| 554 | (3-chloro-4-fluorophenyl)methyl | CH₃ | H | H | H |
| 555 | (2-fluoro-3-chlorophenyl)methyl | CH₃ | H | H | H |
| 556 | (2-fluoro-4-chlorophenyl)methyl | CH₃ | H | H | H |
| 557 | (2-fluoro-5-chlorophenyl)methyl | CH₃ | H | H | H |
| 558 | (3-fluoro-4-chlorophenyl)methyl | CH₃ | H | H | H |
| 559 | (3-fluoro-5-chlorophenyl)methyl | CH₃ | H | H | H |
| 560 | (2-fluoro-6-chlorophenyl)methyl | CH₃ | H | H | H |
| 561 | phenylethyl | CH₃ | H | H | H |
| 562 | 3-trifluoromethyl-4-chlorophenyl | CH₃ | H | H | H |
| 563 | 3-chloro-4-trifluoromethylphenyl | CH₃ | H | H | H |
| 564 | 2-chloro-4-trifluoromethylphenyl | CH₃ | H | H | H |
| 565 | 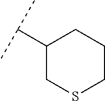 | CH₃ | H | H | H |
| 566 | 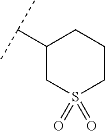 | CH₃ | H | H | H |
| 567 | (3,5-dichloropyridin-2-yl)methyl | CH₃ | H | H | H |
| 568 | (4-trifluoromethylphenyl)methyl | CH₃ | H | H | H |
| 569 | (3-trifluoromethylphenyl)methyl | CH₃ | H | H | H |
| 570 | (2-trifluoromethylphenyl)methyl | CH₃ | H | H | H |
| 571 | (4-trifluoromethoxyphenyl)methyl | CH₃ | H | H | H |
| 572 | (3-trifluoromethoxyphenyl)methyl | CH₃ | H | H | H |
| 573 | (2-trifluoromethoxyphenyl)methyl | CH₃ | H | H | H |
| 574 | (4-methoxyphenyl)methyl | CH₃ | H | H | H |
| 575 | (3-methoxyphenyl)methyl | CH₃ | H | H | H |
| 576 | (2-methoxyphenyl)methyl | CH₃ | H | H | H |
| 577 | (4-methylphenyl)methyl | CH₃ | H | H | H |
| 578 | (3-methylphenyl)methyl | CH₃ | H | H | H |
| 579 | (2-methylphenyl)methyl | CH₃ | H | H | H |
| 580 | (4-cyanophenyl)methyl | CH₃ | H | H | H |
| 581 | (3-cyanophenyl)methyl | CH₃ | H | H | H |
| 582 | (2-cyanophenyl)methyl | CH₃ | H | H | H |
| 583 | (2,4-diethylphenyl)methyl | CH₃ | H | H | H |
| 584 | (3,5-diethylphenyl)methyl | CH₃ | H | H | H |
| 585 | (3,4-dimethylphenyl)methyl | CH₃ | H | H | H |
| 586 | (3,5-dimethoxyphenyl)methyl | CH₃ | H | H | H |
| 587 | 1-phenyleth-1-yl | CH₃ | H | H | H |
| 588 | 1-methylprop-1-yl | CH₃ | H | H | H |
| 589 | 2-methylprop-1-yl | CH₃ | H | H | H |
| 590 | 1-ethylprop-1-yl | CH₃ | H | H | H |
| 591 | 1-methylbut-1-yl | CH₃ | H | H | H |
| 592 | 2-methylbut-1-yl | CH₃ | H | H | H |
| 593 | 3-methylbut-1-yl | CH₃ | H | H | H |
| 594 | 2,2-difluoroethyl | CH₃ | H | H | H |
| 595 | 2,2,2-trifluoroethyl | CH₃ | H | H | H |
| 596 | 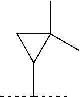 | CH₃ | H | H | H |
| 597 | 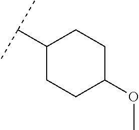 | CH₃ | H | H | H |
| 598 | 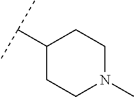 | CH₃ | H | H | H |
| 599 | 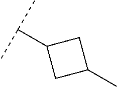 | CH₃ | H | H | H |
| 600 | 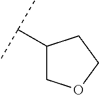 | CH₃ | H | H | H |
| 601 | H | H | H | H | H |
| 602 | CH₃ | H | H | H | H |
| 603 | Et | H | H | H | H |
| 604 | n-Pr | H | H | H | H |
| 605 | i-Pr | H | H | H | H |
| 606 | n-Bu | H | H | H | H |
| 607 | neopentyl | H | H | H | H |
| 608 | t-Bu | H | H | H | H |
| 609 | n-pentyl | H | H | H | H |
| 610 | n-hexyl | H | H | H | H |
| 611 | c-Pr | H | H | H | H |
| 612 | c-Bu | H | H | H | H |
| 613 | c-pentyl | H | H | H | H |
| 614 | c-hexyl | H | H | H | H |
| 615 | 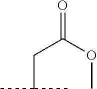 | H | H | H | H |
| 616 | 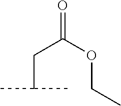 | H | H | H | H |
| 617 | 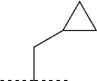 | H | H | H | H |
| 618 | 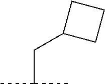 | H | H | H | H |
| 619 | 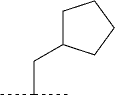 | H | H | H | H |
| 620 | 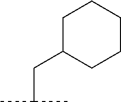 | H | H | H | H |
| 621 | phenyl | H | H | H | H |
| 622 | 2-fluorophenyl | H | H | H | H |
| 623 | 3-fluorophenyl | H | H | H | H |
| 624 | 4-fluorophenyl | H | H | H | H |
| 625 | 2,4-difluorophenyl | H | H | H | H |
| 626 | 2,5-difluorophenyl | H | H | H | H |
| 627 | 2,6-difluorophenyl | H | H | H | H |
| 628 | 2,3-difluorophenyl | H | H | H | H |
| 629 | 3,4-difluorophenyl | H | H | H | H |
| 630 | 3,5-difluorophenyl | H | H | H | H |
| 631 | 2,4,5-trifluorophenyl | H | H | H | H |
| 632 | 3,4,5-trifluorophenyl | H | H | H | H |
| 633 | 2-chlorophenyl | H | H | H | H |
| 634 | 3-chlorophenyl | H | H | H | H |
| 635 | 4-chlorophenyl | H | H | H | H |
| 636 | 2,4-dichlorophenyl | H | H | H | H |
| 637 | 2,5-dichlorophenyl | H | H | H | H |
| 638 | 2,6-dichlorophenyl | H | H | H | H |
| 639 | 2,3-dichlorophenyl | H | H | H | H |
| 640 | 3,4-dichlorophenyl | H | H | H | H |
| 641 | 3,5-dichlorophenyl | H | H | H | H |
| 642 | 2,4,5-trichlorophenyl | H | H | H | H |
| 643 | 3,4,5-trichlorophenyl | H | H | H | H |
| 644 | 2,4,6-trichlorophenyl | H | H | H | H |
| 645 | 2-bromophenyl | H | H | H | H |
| 646 | 3-bromophenyl | H | H | H | H |
| 647 | 4-bromophenyl | H | H | H | H |
| 648 | 2-iodophenyl | H | H | H | H |
| 649 | 3-iodophenyl | H | H | H | H |
| 650 | 4-iodophenyl | H | H | H | H |
| 651 | 2-bromo-4-fluorophenyl | H | H | H | H |
| 652 | 2-bromo-4-chlorophenyl | H | H | H | H |
| 653 | 3-bromo-4-fluorophenyl | H | H | H | H |
| 654 | 3-bromo-4-chlorophenyl | H | H | H | H |
| 655 | 3-bromo-5-fluorophenyl | H | H | H | H |
| 656 | 3-bromo-5-chlorophenyl | H | H | H | H |
| 657 | 2-fluoro-4-bromophenyl | H | H | H | H |
| 658 | 2-chloro-4-bromophenyl | H | H | H | H |
| 659 | 3-fluoro-4-bromophenyl | H | H | H | H |
| 660 | 3-chloro-4-bromophenyl | H | H | H | H |
| 661 | 2-chloro-4-fluorophenyl | H | H | H | H |
| 662 | 3-chloro-4-fluorophenyl | H | H | H | H |
| 663 | 2-fluoro-3-chlorophenyl | H | H | H | H |
| 664 | 2-fluoro-4-chlorophenyl | H | H | H | H |
| 665 | 2-fluoro-5-chlorophenyl | H | H | H | H |
| 666 | 3-fluoro-4-chlorophenyl | H | H | H | H |
| 667 | 3-fluoro-5-chlorophenyl | H | H | H | H |
| 668 | 2-fluoro-6-chlorophenyl | H | H | H | H |
| 669 | 2-methylphenyl | H | H | H | H |
| 670 | 3-methylphenyl | H | H | H | H |
| 671 | 4-methylphenyl | H | H | H | H |
| 672 | 2,4-dimethylphenyl | H | H | H | H |
| 673 | 2,5-dimethylphenyl | H | H | H | H |
| 674 | 2,6-dimethylphenyl | H | H | H | H |
| 675 | 2,3-dimethylphenyl | H | H | H | H |
| 676 | 3,4-dimethylphenyl | H | H | H | H |
| 677 | 3,5-dimethylphenyl | H | H | H | H |
| 678 | 2,4,5-trimethylphenyl | H | H | H | H |
| 679 | 3,4,5-trimethylphenyl | H | H | H | H |
| 680 | 2,4,6-trimethylphenyl | H | H | H | H |
| 681 | 2-methoxyphenyl | H | H | H | H |
| 682 | 3-methoxyphenyl | H | H | H | H |
| 683 | 4-methoxyphenyl | H | H | H | H |
| 684 | 2,4-dimethoxyphenyl | H | H | H | H |
| 685 | 2,5-dimethoxyphenyl | H | H | H | H |
| 686 | 2,6-dimethoxyphenyl | H | H | H | H |
| 687 | 2,3-dimethoxyphenyl | H | H | H | H |
| 688 | 3,4-dimethoxyphenyl | H | H | H | H |
| 689 | 3,5-dimethoxyphenyl | H | H | H | H |
| 690 | 2,4,5-trimethoxyphenyl | H | H | H | H |
| 691 | 3,4,5-trimethoxyphenyl | H | H | H | H |
| 692 | 2,4,6-trimethoxyphenyl | H | H | H | H |
| 693 | 2-trifluoromethoxyphenyl | H | H | H | H |
| 694 | 3-trifluoromethoxyphenyl | H | H | H | H |
| 695 | 4-trifluoromethoxyphenyl | H | H | H | H |
| 696 | 2-difluoromethoxyphenyl | H | H | H | H |
| 697 | 3-difluoromethoxyphenyl | H | H | H | H |
| 698 | 4-difluoromethoxyphenyl | H | H | H | H |
| 699 | 2-trifluoromethylphenyl | H | H | H | H |
| 700 | 3-trifluoromethylphenyl | H | H | H | H |
| 701 | 4-trifluoromethylphenyl | H | H | H | H |
| 702 | 2-difluoromethylphenyl | H | H | H | H |
| 703 | 3-difluoromethylphenyl | H | H | H | H |
| 704 | 4-difluoromethylphenyl | H | H | H | H |
| 705 | 3,5-bis(trifluoromethyl)phenyl | H | H | H | H |
| 706 | 3-trifluoromethyl-5-fluorophenyl | H | H | H | H |
| 707 | 3-trifluoromethyl-5-chlorophenyl | H | H | H | H |
| 706 | 3-methyl-5-fluorophenyl | H | H | H | H |
| 707 | 3-methyl-5-chlorophenyl | H | H | H | H |
| 708 | 3-methoxy-5-fluorophenyl | H | H | H | H |
| 709 | 3-methoxy-5-chlorophenyl | H | H | H | H |
| 710 | 3-trifluoromethoxy-5-chlorophenyl | H | H | H | H |
| 711 | 2-ethoxyphenyl | H | H | H | H |
| 712 | 3-ethoxyphenyl | H | H | H | H |
| 713 | 4-ethoxyphenyl | H | H | H | H |
| 714 | 2-methylthiophenyl | H | H | H | H |
| 715 | 3-methylthiophenyl | H | H | H | H |
| 716 | 4-methylthiophenyl | H | H | H | H |
| 717 | 2-trifluoromethylthiophenyl | H | H | H | H |
| 718 | 3-trifluoromethylthiophenyl | H | H | H | H |
| 719 | 4-trifluoromethylthiophenyl | H | H | H | H |
| 720 | 2-cyanophenyl | H | H | H | H |
| 721 | 3-cyanophenyl | H | H | H | H |
| 722 | 4-cyanophenyl | H | H | H | H |
| 723 | 2-ethoxyphenyl | H | H | H | H |
| 724 | 3-ethylphenyl | H | H | H | H |
| 725 | 4-ethylphenyl | H | H | H | H |
| 726 | 2-methoxycarbonylphenyl | H | H | H | H |
| 727 | 3-methoxycarbonylphenyl | H | H | H | H |
| 728 | 4-methoxycarbonylphenyl | H | H | H | H |
| 729 | 2-ethoxycarbonylphenyl | H | H | H | H |
| 730 | 3-ethoxycarbonylphenyl | H | H | H | H |
| 731 | 4-ethoxycarbonylphenyl | H | H | H | H |
| 732 | pyridin-2-yl | H | H | H | H |
| 733 | pyridin-3-yl | H | H | H | H |
| 734 | pyridin-4-yl | H | H | H | H |
| 735 | pyrazin-2-yl | H | H | H | H |
| 736 | pyridazin-3-yl | H | H | H | H |
| 737 | pyridazin-4-yl | H | H | H | H |
| 738 | pyrimidin-2-yl | H | H | H | H |
| 739 | pyrimidin-5-yl | H | H | H | H |
| 740 | pyrimidin-4-yl | H | H | H | H |
| 741 | pyridazin-3-ylmethyl | H | H | H | H |
| 742 | pyridazin-4-ylmethyl | H | H | H | H |
| 743 | pyrimidin-2-ylmethyl | H | H | H | H |
| 744 | pyrimidin-5-ylmethyl | H | H | H | H |
| 745 | pyrimidin-4-ylmethyl | H | H | H | H |
| 746 | pyrazin-2-ylmethyl | H | H | H | H |
| 747 | 3-chloropyrazin-2-yl | H | H | H | H |
| 748 | 3-bromopyrazin-2-yl | H | H | H | H |
| 749 | 3-methoxypyrazin-2-yl | H | H | H | H |
| 750 | 3-ethoxypyrazin-2-yl | H | H | H | H |
| 751 | 3-trifluoromethylpyrazin-2-yl | H | H | H | H |
| 752 | 3-cyanopyrazin-2-yl | H | H | H | H |
| 753 | naphth-2-yl | H | H | H | H |
| 754 | naphth-1-yl | H | H | H | H |
| 755 | quinolin-4-yl | H | H | H | H |
| 756 | quinolin-6-yl | H | H | H | H |
| 757 | quinolin-8-yl | H | H | H | H |
| 758 | quinolin-2-yl | H | H | H | H |
| 759 | quinoxalin-2-yl | H | H | H | H |
| 760 | 2-naphthylmethyl | H | H | H | H |
| 761 | 1-naphthylmethyl | H | H | H | H |
| 762 | quinolin-4-ylmethyl | H | H | H | H |
| 763 | quinolin-6-ylmethyl | H | H | H | H |
| 764 | quinolin-8-ylmethyl | H | H | H | H |
| 765 | quinolin-2-ylmethyl | H | H | H | H |
| 766 | quinoxalin-2-ylmethyl | H | H | H | H |
| 767 | pyrazin-2-ylmethyl | H | H | H | H |
| 768 | 4-chloropyridin-2-yl | H | H | H | H |
| 769 | 3-chloropyridin-4-yl | H | H | H | H |
| 770 | 2-chloropyridin-3-yl | H | H | H | H |
| 771 | 2-chloropyridin-4-yl | H | H | H | H |
| 772 | 2-chloropyridin-5-yl | H | H | H | H |
| 773 | 2,6-dichloropyridin-4-yl | H | H | H | H |
| 774 | 3-chloropyridin-5-yl | H | H | H | H |
| 775 | 3,5-dichloropyridin-2-yl | H | H | H | H |
| 776 | 3-chloro-5-trifluoromethylpyridin-2- | H | H | H | H |
| | yl | | | | |
| 777 | (4-chloropyridin-2-yl)methyl | H | H | H | H |
| 778 | (3-chloropyridin-4-yl)methyl | H | H | H | H |
| 779 | (2-chloropyridin-3-yl)methyl | H | H | H | H |
| 780 | (2-chloropyridin-4-yl)methyl | H | H | H | H |
| 781 | (2-chloropyridin-5-yl)methyl | H | H | H | H |
| 782 | (2,6-dichloropyridin-4-yl)methyl | H | H | H | H |
| 783 | (3-chloropyridin-5-yl)methyl | H | H | H | H |
| 784 | (3,5-dichloropyridin-2-yl)methyl | H | H | H | H |
| 785 | thiophen-2-yl | H | H | H | H |
| 786 | thiophen-3-yl | H | H | H | H |
| 787 | 5-methylthiophen-2-yl | H | H | H | H |
| 788 | 5-ethylthiophen-2-yl | H | H | H | H |
| 789 | 5-chlorothiophen-2-yl | H | H | H | H |
| 790 | 5-bromothiophen-2-yl | H | H | H | H |
| 791 | 4-methylthiophen-2-yl | H | H | H | H |
| 792 | 3-methylthiophen-2-yl | H | H | H | H |
| 793 | 5-fluorothiophen-3-yl | H | H | H | H |
| 794 | 3,5-dimethylthiophen-2-yl | H | H | H | H |
| 795 | 3-ethylthiophen-2-yl | H | H | H | H |
| 796 | 4,5-dimethylthiophen-2-yl | H | H | H | H |
| 797 | 3,4-dimethylthiophen-2-yl | H | H | H | H |
| 798 | 4-chlorothiophen-2-yl | H | H | H | H |
| 799 | furan-2-yl | H | H | H | H |
| 800 | 5-methylfuran-2-yl | H | H | H | H |
| 801 | 5-ethylfuran-2-yl | H | H | H | H |
| 802 | 5-methoxycarbonylfuran-2-yl | H | H | H | H |
| 803 | 5-chlorofuran-2-yl | H | H | H | H |
| 804 | 5-bromofuran-2-yl | H | H | H | H |
| 805 | 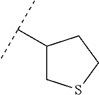 | H | H | H | H |
| 806 | 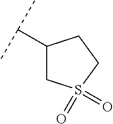 | H | H | H | H |
| 807 | 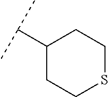 | H | H | H | H |
| 808 | 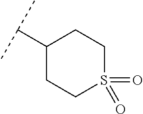 | H | H | H | H |
| 809 | 1-(4-methylphenyl)ethyl | H | H | H | H |
| 810 | 1-(3-methylphenyl)ethyl | H | H | H | H |
| 811 | 1-(2-methylphenyl)ethyl | H | H | H | H |
| 812 | 1-(4-chlorophenyl)ethyl | H | H | H | H |
| 813 | 1-(3-chlorophenyl)ethyl | H | H | H | H |
| 814 | 1-(2-chlorophenyl)ethyl | H | H | H | H |
| 815 | 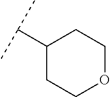 | H | H | H | H |
| 816 | 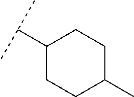 | H | H | H | H |
| 817 | benzyl | H | H | H | H |
| 818 | (4-fluorophenyl)methyl | H | H | H | H |
| 819 | (3-fluorophenyl)methyl | H | H | H | H |
| 820 | (2-fluorophenyl)methyl | H | H | H | H |
| 821 | (2,4-difluorophenyl)methyl | H | H | H | H |
| 822 | (3,5-difluorophenyl)methyl | H | H | H | H |
| 823 | (2,5-difluorophenyl)methyl | H | H | H | H |
| 824 | (2,6-difluorophenyl)methyl | H | H | H | H |
| 825 | (2,4,5-trifluorophenyl)methyl | H | H | H | H |
| 826 | (2,4,6-trifluorophenyl)methyl | H | H | H | H |
| 827 | (4-chlorophenyl)methyl | H | H | H | H |
| 828 | (3-chlorophenyl)methyl | H | H | H | H |
| 829 | (2-chlorophenyl)methyl | H | H | H | H |
| 830 | (2,4-dichlorophenyl)methyl | H | H | H | H |
| 831 | (3,5-dichlorophenyl)methyl | H | H | H | H |
| 832 | (2,5-dichlorophenyl)methyl | H | H | H | H |
| 833 | (2,6-dichlorophenyl)methyl | H | H | H | H |
| 834 | (2,4,5-trichlorophenyl)methyl | H | H | H | H |
| 835 | (2,4,6-trichlorophenyl)methyl | H | H | H | H |
| 836 | (4-bromophenyl)methyl | H | H | H | H |
| 837 | (3-bromophenyl)methyl | H | H | H | H |
| 838 | (2-bromophenyl)methyl | H | H | H | H |
| 839 | (4-iodophenyl)methyl | H | H | H | H |
| 840 | (3-iodophenyl)methyl | H | H | H | H |
| 841 | (2-iodophenyl)methyl | H | H | H | H |
| 842 | (3-chloro-5-trifluoromethylpyridin-2- | H | H | H | H |
| | yl)methyl | | | | |
| 843 | (2-bromo-4-fluorophenyl)methyl | H | H | H | H |
| 844 | (2-bromo-4-chlorophenyl)methyl | H | H | H | H |
| 845 | (3-bromo-4-fluorophenyl)methyl | H | H | H | H |
| 846 | (3-bromo-4-chlorophenyl)methyl | H | H | H | H |
| 847 | (3-bromo-5-fluorophenyl)methyl | H | H | H | H |
| 848 | (3-bromo-5-chlorophenyl)methyl | H | H | H | H |
| 849 | (2-fluoro-4-bromophenyl)methyl | H | H | H | H |
| 850 | (2-chloro-4-bromophenyl)methyl | H | H | H | H |
| 851 | (3-fluoro-4-bromophenyl)methyl | H | H | H | H |
| 852 | (3-chloro-4-bromophenyl)methyl | H | H | H | H |
| 853 | (2-chloro-4-fluorophenyl)methyl | H | H | H | H |
| 854 | (3-chloro-4-fluorophenyl)methyl | H | H | H | H |
| 855 | (2-fluoro-3-chlorophenyl)methyl | H | H | H | H |
| 856 | (2-fluoro-4-chlorophenyl)methyl | H | H | H | H |
| 857 | (2-fluoro-5-chlorophenyl)methyl | H | H | H | H |
| 858 | (3-fluoro-4-chlorophenyl)methyl | H | H | H | H |
| 859 | (3-fluoro-5-chlorophenyl)methyl | H | H | H | H |
| 860 | (2-fluoro-6-chlorophenyl)methyl | H | H | H | H |
| 861 | phenylethyl | H | H | H | H |
| 862 | 3-trifluoromethyl-4-chlorophenyl | H | H | H | H |
| 863 | 3-chloro-4-trifluoromethylphenyl | H | H | H | H |
| 864 | 2-chloro-4-trifluoromethylphenyl | H | H | H | H |
| 865 | 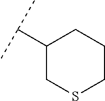 | H | H | H | H |
| 866 | 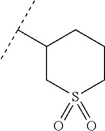 | H | H | H | H |
| 867 | (3,5-dichloropyridin-2-yl)methyl | H | H | H | H |
| 868 | (4-trifluoromethylphenyl)methyl | H | H | H | H |
| 869 | (3-trifluoromethylphenyl)methyl | H | H | H | H |
| 870 | (2-trifluoromethylphenyl)methyl | H | H | H | H |
| 871 | (4-trifluoromethoxyphenyl)methyl | H | H | H | H |
| 872 | (3-trifluoromethoxyphenyl)methyl | H | H | H | H |
| 873 | (2-trifluoromethoxyphenyl)methyl | H | H | H | H |
| 874 | (4-methoxyphenyl)methyl | H | H | H | H |
| 875 | (3-methoxyphenyl)methyl | H | H | H | H |
| 876 | (2-methoxyphenyl)methyl | H | H | H | H |
| 877 | (4-methylphenyl)methyl | H | H | H | H |
| 878 | (3-methylphenyl)methyl | H | H | H | H |
| 879 | (2-methylphenyl)methyl | H | H | H | H |
| 880 | (4-cyanophenyl)methyl | H | H | H | H |
| 881 | (3-cyanophenyl)methyl | H | H | H | H |
| 882 | (2-cyanophenyl)methyl | H | H | H | H |
| 883 | (2,4-diethylphenyl)methyl | H | H | H | H |
| 884 | (3,5-diethylphenyl)methyl | H | H | H | H |
| 885 | (3,4-dimethylphenyl)methyl | H | H | H | H |
| 886 | (3,5-dimethoxyphenyl)methyl | H | H | H | H |
| 887 | 1-phenyleth-1-yl | H | H | H | H |
| 888 | 1-methylprop-1-yl | H | H | H | H |
| 889 | 2-methylprop-1-yl | H | H | H | H |
| 890 | 1-ethylprop-1-yl | H | H | H | H |
| 891 | 1-methylbut-1-yl | H | H | H | H |
| 892 | 2-methylbut-1-yl | H | H | H | H |
| 893 | 3-methylbut-1-yl | H | H | H | H |
| 894 | 2,2-difluoroethyl | H | H | H | H |
| 895 | 2,2,2-trifluoroethyl | H | H | H | H |
| 896 | 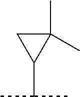 | H | H | H | H |
| 897 | 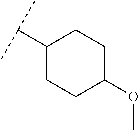 | H | H | H | H |
| 898 | 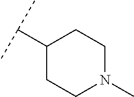 | H | H | H | H |
| 899 | 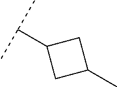 | H | H | H | H |
| 900 | 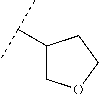 | H | H | H | H |
| 901 | CH₃ | CO₂CH₃ | CH₃ | H | H |
| 902 | Et | CO₂CH₃ | CH₃ | H | H |
| 903 | n-Pr | CO₂CH₃ | CH₃ | H | H |
| 904 | i-Pr | CO₂CH₃ | CH₃ | H | H |
| 905 | n-Bu | CO₂CH₃ | CH₃ | H | H |
| 906 | neopentyl | CO₂CH₃ | CH₃ | H | H |
| 907 | t-Bu | CO₂CH₃ | CH₃ | H | H |
| 908 | n-pentyl | CO₂CH₃ | CH₃ | H | H |
| 909 | n-hexyl | CO₂CH₃ | CH₃ | H | H |
| 910 | c-Pr | CO₂CH₃ | CH₃ | H | H |
| 911 | c-Bu | CO₂CH₃ | CH₃ | H | H |
| 912 | c-pentyl | CO₂CH₃ | CH₃ | H | H |
| 913 | c-hexyl | CO₂CH₃ | CH₃ | H | H |
| 914 | phenyl | CO₂CH₃ | CH₃ | H | H |
| 915 | 2-fluorophenyl | CO₂CH₃ | CH₃ | H | H |
| 916 | 3-fluorophenyl | CO₂CH₃ | CH₃ | H | H |
| 917 | 4-fluorophenyl | CO₂CH₃ | CH₃ | H | H |
| 918 | 2,4-difluorophenyl | CO₂CH₃ | CH₃ | H | H |
| 919 | 2,5-difluorophenyl | CO₂CH₃ | CH₃ | H | H |
| 920 | 2,6-difluorophenyl | CO₂CH₃ | CH₃ | H | H |
| 921 | 2,3-difluorophenyl | CO₂CH₃ | CH₃ | H | H |
| 922 | 3,4-difluorophenyl | CO₂CH₃ | CH₃ | H | H |
| 923 | 3,5-difluorophenyl | CO₂CH₃ | CH₃ | H | H |
| 924 | 2,4,5-trifluorophenyl | CO₂CH₃ | CH₃ | H | H |
| 925 | 3,4,5-trifluorophenyl | CO₂CH₃ | CH₃ | H | H |
| 926 | 2-chlorophenyl | CO₂CH₃ | CH₃ | H | H |
| 927 | 3-chlorophenyl | CO₂CH₃ | CH₃ | H | H |
| 928 | 4-chlorophenyl | CO₂CH₃ | CH₃ | H | H |
| 929 | 2,4-dichlorophenyl | CO₂CH₃ | CH₃ | H | H |
| 930 | 2,5-dichlorophenyl | CO₂CH₃ | CH₃ | H | H |
| 931 | 2,6-dichlorophenyl | CO₂CH₃ | CH₃ | H | H |
| 932 | 2,3-dichlorophenyl | CO₂CH₃ | CH₃ | H | H |
| 933 | 3,4-dichlorophenyl | CO₂CH₃ | CH₃ | H | H |
| 934 | 3,5-dichlorophenyl | CO₂CH₃ | CH₃ | H | H |
| 935 | 2,4,5-trichlorophenyl | CO₂CH₃ | CH₃ | H | H |
| 936 | 3,4,5-trichlorophenyl | CO₂CH₃ | CH₃ | H | H |
| 937 | 2,4,6-trichlorophenyl | CO₂CH₃ | CH₃ | H | H |
| 938 | 2-bromophenyl | CO₂CH₃ | CH₃ | H | H |
| 939 | 3-bromophenyl | CO₂CH₃ | CH₃ | H | H |
| 940 | 4-bromophenyl | CO₂CH₃ | CH₃ | H | H |
| 941 | 2-iodophenyl | CO₂CH₃ | CH₃ | H | H |
| 942 | 3-iodophenyl | CO₂CH₃ | CH₃ | H | H |
| 943 | 4-iodophenyl | CO₂CH₃ | CH₃ | H | H |
| 944 | 2-bromo-4-fluorophenyl | CO₂CH₃ | CH₃ | H | H |
| 945 | 2-bromo-4-chlorophenyl | CO₂CH₃ | CH₃ | H | H |
| 946 | 3-bromo-4-fluorophenyl | CO₂CH₃ | CH₃ | H | H |
| 947 | 3-bromo-4-chlorophenyl | CO₂CH₃ | CH₃ | H | H |
| 948 | 3-bromo-5-fluorophenyl | CO₂CH₃ | CH₃ | H | H |
| 949 | 3-bromo-5-chlorophenyl | CO₂CH₃ | CH₃ | H | H |
| 950 | 2-fluoro-4-bromophenyl | CO₂CH₃ | CH₃ | H | H |
| 951 | 2-chloro-4-bromophenyl | CO₂CH₃ | CH₃ | H | H |
| 952 | 3-fluoro-4-bromophenyl | CO₂CH₃ | CH₃ | H | H |
| 953 | 3-chloro-4-bromophenyl | CO₂CH₃ | CH₃ | H | H |
| 954 | 2-chloro-4-fluorophenyl | CO₂CH₃ | CH₃ | H | H |
| 955 | 3-chloro-4-fluorophenyl | CO₂CH₃ | CH₃ | H | H |
| 956 | 2-fluoro-3-chlorophenyl | CO₂CH₃ | CH₃ | H | H |
| 957 | 2-fluoro-4-chlorophenyl | CO₂CH₃ | CH₃ | H | H |
| 958 | 2-fluoro-5-chlorophenyl | CO₂CH₃ | CH₃ | H | H |
| 959 | 3-fluoro-4-chlorophenyl | CO₂CH₃ | CH₃ | H | H |
| 960 | 3-fluoro-5-chlorophenyl | CO₂CH₃ | CH₃ | H | H |
| 961 | 2-fluoro-6-chlorophenyl | CO₂CH₃ | CH₃ | H | H |
| 962 | 2-methylphenyl | CO₂CH₃ | CH₃ | H | H |
| 963 | 3-methylphenyl | CO₂CH₃ | CH₃ | H | H |
| 964 | 4-methylphenyl | CO₂CH₃ | CH₃ | H | H |
| 965 | 2,4-dimethylphenyl | CO₂CH₃ | CH₃ | H | H |
| 966 | 2,5-dimethylphenyl | CO₂CH₃ | CH₃ | H | H |
| 967 | 2,6-dimethylphenyl | CO₂CH₃ | CH₃ | H | H |
| 968 | 2,3-dimethylphenyl | CO₂CH₃ | CH₃ | H | H |
| 969 | 3,4-dimethylphenyl | CO₂CH₃ | CH₃ | H | H |
| 970 | 3,5-dimethylphenyl | CO₂CH₃ | CH₃ | H | H |
| 971 | 2,4,5-trimethylphenyl | CO₂CH₃ | CH₃ | H | H |
| 972 | 3,4,5-trimethylphenyl | CO₂CH₃ | CH₃ | H | H |
| 973 | 2,4,6-trimethylphenyl | CO₂CH₃ | CH₃ | H | H |
| 974 | 2-methoxyphenyl | CO₂CH₃ | CH₃ | H | H |
| 975 | 3-methoxyphenyl | CO₂CH₃ | CH₃ | H | H |
| 976 | 4-methoxyphenyl | CO₂CH₃ | CH₃ | H | H |
| 977 | 2-trifluoromethoxyphenyl | CO₂CH₃ | CH₃ | H | H |
| 978 | 3-trifluoromethoxyphenyl | CO₂CH₃ | CH₃ | H | H |
| 979 | 4-trifluoromethoxyphenyl | CO₂CH₃ | CH₃ | H | H |
| 980 | 2-difluoromethoxyphenyl | CO₂CH₃ | CH₃ | H | H |
| 981 | 3-difluoromethoxyphenyl | CO₂CH₃ | CH₃ | H | H |
| 982 | 4-difluoromethoxyphenyl | CO₂CH₃ | CH₃ | H | H |
| 983 | 2-trifluoromethylphenyl | CO₂CH₃ | CH₃ | H | H |
| 984 | 3-trifluoromethylphenyl | CO₂CH₃ | CH₃ | H | H |
| 985 | 4-trifluoromethylphenyl | CO₂CH₃ | CH₃ | H | H |
| 986 | 2-difluoromethylphenyl | CO₂CH₃ | CH₃ | H | H |
| 987 | 3-difluoromethylphenyl | CO₂CH₃ | CH₃ | H | H |
| 988 | 4-difluoromethylphenyl | CO₂CH₃ | CH₃ | H | H |
| 989 | 3,5-bis(trifluoromethyl)phenyl | CO₂CH₃ | CH₃ | H | H |
| 990 | 3-trifluoromethyl-5-fluorophenyl | CO₂CH₃ | CH₃ | H | H |
| 991 | 3-trifluoromethyl-5-chlorophenyl | CO₂CH₃ | CH₃ | H | H |
| 992 | 3-methyl-5-fluorophenyl | CO₂CH₃ | CH₃ | H | H |
| 993 | 3-methyl-5-chlorophenyl | CO₂CH₃ | CH₃ | H | H |
| 994 | 3-methoxy-5-fluorophenyl | CO₂CH₃ | CH₃ | H | H |
| 995 | 3-methoxy-5-chlorophenyl | CO₂CH₃ | CH₃ | H | H |
| 996 | 3-trifluoromethoxy-5-chlorophenyl | CO₂CH₃ | CH₃ | H | H |
| 997 | 2-ethoxyphenyl | CO₂CH₃ | CH₃ | H | H |
| 998 | 3-ethoxyphenyl | CO₂CH₃ | CH₃ | H | H |
| 999 | 4-ethoxyphenyl | CO₂CH₃ | CH₃ | H | H |
| 1000 | 2-trifluoromethylthiophenyl | CO₂CH₃ | CH₃ | H | H |
| 1001 | 3-trifluoromethylthiophenyl | CO₂CH₃ | CH₃ | H | H |
| 1002 | 4-trifluoromethylthiophenyl | CO₂CH₃ | CH₃ | H | H |
| 1003 | pyridin-2-yl | CO₂CH₃ | CH₃ | H | H |
| 1004 | pyridin-3-yl | CO₂CH₃ | CH₃ | H | H |
| 1005 | pyridin-4-yl | CO₂CH₃ | CH₃ | H | H |
| 1006 | pyrazin-2-yl | CO₂CH₃ | CH₃ | H | H |
| 1007 | pyridazin-3-yl | CO₂CH₃ | CH₃ | H | H |
| 1008 | pyridazin-4-yl | CO₂CH₃ | CH₃ | H | H |
| 1009 | pyrimidin-2-yl | CO₂CH₃ | CH₃ | H | H |
| 1010 | pyrimidin-5-yl | CO₂CH₃ | CH₃ | H | H |
| 1011 | pyrimidin-4-yl | CO₂CH₃ | CH₃ | H | H |
| 1012 | 3-chloropyrazin-2-yl | CO₂CH₃ | CH₃ | H | H |
| 1013 | 3-bromopyrazin-2-yl | CO₂CH₃ | CH₃ | H | H |
| 1014 | 3-methoxypyrazin-2-yl | CO₂CH₃ | CH₃ | H | H |
| 1015 | 3-ethoxypyrazin-2-yl | CO₂CH₃ | CH₃ | H | H |
| 1016 | 3-trifluoromethylpyrazin-2-yl | CO₂CH₃ | CH₃ | H | H |
| 1017 | 3-cyanopyrazin-2-yl | CO₂CH₃ | CH₃ | H | H |
| 1018 | naphth-2-yl | CO₂CH₃ | CH₃ | H | H |
| 1019 | naphth-1-yl | CO₂CH₃ | CH₃ | H | H |
| 1020 | quinolin-4-yl | CO₂CH₃ | CH₃ | H | H |
| 1021 | quinolin-6-yl | CO₂CH₃ | CH₃ | H | H |
| 1022 | quinolin-8-yl | CO₂CH₃ | CH₃ | H | H |
| 1023 | quinolin-2-yl | CO₂CH₃ | CH₃ | H | H |
| 1024 | quinoxalin-2-yl | CO₂CH₃ | CH₃ | H | H |
| 1025 | 4-chloropyridin-2-yl | CO₂CH₃ | CH₃ | H | H |
| 1026 | 3-chloropyridin-4-yl | CO₂CH₃ | CH₃ | H | H |
| 1027 | 2-chloropyridin-3-yl | CO₂CH₃ | CH₃ | H | H |
| 1028 | 2-chloropyridin-4-yl | CO₂CH₃ | CH₃ | H | H |
| 1029 | 2-chloropyridin-5-yl | CO₂CH₃ | CH₃ | H | H |
| 1030 | 2,6-dichloropyridin-4-yl | CO₂CH₃ | CH₃ | H | H |
| 1031 | 3-chloropyridin-5-yl | CO₂CH₃ | CH₃ | H | H |
| 1032 | 3,5-dichloropyridin-2-yl | CO₂CH₃ | CH₃ | H | H |
| 1033 | 3-chloro-5-trifluoromethylpyridin-2- | CO₂CH₃ | CH₃ | H | H |
| | yl | | | | |
| 1034 | thiophen-2-yl | CO₂CH₃ | CH₃ | H | H |
| 1035 | thiophen-3-yl | CO₂CH₃ | CH₃ | H | H |
| 1036 | 5-methylthiophen-2-yl | CO₂CH₃ | CH₃ | H | H |
| 1037 | 5-ethylthiophen-2-yl | CO₂CH₃ | CH₃ | H | H |
| 1038 | 5-chlorothiophen-2-yl | CO₂CH₃ | CH₃ | H | H |
| 1039 | 5-bromothiophen-2-yl | CO₂CH₃ | CH₃ | H | H |
| 1040 | 4-methylthiophen-2-yl | CO₂CH₃ | CH₃ | H | H |
| 1041 | 3-methylthiophen-2-yl | CO₂CH₃ | CH₃ | H | H |
| 1042 | 4-chlorothiophen-2-yl | CO₂CH₃ | CH₃ | H | H |
| 1043 | furan-2-yl | CO₂CH₃ | CH₃ | H | H |
| 1044 | 5-methylfuran-2-yl | CO₂CH₃ | CH₃ | H | H |
| 1045 | 5-ethylfuran-2-yl | CO₂CH₃ | CH₃ | H | H |
| 1046 | H | 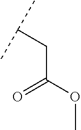 | CH₃ | H | H |
| 1047 | CH₃ | 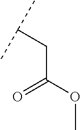 | CH₃ | H | H |
| 1048 | Et | 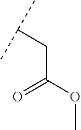 | CH₃ | H | H |
| 1049 | n-Pr | 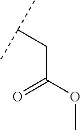 | CH₃ | H | H |
| 1050 | i-Pr | 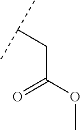 | CH₃ | H | H |
| 1051 | n-Bu | 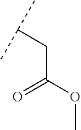 | CH₃ | H | H |
| 1052 | neopentyl | 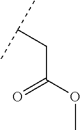 | CH₃ | H | H |
| 1053 | t-Bu | 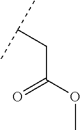 | CH₃ | H | H |
| 1054 | n-pentyl | 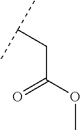 | CH₃ | H | H |
| 1055 | n-hexyl | 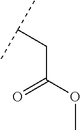 | CH₃ | H | H |
| 1056 | c-Pr | 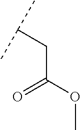 | CH₃ | H | H |
| 1057 | c-Bu | 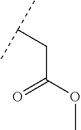 | CH₃ | H | H |
| 1058 | c-pentyl | 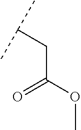 | CH₃ | H | H |
| 1059 | c-hexyl | 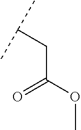 | CH₃ | H | H |
| 1060 | phenyl | 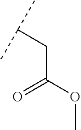 | CH₃ | H | H |
| 1061 | benzyl | 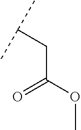 | CH₃ | H | H |
| 1062 | 2-chlorophenyl | 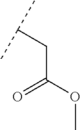 | CH₃ | H | H |
| 1063 | 3-chlorophenyl | 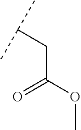 | CH₃ | H | H |
| 1064 | 4-chlorophenyl | 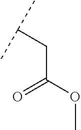 | CH₃ | H | H |
| 1065 | 2-fluorophenyl | 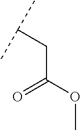 | CH₃ | H | H |
| 1066 | 3-fluorophenyl | 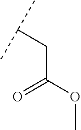 | CH₃ | H | H |
| 1067 | 4-fluorophenyl | 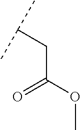 | CH₃ | H | H |
| 1068 | pyridin-2-yl | 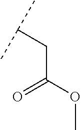 | CH₃ | H | H |
| 1069 | pyridin-3-yl | 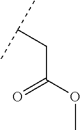 | CH₃ | H | H |
| 1070 | 4-trifluoromethylpyridin-2-yl | 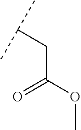 | CH₃ | H | H |
| 1071 | 4-trifluoromethylpyridin-2-yl | CH₃ | CH₃ | H | H |
| 1072 | H | 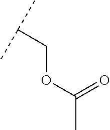 | CH₃ | H | H |
| 1073 | CH₃ | 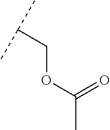 | CH₃ | H | H |
| 1074 | Et | 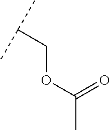 | CH₃ | H | H |
| 1075 | n-Pr | 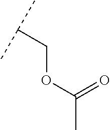 | CH₃ | H | H |
| 1076 | i-Pr | 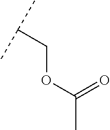 | CH₃ | H | H |
| 1077 | n-Bu | 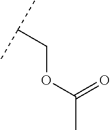 | CH₃ | H | H |
| 1078 | neopentyl | 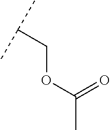 | CH₃ | H | H |
| 1079 | t-Bu | 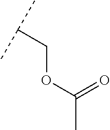 | CH₃ | H | H |
| 1080 | n-pentyl | 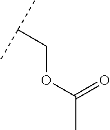 | CH₃ | H | H |
| 1081 | n-hexyl | 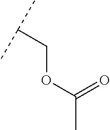 | CH₃ | H | H |
| 1082 | c-Pr | 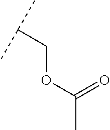 | CH₃ | H | H |
| 1083 | c-Bu | 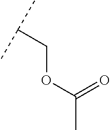 | CH₃ | H | H |
| 1084 | c-pentyl | 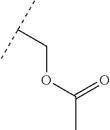 | CH₃ | H | H |
| 1085 | c-hexyl | 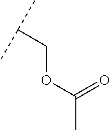 | CH₃ | H | H |
| 1086 | phenyl | 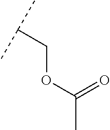 | CH₃ | H | H |
| 1087 | benzyl | 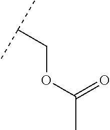 | CH₃ | H | H |
| 1088 | 2-chlorophenyl | 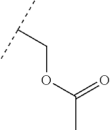 | CH₃ | H | H |
| 1089 | 3-chlorophenyl | 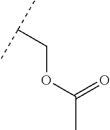 | CH₃ | H | H |
| 1090 | 4-chlorophenyl | 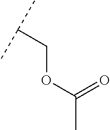 | CH₃ | H | H |
| 1091 | 2-fluorophenyl | 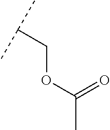 | CH₃ | H | H |
| 1092 | 3-fluorophenyl | 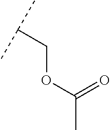 | CH₃ | H | H |
| 1093 | 4-fluorophenyl | 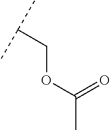 | CH₃ | H | H |
| 1094 | pyridin-2-yl | 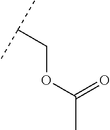 | CH₃ | H | H |
| 1095 | pyridin-3-yl | 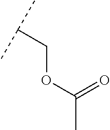 | CH₃ | H | H |
| 1096 | 4-trifluoromethylpyridin-2-yl | 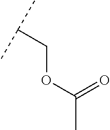 | CH₃ | H | H |
| 1122 | 4-trifluoromethylpyridin-2-yl | CH₃ | H | H | H |
| 1123 | 4-trifluoromethylpyridin-2-yl | H | H | H | H |
| 1124 | 4-trifluoromethylpyridin-2-yl | CO₂CH₃ | H | H | H |
| 1125 | H | Ph | H | H | H |
| 1126 | CH₃ | Ph | H | H | H |
| 1127 | Et | Ph | H | H | H |
| 1128 | n-Pr | Ph | H | H | H |
| 1129 | i-Pr | Ph | H | H | H |
| 1130 | n-Bu | Ph | H | H | H |
| 1131 | neopentyl | Ph | H | H | H |
| 1132 | t-Bu | Ph | H | H | H |
| 1133 | n-pentyl | Ph | H | H | H |
| 1134 | n-hexyl | Ph | H | H | H |
| 1135 | c-Pr | Ph | H | H | H |
| 1136 | c-Bu | Ph | H | H | H |
| 1137 | c-pentyl | Ph | H | H | H |
| 1138 | c-hexyl | Ph | H | H | H |
| 1139 | phenyl | Ph | H | H | H |
| 1140 | 2-fluorophenyl | Ph | H | H | H |
| 1141 | 3-fluorophenyl | Ph | H | H | H |
| 1142 | 4-fluorophenyl | Ph | H | H | H |
| 1143 | 2,4-difluorophenyl | Ph | H | H | H |
| 1144 | 2,5-difluorophenyl | Ph | H | H | H |
| 1145 | 2,6-difluorophenyl | Ph | H | H | H |
| 1146 | 2,3-difluorophenyl | Ph | H | H | H |
| 1147 | 3,4-difluorophenyl | Ph | H | H | H |
| 1148 | 3,5-difluorophenyl | Ph | H | H | H |
| 1149 | 2,4,5-trifluorophenyl | Ph | H | H | H |
| 1150 | 3,4,5-trifluorophenyl | Ph | H | H | H |
| 1151 | 2-chlorophenyl | Ph | H | H | H |
| 1152 | 3-chlorophenyl | Ph | H | H | H |
| 1153 | 4-chlorophenyl | Ph | H | H | H |
| 1154 | 2,4-dichlorophenyl | Ph | H | H | H |
| 1155 | 2,5-dichlorophenyl | Ph | H | H | H |
| 1156 | 2,6-dichlorophenyl | Ph | H | H | H |
| 1157 | 2,3-dichlorophenyl | Ph | H | H | H |
| 1158 | 3,4-dichlorophenyl | Ph | H | H | H |
| 1159 | 3,5-dichlorophenyl | Ph | H | H | H |
| 1160 | 2,4,5-trichlorophenyl | Ph | H | H | H |
| 1161 | 3,4,5-trichlorophenyl | Ph | H | H | H |
| 1162 | 2,4,6-trichlorophenyl | Ph | H | H | H |
| 1163 | 2-bromophenyl | Ph | H | H | H |
| 1164 | 3-bromophenyl | Ph | H | H | H |
| 1165 | 4-bromophenyl | Ph | H | H | H |
| 1166 | 2-iodophenyl | Ph | H | H | H |
| 1167 | 3-iodophenyl | Ph | H | H | H |
| 1168 | 4-iodophenyl | Ph | H | H | H |
| 1169 | 2-bromo-4-fluorophenyl | Ph | H | H | H |
| 1170 | 2-bromo-4-chlorophenyl | Ph | H | H | H |
| 1171 | 3-bromo-4-fluorophenyl | Ph | H | H | H |
| 1172 | 3-bromo-4-chlorophenyl | Ph | H | H | H |
| 1173 | 3-bromo-5-fluorophenyl | Ph | H | H | H |
| 1174 | 3-bromo-5-chlorophenyl | Ph | H | H | H |
| 1175 | 2-fluoro-4-bromophenyl | Ph | H | H | H |
| 1176 | 2-chloro-4-bromophenyl | Ph | H | H | H |
| 1177 | 3-fluoro-4-bromophenyl | Ph | H | H | H |
| 1178 | 3-chloro-4-bromophenyl | Ph | H | H | H |
| 1179 | 2-chloro-4-fluorophenyl | Ph | H | H | H |
| 1180 | 3-chloro-4-fluorophenyl | Ph | H | H | H |
| 1181 | 2-fluoro-3-chlorophenyl | Ph | H | H | H |
| 1182 | 2-fluoro-4-chlorophenyl | Ph | H | H | H |
| 1183 | 2-fluoro-5-chlorophenyl | Ph | H | H | H |
| 1184 | 3-fluoro-4-chlorophenyl | Ph | H | H | H |
| 1185 | 3-fluoro-5-chlorophenyl | Ph | H | H | H |
| 1186 | 2-fluoro-6-chlorophenyl | Ph | H | H | H |
| 1187 | 2-methylphenyl | Ph | H | H | H |
| 1188 | 3-methylphenyl | Ph | H | H | H |
| 1189 | 4-methylphenyl | Ph | H | H | H |
| 1190 | 2,4-dimethylphenyl | Ph | H | H | H |
| 1191 | 2,5-dimethylphenyl | Ph | H | H | H |
| 1192 | 2,6-dimethylphenyl | Ph | H | H | H |
| 1193 | 2,3-dimethylphenyl | Ph | H | H | H |
| 1194 | 3,4-dimethylphenyl | Ph | H | H | H |
| 1195 | 3,5-dimethylphenyl | Ph | H | H | H |
| 1196 | 2,4,5-trimethylphenyl | Ph | H | H | H |
| 1197 | 3,4,5-trimethylphenyl | Ph | H | H | H |
| 1198 | 2,4,6-trimethylphenyl | Ph | H | H | H |
| 1199 | 2-methoxyphenyl | Ph | H | H | H |
| 1200 | 3-methoxyphenyl | Ph | H | H | H |
| 1201 | 4-methoxyphenyl | Ph | H | H | H |
| 1202 | 2-trifluoromethoxyphenyl | Ph | H | H | H |
| 1203 | 3-trifluoromethoxyphenyl | Ph | H | H | H |
| 1204 | 4-trifluoromethoxyphenyl | Ph | H | H | H |
| 1205 | 2-difluoromethoxyphenyl | Ph | H | H | H |
| 1206 | 3-difluoromethoxyphenyl | Ph | H | H | H |
| 1207 | 4-difluoromethoxyphenyl | Ph | H | H | H |
| 1208 | 2-trifluoromethylphenyl | Ph | H | H | H |
| 1209 | 3-trifluoromethylphenyl | Ph | H | H | H |
| 1210 | 4-trifluoromethylphenyl | Ph | H | H | H |
| 1211 | 2-difluoromethylphenyl | Ph | H | H | H |
| 1212 | 3-difluoromethylphenyl | Ph | H | H | H |
| 1213 | 4-difluoromethylphenyl | Ph | H | H | H |
| 1214 | 3,5-bis(trifluoromethyl)phenyl | Ph | H | H | H |
| 1215 | 3-trifluoromethyl-5-fluorophenyl | Ph | H | H | H |
| 1216 | 3-trifluoromethyl-5-chlorophenyl | Ph | H | H | H |
| 1217 | 3-methyl-5-fluorophenyl | Ph | H | H | H |
| 1218 | 3-methyl-5-chlorophenyl | Ph | H | H | H |
| 1219 | 3-methoxy-5-fluorophenyl | Ph | H | H | H |
| 1220 | 3-methoxy-5-chlorophenyl | Ph | H | H | H |
| 1221 | 3-trifluoromethoxy-5-chlorophenyl | Ph | H | H | H |
| 1222 | 2-ethoxyphenyl | Ph | H | H | H |
| 1223 | 3-ethoxyphenyl | Ph | H | H | H |
| 1224 | 4-ethoxyphenyl | Ph | H | H | H |
| 1225 | 2-trifluoromethylthiophenyl | Ph | H | H | H |
| 1226 | 3-trifluoromethylthiophenyl | Ph | H | H | H |
| 1227 | 4-trifluoromethylthiophenyl | Ph | H | H | H |
| 1228 | pyridin-2-yl | Ph | H | H | H |
| 1229 | pyridin-3-yl | Ph | H | H | H |
| 1230 | pyridin-4-yl | Ph | H | H | H |
| 1231 | pyrazin-2-yl | Ph | H | H | H |
| 1232 | pyridazin-3-yl | Ph | H | H | H |
| 1233 | pyridazin-4-yl | Ph | H | H | H |
| 1234 | pyrimidin-2-yl | Ph | H | H | H |
| 1235 | pyrimidin-5-yl | Ph | H | H | H |
| 1236 | pyrimidin-4-yl | Ph | H | H | H |
| 1250 | H | CH₃ | CH₃ | CH₃ | H |
| 1251 | CH₃ | CH₃ | CH₃ | CH₃ | H |
| 1252 | Et | CH₃ | CH₃ | CH₃ | H |
| 1253 | n-Pr | CH₃ | CH₃ | CH₃ | H |
| 1254 | i-Pr | CH₃ | CH₃ | CH₃ | H |
| 1255 | n-Bu | CH₃ | CH₃ | CH₃ | H |
| 1256 | t-Bu | CH₃ | CH₃ | CH₃ | H |
| 1257 | n-pentyl | CH₃ | CH₃ | CH₃ | H |
| 1258 | n-hexyl | CH₃ | CH₃ | CH₃ | H |
| 1259 | c-Pr | CH₃ | CH₃ | CH₃ | H |
| 1260 | c-Bu | CH₃ | CH₃ | CH₃ | H |
| 1261 | c-pentyl | CH₃ | CH₃ | CH₃ | H |
| 1262 | c-hexyl | CH₃ | CH₃ | CH₃ | H |
| 1263 | phenyl | CH₃ | CH₃ | CH₃ | H |
| 1264 | 2-fluorophenyl | CH₃ | CH₃ | CH₃ | H |
| 1265 | 3-fluorophenyl | CH₃ | CH₃ | CH₃ | H |
| 1266 | 4-fluorophenyl | CH₃ | CH₃ | CH₃ | H |
| 1267 | 2,4-difluorophenyl | CH₃ | CH₃ | CH₃ | H |
| 1268 | 2,5-difluorophenyl | CH₃ | CH₃ | CH₃ | H |
| 1269 | 2,6-difluorophenyl | CH₃ | CH₃ | CH₃ | H |
| 1270 | 3,5-difluorophenyl | CH₃ | CH₃ | CH₃ | H |
| 1271 | 2-chlorophenyl | CH₃ | CH₃ | CH₃ | H |
| 1272 | 3-chlorophenyl | CH₃ | CH₃ | CH₃ | H |
| 1273 | 4-chlorophenyl | CH₃ | CH₃ | CH₃ | H |
| 1274 | 2,4-dichlorophenyl | CH₃ | CH₃ | CH₃ | H |
| 1275 | 2,5-dichlorophenyl | CH₃ | CH₃ | CH₃ | H |
| 1276 | 2,6-dichlorophenyl | CH₃ | CH₃ | CH₃ | H |
| 1277 | 3,5-dichlorophenyl | CH₃ | CH₃ | CH₃ | H |
| 1278 | 4-bromophenyl | CH₃ | CH₃ | CH₃ | H |
| 1279 | 4-iodophenyl | CH₃ | CH₃ | CH₃ | H |
| 1280 | 4-methylphenyl | CH₃ | CH₃ | CH₃ | H |
| 1281 | 4-methoxyphenyl | CH₃ | CH₃ | CH₃ | H |
| 1282 | 4-trifluoromethoxyphenyl | CH₃ | CH₃ | CH₃ | H |
| 1283 | 2-trifluoromethylphenyl | CH₃ | CH₃ | CH₃ | H |
| 1284 | 3-trifluoromethylphenyl | CH₃ | CH₃ | CH₃ | H |
| 1285 | 4-trifluoromethylphenyl | CH₃ | CH₃ | CH₃ | H |
| 1286 | pyridin-2-yl | CH₃ | CH₃ | CH₃ | H |
| 1287 | pyridin-3-yl | CH₃ | CH₃ | CH₃ | H |
| 1288 | pyridin-4-yl | CH₃ | CH₃ | CH₃ | H |
| 1289 | pyrazin-2-yl | CH₃ | CH₃ | CH₃ | H |
| 1290 | pyrimidin-2-yl | CH₃ | CH₃ | CH₃ | H |
| 1291 | pyrimidin-5-yl | CH₃ | CH₃ | CH₃ | H |
| 1292 | pyrimidin-4-yl | CH₃ | CH₃ | CH₃ | H |
| 1293 | 4-trifluoromethylpyridin-2-yl | CH₃ | CH₃ | CH₃ | H |
| 1294 | 4-chloropyridin-2-yl | CH₃ | CH₃ | CH₃ | H |
| 1295 | 3-chloropyridin-4-yl | CH₃ | CH₃ | CH₃ | H |
| 1296 | 2-chloropyridin-3-yl | CH₃ | CH₃ | CH₃ | H |
| 1297 | 2-chloropyridin-4-yl | CH₃ | CH₃ | CH₃ | H |
| 1298 | 2-chloropyridin-5-yl | CH₃ | CH₃ | CH₃ | H |
| 1299 | 3-chloro-5-trifluoromethylpyridin-2- | CH₃ | CH₃ | CH₃ | H |
| | yl | | | | |
| 1300 | H | pyridin-2-yl | H | H | H |
| 1301 | CH₃ | pyridin-2-yl | H | H | H |
| 1302 | Et | pyridin-2-yl | H | H | H |
| 1303 | n-Pr | pyridin-2-yl | H | H | H |
| 1304 | i-Pr | pyridin-2-yl | H | H | H |
| 1305 | n-Bu | pyridin-2-yl | H | H | H |
| 1306 | t-Bu | pyridin-2-yl | H | H | H |
| 1307 | n-pentyl | pyridin-2-yl | H | H | H |
| 1308 | n-hexyl | pyridin-2-yl | H | H | H |
| 1309 | c-Pr | pyridin-2-yl | H | H | H |
| 1310 | c-Bu | pyridin-2-yl | H | H | H |
| 1311 | c-pentyl | pyridin-2-yl | H | H | H |
| 1312 | c-hexyl | pyridin-2-yl | H | H | H |
| 1313 | phenyl | pyridin-2-yl | H | H | H |
| 1314 | 2-fluorophenyl | pyridin-2-yl | H | H | H |
| 1315 | 3-fluorophenyl | pyridin-2-yl | H | H | H |
| 1316 | 4-fluorophenyl | pyridin-2-yl | H | H | H |
| 1317 | 2,4-difluorophenyl | pyridin-2-yl | H | H | H |
| 1318 | 2,5-difluorophenyl | pyridin-2-yl | H | H | H |
| 1319 | 2,6-difluorophenyl | pyridin-2-yl | H | H | H |
| 1320 | 3,5-difluorophenyl | pyridin-2-yl | H | H | H |
| 1321 | 2-chlorophenyl | pyridin-2-yl | H | H | H |
| 1322 | 3-chlorophenyl | pyridin-2-yl | H | H | H |
| 1323 | 4-chlorophenyl | pyridin-2-yl | H | H | H |
| 1324 | 2,4-dichlorophenyl | pyridin-2-yl | H | H | H |
| 1325 | 2,5-dichlorophenyl | pyridin-2-yl | H | H | H |
| 1326 | 2,6-dichlorophenyl | pyridin-2-yl | H | H | H |
| 1327 | 3,5-dichlorophenyl | pyridin-2-yl | H | H | H |
| 1328 | 4-bromophenyl | pyridin-2-yl | H | H | H |
| 1329 | 4-iodophenyl | pyridin-2-yl | H | H | H |
| 1330 | 4-methylphenyl | pyridin-2-yl | H | H | H |
| 1331 | 4-methoxyphenyl | pyridin-2-yl | H | H | H |
| 1332 | 4-trifluoromethoxyphenyl | pyridin-2-yl | H | H | H |
| 1333 | 2-trifluoromethylphenyl | pyridin-2-yl | H | H | H |
| 1334 | 3-trifluoromethylphenyl | pyridin-2-yl | H | H | H |
| 1335 | 4-trifluoromethylphenyl | pyridin-2-yl | H | H | H |
| 1336 | pyridin-2-yl | pyridin-2-yl | H | H | H |
| 1337 | pyridin-3-yl | pyridin-2-yl | H | H | H |
| 1338 | pyridin-4-yl | pyridin-2-yl | H | H | H |
| 1339 | pyrazin-2-yl | pyridin-2-yl | H | H | H |
| 1340 | pyrimidin-2-yl | pyridin-2-yl | H | H | H |
| 1341 | pyrimidin-5-yl | pyridin-2-yl | H | H | H |
| 1342 | pyrimidin-4-yl | pyridin-2-yl | H | H | H |
| 1343 | 4-trifluoromethylpyridin-2-yl | pyridin-2-yl | H | H | H |
| 1344 | 4-chloropyridin-2-yl | pyridin-2-yl | H | H | H |
| 1345 | 3-chloropyridin-4-yl | pyridin-2-yl | H | H | H |
| 1346 | 2-chloropyridin-3-yl | pyridin-2-yl | H | H | H |
| 1347 | 2-chloropyridin-4-yl | pyridin-2-yl | H | H | H |
| 1348 | 2-chloropyridin-5-yl | pyridin-2-yl | H | H | H |
| 1349 | 3-chloro-5-trifluoromethylpyridin-2- | pyridin-2-yl | H | H | H |
| | yl | | | | |
| 1350 | H | 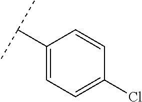 | H | H | H |
| 1351 | CH₃ | 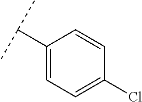 | H | H | H |
| 1352 | Et | 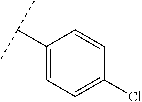 | H | H | H |
| 1353 | n-Pr | 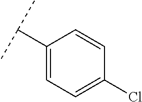 | H | H | H |
| 1354 | i-Pr | 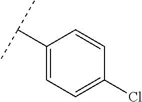 | H | H | H |
| 1355 | n-Bu | 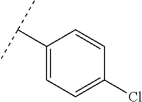 | H | H | H |
| 1356 | t-Bu | 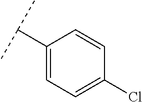 | H | H | H |
| 1357 | n-pentyl | 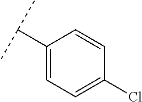 | H | H | H |
| 1358 | n-hexyl | 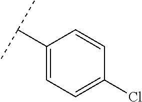 | H | H | H |
| 1359 | c-Pr | 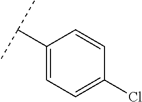 | H | H | H |
| 1360 | c-Bu | 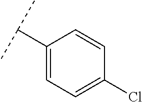 | H | H | H |
| 1361 | c-pentyl | 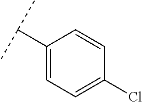 | H | H | H |
| 1362 | c-hexyl | 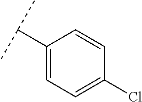 | H | H | H |
| 1363 | phenyl | 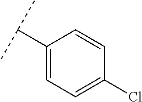 | H | H | H |
| 1364 | 2-fluorophenyl | 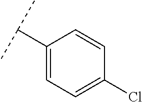 | H | H | H |
| 1365 | 3-fluorophenyl | 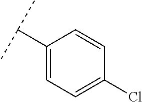 | H | H | H |
| 1366 | 4-fluorophenyl | 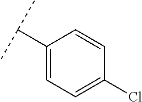 | H | H | H |
| 1367 | 2,4-difluorophenyl | 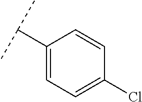 | H | H | H |
| 1368 | 2,5-difluorophenyl | 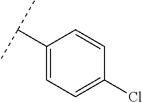 | H | H | H |
| 1369 | 2,6-difluorophenyl | 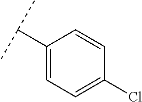 | H | H | H |
| 1370 | 3,5-difluorophenyl | 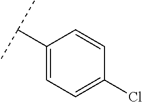 | H | H | H |
| 1371 | 2-chlorophenyl | 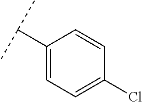 | H | H | H |
| 1372 | 3-chlorophenyl | 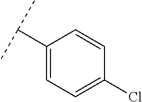 | H | H | H |
| 1373 | 4-chlorophenyl | 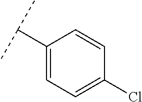 | H | H | H |
| 1374 | 2,4-dichlorophenyl | 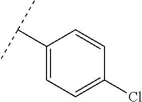 | H | H | H |
| 1375 | 2,5-dichlorophenyl | 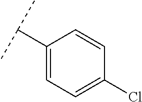 | H | H | H |
| 1376 | 2,6-dichlorophenyl | 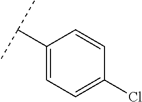 | H | H | H |
| 1377 | 3,5-dichlorophenyl | 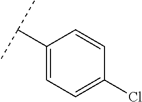 | H | H | H |
| 1378 | 4-bromophenyl | 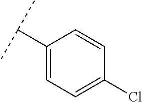 | H | H | H |
| 1379 | 4-iodophenyl | 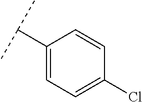 | H | H | H |
| 1380 | 4-methylphenyl | 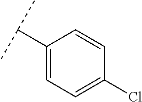 | H | H | H |
| 1381 | 4-methoxyphenyl | 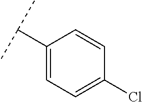 | H | H | H |
| 1382 | 4-trifluoromethoxyphenyl | 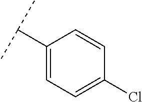 | H | H | H |
| 1383 | 2-trifluoromethylphenyl | 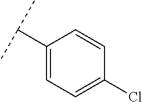 | H | H | H |
| 1384 | 3-trifluoromethylphenyl | 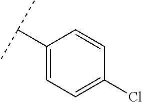 | H | H | H |
| 1385 | 4-trifluoromethylphenyl | 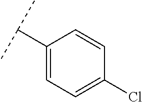 | H | H | H |
| 1386 | pyridin-2-yl | 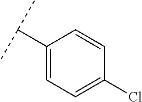 | H | H | H |
| 1387 | pyridin-3-yl | 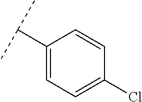 | H | H | H |
| 1388 | pyridin-4-yl | 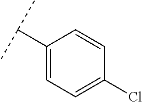 | H | H | H |
| 1389 | pyrazin-2-yl | 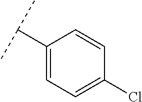 | H | H | H |
| 1390 | pyrimidin-2-yl | 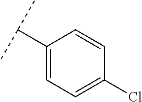 | H | H | H |
| 1391 | pyrimidin-5-yl | 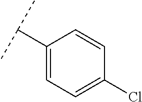 | H | H | H |
| 1392 | pyrimidin-4-yl | 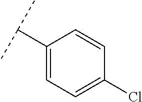 | H | H | H |
| 1393 | 4-trifluoromethylpyridin-2-yl | 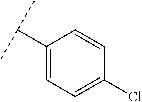 | H | H | H |
| 1394 | 4-chloropyridin-2-yl | 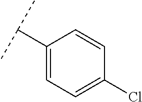 | H | H | H |
| 1395 | 3-chloropyridin-4-yl | 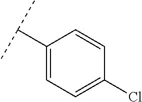 | H | H | H |
| 1396 | 2-chloropyridin-3-yl | 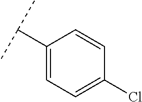 | H | H | H |
| 1397 | 2-chloropyridin-4-yl | 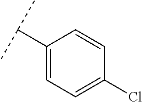 | H | H | H |
| 1398 | 2-chloropyridin-5-yl | 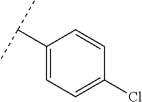 | H | H | H |
| 1399 | 3-chloro-5-trifluoromethylpyridin-2- yl | 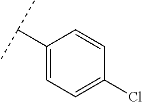 | H | H | H |
| 1400 | H | CF₃ | H | H | H |
| 1401 | CH₃ | CF₃ | H | H | H |
| 1402 | Et | CF₃ | H | H | H |
| 1403 | n-Pr | CF₃ | H | H | H |
| 1404 | i-Pr | CF₃ | H | H | H |
| 1405 | n-Bu | CF₃ | H | H | H |
| 1406 | t-Bu | CF₃ | H | H | H |
| 1407 | n-pentyl | CF₃ | H | H | H |
| 1408 | n-hexyl | CF₃ | H | H | H |
| 1409 | c-Pr | CF₃ | H | H | H |
| 1410 | c-Bu | CF₃ | H | H | H |
| 1411 | c-pentyl | CF₃ | H | H | H |
| 1412 | c-hexyl | CF₃ | H | H | H |
| 1413 | phenyl | CF₃ | H | H | H |
| 1414 | 2-fluorophenyl | CF₃ | H | H | H |
| 1415 | 3-fluorophenyl | CF₃ | H | H | H |
| 1416 | 4-fluorophenyl | CF₃ | H | H | H |
| 1417 | 2,4-difluorophenyl | CF₃ | H | H | H |
| 1418 | 2,5-difluorophenyl | CF₃ | H | H | H |
| 1419 | 2,6-difluorophenyl | CF₃ | H | H | H |
| 1420 | 3,5-difluorophenyl | CF₃ | H | H | H |
| 1421 | 2-chlorophenyl | CF₃ | H | H | H |
| 1422 | 3-chlorophenyl | CF₃ | H | H | H |
| 1423 | 4-chlorophenyl | CF₃ | H | H | H |
| 1424 | 2,4-dichlorophenyl | CF₃ | H | H | H |
| 1425 | 2,5-dichlorophenyl | CF₃ | H | H | H |
| 1426 | 2,6-dichlorophenyl | CF₃ | H | H | H |
| 1427 | 3,5-dichlorophenyl | CF₃ | H | H | H |
| 1428 | 4-bromophenyl | CF₃ | H | H | H |
| 1429 | 4-iodophenyl | CF₃ | H | H | H |
| 1430 | 4-methylphenyl | CF₃ | H | H | H |
| 1431 | 4-methoxyphenyl | CF₃ | H | H | H |
| 1432 | 4-trifluoromethoxyphenyl | CF₃ | H | H | H |
| 1433 | 2-trifluoromethylphenyl | CF₃ | H | H | H |
| 1434 | 3-trifluoromethylphenyl | CF₃ | H | H | H |
| 1435 | 4-trifluoromethylphenyl | CF₃ | H | H | H |
| 1436 | pyridin-2-yl | CF₃ | H | H | H |
| 1437 | pyridin-3-yl | CF₃ | H | H | H |
| 1438 | pyridin-4-yl | CF₃ | H | H | H |
| 1439 | pyrazin-2-yl | CF₃ | H | H | H |
| 1440 | pyrimidin-2-yl | CF₃ | H | H | H |
| 1441 | pyrimidin-5-yl | CF₃ | H | H | H |
| 1442 | pyrimidin-4-yl | CF₃ | H | H | H |
| 1443 | 4-trifluoromethylpyridin-2-yl | CF₃ | H | H | H |
| 1444 | 4-chloropyridin-2-yl | CF₃ | H | H | H |
| 1445 | 3-chloropyridin-4-yl | CF₃ | H | H | H |
| 1446 | 2-chloropyridin-3-yl | CF₃ | H | H | H |
| 1447 | 2-chloropyridin-4-yl | CF₃ | H | H | H |
| 1448 | 2-chloropyridin-5-yl | CF₃ | H | H | H |
| 1449 | 3-chloro-5-trifluoromethylpyridin-2- | CF₃ | H | H | H |
| | yl | | | | |
| 1450 | H | 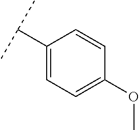 | H | H | H |
| 1451 | CH₃ | 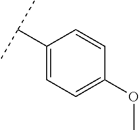 | H | H | H |
| 1452 | Et | 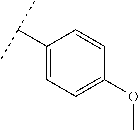 | H | H | H |
| 1453 | n-Pr | 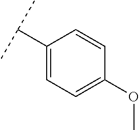 | H | H | H |
| 1454 | i-Pr | 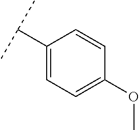 | H | H | H |
| 1455 | n-Bu | 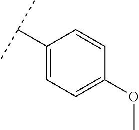 | H | H | H |
| 1456 | t-Bu | 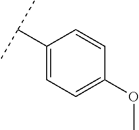 | H | H | H |
| 1457 | n-pentyl | 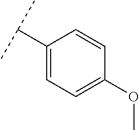 | H | H | H |
| 1458 | n-hexyl | 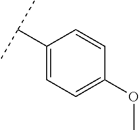 | H | H | H |
| 1459 | c-Pr | 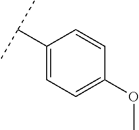 | H | H | H |
| 1460 | c-Bu | 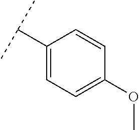 | H | H | H |
| 1461 | c-pentyl | 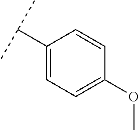 | H | H | H |
| 1462 | c-hexyl | 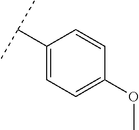 | H | H | H |
| 1463 | phenyl | 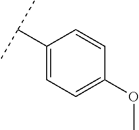 | H | H | H |
| 1464 | 2-fluorophenyl | 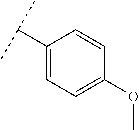 | H | H | H |
| 1465 | 3-fluorophenyl | 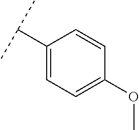 | H | H | H |
| 1466 | 4-fluorophenyl | 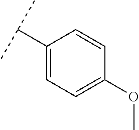 | H | H | H |
| 1467 | 2,4-difluorophenyl | 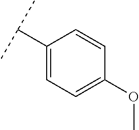 | H | H | H |
| 1468 | 2,5-difluorophenyl | 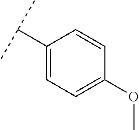 | H | H | H |
| 1469 | 2,6-difluorophenyl | 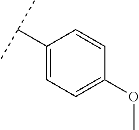 | H | H | H |
| 1470 | 3,5-difluorophenyl | 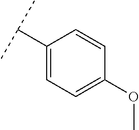 | H | H | H |
| 1471 | 2-chlorophenyl | 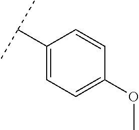 | H | H | H |
| 1472 | 3-chlorophenyl | 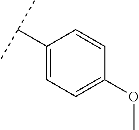 | H | H | H |
| 1473 | 4-chlorophenyl | 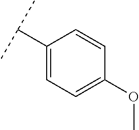 | H | H | H |
| 1474 | 2,4-dichlorophenyl | 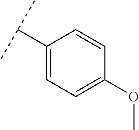 | H | H | H |
| 1475 | 2,5-dichlorophenyl | 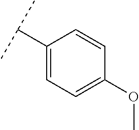 | H | H | H |
| 1476 | 2,6-dichlorophenyl | 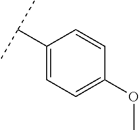 | H | H | H |
| 1477 | 3,5-dichlorophenyl | 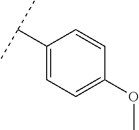 | H | H | H |
| 1478 | 4-bromophenyl | 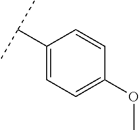 | H | H | H |
| 1479 | 4-iodophenyl | 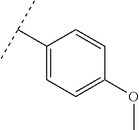 | H | H | H |
| 1480 | 4-methylphenyl | 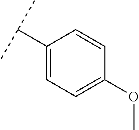 | H | H | H |
| 1481 | 4-methoxyphenyl | 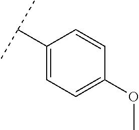 | H | H | H |
| 1482 | 4-trifluoromethoxyphenyl | 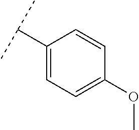 | H | H | H |
| 1483 | 2-trifluoromethylphenyl | 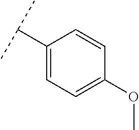 | H | H | H |
| 1484 | 3-trifluoromethylphenyl | 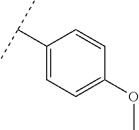 | H | H | H |
| 1485 | 4-trifluoromethylphenyl | 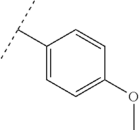 | H | H | H |
| 1486 | pyridin-2-yl | 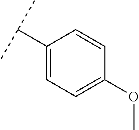 | H | H | H |
| 1487 | pyridin-3-yl | 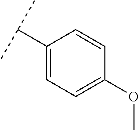 | H | H | H |
| 1488 | pyridin-4-yl | 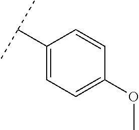 | H | H | H |
| 1489 | pyrazin-2-yl | 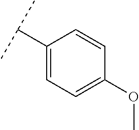 | H | H | H |
| 1490 | pyrimidin-2-yl | 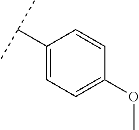 | H | H | H |
| 1491 | pyrimidin-5-yl | 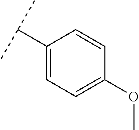 | H | H | H |
| 1492 | pyrimidin-4-yl | 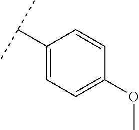 | H | H | H |
| 1493 | 4-trifluoromethylpyridin-2-yl | 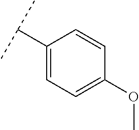 | H | H | H |
| 1494 | 4-chloropyridin-2-yl | 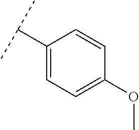 | H | H | H |
| 1495 | 3-chloropyridin-4-yl | 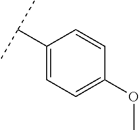 | H | H | H |
| 1496 | 2-chloropyridin-3-yl | 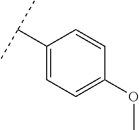 | H | H | H |
| 1497 | 2-chloropyridin-4-yl | 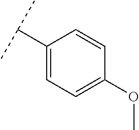 | H | H | H |
| 1498 | 2-chloropyridin-5-yl | 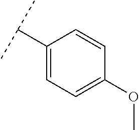 | H | H | H |
| 1499 | 3-chloro-5-trifluoromethylpyridin-2- yl | 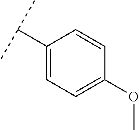 | H | H | H |

Preferred compounds according to the invention correspond to formula (I.1) below, where R⁴, R⁵, R⁶, R⁷ and R⁸ each have the meaning mentioned above or the preferred or particularly preferred meaning mentioned above.

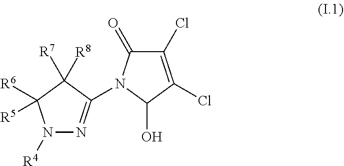

Table I.1: Preferred compounds of the formula (I.1) are the compounds I.1-1 to I.1-1499 in which R⁴, R⁵, R⁶, R⁷ and R⁸ have the meaning of Table 1 indicated in the respective row. Thus, the compounds I.1-1 to I.1-1499 of Table I.1 are defined by the meaning of the respective entries Nos. 1 to 1499 for R⁴, R⁵, R⁶, R⁷ and R⁸ of Table 1.

Preferred compounds according to the invention correspond to formula (I.2) below, where R⁴, R⁵, R⁶, R⁷ and R⁸ each have the meaning mentioned above or the preferred or particularly preferred meaning

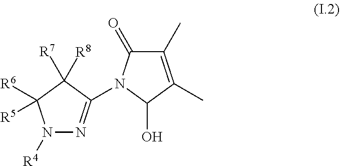

Table I.2: Preferred compounds of the formula (I.2) are the compounds I.2-1 to I.2-1499 in which R⁴, R⁵, R⁶, R⁷ and R⁸ have the meaning of Table 1 indicated in the respective row. Thus, the compounds I.2-1 to I.2-1499 of Table I.2 are defined by the meaning of the respective entries Nos. 1 to 1499 for R⁴, R⁵, R⁶, R⁷ and R⁸ of Table 1.

Preferred compounds according to the invention correspond to formula (I.3) below, where R⁴, R⁵, R⁶, R⁷ and R⁸ each have the meaning mentioned above or the preferred or particularly preferred meaning mentioned above.

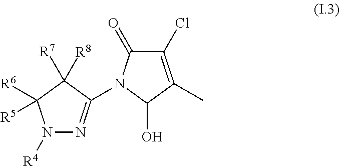

Table I.3: Preferred compounds of the formula (I.3) are the compounds I.3-1 to I.3-1499 in which R⁴, R⁵, R⁶, R⁷ and R⁸ have the meaning of Table 1 indicated in the respective row. Thus, the compounds I.3-1 to I.3-1499 of Table I.3 are defined by the meaning of the respective entries Nos. 1 to 1499 for R⁴, R⁵, R⁶, R⁷ and R⁸ of Table 1.

Preferred compounds according to the invention correspond to formula (I.4) below, where R⁴, R⁵, R⁶, R⁷ and R⁸ each have the meaning mentioned above or the preferred or particularly preferred meaning mentioned above.

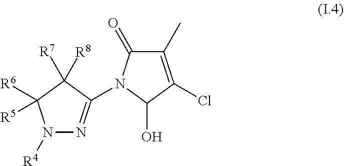

Table I.4: Preferred compounds of the formula (I.4) are the compounds I.4-1 to I.4-1499 in which R⁴, R⁵, R⁶, R⁷ and R⁸ have the meaning of Table 1 indicated in the respective row. Thus, the compounds I.4-1 to I.4-1499 of Table I.4 are defined by the meaning of the respective entries Nos. 1 to 1499 for R⁴, R⁵, R⁶, R⁷ and R⁸ of Table 1.

Preferred compounds according to the invention correspond to formula (I.5) below, where R⁴, R⁵, R⁶, R⁷ and R⁸ each have the meaning mentioned above or the preferred or particularly preferred meaning mentioned above.

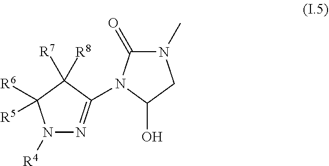

Table I.5: Preferred compounds of the formula (I.5) are the compounds I.5-1 to I.5-1499 in which R⁴, R⁵, R⁶, R⁷ and R⁸ have the meaning of Table 1 indicated in the respective row. Thus, the compounds I.5-1 to I.5-1499 of Table I.5 are defined by the meaning of the respective entries Nos. 1 to 1499 for R⁴, R⁵, R⁶, R⁷ and R⁸ of Table 1.

Preferred compounds according to the invention correspond to formula (I.6) below, where R⁴, R⁵, R⁶, R⁷ and R⁸ each have the meaning mentioned above or the preferred or particularly preferred meaning mentioned above.

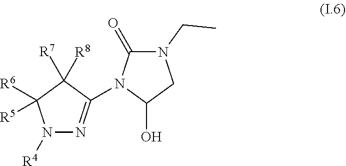

Table I.6: Preferred compounds of the formula (I.6) are the compounds I.6-1 to I.6-1499 in which R⁴, R⁵, R⁶, R⁷ and R⁸ have the meaning of Table 1 indicated in the respective row. Thus, the compounds I.6-1 to I.6-1499 of Table I.6 are defined by the meaning of the respective entries Nos. 1 to 1499 for R⁴, R⁵, R⁶, R⁷ and R⁸ of Table 1.

Preferred compounds according to the invention correspond to formula (I.7) below, where R⁴, R⁵, R⁶, R⁷ and R⁸ each have the meaning mentioned above or the preferred or particularly preferred meaning mentioned above.

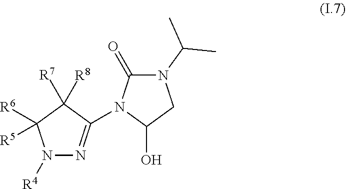

Table I.7: Preferred compounds of the formula (I.7) are the compounds I.7-1 to I.7-1499 in which R⁴, R⁵, R⁶, R⁷ and R⁸ have the meaning of Table 1 indicated in the respective row. Thus, the compounds I.7-1 to I.7-1499 of Table I.7 are defined by the meaning of the respective entries Nos. 1 to 1499 for R⁴, R⁵, R⁶, R⁷ and R⁸ of Table 1.

Preferred compounds according to the invention correspond to formula (I.8) below, where R⁴, R⁵, R⁶, R⁷ and R⁸ each have the meaning mentioned above or the preferred or particularly preferred meaning mentioned above.

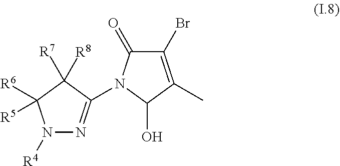

Table I.8: Preferred compounds of the formula (I.8) are the compounds I.8-1 to I.8-1499 in which R⁴, R⁵, R⁶, R⁷ and R⁸ have the meaning of Table 1 indicated in the respective row. Thus, the compounds I.8-1 to I.8-1499 of Table I.8 are defined by the meaning of the respective entries Nos. 1 to 1499 for R⁴, R⁵, R⁶, R⁷ and R⁸ of Table 1.

Preferred compounds according to the invention correspond to formula (I.9) below, where R⁴, R⁵, R⁶, R⁷ and R⁸ each have the meaning mentioned above or the preferred or particularly preferred meaning mentioned above.

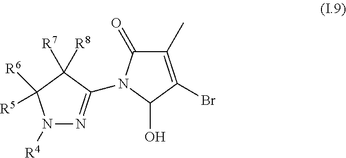

Table I.9: Preferred compounds of the formula (I.9) are the compounds I.9-1 to I.9-1499 in which R⁴, R⁵, R⁶, R⁷ and R⁸ have the meaning of Table 1 indicated in the respective row. Thus, the compounds I.9-1 to I.9-1499 of Table I.9 are defined by the meaning of the respective entries Nos. 1 to 1499 for R⁴, R⁵, R⁶, R⁷ and R⁸ of Table 1.

Preferred compounds according to the invention correspond to formula (I.10) below, where R⁴, R⁵, R⁶, R⁷ and R⁸ each have the meaning mentioned above or the preferred or particularly preferred meaning mentioned above.

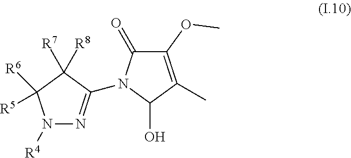

Table I.10: Preferred compounds of the formula (I.10) are the compounds I.10-1 to I.10-1499 in which R⁴, R⁵, R⁶, R⁷ and R⁸ have the meaning of Table 1 indicated in the respective row. Thus, the compounds I.10-1 to I.10-1499 of Table I.10 are defined by the meaning of the respective entries Nos. 1 to 1499 for R⁴, R⁵, R⁶, R⁷ and R⁸ of Table 1.

Preferred compounds according to the invention correspond to formula (I.11) below, where R⁴, R⁵, R⁶, R⁷ and R⁸ each have the meaning mentioned above or the preferred or particularly preferred meaning mentioned above.

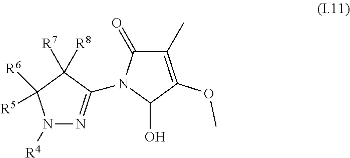

Table I.11: Preferred compounds of the formula (I.11) are the compounds I.11-1 to I.11-1499 in which R⁴, R⁵, R⁶, R⁷ and R⁸ have the meaning of Table 1 indicated in the respective row. Thus, the compounds I.11-1 to I.11-1499 of Table I.11 are defined by the meaning of the respective entries Nos. 1 to 1499 for R⁴, R⁵, R⁶, R⁷ and R⁸ of Table 1.

Preferred compounds according to the invention correspond to formula (I.12) below, where R⁴, R⁵, R⁶, R⁷ and R⁸ each have the meaning mentioned above or the preferred or particularly preferred meaning mentioned above.

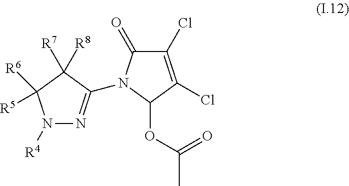

Table I.12: Preferred compounds of the formula (I.12) are the compounds I.12-1 to I.12-1499 in which R⁴, R⁵, R⁶, R⁷ and R⁸ have the meaning of Table 1 indicated in the respective row. Thus, the compounds I.12-1 to I.12-1499 of Table I.12 are defined by the meaning of the respective entries Nos. 1 to 1499 for R⁴, R⁵, R⁶, R⁷ and R⁸ of Table 1.

Preferred compounds according to the invention correspond to formula (I.13) below, where R⁴, R⁵, R⁶, R⁷ and R⁸ each have the meaning mentioned above or the preferred or particularly preferred meaning mentioned above.

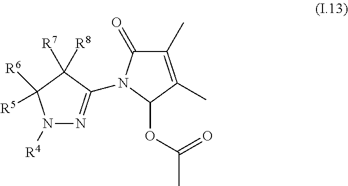

Table I.13: Preferred compounds of the formula (I.13) are the compounds I.13-1 to I.13-1499 in which R⁴, R⁵, R⁶, R⁷ and R⁸ have the meaning of Table 1 indicated in the respective row. Thus, the compounds I.13-1 to I.13-1499 of Table I.13 are defined by the meaning of the respective entries Nos. 1 to 1499 for R⁴, R⁵, R⁶, R⁷ and R⁸ of Table 1.

Preferred compounds according to the invention correspond to formula (I.14) below, where R⁴, R⁵, R⁶, R⁷ and R⁸ each have the meaning mentioned above or the preferred or particularly preferred meaning mentioned above.

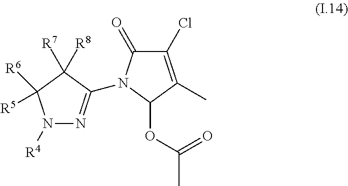

Table I.14: Preferred compounds of the formula (I.14) are the compounds I.14-1 to I.14-1499 in which R⁴, R⁵, R⁶, R⁷ and R⁸ have the meaning of Table 1 indicated in the respective row. Thus, the compounds I.14-1 to I.14-1499 of Table I.14 are defined by the meaning of the respective entries Nos. 1 to 1499 for R⁴, R⁵, R⁶, R⁷ and R⁸ of Table 1.

Preferred compounds according to the invention correspond to formula (I.15) below, where R⁴, R⁵, R⁶,

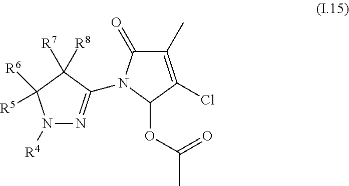

Table I.15: Preferred compounds of the formula (I.15) are the compounds I.15-1 to I.15-1499 in which R⁴, R⁵, R⁶, R⁷ and R⁸ have the meaning of Table 1 indicated in the respective row. Thus, the compounds I.15-1 to I.15-1499 of Table I.15 are defined by the meaning of the respective entries Nos. 1 to 1499 for R⁴, R⁵, R⁶, R⁷ and R⁸ of Table 1.

Preferred compounds according to the invention correspond to formula (I.16) below, where R⁴, R⁵, R⁶, R⁷ and R⁸ each have the meaning mentioned above or the preferred or particularly preferred meaning mentioned above.

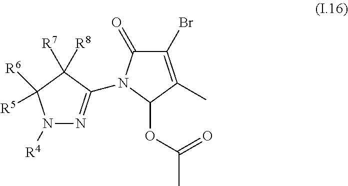

Table I.16: Preferred compounds of the formula (I.16) are the compounds I.16-1 to I.16-1499 in which R⁴, R⁵, R⁶, R⁷ and R⁸ have the meaning of Table 1 indicated in the respective row. Thus, the compounds I.16-1 to I.16-1499 of Table I.16 are defined by the meaning of the respective entries Nos. 1 to 1499 for R⁴, R⁵, R⁶, R⁷ and R⁸ of Table 1.

Preferred compounds according to the invention correspond to formula (I.17) below, where R⁴, R⁵, R⁶, R⁷ and R⁸ each have the meaning mentioned above or the preferred or particularly preferred meaning mentioned above.

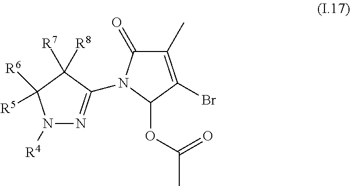

Table I.17: Preferred compounds of the formula (I.17) are the compounds I.17-1 to I.17-1499 in which R⁴, R⁵, R⁶, R⁷ and R⁸ have the meaning of Table 1 indicated in the respective row. Thus, the compounds I.17-1 to I.17-1499 of Table I.17 are defined by the meaning of the respective entries Nos. 1 to 1499 for R⁴, R⁵, R⁶, R⁷ and R⁸ of Table 1.

Preferred compounds according to the invention correspond to formula (I.18) below, where R⁴, R⁵, R⁶, R⁷ and R⁸ each have the meaning mentioned above or the preferred or particularly preferred meaning mentioned above.

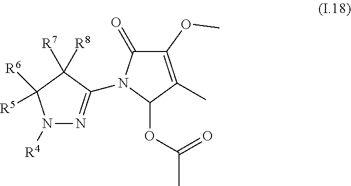

Table I.18: Preferred compounds of the formula (I.18) are the compounds I.18-1 to I.18-1499 in which R⁴, R⁵, R⁶, R⁷ and R⁸ have the meaning of Table 1 indicated in the respective row. Thus, the compounds I.18-1 to I.18-1499 of Table I.18 are defined by the meaning of the respective entries Nos. 1 to 1499 for R⁴, R⁵, R⁶, R⁷ and R⁸ of Table 1.

Preferred compounds according to the invention correspond to formula (I.19) below, where R⁴, R⁵, R⁶, R⁷ and R⁸ each have the meaning mentioned above or the preferred or particularly preferred meaning mentioned above.

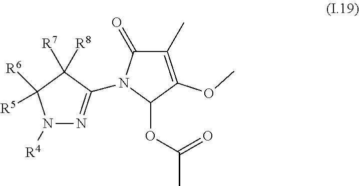

Table I.19: Preferred compounds of the formula (I.19) are the compounds I.19-1 to I.19-1499 in which R⁴, R⁵, R⁶, R⁷ and R⁸ have the meaning of Table 1 indicated in the respective row. Thus, the compounds I.19-1 to I.19-1499 of Table I.19 are defined by the meaning of the respective entries Nos. 1 to 1499 for R⁴, R⁵, R⁶, R⁷ and R⁸ of Table 1.

Preferred compounds according to the invention correspond to formula (I.20) below, where R⁴, R⁵, R⁶,

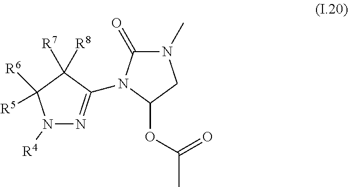

Table I.20: Preferred compounds of the formula (I.20) are the compounds I.20-1 to I.20-1499 in which R⁴, R⁵, R⁶, R⁷ and R⁸ have the meaning of Table 1 indicated in the respective row. Thus, the compounds I.20-1 to I.20-1499 of Table I.20 are defined by the meaning of the respective entries Nos. 1 to 1499 for R⁴, R⁵, R⁶, R⁷ and R⁸ of Table 1.

Preferred compounds according to the invention correspond to formula (I.21) below, where R⁴, R⁵, R⁶, R⁷ and R⁸ each have the meaning mentioned above or the preferred or particularly preferred meaning mentioned above.

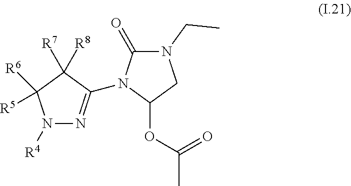

Table I.21: Preferred compounds of the formula (I.21) are the compounds I.21-1 to I.21-1499 in which R⁴, R⁵, R⁶, R⁷ and R⁸ have the meaning of Table 1 indicated in the respective row. Thus, the compounds I.21-1 to I.21-1499 of Table I.21 are defined by the meaning of the respective entries Nos. 1 to 1499 for R⁴, R⁵, R⁶, R⁷ and R⁸ of Table 1.

Preferred compounds according to the invention correspond to formula (I.22) below, where R⁴, R⁵, R⁶, R⁷ and R⁸ each have the meaning mentioned above or the preferred or particularly preferred meaning mentioned above.

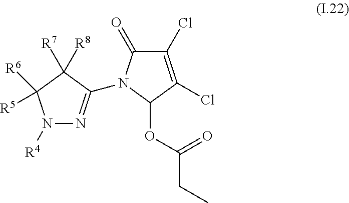

Table I.22: Preferred compounds of the formula (I.22) are the compounds I.22-1 to I.22-1499 in which R⁴, R⁵, R⁶, R⁷ and R⁸ have the meaning of Table 1 indicated in the respective row. Thus, the compounds I.22-1 to I.22-1499 of Table I.22 are defined by the meaning of the respective entries Nos. 1 to 1499 for R⁴, R⁵, R⁶, R⁷ and R⁸ of Table 1.

Preferred compounds according to the invention correspond to formula (I.23) below, where R⁴, R⁵, R⁶, R⁷ and R⁸ each have the meaning mentioned above or the preferred or particularly preferred meaning mentioned above.

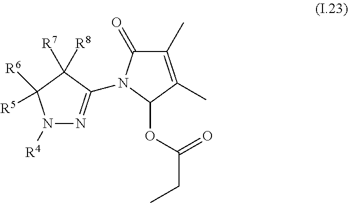

Table I.23: Preferred compounds of the formula (I.23) are the compounds I.23-1 to I.23-1499 in which R⁴, R⁵, R⁶, R⁷ and R have the meaning of Table 1 indicated in the respective row. Thus, the compounds I.23-1 to I.23-1499 of Table I.23 are defined by the meaning of the respective entries Nos. 1 to 1499 for R⁴, R⁵, R⁶, R⁷ and R⁸ of Table 1.

Preferred compounds according to the invention correspond to formula (I.24) below, where R⁴, R⁵, R⁶, R⁷ and R⁸ each have the meaning mentioned above or the preferred or particularly preferred meaning mentioned above.

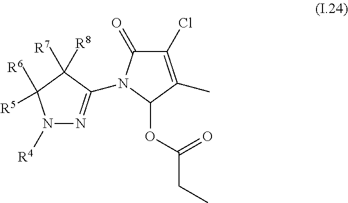

Table I.24: Preferred compounds of the formula (I.24) are the compounds I.24-1 to I.24-1499 in which R⁴, R⁵, R⁶, R⁷ and R⁸ have the meaning of Table 1 indicated in the respective row. Thus, the compounds I.24-1 to I.24-1499 of Table I.24 are defined by the meaning of the respective entries Nos. 1 to 1499 for R⁴, R⁵, R⁶, R⁷ and R of Table 1.

Preferred compounds according to the invention correspond to formula (I.25) below, where R⁴, R⁵, R⁶, R⁷ and R⁸ each have the meaning mentioned above or the preferred or particularly preferred meaning mentioned above.

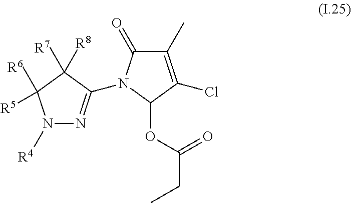

Table I.25: Preferred compounds of the formula (I.25) are the compounds I.25-1 to I.25-1499 in which R⁴, R⁵, R⁶, R⁷ and R⁸ have the meaning of Table 1 indicated in the respective row. Thus, the compounds I.25-1 to I.25-1499 of Table I.25 are defined by the meaning of the respective entries Nos. 1 to 1499 for R⁴, R⁵, R⁶, R⁷ and R⁸ of Table 1.

Preferred compounds according to the invention correspond to formula (I.26) below, where R⁴, R⁵, R⁶, R⁷ and R⁸ each have the meaning mentioned above or the preferred or particularly preferred meaning mentioned above.

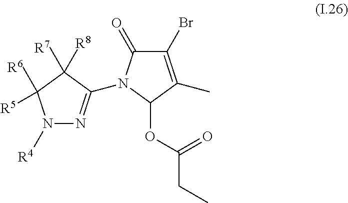

Table I.26: Preferred compounds of the formula (I.26) are the compounds I.26-1 to I.26-1499 in which R⁴, R⁵, R⁶, R⁷ and R⁸ have the meaning of Table 1 indicated in the respective row. Thus, the compounds I.26-1 to I.26-1499 of Table I.26 are defined by the meaning of the respective entries Nos. 1 to 1499 for R⁴, R⁵, R⁶, R⁷ and R⁸ of Table 1.

Preferred compounds according to the invention correspond to formula (I.27) below, where R⁴, R⁵, R⁶,

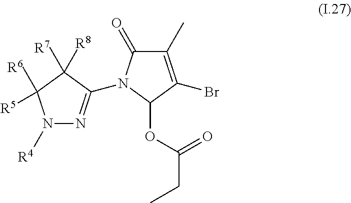

Table I.27: Preferred compounds of the formula (I.27) are the compounds I.27-1 to I.27-1499 in which R⁴, R⁵, R⁶, R⁷ and R⁸ have the meaning of Table 1 indicated in the respective row. Thus, the compounds I.27-1 to I.27-1499 of Table I.27 are defined by the meaning of the respective entries Nos. 1 to 1499 for R⁴, R⁵, R⁶, R⁷ and R⁸ of Table 1.

Preferred compounds according to the invention correspond to formula (I.28) below, where R⁴, R⁵, R⁶, R⁷ and R⁸ each have the meaning mentioned above or the preferred or particularly preferred meaning mentioned above.

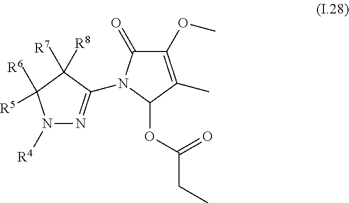

Table I.28: Preferred compounds of the formula (I.28) are the compounds I.28-1 to I.28-1499 in which R⁴, R⁵, R⁶, R⁷ and R⁸ have the meaning of Table 1 indicated in the respective row. Thus, the compounds I.28-1 to I.28-1499 of Table I.28 are defined by the meaning of the respective entries Nos. 1 to 1499 for R⁴, R⁵, R⁶, R⁷ and R⁸ of Table 1.

Preferred compounds according to the invention correspond to formula (I.29) below, where R⁴, R⁵, R⁶, R⁷ and R⁸ each have the meaning mentioned above or the preferred or particularly preferred meaning mentioned above.

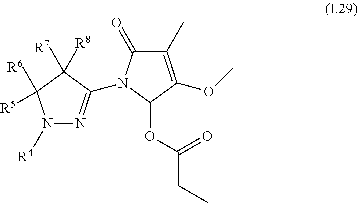

Table I.29: Preferred compounds of the formula (I.29) are the compounds I.29-1 to I.29-1499 in which R⁴, R⁵, R⁶, R⁷ and R⁸ have the meaning of Table 1 indicated in the respective row. Thus, the compounds I.29-1 to I.29-1499 of Table I.29 are defined by the meaning of the respective entries Nos. 1 to 1499 for R⁴, R⁵, R⁶, R⁷ and R⁸ of Table 1.

Preferred compounds according to the invention correspond to formula (I.30) below, where R⁴, R⁵, R⁶, R⁷ and R⁸ each have the meaning mentioned above or the preferred or particularly preferred meaning mentioned above.

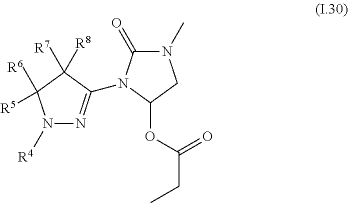

Table I.30: Preferred compounds of the formula (I.30) are the compounds I.30-1 to I.30-1499 in which R⁴, R⁵, R⁶, R⁷ and R have the meaning of Table 1 indicated in the respective row. Thus, the compounds I.30-1 to I.30-1499 of Table I.30 are defined by the meaning of the respective entries Nos. 1 to 1499 for R⁴, R⁵, R⁶, R⁷ and R⁸ of Table 1.

Preferred compounds according to the invention correspond to formula (I.31) below, where R⁴, R⁵, R⁶, R⁷ and R⁸ each have the meaning mentioned above or the preferred or particularly preferred meaning mentioned above.

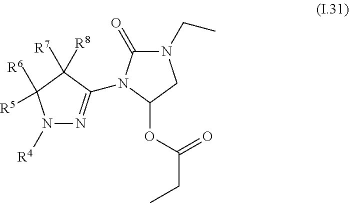

Table I.31: Preferred compounds of the formula (I.31) are the compounds I.31-1 to I.31-1499 in which R⁴, R⁵, R⁶, R⁷ and R⁸ have the meaning of Table 1 indicated in the respective row. Thus, the compounds I.31-1 to I.31-1499 of Table I.31 are defined by the meaning of the respective entries Nos. 1 to 1499 for R⁴, R⁵, R⁶, R⁷ and R⁸ of Table 1.

Preferred compounds according to the invention correspond to formula (I.32) below, where R⁴, R⁵, R⁶,

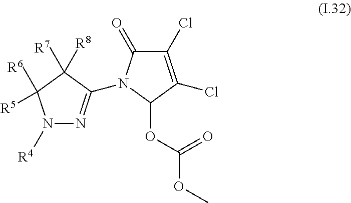

Table I.32: Preferred compounds of the formula (I.32) are the compounds I.32-1 to I.32-1499 in which R⁴, R⁵, R⁶, R⁷ and R⁸ have the meaning of Table 1 indicated in the respective row. Thus, the compounds I.32-1 to I.32-1499 of Table I.32 are defined by the meaning of the respective entries Nos. 1 to 1499 for R⁴, R⁵, R⁶, R⁷ and R⁸ of Table 1.

Preferred compounds according to the invention correspond to formula (I.33) below, where R⁴, R⁵, R⁶, R⁷ and R⁸ each have the meaning mentioned above or the preferred or particularly preferred meaning mentioned above.

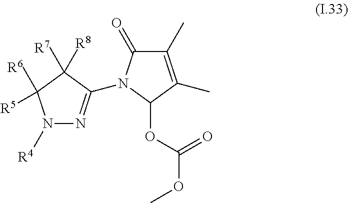

Table I.33: Preferred compounds of the formula (I.33) are the compounds I.33-1 to I.33-1499 in which R⁴, R⁵, R⁶, R⁷ and R⁸ have the meaning of Table 1 indicated in the respective row. Thus, the compounds I.33-1 to I.33-1499 of Table I.33 are defined by the meaning of the respective entries Nos. 1 to 1499 for R⁴, R⁵, R⁶, R⁷ and R⁸ of Table 1.

Preferred compounds according to the invention correspond to formula (I.34) below, where R⁴, R⁵, R⁶, R⁷ and R⁸ each have the meaning mentioned above or the preferred or particularly preferred meaning mentioned above.

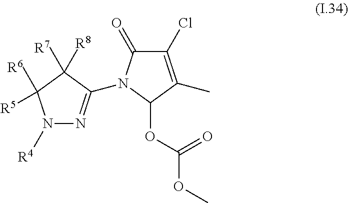

Table I.34: Preferred compounds of the formula (I.34) are the compounds I.34-1 to I.34-1499 in which R⁴, R⁵, R⁶, R⁷ and R⁸ have the meaning of Table 1 indicated in the respective row. Thus, the compounds I.34-1 to I.34-1499 of Table I.34 are defined by the meaning of the respective entries Nos. 1 to 1499 for R⁴, R⁵, R⁶, R⁷ and R⁸ of Table 1.

Preferred compounds according to the invention correspond to formula (I.35) below, where R⁴, R⁵, R⁶, R⁷ and R⁸ each have the meaning mentioned above or the preferred or particularly preferred meaning mentioned above.

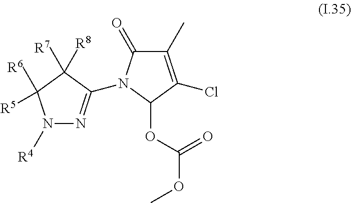

Table I.35: Preferred compounds of the formula (I.35) are the compounds I.35-1 to I.35-1499 in which R⁴, R⁵, R⁶, R⁷ and R⁸ have the meaning of Table 1 indicated in the respective row. Thus, the compounds I.35-1 to I.35-1499 of Table I.35 are defined by the meaning of the respective entries Nos. 1 to 1499 for R⁴, R⁵, R⁶, R⁷ and R⁸ of Table 1.

Preferred compounds according to the invention correspond to formula (I.36) below, where R⁴, R⁵, R⁶, R⁷ and R⁸ each have the meaning mentioned above or the preferred or particularly preferred meaning mentioned above.

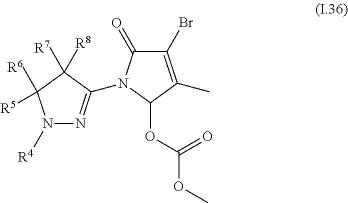

Table I.36: Preferred compounds of the formula (I.36) are the compounds I.36-1 to I.36-1499 in which R⁴, R⁵, R⁶, R⁷ and R⁸ have the meaning of Table 1 indicated in the respective row. Thus, the compounds I.36-1 to I.36-1499 of Table I.36 are defined by the meaning of the respective entries Nos. 1 to 1499 for R⁴, R⁵, R⁶, R⁷ and R⁸ of Table 1.

Preferred compounds according to the invention correspond to formula (I.37) below, where R⁴, R⁵, R⁶, R⁷ and R⁸ each have the meaning mentioned above or the preferred or particularly preferred meaning mentioned above.

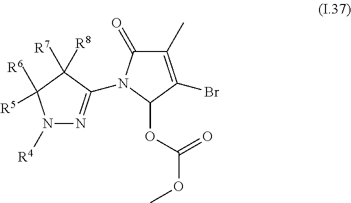

Table I.37: Preferred compounds of the formula (I.37) are the compounds I.37-1 to I.37-1499 in which R⁴, R⁵, R⁶, R⁷ and R⁸ have the meaning of Table 1 indicated in the respective row. Thus, the compounds I.37-1 to I.37-1499 of Table I.37 are defined by the meaning of the respective entries Nos. 1 to 1499 for R⁴, R⁵, R⁶, R⁷ and R⁸ of Table 1.

Preferred compounds according to the invention correspond to formula (I.38) below, where R⁴, R⁵, R⁶, R⁷ and R⁸ each have the meaning mentioned above or the preferred or particularly preferred meaning mentioned above.

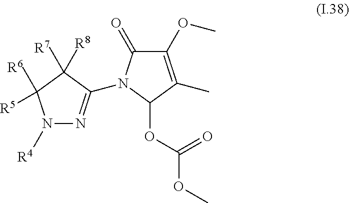

Table I.38: Preferred compounds of the formula (I.38) are the compounds I.38-1 to I.38-1499 in which R⁴, R⁵, R⁶, R⁷ and R⁸ have the meaning of Table 1 indicated in the respective row. Thus, the compounds I.38-1 to I.38-1499 of Table I.38 are defined by the meaning of the respective entries Nos. 1 to 1499 for R⁴, R⁵, R⁶, R⁷ and R⁸ of Table 1.

Preferred compounds according to the invention correspond to formula (I.39) below, where R⁴, R⁵, R⁶,

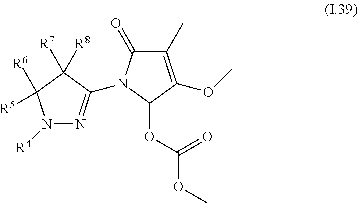

Table I.39: Preferred compounds of the formula (I.39) are the compounds I.39-1 to I.39-1499 in which R⁴, R⁵, R⁶, R⁷ and R⁸ have the meaning of Table 1 indicated in the respective row. Thus, the compounds I.39-1 to I.39-1499 of Table I.39 are defined by the meaning of the respective entries Nos. 1 to 1499 for R⁴, R⁵, R⁶, R⁷ and R⁸ of Table 1.

Preferred compounds according to the invention correspond to formula (I.40) below, where R⁴, R⁵, R⁶, R⁷ and R⁸ each have the meaning mentioned above or the preferred or particularly preferred meaning mentioned above.

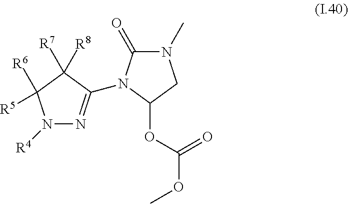

Table I.40: Preferred compounds of the formula (I.40) are the compounds I.40-1 to I.40-1499 in which R⁴, R⁵, R⁶, R⁷ and R⁸ have the meaning of Table 1 indicated in the respective row. Thus, the compounds I.40-1 to I.40-1499 of Table I.40 are defined by the meaning of the respective entries Nos. 1 to 1499 for R⁴, R⁵, R⁶, R⁷ and R⁸ of Table 1.

Preferred compounds according to the invention correspond to formula (I.41) below, where R⁴, R⁵, R⁶, R⁷ and R⁸ each have the meaning mentioned above or the preferred or particularly preferred meaning mentioned above.

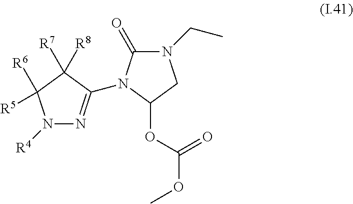

Table I.41: Preferred compounds of the formula (I.41) are the compounds I.41-1 to I.41-1499 in which R⁴, R⁵, R⁶, R⁷ and R⁸ have the meaning of Table 1 indicated in the respective row. Thus, the compounds I.41-1 to I.41-1499 of Table I.41 are defined by the meaning of the respective entries Nos. 1 to 1499 for R⁴, R⁵, R⁶, R⁷ and R⁸ of Table 1.

Preferred compounds according to the invention correspond to formula (I.42) below, where R⁴, R⁵, R⁶, R⁷ and R⁸ each have the meaning mentioned above or the preferred or particularly preferred meaning mentioned above.

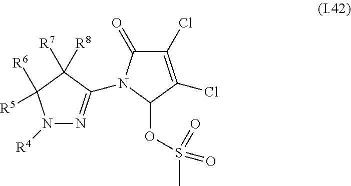

Table I.42: Preferred compounds of the formula (I.42) are the compounds I.42-1 to I.42-1499 in which R⁴, R⁵, R⁶, R⁷ and R⁸ have the meaning of Table 1 indicated in the respective row. Thus, the compounds I.42-1 to I.42-1499 of Table I.42 are defined by the meaning of the respective entries Nos. 1 to 1499 for R⁴, R⁵, R⁶, R⁷ and R⁸ of Table 1.

Preferred compounds according to the invention correspond to formula (I.43) below, where R⁴, R⁵, R⁶, R⁷ and R⁸ each have the meaning mentioned above or the preferred or particularly preferred meaning mentioned above.

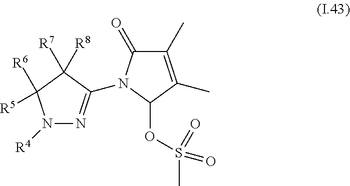

Table I.43: Preferred compounds of the formula (I.43) are the compounds I.43-1 to I.43-1499 in which R⁴, R⁵, R⁶, R⁷ and R⁸ have the meaning of Table 1 indicated in the respective row. Thus, the compounds I.43-1 to I.43-1499 of Table I.43 are defined by the meaning of the respective entries Nos. 1 to 1499 for R⁴, R⁵, R⁶, R⁷ and R⁸ of Table 1.

Preferred compounds according to the invention correspond to formula (I.44) below, where R⁴, R⁵, R⁶,

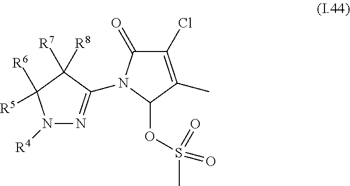

Table I.44: Preferred compounds of the formula (I.44) are the compounds I.44-1 to I.44-1499 in which R⁴, R⁵, R⁶, R⁷ and R⁸ have the meaning of Table 1 indicated in the respective row. Thus, the compounds I.44-1 to I.44-1499 of Table I.44 are defined by the meaning of the respective entries Nos. 1 to 1499 for R⁴, R⁵, R⁶, R⁷ and R⁸ of Table 1.

Preferred compounds according to the invention correspond to formula (I.45) below, where R⁴, R⁵, R⁶, R⁷ and R⁸ each have the meaning mentioned above or the preferred or particularly preferred meaning mentioned above.

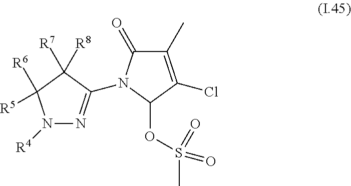

Table I.45: Preferred compounds of the formula (I.45) are the compounds I.45-1 to I.45-1499 in which R⁴, R⁵, R⁶, R⁷ and R⁸ have the meaning of Table 1 indicated in the respective row. Thus, the compounds I.45-1 to I.45-1499 of Table I.45 are defined by the meaning of the respective entries Nos. 1 to 1499 for R⁴, R⁵, R⁶, R⁷ and R⁸ of Table 1.

Preferred compounds according to the invention correspond to formula (I.46) below, where R⁴, R⁵, R⁶, R⁷ and R⁸ each have the meaning mentioned above or the preferred or particularly preferred meaning mentioned above.

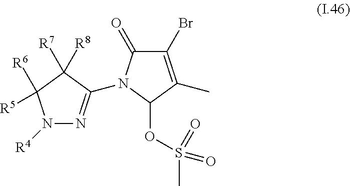

Table I.46: Preferred compounds of the formula (I.46) are the compounds I.46-1 to I.46-1499 in which R⁴, R⁵, R⁶, R⁷ and R⁸ have the meaning of Table 1 indicated in the respective row. Thus, the compounds 1.46-1 to I.46-1499 of Table I.46 are defined by the meaning of the respective entries Nos. 1 to 1499 for R⁴, R⁵, R⁶, R⁷ and R⁸ of Table 1.

Preferred compounds according to the invention correspond to formula (I.47) below, where R⁴, R⁵, R⁶, R⁷ and R⁸ each have the meaning mentioned above or the preferred or particularly preferred meaning mentioned above.

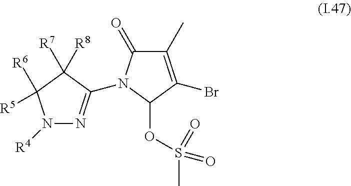

Table I.47: Preferred compounds of the formula (I.47) are the compounds I.47-1 to I.47-1499 in which R⁴, R⁵, R⁶, R⁷ and R⁸ have the meaning of Table 1 indicated in the respective row. Thus, the compounds I.47-1 to I.47-1499 of Table I.47 are defined by the meaning of the respective entries Nos. 1 to 1499 for R⁴, R⁵, R⁶, R⁷ and R⁸ of Table 1.

Preferred compounds according to the invention correspond to formula (I.48) below, where R⁴, R⁵, R⁶, R⁷ and R⁸ each have the meaning mentioned above or the preferred or particularly preferred meaning mentioned above.

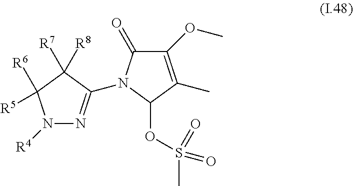

Table I.48: Preferred compounds of the formula (I.48) are the compounds I.48-1 to I.48-1499 in which R⁴, R⁵, R⁶, R⁷ and R⁸ have the meaning of Table 1 indicated in the respective row. Thus, the compounds I.48-1 to I.48-1499 of Table I.48 are defined by the meaning of the respective entries Nos. 1 to 1499 for R⁴, R⁵, R⁶, R⁷ and R⁸ of Table 1.

Preferred compounds according to the invention correspond to formula (I.49) below, where R⁴, R⁵, R⁶,

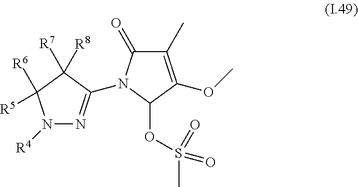

Table I.49: Preferred compounds of the formula (I.49) are the compounds I.49-1 to I.49-1499 in which R⁴, R⁵, R⁶, R⁷ and R⁸ have the meaning of Table 1 indicated in the respective row. Thus, the compounds I.49-1 to I.49-1499 of Table I.49 are defined by the meaning of the respective entries Nos. 1 to 1499 for R⁴, R⁵, R⁶, R⁷ and R⁸ of Table 1.

Preferred compounds according to the invention correspond to formula (I.50) below, where R⁴, R⁵, R⁶, R⁷ and R⁸ each have the meaning mentioned above or the preferred or particularly preferred meaning mentioned above.

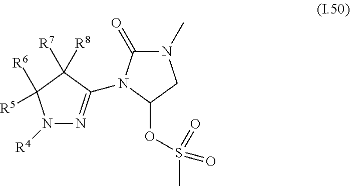

Table I.50: Preferred compounds of the formula (I.50) are the compounds I.50-1 to I.50-1499 in which R⁴, R⁵, R⁶, R⁷ and R⁸ have the meaning of Table 1 indicated in the respective row. Thus, the compounds I.50-1 to I.50-1499 of Table I.50 are defined by the meaning of the respective entries Nos. 1 to 1499 for R⁴, R⁵, R⁶, R⁷ and R⁸ of Table 1.

Preferred compounds according to the invention correspond to formula (I.51) below, where R⁴, R⁵, R⁶, R⁷ and R⁸ each have the meaning mentioned above or the preferred or particularly preferred meaning mentioned above.

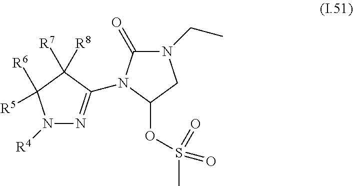

Table I.51: Preferred compounds of the formula (I.51) are the compounds I.51-1 to I.51-1499 in which R⁴, R⁵, R⁶, R⁷ and R⁸ have the meaning of Table 1 indicated in the respective row. Thus, the compounds I.51-1 to I.51-1499 of Table I.51 are defined by the meaning of the respective entries Nos. 1 to 1499 for R⁴, R⁵, R⁶, R⁷ and R⁸ of Table 1.

Preferred compounds according to the invention correspond to formula (I.52) below, where R⁴, R⁵, R⁶, R⁷ and R⁸ each have the meaning mentioned above or the preferred or particularly preferred meaning mentioned above.

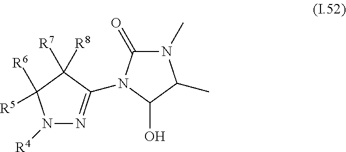

Table I.52: Preferred compounds of the formula (I.52) are the compounds I.52-1 to I.52-1499 in which R⁴, R⁵, R⁶, R⁷ and R⁸ have the meaning of Table 1 indicated in the respective row. Thus, the compounds I.52-1 to I.52-1499 of Table I.52 are defined by the meaning of the respective entries Nos. 1 to 1499 for R⁴, R⁵, R⁶, R⁷ and R⁸ of Table 1.

Preferred compounds according to the invention correspond to formula (I.53) below, where R⁴, R⁵, R⁶, R⁷ and R⁸ each have the meaning mentioned above or the preferred or particularly preferred meaning mentioned above.

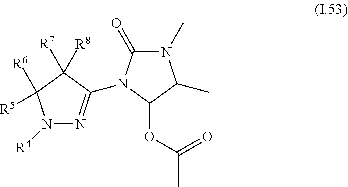

Table I.53: Preferred compounds of the formula (I.53) are the compounds I.53-1 to I.53-1499 in which R⁴, R⁵, R⁶, R⁷ and R⁸ have the meaning of Table 1 indicated in the respective row. Thus, the compounds I.53-1 to I.53-1499 of Table I.53 are defined by the meaning of the respective entries Nos. 1 to 1499 for R⁴, R⁵, R⁶, R⁷ and R⁸ of Table 1.

Preferred compounds according to the invention correspond to formula (I.54) below, where R⁴, R⁵, R⁶,

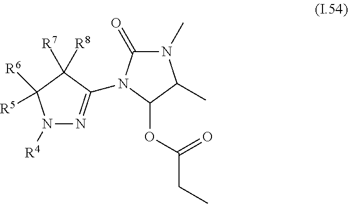

Table I.54: Preferred compounds of the formula (I.54) are the compounds I.54-1 to I.54-1499 in which R⁴, R⁵, R⁶, R⁷ and R⁸ have the meaning of Table 1 indicated in the respective row. Thus, the compounds I.54-1 to I.54-1499 of Table I.54 are defined by the meaning of the respective entries Nos. 1 to 1499 for R⁴, R⁵, R⁶, R⁷ and R⁸ of Table 1.

Preferred compounds according to the invention correspond to formula (I.55) below, where R⁴, R⁵, R⁶, R⁷ and R⁸ each have the meaning mentioned above or the preferred or particularly preferred meaning mentioned above.

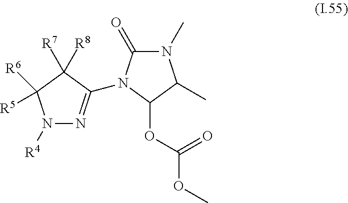

Table I.55: Preferred compounds of the formula (I.55) are the compounds I.55-1 to I.55-1499 in which R⁴, R⁵, R⁶, R⁷ and R⁸ have the meaning of Table 1 indicated in the respective row. Thus, the compounds I.55-1 to I.55-1499 of Table I.55 are defined by the meaning of the respective entries Nos. 1 to 1499 for R⁴, R⁵, R⁶, R⁷ and R⁸ of Table 1.

Preferred compounds according to the invention correspond to formula (I.56) below, where R⁴, R⁵, R⁶, R⁷ and R⁸ each have the meaning mentioned above or the preferred or particularly preferred meaning mentioned above.

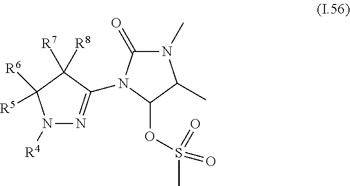

Table I.56: Preferred compounds of the formula (I.56) are the compounds I.56-1 to I.56-1499 in which R⁴, R⁵, R⁶, R⁷ and R⁸ have the meaning of Table 1 indicated in the respective row. Thus, the compounds I.56-1 to I.56-1499 of Table I.56 are defined by the meaning of the respective entries Nos. 1 to 1499 for R⁴, R⁵, R⁶, R⁷ and R⁸ of Table 1.

Preferred compounds according to the invention correspond to formula (I.57) below, where R⁴, R⁵, R⁶, R⁷ and R⁸ each have the meaning mentioned above or the preferred or particularly preferred meaning mentioned above.

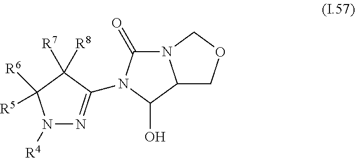

Table I.57: Preferred compounds of the formula (I.57) are the compounds I.57-1 to I.57-1499 in which R⁴, R⁵, R⁶, R⁷ and R⁸ have the meaning of Table 1 indicated in the respective row. Thus, the compounds I.57-1 to I.57-1499 of Table I.57 are defined by the meaning of the respective entries Nos. 1 to 1499 for R⁴, R⁵, R⁶, R⁷ and R⁸ of Table 1.

Preferred compounds according to the invention correspond to formula (I.58) below, where R⁴, R⁵, R⁶, R⁷ and R⁸ each have the meaning mentioned above or the preferred or particularly preferred meaning mentioned above.

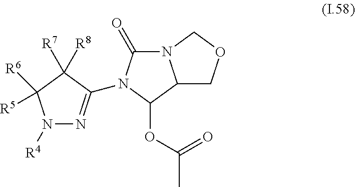

Table I.58: Preferred compounds of the formula (I.58) are the compounds I.58-1 to I.58-1499 in which R⁴, R⁵, R⁶, R⁷ and R⁸ have the meaning of Table 1 indicated in the respective row. Thus, the compounds I.58-1 to I.58-1499 of Table I.58 are defined by the meaning of the respective entries Nos. 1 to 1499 for R⁴, R⁵, R⁶, R⁷ and R⁸ of Table 1.

Preferred compounds according to the invention correspond to formula (I.59) below, where R⁴, R⁵, R⁶,

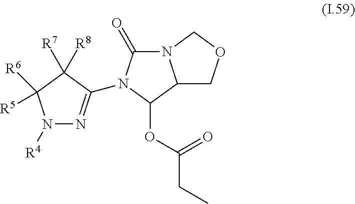

Table I.59: Preferred compounds of the formula (I.59) are the compounds I.59-1 to I.59-1499 in which R⁴, R⁵, R⁶, R⁷ and R⁸ have the meaning of Table 1 indicated in the respective row. Thus, the compounds I.59-1 to I.59-1499 of Table I.59 are defined by the meaning of the respective entries Nos. 1 to 1499 for R⁴, R⁵, R⁶, R⁷ and R⁸ of Table 1.

Preferred compounds according to the invention correspond to formula (I.60) below, where R⁴, R⁵, R⁶, R⁷ and R⁸ each have the meaning mentioned above or the preferred or particularly preferred meaning mentioned above.

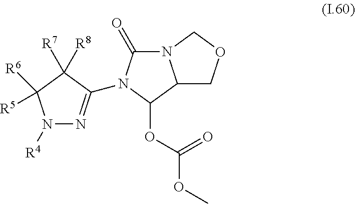

Table I.60: Preferred compounds of the formula (I.60) are the compounds I.60-1 to I.60-1499 in which R⁴, R⁵, R⁶, R⁷ and R⁸ have the meaning of Table 1 indicated in the respective row. Thus, the compounds I.60-1 to I.60-1499 of Table I.60 are defined by the meaning of the respective entries Nos. 1 to 1499 for R⁴, R⁵, R⁶, R⁷ and R⁸ of Table 1.

Preferred compounds according to the invention correspond to formula (I.61) below, where R⁴, R⁵, R⁶, R⁷ and R⁸ each have the meaning mentioned above or the preferred or particularly preferred meaning mentioned above.

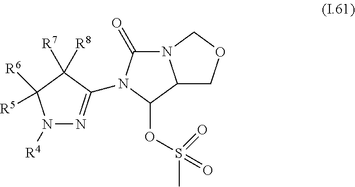

Table I.61: Preferred compounds of the formula (I.61) are the compounds I.61-1 to I.61-1499 in which R⁴, R⁵, R⁶, R⁷ and R⁸ have the meaning of Table 1 indicated in the respective row. Thus, the compounds I.61-1 to I.61-1499 of Table I.61 are defined by the meaning of the respective entries Nos. 1 to 1499 for R⁴, R⁵, R⁶, R⁷ and R⁸ of Table 1.

Preferred compounds according to the invention correspond to formula (I.62) below, where R⁴, R⁵, R⁶, R⁷ and R⁸ each have the meaning mentioned above or the preferred or particularly preferred meaning mentioned above.

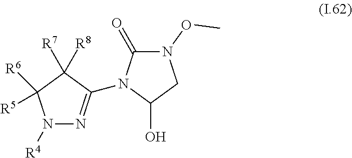

Table I.62: Preferred compounds of the formula (I.62) are the compounds I.62-1 to I.62-1499 in which R⁴, R⁵, R⁶, R⁷ and R⁸ have the meaning of Table 1 indicated in the respective row. Thus, the compounds I.62-1 to I.62-1499 of Table I.62 are defined by the meaning of the respective entries Nos. 1 to 1499 for R⁴, R⁵, R⁶, R⁷ and R⁸ of Table 1.

Preferred compounds according to the invention correspond to formula (I.63) below, where R⁴, R⁵, R⁶, R⁷ and R⁸ each have the meaning mentioned above or the preferred or particularly preferred meaning mentioned above.

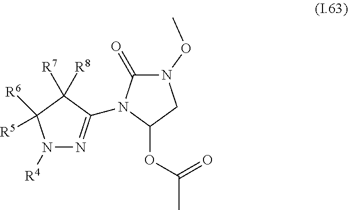

Table I.63: Preferred compounds of the formula (I.63) are the compounds I.63-1 to I.63-1499 in which R⁴, R⁵, R⁶, R⁷ and R⁸ have the meaning of Table 1 indicated in the respective row. Thus, the compounds I.63-1 to I.63-1499 of Table I.63 are defined by the meaning of the respective entries Nos. 1 to 1499 for R⁴, R⁵, R⁶, R⁷ and R⁸ of Table 1.

Preferred compounds according to the invention correspond to formula (I.64) below, where R⁴, R⁵, R⁶,

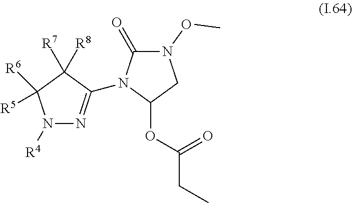

Table I.64: Preferred compounds of the formula (I.64) are the compounds I.64-1 to I.64-1499 in which R⁴, R⁵, R⁶, R⁷ and R⁸ have the meaning of Table 1 indicated in the respective row. Thus, the compounds I.64-1 to I.64-1499 of Table I.64 are defined by the meaning of the respective entries Nos. 1 to 1499 for R⁴, R⁵, R⁶, R⁷ and R⁸ of Table 1.

Preferred compounds according to the invention correspond to formula (I.65) below, where R⁴, R⁵, R⁶, R⁷ and R⁸ each have the meaning mentioned above or the preferred or particularly preferred meaning mentioned above.

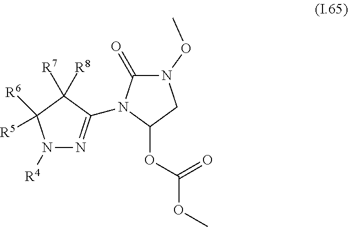

Table I.65: Preferred compounds of the formula (I.65) are the compounds I.65-1 to I.65-1499 in which R⁴, R⁵, R⁶, R⁷ and R⁸ have the meaning of Table 1 indicated in the respective row. Thus, the compounds I.65-1 to I.65-1499 of Table I.65 are defined by the meaning of the respective entries Nos. 1 to 1499 for R⁴, R⁵, R⁶, R⁷ and R⁸ of Table 1.

Preferred compounds according to the invention correspond to formula (I.66) below, where R⁴, R⁵, R⁶, R⁷ and R⁸ each have the meaning mentioned above or the preferred or particularly preferred meaning mentioned above.

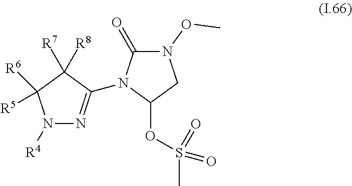

Table I.66: Preferred compounds of the formula (I.66) are the compounds I.66-1 to I.66-1499 in which R⁴, R⁵, R⁶, R⁷ and R⁸ have the meaning of Table 1 indicated in the respective row. Thus, the compounds I.66-1 to I.66-1499 of Table I.66 are defined by the meaning of the respective entries Nos. 1 to 1499 for R⁴, R⁵, R⁶, R⁷ and R⁸ of Table 1.

Preferred compounds according to the invention correspond to formula (I.67) below, where R⁴, R⁵, R⁶, R⁷ and R⁸ each have the meaning mentioned above or the preferred or particularly preferred meaning mentioned above.

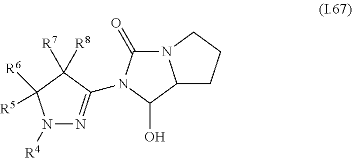

Table I.67: Preferred compounds of the formula (I.67) are the compounds I.67-1 to I.67-1499 in which R⁴, R⁵, R⁶, R⁷ and R⁸ have the meaning of Table 1 indicated in the respective row. Thus, the compounds I.67-1 to I.67-1499 of Table I.67 are defined by the meaning of the respective entries Nos. 1 to 1499 for R⁴, R⁵, R⁶, R⁷ and R⁸ of Table 1.

Preferred compounds according to the invention correspond to formula (I.68) below, where R⁴, R⁵, R⁶, R⁷ and R⁸ each have the meaning mentioned above or the preferred or particularly preferred meaning mentioned above.

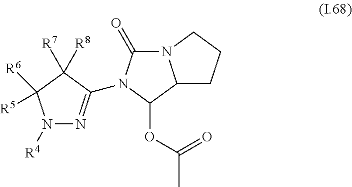

Table I.68: Preferred compounds of the formula (I.68) are the compounds I.68-1 to I.68-1499 in which R⁴, R⁵, R⁶, R⁷ and R⁸ have the meaning of Table 1 indicated in the respective row. Thus, the compounds I.68-1 to I.68-1499 of Table I.68 are defined by the meaning of the respective entries Nos. 1 to 1499 for R⁴, R⁵, R⁶, R⁷ and R⁸ of Table 1.

Preferred compounds according to the invention correspond to formula (I.69) below, where R⁴, R⁵, R⁶,

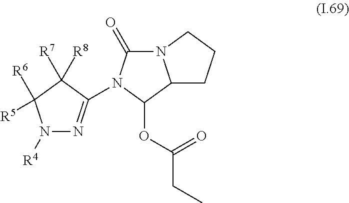

Table I.69: Preferred compounds of the formula (I.69) are the compounds I.69-1 to I.69-1499 in which R⁴, R⁵, R⁶, R⁷ and R⁸ have the meaning of Table 1 indicated in the respective row. Thus, the compounds I.69-1 to I.69-1499 of Table I.69 are defined by the meaning of the respective entries Nos. 1 to 1499 for R⁴, R⁵, R⁶, R⁷ and R⁸ of Table 1.

Preferred compounds according to the invention correspond to formula (I.70) below, where R⁴, R⁵, R⁶, R⁷ and R⁸ each have the meaning mentioned above or the preferred or particularly preferred meaning mentioned above.

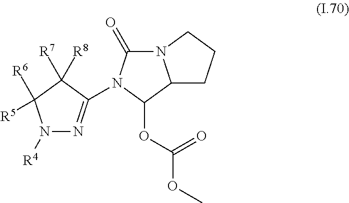

Table I.70: Preferred compounds of the formula (I.70) are the compounds I.70-1 to I.70-1499 in which R⁴, R⁵, R⁶, R⁷ and R⁸ have the meaning of Table 1 indicated in the respective row. Thus, the compounds I.70-1 to I.70-1499 of Table I.70 are defined by the meaning of the respective entries Nos. 1 to 1499 for R⁴, R⁵, R⁶, R⁷ and R⁸ of Table 1.

Preferred compounds according to the invention correspond to formula (I.71) below, where R⁴, R⁵, R⁶, R⁷ and R⁸ each have the meaning mentioned above or the preferred or particularly preferred meaning mentioned above.

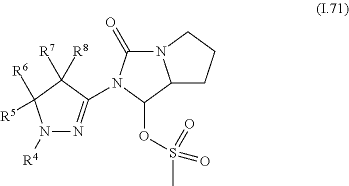

Table I.71: Preferred compounds of the formula (I.71) are the compounds I.71-1 to I.71-1499 in which R⁴, R⁵, R⁶, R⁷ and R⁸ have the meaning of Table 1 indicated in the respective row. Thus, the compounds I.71-1 to I.71-1499 of Table I.71 are defined by the meaning of the respective entries Nos. 1 to 1499 for R⁴, R⁵, R⁶, R⁷ and R⁸ of Table 1.

Preferred compounds according to the invention correspond to formula (I.72) below, where R⁴, R⁵, R⁶, R⁷ and R⁸ each have the meaning mentioned above or the preferred or particularly preferred meaning mentioned above.

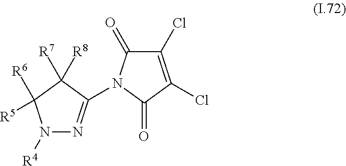

Table I.72: Preferred compounds of the formula (I.72) are the compounds I.72-1 to I.72-1499 in which R⁴, R⁵, R⁶, R⁷ and R⁸ have the meaning of Table 1 indicated in the respective row. Thus, the compounds I.72-1 to I.72-1499 of Table I.72 are defined by the meaning of the respective entries Nos. 1 to 1499 for R⁴, R⁵, R⁶, R⁷ and R⁸ of Table 1.

Preferred compounds according to the invention correspond to formula (I.73) below, where R⁴, R⁵, R⁶, R⁷ and R⁸ each have the meaning mentioned above or the preferred or particularly preferred meaning mentioned above.

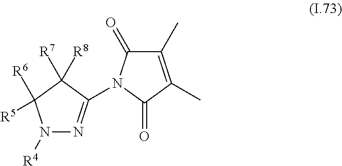

Table I.73: Preferred compounds of the formula (I.73) are the compounds I.73-1 to I.73-1499 in which R⁴, R⁵, R⁶, R⁷ and R⁸ have the meaning of Table 1 indicated in the respective row. Thus, the compounds I.73-1 to I.73-1499 of Table I.73 are defined by the meaning of the respective entries Nos. 1 to 1499 for R⁴, R⁵, R⁶, R⁷ and R⁸ of Table 1.

Preferred compounds according to the invention correspond to formula (I.74) below, where R⁴, R⁵, R⁶,

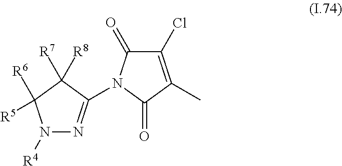

Table I.74: Preferred compounds of the formula (I.74) are the compounds I.74-1 to I.74-1499 in which R⁴, R⁵, R⁶, R⁷ and R⁸ have the meaning of Table 1 indicated in the respective row. Thus, the compounds I.74-1 to I.74-1499 of Table I.74 are defined by the meaning of the respective entries Nos. 1 to 1499 for R⁴, R⁵, R⁶, R⁷ and R⁸ of Table 1.

Preferred compounds according to the invention correspond to formula (I.75) below, where R⁴, R⁵, R⁶, R⁷ and R⁸ each have the meaning mentioned above or the preferred or particularly preferred meaning mentioned above.

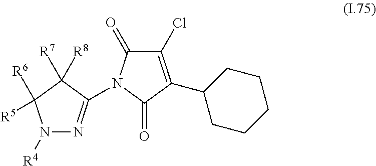

Table I.75: Preferred compounds of the formula (I.75) are the compounds I.75-1 to I.75-1499 in which R⁴, R⁵, R⁶, R⁷ and R⁸ have the meaning of Table 1 indicated in the respective row. Thus, the compounds I.75-1 to I.75-1499 of Table I.75 are defined by the meaning of the respective entries Nos. 1 to 1499 for R⁴, R⁵, R⁶, R⁷ and R⁸ of Table 1.

Preferred compounds according to the invention correspond to formula (I.76) below, where R⁴, R⁵, R⁶, R⁷ and R⁸ each have the meaning mentioned above or the preferred or particularly preferred meaning mentioned above.

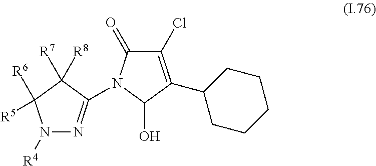

Table I.76: Preferred compounds of the formula (I.76) are the compounds I.76-1 to I.76-1499 in which R⁴, R⁵, R⁶, R⁷ and R⁸ have the meaning of Table 1 indicated in the respective row. Thus, the compounds I.76-1 to I.76-1499 of Table I.76 are defined by the meaning of the respective entries Nos. 1 to 1499 for R⁴, R⁵, R⁶, R⁷ and R⁸ of Table 1.

Preferred compounds according to the invention correspond to formula (I.77) below, where R⁴, R⁵, R⁶, R⁷ and R⁸ each have the meaning mentioned above or the preferred or particularly preferred meaning mentioned above.

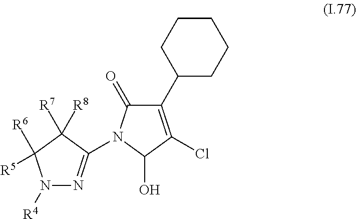

Table I.77: Preferred compounds of the formula (I.77) are the compounds I.77-1 to I.77-1499 in which R⁴, R⁵, R⁶, R⁷ and R⁸ have the meaning of Table 1 indicated in the respective row. Thus, the compounds I.77-1 to I.77-1499 of Table I.77 are defined by the meaning of the respective entries Nos. 1 to 1499 for R⁴, R⁵, R⁶, R⁷ and R⁸ of Table 1.

Synthesis of pyrazolinylpyrrolones and pyrazolinylhydantoins of the formula (I) according to the invention:

Substituted pyrazolinylpyrrolones and pyrazolinylhydantoins of the general formula (I) can be prepared by the processes described below. The starting materials for preparing the compounds mentioned are either commercially available or can be prepared by the synthesis methods listed below. In the schemes below, R¹, R², R³, R⁴, R⁵, R⁶, R⁷, R⁸, R⁹, R¹⁰, R¹¹, R¹², R¹³ and R¹⁴ each have the meanings defined above in connection with the compounds of the formula (I) according to the invention, unless the scheme gives exemplary, but not limiting, definitions.

The aminopyrazolines of type (IIa) (in this case, R⁵, R⁶ and the atom to which they are attached do not form a carbonyl group) are either commercially available or they can be prepared by the synthesis methods listed below according to processes A1, A2 or A3.

Process A1:

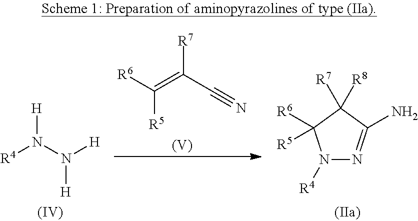

The aminopyrazolines of type (IIa) are prepared, for example, by a cyclization reaction of substituted acrylonitriles of type (V) and hydrazines of type (IV) or salts thereof (cf. Scheme 1). This reaction is carried out in the presence of a strong base, for example sodium ethoxide or sodium methoxide, using a suitable solvent, for example ethanol or methanol, as described in Scheme 1. This synthesis method is described inter alia in the literature references below: U.S. Pat. No. 4,451,479; WO1999/015505; U.S. Pat. No. 9,199,964; US2013/0085132; WO2011/033018; WO2015/058021; DE1944054; Eur. J. Med. Chem. 1989, 24, 435-445. The acrylonitriles of type (V) are commercially available or can be prepared as described in WO2011/033018 by reacting commercially available diethyl (cyanomethyl)phosphonate with a suitable aldehyde.

Process A2:

Alternatively, the preparation of the aminopyrazolines of type (IIa-1) in which R⁵, R⁶, R⁷ and R⁸, by way of example, but not by way of limitation, each represent hydrogen, can take place via the synthesis of acylaminopyrazolines of type (XII) with subsequent hydrolysis, as described in Scheme 2. The synthesis is carried out by reacting a suitable ester of type (X) either in one step starting with β-aminopropionamidoximes of type (IX) or in two steps via formation of aminooxadiazoles of type (XI), followed by an azole azoline rearrangement. The β-aminopropionamidoximes of type (IX) are accessible by reaction of amines of type (VI) with unsubstituted acrylonitrile (VII) and subsequent reaction with hydroxylamine H₂NOH, according to J. Chem Research (S) 1979, 64-65.

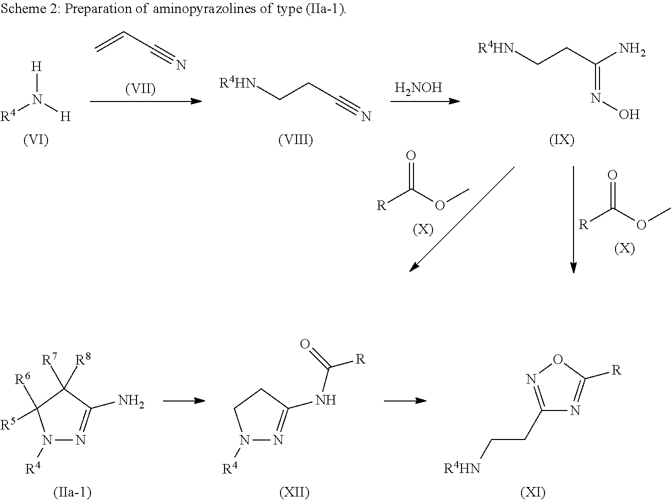

Process A3:

Preparation of the aminopyrazolines of type (IIa-3) and (IIa-4) in which R⁴, by way of example, but not by way of limitation, represents C(O)R¹² or SO₂R¹³, is carried out as described in Scheme 3 by acylation or sulfonylation starting with the aminopyrazolines of type (IIa-2) in which R⁴ represents hydrogen. These synthesis methods are described inter alia in the literature references below: H. Dorn, G. Hilgetag, A. Zubek, Chem. Ber. 1965, 98, 3368; Eur. J. Med. Chem. 1989, 24, 435-445.

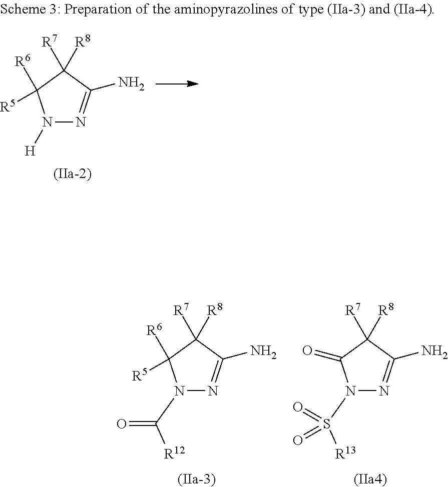

The aminopyrazolinones of type (IIb) are either commercially available or can be prepared by the synthesis methods listed below according to processes B1, B2 or B3.

Process B1:

Aminopyrazolinones of type (IIb) are, as described in Scheme 4, prepared via a cyclization reaction starting with hydrazines of type (IV) and suitable ethyl cyanoacetates of type (XIII) or aminomethyl acrylates of type (XIV) (R⁷═H, alkyl). These synthesis methods are described inter alia in the literature references below: Organic Syntheses 1948, 28, 87-89; Journal of Heterocyclic Chemistry 1990, 27, 683-686; Archiv der Pharmazie (Weinheim, Germany) 1978, 311, 446-453; JP 2008246906; DE3436383A.

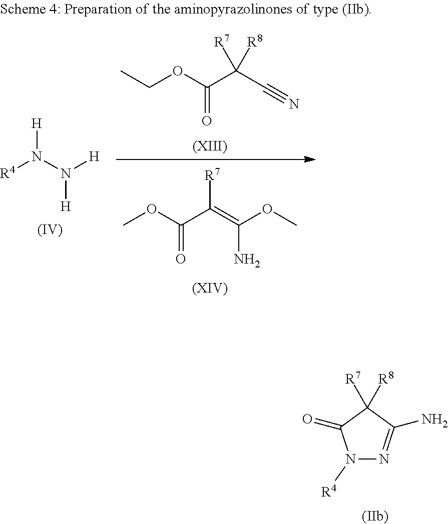

Process B2:

Alternatively, the aminopyrazolinones of type (IIb-1) in which R⁷ and R⁸ represent hydrogen can be prepared from methyl 3-nitropropionate (XV) using NaOEt in EtOH and a suitable hydrazine of type (IV) (R⁴=aryl), employing Raney-Ni in alcoholic solution, via formation of a nitrohydrazone (XVI), according to J. Heterocyclic Chem. 1983, 20, 773 (Scheme 5).

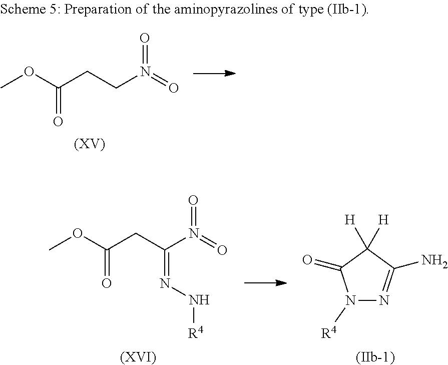

Process B3:

Halogenated aminopyrazolinones of type (IIb-2), (R⁷═Cl, Br and R⁸═H, Cl, Br) are prepared from aminopyrazolinones of type (IIb-1) in which R⁷ and R⁸ represent hydrogen by reaction with SOCl₂ or Br₂/AcOH, as described in Scheme 6. These synthesis methods are described inter alia in the literature references below: Sb. Nauch. Tr. Vses. N.-i. i Proekt. In-t Khim.-fotogr. Prom-sti, 1977, 25, 87-92; Heteroatom Chemistry 2003, 14, 211-217.

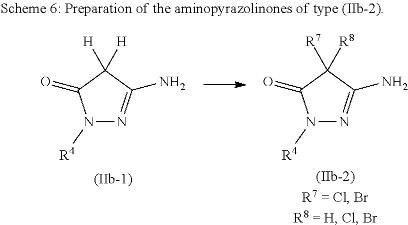

Pyrazolinylpyrrolones of type (Ia) or derivatives thereof of type (Ib) are prepared from aminopyrazolines or aminopyrazolinones of type (II) according to Scheme 7 below.

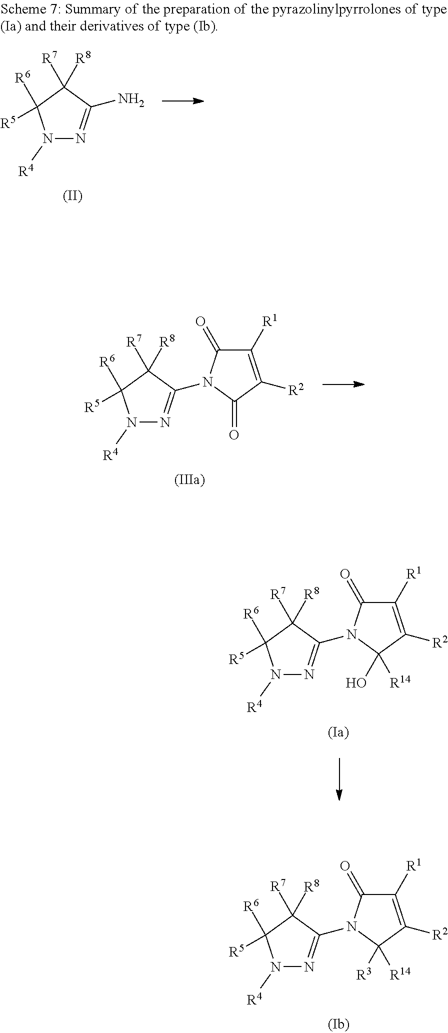

The succinimides of type (IIIa) can be prepared from aminopyrazolin(on)es of type (II), by the synthesis methods listed below according to processes C1 or C2, as described in Scheme 8.

Compounds of type (la) can be prepared from succinimides of type (IIIa) according to Process D described below, whereas compounds of type (la) can be converted into compounds of type (Ib) according to Process E described below.

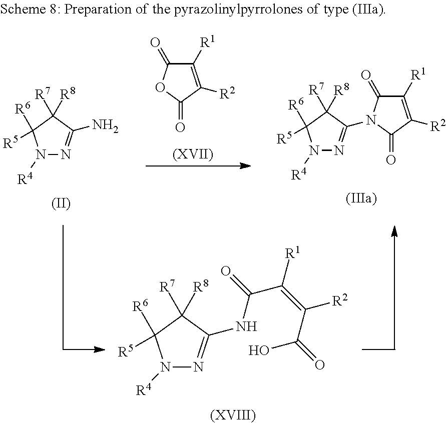

Process C1:

The succinimides of type (IIIa) are prepared, for example, as described in Scheme 8 by reacting aminopyrazolines of type (II) with maleic anhydrides of the general formula (XVII) which are commercially available or can be obtained by literature processes, in the presence of a suitable solvent using a suitable acid or base. These synthesis methods and the preparation of the maleic anhydrides of type (XVII) are described inter alia in the following literature references: EP0297378; EP334133; WO2014/180740; WO2015/018434.

Process C2:

Alternatively, the succinimides of type (IIIa) can be prepared by a two-step synthesis where initially the carboxylic acid (XVIII) is formed and then cyclization with formation of an activated ester intermediate takes place. These synthesis methods are described inter alia in the literature references below: WO2015/018431; WO2015/018432; WO2015/018433.

Process D:

In order to prepare the pyrazolinylpyrrolones of type (Ia), one of the two carbonyl functions of the succinimide of type (IIIa) is reduced using a suitable reducing agent, for example NaBH₄, LiAlH₄ or LiBHEt₃ (superhydrides), as described in Scheme 9, to afford the 2-hydroxy-5-oxy-2-hydropyrrolone of type (Ia). These synthesis methods are described inter alia in the literature references below: EP0297378; EP334133; WO2015/018434; WO2015/018431; WO2015/018432; WO2015/018433; WO2016/071359; WO2016/071360.

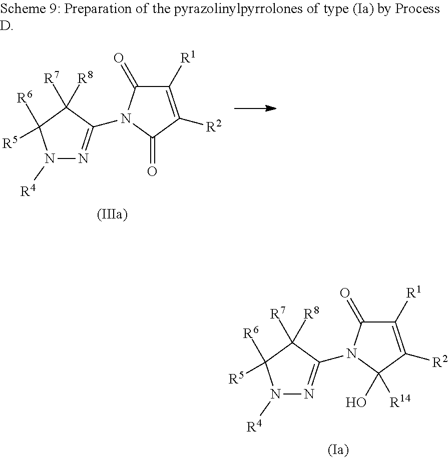

Process E:

The pyrazolinylpyrrolones of type (Ib) are prepared from pyrazolinylpyrrolones of type (Ia), for example by halogenation, alkylation, acylation, sulfonylation or alkoxyacylation. These synthesis methods are described inter alia in the literature references below: EP0297378; EP334133; WO20150/184341; WO2015/018431; WO2015/018432; WO2015/018433; WO2016/071359; WO2016/071360.

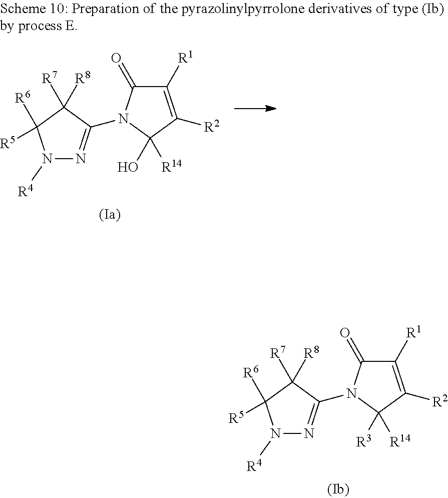

The pyrazolinylhydantoins of type (Ic) and their derivatives of type (Id) can be prepared from aminopyrazolines of type (II) by the synthesis methods listed below according to Processes F and G, as described in Scheme 11.

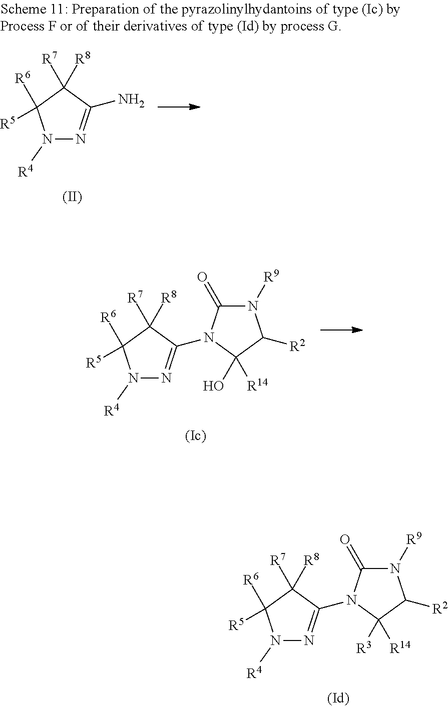

Process F:

The pyrazolinylhydantoins of type (Ic) are prepared in a three-step synthesis as described in Scheme 12, starting with pyrazolines of type (II) by formation of urea (XX) and a subsequent cyclization reaction. These synthesis methods are described inter alia in the literature references below: WO2015/097043; WO2015/059262; WO2015067701; WO2015/024853; WO2015/052076; WO2015/193202; U.S. Pat. No. 4,268,679; WO2015/018431; WO2015/018432; WO2015/018433; WO2016/071361; WO2016/071362; WO2016/071363; WO2016/071364.

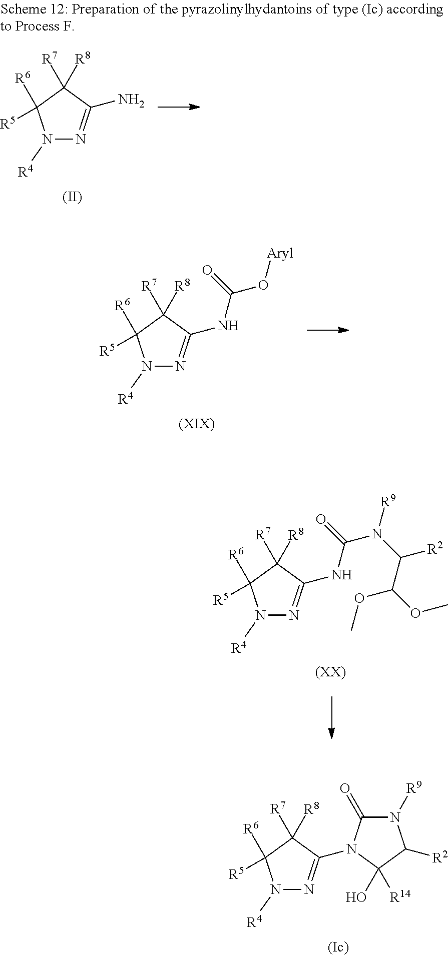

Process G:

The pyrazolinylhydantoins of type (Id) are prepared analogously to the preparation of the pyrazolinylpyrrolones of type (Ib) starting with pyrazolinylhydantoins of type (Ic) by the synthesis method according to Process G as described in Scheme 13. Here, process G is analogous to Process E. These synthesis methods are described inter alia in the literature references below: WO 2015/097043; WO2015/059262; WO2015/067701; WO2015/024853; WO2015/052076; WO2015/193202; U.S. Pat. No. 4,268,679.

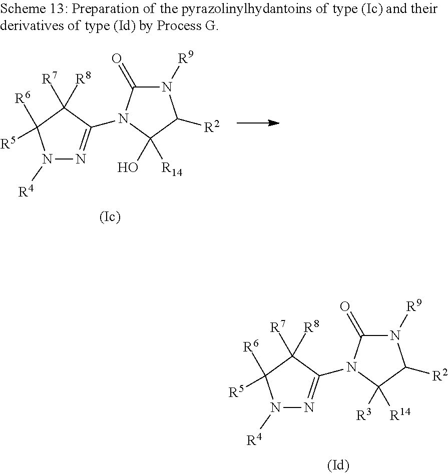

Process H:

The aminopyrazolines of type (IIc) (R⁵ represents, by way of example, but not by way of limitation, an ester group) can be prepared by the synthesis methods listed below according to Process H. In the first step of this process, a hydrazine, optionally with further substitution, is converted with glyoxylic acid in a suitable polar solvent (e.g. water) with addition of a suitable mineral acid (e.g. HCl) into the corresponding hydrazone (XXI) which, in the next step, by reaction with a suitable brominating agent (e.g. N-bromosuccinimide=NBS) in a suitable polar-aprotic solvent (e.g. N,N-dimethylformamide=DMF) forms the corresponding dibromohydrazone (XXII), optionally with further substitution (cf. WO2014/033449). In the subsequent step, a cycloaddition mediated by suitable bases (e.g. triethylamine) with suitable unsaturated carboxylic esters (XXIII), optionally with further substitution, in a suitable polar-aprotic solvent (e.g. dichloromethane or chloroform) leads to the formation of the bromopyrazoline (XXIV), optionally with further substitution (cf. Synthesis 2007, 11, 1730).

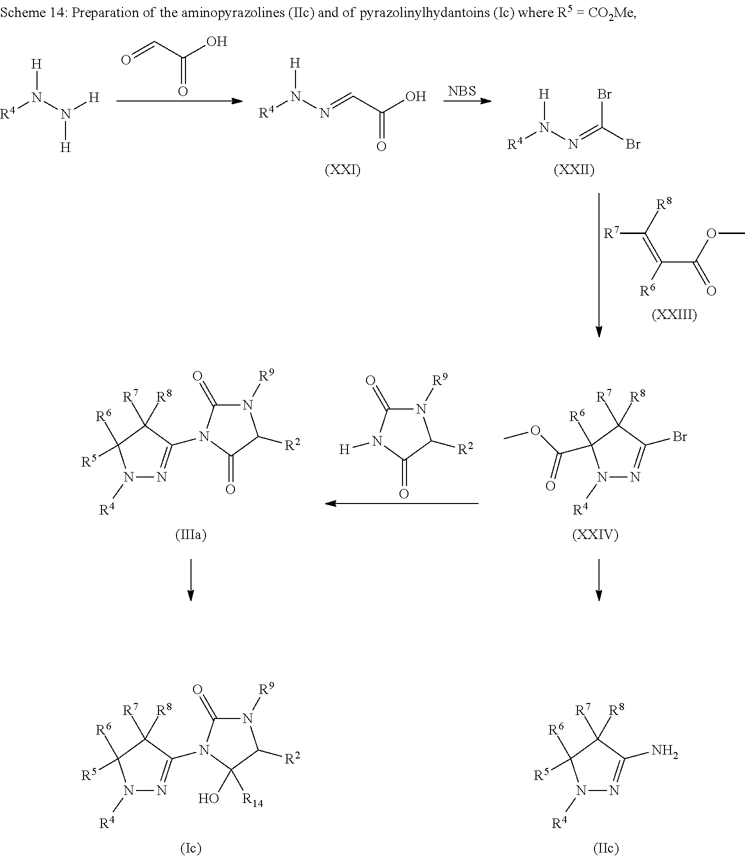

With the aid of a suitable aminating agent, the bromopyrazoline (XXIV) is then converted into the aminopyrazoline (IIc), optionally with further substitution, which can then be converted according to the processes described above into the corresponding pyrazolinylsuccinimides or pyrazolinylhydantoins of the general formulae (Ia) or (Ic), optionally with further substitution. Alternatively, the bromopyrazoline (XXIV), optionally with further substitution, can also be reacted directly with a hydantoin to give the corresponding pyrazolinehydantoin (Ic), optionally with further substitution. Scheme 14 shows the synthesis sequence by way of example, but not by way of limitation, for pyrazolinylhydantoins; here, by way of example, but not by way of limitation, R⁵ represents a CO₂Me group, whereas R¹, R⁴, R⁵, R⁶, R⁷, R⁸ and R⁹ each have the meanings defined above.

Accordingly, the present invention also provides a process for preparing a compound of the formula (I) as defined in the context of the present invention, preferably in an embodiment characterized as preferred, more preferred, even more preferred or particularly preferred, or a salt thereof, characterized in that in this process

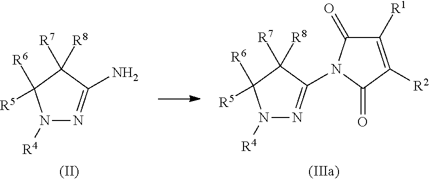

a compound of the formula (II) is converted into a compound of the formula (IIIa), where R¹, R², R⁴, R⁵, R⁶, R⁷ and R⁸ each have the meaning defined above,

or

a compound of the formula (II) is converted into a compound of the formula (XX)

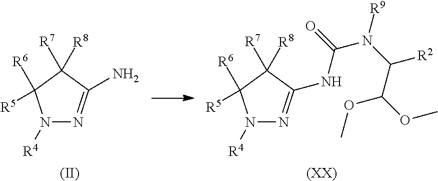

where R², R⁴, R⁵, R⁶, R⁷, R⁸ and R⁹ each have the meaning defined above.

A preferred process according to the invention for preparing a compound of the formula (I) as defined in the context of the present invention, preferably in an embodiment characterized as preferred, more preferred, even more preferred or particularly preferred, or its salt is characterized in that in this process

a compound of the formula (II) is converted into a compound of the formula (IIIa), where R¹, R², R⁴, R⁵, R⁶, R⁷ and R⁸ each have the meaning defined above, where
the reaction is carried out with a compound of the formula (XVII) in the presence of a suitable solvent and using a suitable acid or base, or via a two-step synthesis where initially the compound of the formula (XVIII) is formed and subsequently cyclization takes place,

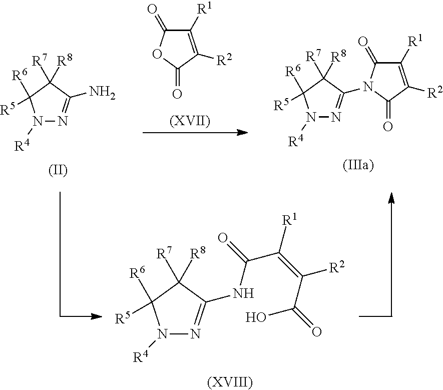

or
a compound of the formula (II) is converted into a compound of the formula (XX) and the latter is then converted further in a cyclization reaction into a compound of the formula (Ic),

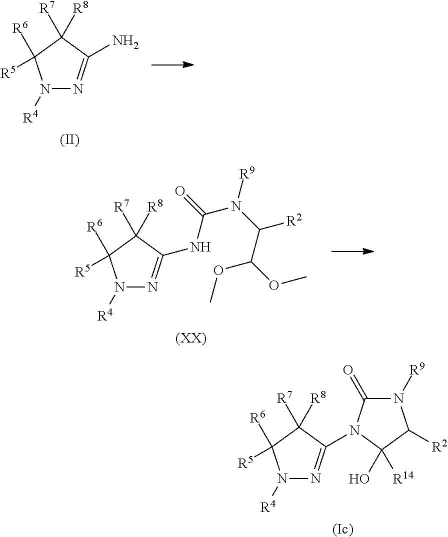

where R², R⁴, R⁵, R⁶, R⁷, R⁸, R⁹ and R¹⁴ each have the meaning defined above.

A collection of compounds of the formula (I) can additionally be prepared in a parallel or combinatorial manner, it being possible for this to take place in a manual, partly automated or completely automated manner. In this connection, is possible to automate the reaction procedure, the work-up or the purification of the products and/or intermediates.

For the parallelized reaction procedure and work-up it is possible to use a range of commercially available instruments, of the kind offered by, for example, the companies Stem Corporation, Woodrolfe Road, Tollesbury, Essex, CM9 8SE, England, or H+P Labortechnik GmbH, Bruckmannring 28, 85764 Oberschleißheim, Germany. For the parallelized purification of compounds (I) or of intermediates produced during the preparation, there are available, inter alia, chromatography apparatuses, for example from ISCO, Inc., 4700 Superior Street, Lincoln, Nebr. 68504, USA. The apparatuses listed allow a modular procedure in which the individual process steps are automated, but between the process steps manual operations have to be carried out. This can be circumvented by using partly or completely manual operations have to be carried out. This can be circumvented by using partly or completely integrated automation systems in which the respective automation modules are operated, for example, by robots. Automation systems of this type can be acquired, for example, from Zymark Corporation, Zymark Center, Hopkinton, Mass. 01748, USA.

Besides the methods described, the preparation of compounds of the formula (I) can take place completely or partially by solid-phase-supported methods. For this purpose, individual intermediates or all intermediates in the synthesis or a synthesis adapted for the corresponding procedure are bonded to a synthesis resin. Solid-phase-supported synthesis methods are described extensively in the specialist literature, for example: Barry A. Bunin in “The Combinatorial Index”, Verlag Academic Press, 1998. The use of solid-phase-supported synthesis methods permits a number of protocols, which are known from the literature and which for their part may be performed manually or in an automated manner. For example, the so-called “tea bag method” (U.S. Pat. No. 4,631,211) in which products from IRORI, 11149 North Torrey Pines Road, La Jolla, Calif. 92037, USA, are employed, may be semiautomated. The automation of solid-phase-supported parallel syntheses is performed successfully, for example, by apparatuses from Argonaut Technologies, Inc., 887 Industrial Road, San Carlos, Calif. 94070, USA or MultiSynTech GmbH, Wullener Feld 4, 58454 Witten, Germany.

The preparation according to the processes described herein produces compounds of the formula (I) in the form of substance collections or libraries. Accordingly, the present invention also provides libraries of compounds of the formula (I) which comprise at least two compounds of the formula (I), and precursors thereof.

The present invention furthermore provides the use of one or more compounds of the formula (I) and/or salts thereof, as defined above, preferably in one of the embodiments identified as preferred or particularly preferred, in particular one or more compounds of the formulae (I.1) to (I.77) and/or salts thereof, in each case as defined above,

as herbicide and/or plant growth regulator, preferably in crops of useful plants and/or ornamental plants.

The present invention furthermore provides a method for controlling harmful plants and/or for regulating the growth of plants, characterized in that an effective amount
of one or more compounds of the formula (I) and/or salts thereof, as defined above, preferably in one of the embodiments identified as preferred or particularly preferred, in particular one or more compounds of the formulae (I.1) to (I.77) and/or salts thereof, in each case as defined above, or
of a composition according to the invention, as defined below,

is applied to the (harmful) plants, seeds of (harmful) plants, the soil in which or on which the (harmful) plants grow or the area under cultivation.

The present invention also provides a method for controlling unwanted plants, preferably in crops of useful plants, characterized in that an effective amount
of one or more compounds of the formula (I) and/or salts thereof, as defined above, preferably in one of the embodiments identified as preferred or particularly preferred, in particular one or more compounds of the formulae (I.1) to (I.77) and/or salts thereof, in each case as defined above,
of a composition according to the invention, as defined below,

is applied to unwanted plants (for example harmful plants such as mono- or dicotyledonous weeds or unwanted crop plants), the seed of the unwanted plants (i.e. plant seeds, for example grains, seeds or vegetative propagation organs such as tubers or shoot parts with buds), the soil in which or on which the unwanted plants grow (for example the soil of crop land or non-crop land) or the area under cultivation (i.e. the area on which the unwanted plants will grow).

The present invention furthermore also provides methods for regulating the growth of plants, preferably of useful plants, characterized in that an effective amount
of one or more compounds of the formula (I) and/or salts thereof, as defined above, preferably in one of the embodiments identified as preferred or particularly preferred, in particular one or more compounds of the formulae (I.1) to (I.77) and/or salts thereof, in each case as defined above, or
of a composition according to the invention, as defined below,

is applied to the plant, the seed of the plant (i.e. plant seed, for example grains, seeds or vegetative propagation organs such as tubers or shoot parts with buds), the soil in which or on which the plants grow (for example the soil of crop land or non-crop land) or the area under cultivation (i.e. the area on which the plants will grow).

In this context, the compounds according to the invention or the compositions according to the invention can be applied for example by pre-sowing (if appropriate also by incorporation into the soil), pre-emergence and/or post-emergence processes. Specific examples of some representatives of the monocotyledonous and dicotyledonous weed flora which can be controlled by the compounds according to the invention are as follows, though there is no intention to restrict the enumeration to particular species.

In a method according to the invention for controlling harmful plants or for regulating the growth of plants, one or more compounds of the formula (I) and/or salts thereof are preferably employed for controlling harmful plants or for regulating growth in crops of useful plants or ornamental plants, where in a preferred embodiment the useful plants or ornamental plants are transgenic plants.

The compounds of the formula (I) according to the invention and/or their salts are suitable for controlling the following genera of monocotyledonous and dicotyledonous harmful plants: Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous harmful plants of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

When the compounds according to the invention are applied to the soil surface before germination of the harmful plants (weed grasses and/or broad-leaved weeds) (pre-emergence method), either the seedlings of the weed grasses or broad-leaved weeds are prevented completely from emerging or they grow until they have reached the cotyledon stage, but then stop growing and eventually, after three to four weeks have elapsed, die completely.

If the active compounds are applied post-emergence to the green parts of the plants, growth stops after the treatment, and the harmful plants remain at the growth stage of the point of time of application, or they die completely after a certain time, so that in this manner competition by the weeds, which is harmful to the crop plants, is eliminated very early and in a sustained manner. Although the compounds according to the invention display an outstanding herbicidal activity against monocotyledonous and dicotyledonous weeds, crop plants of economically important crops, for example dicotyledonous crops of the genera Arachis, Beta, Brassica, Cucumis, Cucurbita, Helianthus, Daucus, Glycine, Gossypium, Ipomoea, Lactuca, Linum, Lycopersicon, Miscanthus, Nicotiana, Phaseolus, Pisum, Solanum, Vicia, or monocotyledonous crops of the genera Allium, Ananas, Asparagus, Avena, Hordeum, Oryza, Panicum, Saccharum, Secale, Sorghum, triticale, triticum, Zea, are damaged only to an insignificant extent, or not at all, depending on the structure of the respective compound according to the invention and its application rate. For these reasons, the present compounds are very suitable for selective control of unwanted plant growth in plant crops such as agriculturally useful plants or ornamental plants.

In addition, the compounds of the invention (depending on their particular structure and the application rate deployed) have outstanding growth-regulating properties in crop plants. They intervene in the plants' own metabolism with regulatory effect, and can thus be used for the controlled influencing of plant constituents and to facilitate harvesting, for example by triggering desiccation and stunted growth. Furthermore, they are also suitable for the general control and inhibition of unwanted vegetative growth without killing the plants in the process. Inhibition of vegetative growth plays a major role for many mono- and dicotyledonous crops since, for example, this can reduce or completely prevent lodging.

By virtue of their herbicidal and plant growth regulatory properties, the active compounds can also be used to control harmful plants in crops of genetically modified plants or plants modified by conventional mutagenesis. In general, the transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material.

It is preferred with a view to transgenic crops to use the compounds according to the invention and/or their salts in economically important transgenic crops of useful plants and ornamentals, for example of cereals such as wheat, barley, rye, oats, millet, rice and maize or else crops of sugar beet, cotton, soybean, oilseed rape, potato, tomato, peas and other vegetables.

It is preferred to employ the compounds according to the invention as herbicides in crops of useful plants which are resistant, or have been made resistant by recombinant means, to the phytotoxic effects of the herbicides.

By virtue of their herbicidal and plant growth regulatory properties, the active compounds can also be used to control harmful plants in crops of genetically modified plants which are known or are yet to be developed. In general, the transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material. Further special properties may be tolerance or resistance to abiotic stressors, for example heat, cold, drought, salinity and ultraviolet radiation.

Preference is given to the use of the compounds of the formula (I) according to the invention or salts thereof in economically important transgenic crops of useful plants and ornamental plants, for example of cereals such as wheat, barley, rye, oats, triticale, millet, rice, cassava and corn, or else crops of sugar beet, cotton, soybean, oilseed rape, potatoes, tomatoes, peas and other vegetables.

The compounds of the formula (I) can preferably be used as herbicides in crops of useful plants which are resistant, or have been made resistant by recombinant means, to the phytotoxic effects of the herbicides.

Conventional ways of producing novel plants which have modified properties in comparison to existing plants consist, for example, in traditional cultivation methods and the generation of mutants. Alternatively, novel plants with altered properties can be generated with the aid of recombinant methods.

A large number of molecular-biological techniques by means of which novel transgenic plants with modified properties can be generated are known to the person skilled in the art. For such recombinant manipulations, nucleic acid molecules which allow mutagenesis or sequence alteration by recombination of DNA sequences can be introduced into plasmids. With the aid of standard methods, it is possible, for example, to undertake base exchanges, remove parts of sequences or add natural or synthetic sequences. To connect the DNA fragments to each other, adapters or linkers may be added to the fragments. For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product.

To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Such sequences are known to those skilled in the art (see, for example, Braun et al., EMBO J. 11 (1992), 3219-3227). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants.

Thus, transgenic plants can be obtained whose properties are altered by overexpression, suppression or inhibition of homologous (=natural) genes or gene sequences or expression of heterologous (=foreign) genes or gene sequences.

It is preferred to employ the compounds (I) according to the invention in transgenic crops which are resistant to growth regulators such as, for example, dicamba, or against herbicides which inhibit essential plant enzymes, for example acetolactate synthases (ALS), EPSP synthases, glutamine synthases (GS) or hydroxyphenylpyruvate dioxygenases (HPPD), or against herbicides from the group of the sulfonylureas, glyphosate, glufosinate or benzoylisoxazoles and analogous active compounds.

When the active compounds of the invention are employed in transgenic crops, not only do the effects toward harmful plants observed in other crops occur, but frequently also effects which are specific to application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also relates to the use of the compounds of the formula (I) according to the invention and/or their salts as herbicides for controlling harmful plants in crops of useful plants or ornamentals, optionally in transgenic crop plants.

Preference is given to the use in cereals, here preferably maize, wheat, barley, rye, oats, millet or rice, by the pre- or post-emergence method.

Preference is also given to the use in soya beans by the pre- or post-emergence method.

The use according to the invention for the control of harmful plants or for growth regulation of plants also includes the case in which the active compound of the formula (I) or its salt is not formed from a precursor substance (“prodrug”) until after application on the plant, in the plant or in the soil.

The invention also provides the use of one or more compounds of the formula (I) or salts thereof or of a composition according to the invention (as defined below) (in a method) for controlling harmful plants or for regulating the growth of plants which comprises applying an effective amount of one or more compounds of the formula (I) or salts thereof onto the plants (harmful plants, if appropriate together with the useful plants), plant seeds, the soil in which or on which the plants grow or the area under cultivation.

The invention also provides a herbicidal and/or plant growth-regulating composition, characterized in that the composition comprises

(a) one or more compounds of the formula (I) and/or salts thereof, as defined above, preferably in one of the embodiments identified as preferred or particularly preferred, in particular one or more compounds of the formulae (I.1) to (I.77) and/or salts thereof, in each case as defined above,

and

(b) one or more further substances selected from groups (i) and/or (ii):
(i) one or more further agrochemically active substances, preferably selected from the group consisting of insecticides, acaricides, nematicides, further herbicides (i.e. those not corresponding to the formula (I) defined above), fungicides, safeners, fertilizers and/or further growth regulators,
(ii) one or more formulation auxiliaries customary in crop protection.

Here, the further agrochemically active substances of component (i) of a composition according to the invention are preferably selected from the group of substances mentioned in “The Pesticide Manual”, 16th edition, The British Crop Protection Council and the Royal Soc. of Chemistry, 2012.

A herbicidal or plant growth-regulating composition according to the invention comprises preferably one, two, three or more formulation auxiliaries (ii) customary in crop protection selected from the group consisting of surfactants, emulsifiers, dispersants, film-formers, thickeners, inorganic salts, dusting agents, carriers solid at 25° C. and 1013 mbar, preferably adsorbant granulated inert materials, wetting agents, antioxidants, stabilizers, buffer substances, antifoam agents, water, organic solvents, preferably organic solvents miscible with water in any ratio at 25° C. and 1013 mbar. The compounds (I) according to the invention can be used in the form of wettable powders, emulsifiable concentrates, sprayable solutions, dusting products or granules in the customary formulations. The invention therefore also provides herbicidal and plant growth-regulating compositions which comprise compounds of the formula (I) and/or salts thereof.

The compounds of the formula (I) and/or salts thereof can be formulated in various ways according to which biological and/or physicochemical parameters are required. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes.

These individual formulation types and the formulation assistants, such as inert materials, surfactants, solvents and further additives, are known to the person skilled in the art and are described, for example, in: Watkins, “Handbook of Insecticide Dust Diluents and Carriers”, 2nd Ed., Darland Books, Caldwell N.J.; H.v. Olphen, “Introduction to Clay Colloid Chemistry”; 2nd Ed., J. Wiley & Sons, N.Y.; C. Marsden, “Solvents Guide”; 2nd Ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schonfeldt, “Grenzflächenaktive Äthylenoxidaddukte” [Interface-active Ethylene Oxide Adducts], Wiss. Verlagsgesellschaft, Stuttgart 1976; Winnacker-Küchler, “Chemische Technologie” [Chemical Technology], volume 7, C. Hanser Verlag Munich, 4th Ed. 1986.

Wettable powders are preparations which can be dispersed uniformly in water and, in addition to the active compound, apart from a diluent or inert substance, also comprise surfactants of the ionic and/or nonionic type (wetting agents, dispersants), for example polyoxyethylated alkylphenols, polyoxyethylated fatty alcohols, polyoxyethylated fatty amines, fatty alcohol polyglycol ether sulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the active herbicidal ingredients are finely ground, for example in customary apparatuses such as hammer mills, blower mills and air-jet mills, and simultaneously or subsequently mixed with the formulation auxiliaries.

Emulsifiable concentrates are produced by dissolving the active compound in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulfonates such as calcium dodecylbenzenesulfonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters, or polyoxyethylene sorbitan esters, for example polyoxyethylene sorbitan fatty acid esters.

Dusting products are obtained by grinding the active compound with finely distributed solids, for example talc, natural clays, such as kaolin, bentonite and pyrophillite, or diatomaceous earth.

Suspension concentrates may be water- or oil-based. They may be prepared, for example, by wet-grinding by means of commercial bead mills and optional addition of surfactants as have, for example, already been listed above for the other formulation types.

Emulsions, for example oil-in-water emulsions (EW), can be produced, for example, by means of stirrers, colloid mills and/or static mixers using aqueous organic solvents and optionally surfactants as already listed above, for example, for the other formulation types.

Granules can be produced either by spraying the active compound onto adsorptive granular inert material or by applying active compound concentrates to the surface of carriers, such as sand, kaolinites or granular inert material, by means of adhesives, for example polyvinyl alcohol, sodium polyacrylate or else mineral oils. Suitable active compounds can also be granulated in the manner customary for the production of fertilizer granules—if desired as a mixture with fertilizers.

Water-dispersible granules are produced generally by the customary processes such as spray-drying, fluidized bed granulation, pan granulation, mixing with high-speed mixers and extrusion without solid inert material.

For the production of pan, fluidized-bed, extruder and spray granules, see e.g. processes in “Spray Drying Handbook” 3rd Ed. 1979, G. Goodwin Ltd., London, J. E. Browning, “Agglomeration”, Chemical and Engineering 1967, pages 147 ff, “Perry's Chemical Engineer's Handbook”, 5th Ed., McGraw Hill, N.Y. 1973, p. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical preparations, preferably herbicidal or plant growth-regulating compositions, of the present invention preferably comprise a total amount of from 0.1 to 99% by weight, preferably 0.5 to 95% by weight, particularly preferably 1 to 90% by weight, especially preferably 2 to 80% by weight, of active compounds of the formula (I) and their salts.

In wettable powders, the active compound concentration is, for example, about 10% to 90% by weight, the remainder to 100% by weight consisting of customary formulation constituents. In emulsifiable concentrates, the active compound concentration may be about 1% to 90% and preferably 5% to 80% by weight. Formulations in the form of dusts comprise 1% to 30% by weight of active compound, preferably usually 5% to 20% by weight of active compound; sprayable solutions contain about 0.05% to 80% by weight, preferably 2% to 50% by weight of active compound. In the case of water-dispersible granules, the active compound content depends partially on whether the active compound is in liquid or solid form and on which granulation auxiliaries, fillers, etc., are used. In the water-dispersible granules, the content of active compound is, for example, between 1% and 95% by weight, preferably between 10% and 80% by weight.

In addition, the active compound formulations mentioned optionally comprise the respective customary stickers, wetters, dispersants, emulsifiers, penetrants, preservatives, antifreeze agents and solvents, fillers, carriers and dyes, defoamers, evaporation inhibitors and agents which influence the pH and the viscosity. Examples of formulation auxiliaries are described inter alia in “Chemistry and Technology of Agrochemical Formulations”, ed. D. A. Knowles, Kluwer Academic Publishers (1998).

The compounds of the formula (I) or salts thereof can be used as such or in the form of their preparations (formulations) in a combination with other pesticidally active substances, for example insecticides, acaricides, nematicides, herbicides, fungicides, safeners, fertilizers and/or growth regulators, for example in the form of a finished formulation or of a tank mix. The combination formulations can be prepared on the basis of the abovementioned formulations, while taking account of the physical properties and stabilities of the active compounds to be combined.

Active compounds which can be employed in combination with the compounds of formula (I) according to the invention in mixture formulations or in a tank mix are, for example, known active compounds based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoendesaturase, photosystem I, photosystem II, protoporphyrinogen oxidase, as described, for example, in Weed Research 26 (1986) 441-445 or “The Pesticide Manual”, 16th edition, The British Crop Protection Council and the Royal Soc. of Chemistry, 2012 and literature cited therein.

Of particular interest is the selective control of harmful plants in crops of useful plants and ornamentals. Although the compounds (I) according to the invention have already demonstrated very good to adequate selectivity in a large number of crops, in principle, in some crops and in particular also in the case of mixtures with other, less selective herbicides, phytotoxicities on the crop plants may occur. In this connection, combinations of compounds (I) according to the invention are of particular interest which comprise the compounds (I) or their combinations with other herbicides or pesticides and safeners. The safeners, which are used in an antidotically effective amount, reduce the phytotoxic side effects of the herbicides/pesticides employed, for example in economically important crops, such as cereals (wheat, barley, rye, corn, rice, millet), sugarbeet, sugarcane, oilseed rape, cotton and soybeans, preferably cereals.

The weight ratios of herbicide (mixture) to safener depend generally on the herbicide application rate and the efficacy of the safener in question and may vary within wide limits, for example in the range from 200:1 to 1:200, preferably 100:1 to 1:100, in particular 20:1 to 1:20. Analogously to the compounds (I) or mixtures thereof, the safeners can be formulated with further herbicides/pesticides and be provided and employed as a finished formulation or tank mix with the herbicides.

For application, the herbicide or herbicide/safener formulations present in commercial form are, if appropriate, diluted in a customary manner, for example in the case of wettable powders, emulsifiable concentrates, dispersions and water-dispersible granules with water. Dust-type preparations, granules for soil application or granules for scattering and sprayable solutions are not normally diluted further with other inert substances prior to application.

The application rate of the compounds of the formula (I) and/or their salts is affected to a certain extent by external conditions such as temperature, humidity, etc. Here, the application rate may vary within wide limits. For the application as a herbicide for controlling harmful plants, the total amount of compounds of the formula (I) and their salts is preferably in the range from 0.001 to 10.0 kg/ha, with preference in the range from 0.005 to 5 kg/ha, more preferably in the range from 0.01 to 1.5 kg/ha, in particular in the range from 0.05 to 1 kg/ha. This applies both to the pre-emergence and the post-emergence application.

When the compounds of the formula (I) and/or their salts are used as plant growth regulator, for example as culm stabilizer for crop plants like those mentioned above, preferably cereal plants, such as wheat, barley, rye, triticale, millet, rice or maize, the total application rate is preferably in the range of from 0.001 to 2 kg/ha, preferably in the range of from 0.005 to 1 kg/ha, in particular in the range of from 10 to 500 g/ha, very particularly in the range from 20 to 250 g/ha. This applies both to the pre-emergence and the post-emergence application.

The application as culm stabilizer may take place at various stages of the growth of the plants. Preferred is, for example, the application after the tillering phase, at the beginning of the longitudinal growth.

As an alternative, application as plant growth regulator is also possible by treating the seed, which includes various techniques for dressing and coating seed. Here, the application rate depends on the particular techniques and can be determined in preliminary tests.

Active compounds which can be employed in combination with the compounds of the formula (I) according to the invention in compositions according to the invention (for example in mixed formulations or in the tank mix) are, for example, known active compounds which are based on the inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II, protoporphyrinogen oxidase, as are described in, for example, Weed Research 26 (1986) 441-445 or “The Pesticide Manual”, 16th edition, The British Crop Protection Council and the Royal Soc. of Chemistry, 2012 and the literature cited therein. Known herbicides or plant growth regulators which can be combined with the compounds according to the invention are, for example, the following active compounds, where the compounds are designated either with the “common name” in accordance with the International Organization for Standardization (ISO) or with the chemical name or with the code number. They always encompass all of the application forms such as, for example, acids, salts, esters and also all isomeric forms such as stereoisomers and optical isomers, even if not explicitly mentioned.

Examples of such herbicidal mixing partners are:

acetochlor, acifluorfen, acifluorfen-sodium, aclonifen, alachlor, allidochlor, alloxydim, alloxydim-sodium, ametryn, amicarbazone, amidochlor, amidosulfuron, 4-amino-3-chloro-6-(4-chloro-2-fluoro-3-methylphenyl)-5-fluoropyridine-2-carboxylic acid, aminocyclopyrachlor, aminocyclopyrachlor-potassium, aminocyclopyrachlor-methyl, aminopyralid, amitrole, ammoniumsulfamate, anilofos, asulam, atrazine, azafenidin, azimsulfuron, beflubutamid, benazolin, benazolin-ethyl, benfluralin, benfuresate, bensulfuron, bensulfuron-methyl, bensulide, bentazone, benzobicyclon, benzofenap, bicyclopyron, bifenox, bilanafos, bilanafos-sodium, bispyribac, bispyribac-sodium, bromacil, bromobutide, bromofenoxim, bromoxynil, bromoxynil-butyrate, -potassium, -heptanoate and -octanoate, busoxinone, butachlor, butafenacil, butamifos, butenachlor, butralin, butroxydim, butylate, cafenstrole, carbetamide, carfentrazone, carfentrazone-ethyl, chloramben, chlorbromuron, chlorfenac, chlorfenac-sodium, chlorfenprop, chlorflurenol, chlorflurenol-methyl, chloridazon, chlorimuron, chlorimuron-ethyl, chlorophthalim, chlorotoluron, chlorthal-dimethyl, chlorsulfuron, cinidon, cinidon-ethyl, cinmethylin, cinosulfuron, clacyfos, clethodim, clodinafop, clodinafop-propargyl, clomazone, clomeprop, clopyralid, cloransulam, cloransulam-methyl, cumyluron, cyanamide, cyanazine, cycloate, cyclopyrimorate, cyclosulfamuron, cycloxydim, cyhalofop, cyhalofop-butyl, cyprazine, 2,4-D, 2,4-D-butotyl, -butyl, -dimethylammonium, -diolamin, -ethyl, 2-ethylhexyl, -isobutyl, -isooctyl, -isopropylammonium, -potassium, -triisopropanolammonium and -trolamine, 2,4-DB, 2,4-DB-butyl, -dimethylammonium, isooctyl, -potassium and -sodium, daimuron (dymron), dalapon, dazomet, n-decanol, desmedipham, detosyl-pyrazolate (DTP), dicamba, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-P, diclofop, diclofop-methyl, diclofop-P-methyl, diclosulam, difenzoquat, diflufenican, diflufenzopyr, diflufenzopyr-sodium, dimefuron, dimepiperate, dimethachlor, dimethametryn, dimethenamid, dimethenamid-P, dimetrasulfuron, dinitramine, dinoterb, diphenamid, diquat, diquat-dibromid, dithiopyr, diuron, DNOC, endothal, EPTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, F-9600, F-5231, i.e. N-[2-chloro-4-fluoro-5-[4-(3-fluoropropyl)-4,5-dihydro-5-oxo-1H-tetrazol-1-yl]phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoxaprop, fenoxaprop-P, fenoxaprop-ethyl, fenoxaprop-P-ethyl, fenoxasulfone, fenquinotrione, fentrazamide, flamprop, flamprop-M-isopropyl, flamprop-M-methyl, flazasulfuron, florasulam, fluazifop, fluazifop-P, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanate, flupyrsulfuron, flupyrsulfuron-methyl-sodium, fluridone, flurochloridone, fluroxypyr, fluroxypyr-meptyl, flurtamone, fluthiacet, fluthiacet-methyl, fomesafen, fomesafen-sodium, foramsulfuron, fosamine, glufosinate, glufosinate-ammonium, glufosinate-P-sodium, glufosinate-P-ammonium, glufosinate-P-sodium, glyphosate, glyphosate-ammonium, -isopropylammonium, -diammonium, -dimethylammonium, -potassium, -sodium and -trimesium, H-9201, i.e. O-2,4-dimethyl-6-nitrophenyl O-ethyl isopropylphosphoramidothioate, halauxifen, halauxifen-methyl, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-dimethoxyphosphorylethyl 2,4-dichlorophenoxyacetate, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazethapyr, imazethapyr-immonium, imazosulfuron, indanofan, indaziflam, iodosulfuron, iodosulfuron-methyl-sodium, ioxynil, ioxynil-octanoate, -potassium and -sodium, ipfencarbazone, isoproturon, isouron, isoxaben, isoxaflutole, karbutilate, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, ketospiradox, lactofen, lenacil, linuron, MCPA, MCPA-butotyl, -dimethylammonium, -2-ethylhexyl, -isopropylammonium, -potassium and -sodium, MCPB, MCPB-methyl, -ethyl and -sodium, mecoprop, mecoprop-sodium, and -butotyl, mecoprop-P, mecoprop-P-butotyl, -dimethylammonium, -2-ethylhexyl and -potassium, mefenacet, mefluidide, mesosulfuron, mesosulfuron-methyl, mesotrione, methabenzthiazuron, metam, metamifop, metamitron, metazachlor, metazosulfuron, methabenzthiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinate, monolinuron, monosulfuron, monosulfuron-ester, MT-5950, i.e. N-[3-chlor-4-(1-methylethyl)-phenyl]-2-methylpentanamid, NGGC-011, napropamide, NC-310, i.e. 4-(2,4-dichlorobenzoyl)-1-methyl-5-benzyloxypyrazole, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefon, oxyfluorfen, paraquat, paraquat dichloride, pebulate, pendimethalin, penoxsulam, pentachlorphenol, pentoxazone, pethoxamid, petroleum oils, phenmedipham, picloram, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, sethoxydim, siduron, simazine, simetryn, SL-261, sulcotrion, sulfentrazone, sulfometuron, sulfometuron-methyl, sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, 2,3,6-TBA, TCA (trichloroacetic acid), TCA-sodium, tebuthiuron, tefuryltrione, tembotrione, tepraloxydim, terbacil, terbucarb, terbumeton, terbuthylazin, terbutryn, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, trietazine, trifloxysulfuron, trifloxysulfuron-sodium, trifludimoxazin, trifluralin, triflusulfuron, triflusulfuron-methyl, tritosulfuron, urea sulfate, vernolate, XDE-848, ZJ-0862, i.e. 3,4-dichloro-N-{2-[(4,6-dimethoxypyrimidin-2-yl)oxy]benzyl} aniline, and also the following compounds:

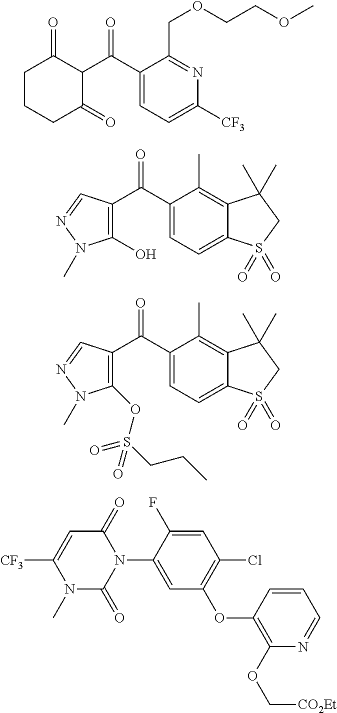

Examples of plant growth regulators as possible mixing partners are:

acibenzolar, acibenzolar-S-methyl, 5-aminolevulinic acid, ancymidol, 6-benzylaminopurine, brassinolide, catechol, chlormequat chloride, cloprop, cyclanilide, 3-(cycloprop-1-enyl)propionic acid, daminozide, dazomet, n-decanol, dikegulac, dikegulac-sodium, endothal, endothal-dipotassium, -disodium, and mono(N,N-dimethylalkylammonium), ethephon, flumetralin, flurenol, flurenol-butyl, flurprimidol, forchlorfenuron, gibberellic acid, inabenfide, indole-3-acetic acid (IAA), 4-indol-3-ylbutyric acid, isoprothiolane, probenazole, jasmonic acid, jasmonic acid methyl ester, maleic hydrazide, mepiquat chloride, 1-methylcyclopropene, 2-(1-naphthyl)acetamide, 1-naphthylacetic acid, 2-naphthyloxyacetic acid, nitrophenoxide mixture, 4-oxo-4[(2-phenylethyl)amino]butyric acid, paclobutrazole, N-phenylphthalamic acid, prohexadione, prohexadione-calcium, prohydrojasmone, salicylic acid, strigolactone, tecnazene, thidiazuron, triacontanol, trinexapac, trinexapac-ethyl, tsitodef, uniconazole, uniconazole-P.

Suitable combination partners for the compounds of the formula (I) according to the invention also include, for example, the following safeners:
S1) Compounds from the group of heterocyclic carboxylic acid derivatives:
S1^{a}) Compounds of the dichlorophenylpyrazoline-3-carboxylic acid type (S1^{a}), preferably compounds such as
   1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described in WO-A-91/07874;
S1^{b}) Derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds as described in EP-A-333 131 and EP-A-269 806;
S1^{c}) Derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds as described, for example, in EP-A-268554;
S1^{d}) Compounds of the triazolecarboxylic acid type (S1^{d}), preferably compounds such as fenchlorazole (ethyl ester), i.e. ethyl 1-(2,4-dichlorophenyl)-5-trichloromethyl-1H-1,2,4-triazole-3-carboxylate (S1-7), and related compounds, as described in EP-A-174 562 and EP-A-346 620;
S1^{e}) Compounds of the 5-benzyl- or 5-phenyl-2-isoxazoline-3-carboxylic acid or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds as described in WO-A-91/08202, or 5,5-diphenyl-2-isoxazolinecarboxylic acid (S1-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13), as described in patent application WO-A-95/07897.
S2) Compounds from the group of the 8-quinolinyloxy derivatives (S2):
S2^{a}) Compounds of the 8-quinolinoxyacetic acid type (S2^{a}), preferably 1-methylhexyl (5-chloro-8-quinolinoxy)acetate (“cloquintocet-mexyl”) (S2-1), 1,3-dimethylbut-1-yl (5-chloro-8-quinolinoxy)acetate (S2-2), 4-allyloxybutyl (5-chloro-8-quinolinoxy)acetate (S2-3), 1-allyloxyprop-2-yl (5-chloro-8-quinolinoxy)acetate (S2-4), ethyl (5-chloro-8-quinolinoxy)acetate (S2-5), methyl 5-chloro-8-quinolinoxyacetate (S2-6), allyl (5-chloro-8-quinolinoxy)acetate (S2-7), 2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86 750, EP-A-94 349 and EP-A-191 736 or EP-A-0 492 366, and also (5-chloro-8-quinolinoxy)acetic acid (S2-10), hydrates and salts thereof, for example the lithium, sodium, potassium, calcium, magnesium, aluminium, iron, ammonium, quaternary ammonium, sulfonium or phosphonium salts thereof, as described in WO-A-2002/34048;
S2^{b}) Compounds of the (5-chloro-8-quinolinoxy)malonic acid type (S2^{b}), preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.
S3) Active compounds of the dichloroacetamide type (S3), which are frequently used as pre-emergence safeners (soil-acting safeners), for example
   “dichlormid” (N,N-diallyl-2,2-dichloroacetamide) (S3-1),
   “R-29148” (3-dichloroacetyl-2,2,5-trimethyl-1,3-oxazolidine) from Stauffer (S3-2),
   “R-28725” (3-dichloroacetyl-2,2-dimethyl-1,3-oxazolidine) from Stauffer (S3-3),
   “benoxacor” (4-dichloroacetyl-3,4-dihydro-3-methyl-2H-1,4-benzoxazine) (S3-4),
   “PPG-1292” (N-allyl-N-[(1,3-dioxolan-2-yl)methyl]dichloroacetamide) from PPG Industries (S3-5),
   “DKA-24” (N-allyl-N-[(allylaminocarbonyl)methyl]dichloroacetamide) from Sagro-Chem (S3-6),
   “AD-67” or “MON 4660” (3-dichloroacetyl-1-oxa-3-azaspiro[4.5]decane) from Nitrokemia or Monsanto (S3-7),
   “TI-35” (1-dichloroacetylazepane) from TRI-Chemical RT (S3-8),
   “Diclonon” (Dicyclonon) or “BAS145138” or “LAB 145138” (S3-9)
   ((RS)-1-dichloroacetyl-3,3,8a-trimethylperhydropyrrolo[1,2-a]pyrimidin-6-one) from BASF,
   “furilazole” or “MON 13900” ((RS)-3-dichloroacetyl-5-(2-furyl)-2,2-dimethyloxazolidine) (S3-10), and the (R) isomer thereof (S3-11).
S4) Compounds from the class of the acylsulfonamides (S4):
S4^{a})N-Acylsulfonamides of the formula (S4^{a}) and salts thereof, as described in WO-A-97/45016,

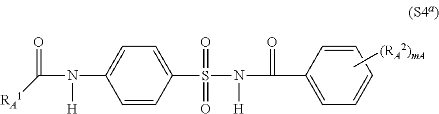

in which
R_{A}¹ is (C₁-C₆)alkyl, (C₃-C₆)cycloalkyl, cycloalkyl, where the 2 latter radicals are substituted by v_{A} substituents from the group of halogen, (C₁-C₄)alkoxy, (C₁-C₆)haloalkoxy and (C₁-C₄)alkylthio and, in the case of cyclic radicals, also by (C₁-C₄)alkyl and (C₁-C₄)haloalkyl;
R_{A}² is halogen, (C₁-C₄)alkyl, (C₁-C₄)alkoxy, CF₃;
m_{A} is 1 or 2;
v_{A} is 0, 1, 2 or 3;
S4^{b}) Compounds of the 4-(benzoylsulfamoyl)benzamide type of the formula (S4^{b}) and salts thereof, as described in WO-A-99/16744,

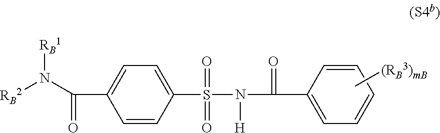

in which
R_{B}¹, R_{B}² are independently hydrogen, (C₁-C₆)alkyl, (C₃-C₆)cycloalkyl, (C₃-C₆)alkenyl, (C₃-C₆)alkynyl,
R_{B}³ is halogen, (C₁-C₄)alkyl, (C₁-C₄)haloalkyl or (C₁-C₄)alkoxy and
m_{B} is 1 or 2,
for example those in which
R_{B}¹=cyclopropyl, R_{B}²=hydrogen and (R_{B}³)=2-OMe (“cyprosulfamide”, S4-1),
R_{B}¹=cyclopropyl, R_{B}²=hydrogen and (R_{B}³)=5-Cl-2-OMe (S4-2),
R_{B}¹=ethyl, R_{B}²=hydrogen and (R_{B}³)=2-OMe (S4-3),
R_{B}¹=isopropyl, R_{B}²=hydrogen and (R_{B}³)=5-Cl-2-OMe (S4-4) and
R_{B}¹=isopropyl, R_{B}²=hydrogen and (R_{B}³)=2-OMe (S4-5).
S4^{c}) Compounds from the class of the benzoylsulfamoylphenylureas of the formula (S4c), as described in EP-A-365484,

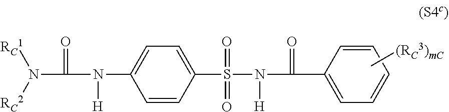

in which
R_{C}¹, R_{C}² are independently hydrogen, (C₁-C₈)alkyl, (C₃-C₈)cycloalkyl, (C₃-C₆)alkenyl, (C₃-C₆)alkynyl,
R_{C}³ is halogen, (C₁-C₄)alkyl, (C₁-C₄)alkoxy, CF₃ and
m^{C} is 1 or 2;
for example
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea;
S4^{d}) Compounds of the N-phenylsulfonylterephthalamide type of the formula (S4^{d}) and salts thereof, which are known, for example, from CN 101838227,

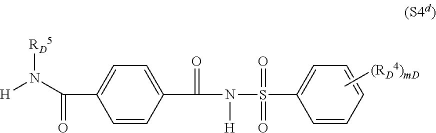

in which
R_{D}⁴ is halogen, (C₁-C₄)-alkyl, (C₁-C₄)-alkoxy, CF₃;
m_{D} is 1 or 2;
R_{D}⁵ is hydrogen, (C₁-C₆)-alkyl, (C₃-C₆)-cycloalkyl, (C₂-C₆)-alkenyl, (C₂-C₆)-alkynyl or (C₅-C₆)-cycloalkenyl.
S5) Active compounds from the class of the hydroxyaromatics and the aromatic-aliphatic carboxylic acid derivatives (S5), for example
   ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicyclic acid, 2-hydroxycinnamic acid, 2,4-dichloro-cinnamic acid, as described in WO-A-2004/084631, WO-A-2005/015994, WO-A-2005/016001.
S6) Active compounds from the class of the 1,2-dihydroquinoxalin-2-ones (S6), for example 1-methyl-3-(2-thienyl)-1,2-dihydroquinoxalin-2-one, 1-methyl-3-(2-thienyl)-1,2-dihydro-quinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.
S7) Compounds from the class of the diphenylmethoxyacetic acid derivatives (S7), for example methyl diphenylmethoxyacetate (CAS Reg. No. 41858-19-9) (S7-1), ethyl diphenylmethoxyacetate or diphenylmethoxyacetic acid, as described in WO-A-98/38856.
S8) Compounds of the formula (S8), as described in WO-A-98/27049,

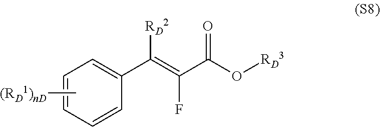

where the symbols and indices are each defined as follows:
R_{D}¹ is halogen, (C₁-C₄)-alkyl, (C₁-C₄)-haloalkyl, (C₁-C₄)-alkoxy, (C₁-C₄)-haloalkoxy,
R_{D}² is hydrogen or (C₁-C₄)-alkyl,
R_{D}³ is hydrogen, (C₁-C₈)-alkyl, (C₂-C₄)-alkenyl, (C₂-C₄)-alkynyl or aryl, where each of the aforementioned carbon-containing radicals is unsubstituted or substituted by one or more, preferably up to three identical or different radicals from the group consisting of halogen and alkoxy; or salts thereof,
n_{D} is an integer from 0 to 2.
S9) Active compounds from the class of the 3-(5-tetrazolylcarbonyl)-2-quinolones (S9), for example 1,2-dihydro-4-hydroxy-1-ethyl-3-(5-tetrazolylcarbonyl)-2-quinolone (CAS Reg. No. 219479-18-2), 1,2-dihydro-4-hydroxy-1-methyl-3-(5-tetrazolylcarbonyl)-2-quinolone (CAS Reg. No. 95855-00-8), as described in WO-A-199/000020;
S10) Compounds of the formula (S10^{a}) or (S10^{b})
   as described in WO-A-2007/023719 and WO-A-2007/023764

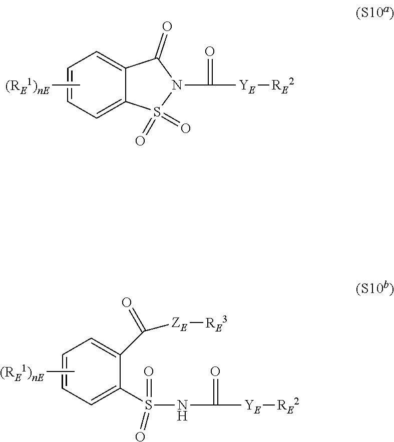

in which
R_{E}¹ is halogen, (C₁-C₄)-alkyl, methoxy, nitro, cyano, CF₃, OCF₃
Y_{E}, Z_{E} are independently O or S,
n_{E} is an integer from 0 to 4,
R_{E}² is (C₁-C₁₆)-alkyl, (C₂-C₆)-alkenyl, (C₃-C₆)-cycloalkyl, aryl; benzyl, halobenzyl,
R_{E}³ is hydrogen or (C₁-C₆)-alkyl.
S11) Active compounds of the oxyimino compound type (S11), which are known as seed-dressing agents, for example
   “oxabetrinil” ((Z)-1,3-dioxolan-2-ylmethoxyimino(phenyl)acetonitrile) (S11-1), which is known as a seed-dressing safener for millet/sorghum against metolachlor damage,
   “fluxofenim” (1-(4-chlorophenyl)-2,2,2-trifluoro-1-ethanone 0-(1,3-dioxolan-2-ylmethyl)oxime) (S11-2), which is known as a seed-dressing safener for millet/sorghum against metolachlor damage, and
   “cyometrinil” or “CGA-43089” ((Z)-cyanomethoxyimino(phenyl)acetonitrile) (S11-3), which is known as a seed-dressing safener for millet/sorghum against metolachlor damage.
S12) Active compounds from the class of the isothiochromanones (S12), for example methyl [(3-oxo-1H-2-benzothiopyran-4(3H)-ylidene)methoxy]acetate (CAS Reg. No. 205121-04-6) (S12-1) and related compounds from WO-A-1998/13361.
S13) One or more compounds from group (S13):
   “naphthalic anhydride” (1,8-naphthalenedicarboxylic anhydride) (S13-1), which is known as a seed-dressing safener for corn against thiocarbamate herbicide damage,
   “fenclorim” (4,6-dichloro-2-phenylpyrimidine) (S13-2), which is known as a safener for pretilachlor in sown rice,
   “flurazole” (benzyl 2-chloro-4-trifluoromethyl-1,3-thiazole-5-carboxylate) (S13-3), which is known as a seed-dressing safener for millet/sorghum against alachlor and metolachlor damage,
   “CL 304415” (CAS Reg. No. 31541-57-8)
   (4-carboxy-3,4-dihydro-2H-1-benzopyran-4-acetic acid) (S13-4) from American Cyanamid, which is known as a safener for corn against damage by imidazolinones,
   “MG 191” (CAS Reg. No. 96420-72-3) (2-dichloromethyl-2-methyl-1,3-dioxolane) (S13-5) from Nitrokemia, which is known as a safener for corn,
   “MG 838” (CAS Reg. No. 133993-74-5) (2-propenyl 1-oxa-4-azaspiro[4.5]decane-4-carbodithioate) (S13-6) from Nitrokemia
   “disulfoton” (O,O-diethyl S-2-ethylthioethyl phosphorodithioate) (S13-7),
   “dietholate” (O,O-diethyl O-phenyl phosphorothioate) (S13-8),
   “mephenate” (4-chlorophenyl methylcarbamate) (S13-9).
S14) Active compounds which, in addition to herbicidal action against weeds, also have safener action on crop plants such as rice, for example
   “dimepiperate” or “MY-93” (S-1-methyl 1-phenylethylpiperidine-1-carbothioate), which is known as a safener for rice against damage by the herbicide molinate,
   “daimuron” or “SK 23” (1-(1-methyl-1-phenylethyl)-3-p-tolylurea), which is known as safener for rice against imazosulfuron herbicide damage,
   “cumyluron”=“JC-940” (3-(2-chlorophenylmethyl)-1-(1-methyl-1-phenylethyl)urea, see JP-A-60087254), which is known as safener for rice against damage by some herbicides,
   “methoxyphenone” or “NK 049” (3,3′-dimethyl-4-methoxybenzophenone), which is known as a safener for rice against damage by some herbicides,
   “CSB” (1-bromo-4-(chloromethylsulfonyl)benzene) from Kumiai, (CAS Reg. No. 54091-06-4), which is known as a safener against damage by some herbicides in rice.
S15) Compounds of the formula (S15) or tautomers thereof

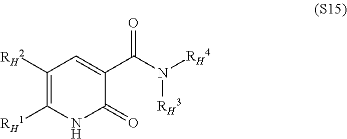

as described in WO-A-2008/131861 and WO-A-2008/131860
in which
R_{H}¹ is a (C₁-C₆)-haloalkyl radical and
R_{H}² is hydrogen or halogen and
R_{H}³, R_{H}⁴ are each independently hydrogen, (C₁-C₁₆)-alkyl, (C₂-C₁₆)-alkenyl or (C₂-C₁₆)-alkynyl,
   where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
   or (C₃-C₆)-cycloalkyl, (C₄-C₆)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring,
   where each of the 4 latter radicals is unsubstituted or substituted by one or more radicals from the group of halogen, hydroxyl, cyano, (C₁-C₄)alkyl, (C₁-C₄)haloalkyl, (C₁-C₄)alkoxy, (C₁-C₄)haloalkoxy, (C₁-C₄)alkylthio, (C₁-C₄)alkylamino, di[(C₁-C₄)alkyl]amino, [(C₁-C₄)alkoxy]carbonyl, [(C₁-C₄)haloalkoxy]carbonyl, (C₃-C₆)cycloalkyl which is unsubstituted or substituted, phenyl which is unsubstituted or substituted, and heterocyclyl which is unsubstituted or substituted,
   or
R_{H}³ is (C₁-C₄)-alkoxy, (C₂-C₄)-alkenyloxy, (C₂-C₆)-alkynyloxy or (C₂-C₄)-haloalkoxy and
R_{H}⁴ is hydrogen or (C₁-C₄)-alkyl or
R_{H}³ and R_{H}⁴ together with the directly bonded nitrogen atom are a four- to eight-membered heterocyclic ring which, as well as the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio.
S16) Active compounds which are used primarily as herbicides but also have safener action on crop plants, for example
   (2,4-dichlorophenoxy)acetic acid (2,4-D),
   (4-chlorophenoxy)acetic acid,
   (R,S)-2-(4-chloro-o-tolyloxy)propionic acid (mecoprop),
   4-(2,4-dichlorophenoxy)butyric acid (2,4-DB),
   (4-chloro-o-tolyloxy)acetic acid (MCPA),
   4-(4-chloro-o-tolyloxy)butyric acid,
   4-(4-chlorophenoxy)butyric acid,
   3,6-dichloro-2-methoxybenzoic acid (dicamba),
   1-(ethoxycarbonyl)ethyl 3,6-dichloro-2-methoxybenzoate (lactidichlor-ethyl).

Preferred safeners in combination with the compounds of the formula (I) according to the invention and/or salts thereof, in particular with the compounds of the formulae (I.1) to (I.77) and/or salts thereof, are: cloquintocet-mexyl, cyprosulfamide, fenchlorazole-ethyl, isoxadifen-ethyl, mefenpyr-diethyl, fenclorim, cumyluron, S4-1 and S4-5, and particularly preferred safeners are: cloquintocet-mexyl, cyprosulfamide, isoxadifen-ethyl and mefenpyr-diethyl.

### SYNTHESIS EXAMPLES

Selected detailed synthesis examples for the compounds of the general formula (I) according to the invention are given below. The example numbers mentioned correspond to the numbering scheme in Tables I.1 to I.77. The ¹H NMR, ¹³C-NMR and ¹⁹F-NMR spectroscopy data reported for the chemical examples described in the sections which follow (400 MHz for ¹H-NMR and 150 MHz for ¹³C-NMR and 375 MHz for ¹⁹F-NMR, solvent CDCl₃, CD₃OD or d₆-DMSO, internal standard: tetramethylsilane δ =0.00 ppm), were obtained on a Bruker instrument, and the signals listed have the meanings given below: br=broad; s=singlet, d=doublet, t=triplet, dd=doublet of doublets, ddd=doublet of a doublet of doublets, m=multiplet, q=quartet, quint=quintet, sext=sextet, sept=septet, dq=doublet of quartets, dt=doublet of triplets. In the case of diastereomer mixtures, either the significant signals for each of the two diastereomers are reported or the characteristic signal of the main diastereomer is reported. The abbreviations used for chemical groups have, for example, the following meanings: Me=CH₃, Et=CH₂CH₃, t-Hex=C(CH₃)₂CH(CH₃)₂, t-Bu=C(CH₃)₃, n-Bu=unbranched butyl, n-Pr=unbranched propyl, i-Pr=isopropyl, c-Pr=cyclopropyl, c-Hex=cyclohexyl, Ac=acetyl.

### Example No. I.73-732: 3,4-Dimethyl-1-[2-(2-pyridyl)-3,4-dihydropyrazol-5-yl]pyrrole-2,5-dione

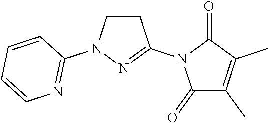 [SMILES: CC1=C(C)C(=O)N(C2=NN(c3ccccn3)CC2)C1=O]

2-Hydrazinopyridine (2.0 g, 18.3 mmol) and acrylonitrile (972 mg, 18.3 mmol) were dissolved in ethanol (50 ml), and sodium ethoxide (21% in ethanol, 27.4 ml, 73.3 mmol) was added. The reaction solution was heated under reflux for 24 h and the solvent was removed under reduced pressure. Water was added to the residue and the aqueous phase was extracted with CH₂Cl₂. The combined organic phases were dried over magnesium sulfate and the solvent was removed under reduced pressure. A mixture of CH₂Cl₂/n-heptane was added to the residue and the precipitated solid was filtered off. This gave 2-(pyridin-2-yl)-3,4-dihydropyrazole-5-amine (1.45 g, 44% of theory) as a colourless solid. ¹H-NMR (400 MHz, CDCl₃ 6, ppm) 8.13 (m, 1H), 7.45-7.41 (m, 1H), 7.01-6.97 (m, 1H), 6.57-6.54 (d, 1H), 4.09 (br s, NH₂), 3.95 (m, 2H), 2.91 (m, 2H). 2-(Pyridin-2-yl)-3,4-dihydropyrazole-5-amine (1.5 g, 9.24 mmol) and 2,3-dimethylmaleic anhydride (1.28 g, 10.1 mmol) were dissolved in MeCN (10 ml), and pyridine (0.3 ml, 3.69 mmol) was added. The reaction mixture was heated under reflux for 22 h. The mixture was cooled to room temperature, water was added and the aqueous phase was extracted with EtOAc. The combined organic phases were washed successively with water and saturated NaCl solution and dried over sodium sulfate, and the solvent was removed under reduced pressure. Purification by column chromatography (gradient ethyl acetate/n-heptane) gave 3,4-dimethyl-1-[2-(2-pyridyl)-3,4-dihydropyrazol-5-yl]pyrrole-2,5-dione I.73-732 (1.97 g, 78% of theory) as a colourless solid.

### Example No. I.73-1126: 3,4-Dimethyl-1-(2-methyl-3-phenyl-3,4-dihydropyrazol-5-yl)pyrrole-2,5-dione

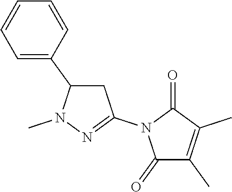 [SMILES: CC1=C(C)C(=O)N(C2=NN(C)C(c3ccccc3)C2)C1=O]

Methylhydrazine (0.82 ml, 15.49 mmol) and trans-cinnamonitrile (1.95 ml, 15.49 mmol) were dissolved in ethanol (50 ml), and sodium ethoxide (21% in ethanol, 22.8 ml, 61.94 mmol) was added. The reaction solution was heated under reflux for 24 h and the solvent was removed under reduced pressure. Water was added to the residue and the aqueous phase was extracted with CH₂Cl₂. The combined organic phases were dried over magnesium sulfate and the solvent was removed under reduced pressure. A mixture of CH₂Cl₂/n-heptane was added to the residue and the precipitated solid was filtered off. This gave 3-phenyl-2-methyl-3,4-dihydropyrazole-5-amine (1.88 g, 66% of theory) as a colourless solid. ¹H-NMR (400 MHz, CDCl₃ δ, ppm) 7.46-7.44 (m, 2H), 7.37-7.28 (m, 3H), 3.87 (br. s, 2H, NH₂), 3.81-3.75 (m, 2H), 2.88-2.83 (m, 2H), 2.56 (s, 3H). 3-Phenyl-2-methyl-3,4-dihydropyrazole-5-amine (0.40 g, 2.28 mmol) and 2,3-dimethylmaleic anhydride (0.32 g, 2.51 mmol) were dissolved in concentrated acetic acid (5 ml) and heated under reflux for 7 h. Water was added to the mixture and the aqueous phase was extracted with EtOAc. The combined organic phases were washed with water and dried over sodium sulfate, and the solvent was removed under reduced pressure. Purification by column chromatography (gradient ethyl acetate/n-heptane) gave 3,4-dimethyl-1-(2-methyl-3-phenyl-3,4-dihydropyrazol-5-yl)pyrrole-2,5-dione I.73-1126 (58 mg, 9% of theory) as a colourless solid.

### Example No. I.73-1228: 3,4-Dimethyl-1-[3-phenyl-2-(2-pyridyl)-3,4-dihydropyrazol-5-yl]pyrrole-2,5-dione

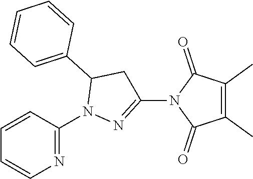 [SMILES: CC1=C(C)C(=O)N(C2=NN(c3ccccn3)C(c3ccccc3)C2)C1=O]

2-Hydrazinopyridine (2.0 g, 18.3 mmol) and trans-cinnamonitrile (2.4 g, 18.3 mmol) were dissolved in ethanol (30 ml), and sodium ethoxide (21% in ethanol, 27.4 ml, 73.3 mmol) was added. The reaction solution was heated under reflux for 24 h and the solvent was removed under reduced pressure. Water was added to the residue and the aqueous phase was extracted with CH₂Cl₂. The combined organic phases were dried over magnesium sulfate and the solvent was removed under reduced pressure. A mixture of CH₂Cl₂/n-heptane was added to the residue and the precipitated solid was filtered off. This gave 3-phenyl-2-(2-pyridyl)-3,4-dihydropyrazole-5-amine (1.44 g, 31% of theory) as a colourless solid.

¹H-NMR (400 MHz, CDCl₃ 8, ppm) 7.79-7.81 (m, 1H), 7.37-7.41 (m, 1H), 7.18-7.29 (m, 5H), 6.92 (d, 1H), 6.37-6.40 (m, 1H), 5.92 (br s, NH₂), 4.47 (dd, 1H), 3.46 (dd, 1H), 2.48 (dd, 1H). 3-Phenyl-2-(2-pyridyl)-3,4-dihydropyrazole-5-amine (1.50 g, 6.29 mmol) and 2,3-dimethylmaleic anhydride (0.87 g, 6.29 mmol) were dissolved in concentrated acetic acid (20 ml) and heated under reflux for 20 h. Water was added to the mixture and the aqueous phase was extracted with EtOAc. The combined organic phases were washed with water, and the solvent was removed under reduced pressure. Purification by column chromatography (gradient ethyl acetate/n-heptane) gave 3,4-dimethyl-1-[3-phenyl-2-(2-pyridyl)-3,4-dihydropyrazol-5-yl]pyrrole-2,5-dione I.73-1228 (0.51 g, 21% of theory) as a colourless solid.

### Example No. I.2-1228: 2-Hydroxy-3,4-dimethyl-1-[3-phenyl-2-(2-pyridyl)-3,4-dihydropyrazol-5-yl]-2H-pyrrol-5-one

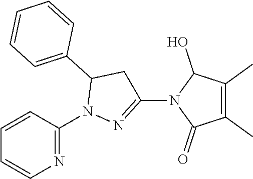 [SMILES: CC1=C(C)C(O)N(C2=NN(c3ccccn3)C(c3ccccc3)C2)C1=O]

3,4-Dimethyl-1-[3-phenyl-2-(2-pyridyl)-3,4-dihydropyrazol-5-yl]pyrrole-2,5-dione (0.86 g, 2.48 mmol) was dissolved in THF (tetrahydrofuran)/MeOH (1:1, 10 ml), and sodium borohydride (0.094 g, 2.48 mmol) was added at −30° C. The reaction mixture was stirred at −30° C. for 2 h and then warmed to room temperature. Using dilute 5% strength acetic acid, the mixture was adjusted to a pH of 3-4 and the aqueous phase was extracted with EtOAc. The combined organic phases were washed with water and dried over magnesium sulfate, and the solvent was removed under reduced pressure. Purification by column chromatography (gradient ethyl acetate/heptane) gave 2-hydroxy-3,4-dimethyl-1-[3-phenyl-2-(2-pyridyl)-3,4-dihydropyrazol-5-yl]-2H-pyrrol-5-one I.2-1228 (0.85 g, 93% of theory) as a colourless solid.

### Example No. I.23-1228: 3,4-Dimethyl-5-oxo-1-[3-phenyl-2-(2-pyridyl)-3,4-dihydropyrazol-5-yl]-2H-pyrrol-2-yl propanoate

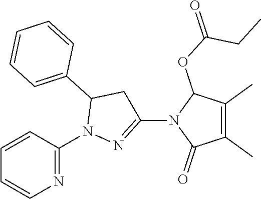 [SMILES: CCC(=O)OC1C(C)=C(C)C(=O)N1C1=NN(c2ccccn2)C(c2ccccc2)C1]

2-Hydroxy-3,4-dimethyl-1-[3-phenyl-2-(2-pyridyl)-3,4-dihydropyrazol-5-yl]-2H-pyrrol-5-one (0.28 g, 0.81 mmol) was dissolved in THF (10 ml), and NaH (0.036 g, 0.89 mmol) was added. The reaction mixture was stirred at room temperature for 30 min, and propionyl chloride (0.078 ml, 0.89 mmol) was then added dropwise. The mixture was stirred at room temperature for 4 h. Water was added to the reaction mixture to terminate the reaction. The aqueous phase was extracted with dichloromethane, the combined organic phases were dried over magnesium sulfate and the solvent was removed under reduced pressure. Purification by column chromatography (gradient ethyl acetate/heptane) gave 3,4-dimethyl-5-oxo-1-[3-phenyl-2-(2-pyridyl)-3,4-dihydropyrazol-5-yl]-2H-pyrrol-2-yl propanoate I.2-1228 (0.13 g, 37% of theory) as an oil.

### Example No. I.5-1228: 4-Hydroxy-1-methyl-3-[3-phenyl-2-(2-pyridyl)-3,4-dihydropyrazol-5-yl]imidazolidin-2-one

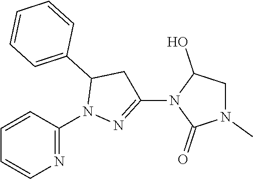 [SMILES: CN1CC(O)N(C2=NN(c3ccccn3)C(c3ccccc3)C2)C1=O]

3-Phenyl-2-(2-pyridyl)-3,4-dihydropyrazole-5-amine (0.50 g, 2.09 mmol) was dissolved in THF (15 ml), and pyridine (0.34 ml, 4.19 mmol) was added. The reaction mixture was cooled to 0° C., and a solution of phenyl chloroformate (0.31 ml, 0.32 mmol) in THF (5 ml) was slowly added dropwise over 15 min. The reaction mixture was stirred at 0° C. for 15 min, warmed to room temperature and stirred for a further 2 h. The precipitated solid was filtered off, washed with MeCN and then dried over magnesium sulfate, and the crude product (4-nitrophenyl)N-[3-phenyl-2-(2-pyridyl)-3,4-dihydropyrazol-5-yl]carbamate (0.13 g, 17% of theory) was obtained as a solid. ¹H-NMR (400 MHz, CDCl₃ 8, ppm) 8.06-8.07 (s, 1H), 7.12-7.46 (m, 11H), 6.91-7.00 (1H), 6.59-6.62 (1H), 5.61-5.65 (1H), 3.95-4.03 (1H), 3.38-3.44 (1H).

4-Nitrophenyl N-[3-phenyl-2-(2-pyridyl)-3,4-dihydropyrazol-5-yl]carbamate (0.13 g, 0.36 mmol) was dissolved in 1,4-dioxane (10 ml), and 2,2-dimethoxyethylmethylamine (0.05 ml, 0.39 mmol) was added. The reaction mixture was stirred at room temperature for 48 h. The mixture was concentrated under reduced pressure and the residue was dissolved in EtOAc. The organic phase was washed with water and dried over magnesium sulfate, and the solvent was removed under reduced pressure. Purification by column chromatography (gradient ethyl acetate/heptane) gave 1-(2,2-dimethoxyethyl)-1-methyl-3-[3-phenyl-2-(2-pyridyl)-3,4-dihydropyrazol-5-yl]urea (0.11 g, 70% of theory) as an oil. ¹H-NMR (400 MHz, CDCl₃ 8, ppm) 8.05-8.06 (1H), 7.19-7.42 (5H), 6.82-6.94 (2H), 6.53-6.56 (1H), 5.53-5.57 (1H), 4.44-4.46 (1H), 4.00-4.05 (1H), 3.48 (3H), 3.46 (3H), 3.32-3.44 (3H), 3.00 (3H). 1-(2,2-Dimethoxyethyl)-1-methyl-3-[3-phenyl-2-(2-pyridyl)-3,4-dihydropyrazol-5-yl]urea (0.11 g, 0.28 mmol) was dissolved in concentrated acetic acid (1.0 ml) and water (1.0 ml) and stirred under microwave conditions at 70° C. for 2.5 h. The mixture was cooled to room temperature and the solvent was removed under reduced pressure. Purification by column chromatography (gradient ethyl acetate/heptane) gave 4-hydroxy-1-methyl-3-[3-phenyl-2-(2-pyridyl)-3,4-dihydropyrazol-5-yl]imidazolidin-2-one 1.5-1228 (0.03 g, 28% of theory) as a solid.

Substituted pyrazolinylpyrrolones and pyrazolinylhydantoins of the general formula (I) can be prepared analogously to the preparation examples above, taking into account the general statements above.

The table below lists the NMR data of selected compounds of the general formula (I) according to the invention, with the numbering scheme of Tables I.1 to I.77 also applying here.

| No. | Structure | Mass | NMR |
|---|---|---|---|
| I.73-321 | 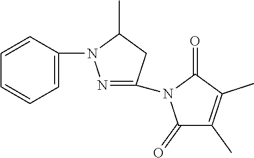 [SMILES: CC1=C(C)C(=O)N(C2=NN(c3ccccc3)C(C)C2)C1=O] | MS: 283.13 | ¹H-NMR (400 MHz, CDCl₃ δ, ppm) 7.23-7.27 (2H), 7.07-7.09 (2H), 6.84 (1H), 4.42-4.47 (1H), 3.45-3.52 (1H), 2.92-2.98 (1H), 2.03 (6H), 1.34 (3H) |
| I.73-621 | 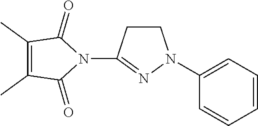 [SMILES: CC1=C(C)C(=O)N(C2=NN(c3ccccc3)CC2)C1=O] | MS: — | ¹H-NMR (400 MHz, CDCl₃ δ, ppm) 7.29-7.25 (2H), 7.05-7.03 (2H), 6.85 (1H), 3.87-3.92 (2H), 3.28-3.33 (2H), 2.03 (6H) |
| I.73-1363 | 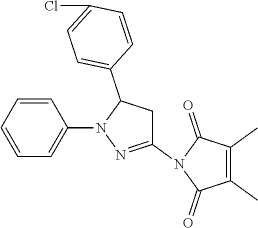 [SMILES: CC1=C(C)C(=O)N(C2=NN(c3ccccc3)C(c3ccc(Cl)cc3)C2)C1=O] | MS: 379.11 | ¹H-NMR (400 MHz, CDCl₃ δ, ppm) 7.26-7.32 (4H), 7.13-7.17 (2H), 6.92-6.95 (2H), 6.80 (1H), 5.14-5.19 (1H), 3.84-3.92 (1H), 3.09-3.15 (1H), 2.03 (6H) |
| I.73-1413 | 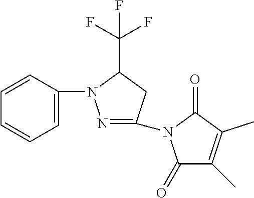 [SMILES: CC1=C(C)C(=O)N(C2=NN(c3ccccc3)C(C(F)(F)F)C2)C1=O] | MS: 337.10 | ¹H-NMR (400 MHz, CDCl₃ δ, ppm) 7.27-7.31 (2H), 7.12-7.14 (2H), 6.91 (1H), 5.26-5.31 (1H), 3.68-3.75 (1H), 3.45-3.50 (1H), 1.97 (6H) |
| I.73-1139 | 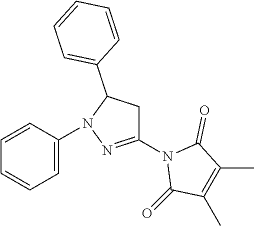 [SMILES: CC1=C(C)C(=O)N(C2=NN(c3ccccc3)C(c3ccccc3)C2)C1=O] | MS: 345.15 | ¹H-NMR (400 MHz, CDCl₃ δ, ppm) 7.29-7.38 (2H), 7.12-7.16 (2H), 6.95-6.99 (2H), 6.77-6.80 (1H), 5.16-5.21 (1H), 3.84-3.92 (1H), 3.13-3.20 (1H), 2.07 (3H), 2.03 (3H) |
| I.73-1486 | 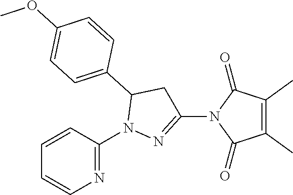 | MS: 376.15 | ¹H-NMR (400 MHz, CDCl₃ δ, ppm) 8.01-8.03 (1H), 7.46-7.49 (1H), 7.44-7.30 (3H), 6.81-6.83 (2H), 6.60-6.63 (1H), 5.68-5.73 (1H), 3.81-3.88 (1H), 3.76 (3H), 3.17-3.23 (1H), 2.03 (6H) |
| I.73-732 | 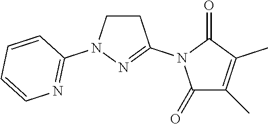 [SMILES: CC1=C(C)C(=O)N(C2=NN(c3ccccn3)CC2)C1=O] | MS: 270.11 | ¹H-NMR (400 MHz, CDCl₃ δ, ppm) 8.17-8.19 (1H), 7.50-7.53 (1H), 7.27-7.30 (1H), 6.68-6.71 (1H), 4.12-4.17 (2H), 3.30-3.35 (2H), 2.04 (6H) |
| I.73-1126 | 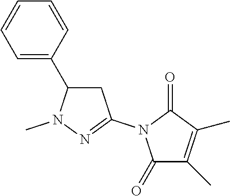 [SMILES: CC1=C(C)C(=O)N(C2=NN(C)C(c3ccccc3)C2)C1=O] | MS: 283.13 | ¹H-NMR (400 MHz, CDCl₃ δ, ppm) 7.246-7.48 (2H), 7.26-7.37 (3H), 4.08-4.14 (1H), 3.48-3.54 (1H), 2.95-2.99 (1H), 2.76 (3H), 2.01 (6H) |
| I.73-1228 | 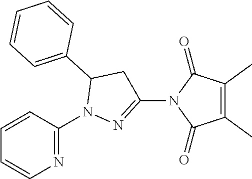 [SMILES: CC1=C(C)C(=O)N(C2=NN(c3ccccn3)C(c3ccccc3)C2)C1=O] | MS: 346.14 | ¹H-NMR (400 MHz, CDCl₃ δ, ppm) 8.00-8.01 (1H), 7.46-7.49 (1H), 7.21-7.34 (1H), 6.60-6.63 (1H), 5.73-5.76 (1H), 3.86-3.91 (1H), 3.21-3.25 (1H), 2.04 (3H), 2.09 (3H) |
| I.73-132 | 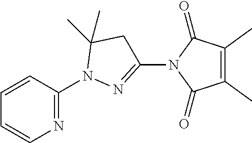 [SMILES: CC1=C(C)C(=O)N(C2=NN(c3ccccn3)C(C)(C)C2)C1=O] | MS: 298.14 | ¹H-NMR (400 MHz, CDCl₃ δ, ppm) 8.12-8.13 (1H), 7.43-7.47 (1H), 7.23-7.26 (1H), 6.64-6.67 (1H), 3.19 (2H), 2.03 (6H), 1.72 (6H) |
| I.73-432 | 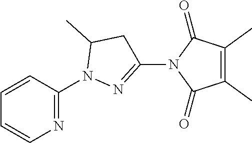 [SMILES: CC1=C(C)C(=O)N(C2=NN(c3ccccn3)C(C)C2)C1=O] | MS: 284.13 | ¹H-NMR (400 MHz, CDCl₃ δ, ppm) 8.15-8.16 (1H), 7.48-7.50 (1H), 7.23-7.25 (1H), 6.66-6.68 (1H), 4.78-4.84 (1H), 3.47-3.54 (1H), 2.94-3.00 (1H), 2.04 (6H), 1.46-1.47 (3H) |
| I.73-1351 | 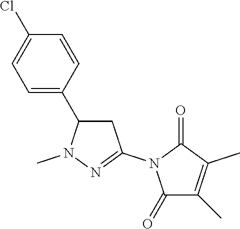 [SMILES: CC1=C(C)C(=O)N(C2=NN(C)C(c3ccc(Cl)cc3)C2)C1=O] | MS: 317.09 | ¹H-NMR (400 MHz, CDCl₃ δ, ppm) 7.40-7.42 (2H), 7.33-7.35 (2H), 4.05-4.11 (1H), 3.49-3.55 (1H), 2.89-2.93 (1H), 2.74 (3H), 2.01 (6H) |
| I.73-217 | 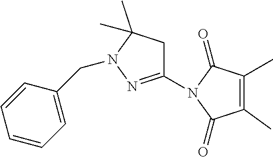 [SMILES: CC1=C(C)C(=O)N(C2=NN(Cc3ccccc3)C(C)(C)C2)C1=O] | MS: 311.16 | ¹H-NMR (400 MHz, CDCl₃ δ, ppm) 7.22-7.45 (5H), 4.14 (2H), 2.87 (2H), 1.97 (6H), 1.27 (6H) |
| I.72-21 | 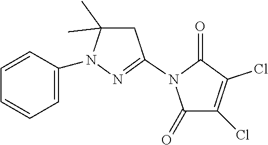 [SMILES: CC1(C)CC(N2C(=O)C(Cl)=C(Cl)C2=O)=NN1c1ccccc1] | MS: 337.04 | ¹H-NMR (400 MHz, CDCl₃ δ, ppm) 7.00-7.52 (5H), 3.11 (2H), 1.43 (6H) |
| I.73-321 | 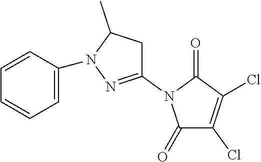 [SMILES: CC1CC(N2C(=O)C(Cl)=C(Cl)C2=O)=NN1c1ccccc1] | MS: 323.02 | ¹H-NMR (400 MHz, CDCl₃ δ, ppm) 7.25-7.31 (2), 7.00-7.08 (2), 6.87-6.90 (1H), 4.51-4.57 (1H), 3.45-3.52 (1H), 2.89-2.94 (1H), 1.36 (6H) |
| I.73-21 | 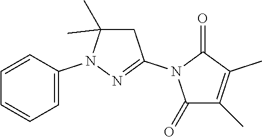 [SMILES: CC1=C(C)C(=O)N(C2=NN(c3ccccc3)C(C)(C)C2)C1=O] | MS: 297.15 | ¹H-NMR (400 MHz, CDCl₃ δ, ppm) 7.24-7.26 (4), 7.00 (1H), 3.13 (2H), 2.03 (6H), 1.41 (6H) |
| I.73-1386 | 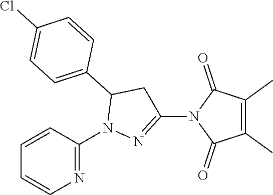 [SMILES: CC1=C(C)C(=O)N(C2=NN(c3ccccn3)C(c3ccc(Cl)cc3)C2)C1=O] | MS: 381.1 | ¹H-NMR (400 MHz, CDCl₃ δ, ppm) 7.98-8.00 (1H), 7.49-7.52 (1H), 7.25-7.33 (5H), 6.62-6.65 (1H), 5.68-5.73 (1H), 3.83-3.90 (1H), 3.16-3.22 (1H), 2.03 (6H) |
| I.2-1413 | 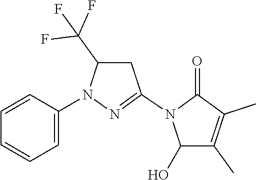 [SMILES: CC1=C(C)C(O)N(C2=NN(c3ccccc3)C(C(F)(F)F)C2)C1=O] | MS: 339.12 | ¹H-NMR (400 MHz, CDCl₃ δ, ppm) 7.26-7.32 (2H), 7.06-7.12 (2H), 6.94-7.00 (1H), 5.85 (1H), 4.50-4.54 (1H), 4.35 (1H), 3.75- 3.79 (2H), 2.09 (3H), 1.85 (3H) |
| I.2-321 | 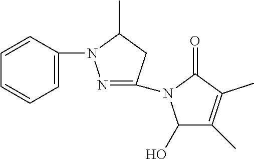 [SMILES: CC1=C(C)C(O)N(C2=NN(c3ccccc3)C(C)C2)C1=O] | MS: 285.15 | ¹H-NMR (400 MHz, CDCl₃ δ, ppm) 7.24-7.31 (2H), 6.98-7.00 (2H), 6.81-6.84 (1H), 5.79-5.82 (1H), 4.56-5.65 (1H), 4.25-4.35 (1H), 3.65-3.72 (1H), 3.24-3.31 (1H), 2.06-2.10 (3H), 1.84 (3H), 1.26-1.37 (3H) |
| I.2-621 | 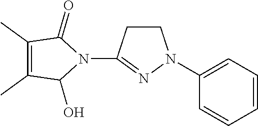 [SMILES: CC1=C(C)C(O)N(C2=NN(c3ccccc3)CC2)C1=O] | MS: 271.13 | ¹H-NMR (400 MHz, CDCl₃ δ, ppm) 7.24-7.29 (2H), 6.95-6.97 (2H), 6.82-6.86 (1H), 5.81 (1H), 4.59 (1H), 3.74-3.84 (2H), 3.52- 3.57 (2H), 2.06 (3H), 1.84 (3H) |
| I.2-1386 | 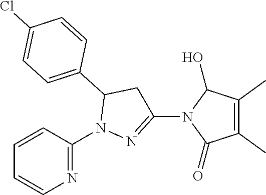 [SMILES: CC1=C(C)C(O)N(C2=NN(c3ccccn3)C(c3ccc(Cl)cc3)C2)C1=O] | MS: 382.12 | ¹H-NMR (400 MHz, DMSO δ, ppm) 7.89-7.91 (1H), 7.53-7.55 (1H), 7.34-7.39 (2H), 7.23-7.26 (3H), 6.71-6.73 (1H), 6.58-6.63 (1H), 5.77-5.82 (1H), 5.58-5.63 (1H), 3.82-4.11 (1H), 3.24-3.38 (1H), 1.97 (3H), 1.36 (3H) |
| I.2-732 | 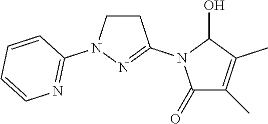 [SMILES: CC1=C(C)C(O)N(C2=NN(c3ccccn3)CC2)C1=O] | MS: 272.13 | ¹H-NMR (400 MHz, CDCl₃ δ, ppm) 8.33-8.34 (1H), 7.52-7.57 (1H), 6.79-6.78 (1H), 6.68-6.71 (1H), 5.82 (1H), 3.98-4.04 (1H), 3.57-3.64 (1H), 2.06 (3H), 1.84 (3H) |
| I.2-1486 | 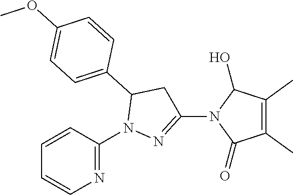 | MS: 378.17 | ¹H-NMR (400 MHz, DMSO δ, ppm) 8.07 (1H), 7.21-7.27 (1H), 6.93-6.95 (1H), 5.93 (1H), 5.75 (1H), 4.19 (1H), 3.45-3.50 (1H), 2.45-2.52 (1H), 2.02 (3H), 1.75 (3H) |
| I.2-1228 | 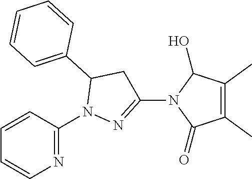 [SMILES: CC1=C(C)C(O)N(C2=NN(c3ccccn3)C(c3ccccc3)C2)C1=O] | MS: 348.16 | ¹H-NMR (400 MHz, CDCl₃ δ, ppm) 8.04 (1H), 7.44-7.52 (1H), 7.21-7.37 (5H), 6.86 (1H), 6.60 (1H), 5.89 (1H), 5.59 (1H), 4.00- 4.07 (1H), 3.55-3.58 (1H), 2.06 (3H), 1.81 (3H) |
| I.2-432 | 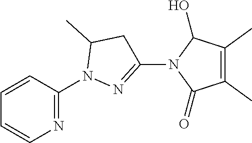 [SMILES: CC1=C(C)C(O)N(C2=NN(c3ccccn3)C(C)C2)C1=O] | MS: 286.14 | ¹H-NMR (400 MHz, CDCl₃ δ, ppm) 8.30-8.35 (1H), 7.55-7.57 (1H), 6.76-6.83 (1H), 6.66-6.69 (1H), 5.83-5.87 (1H), 4.60-4.65 (1H), 3.67-3.76 (1H), 3.37-3.38 (1H), 2.05-2.07 (3H), 1.84-1.88 (3H), 1.39-1.43 (3H) |
| I.2-1126 | 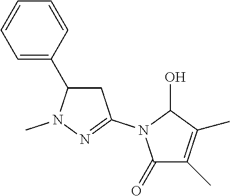 [SMILES: CC1=C(C)C(O)N(C2=NN(C)C(c3ccccc3)C2)C1=O] | MS: 285.15 | ¹H-NMR (400 MHz, CDCl₃ δ, ppm) 7.26-7.45 (5H), 5.72-5.74 (1H), 4.66-4.70 (1H), 3.80-3.96 (2H), 3.06-3.19 (1H), 2.64-2.65 (3H), 2.03 (3H), 1.81 (3H) |
| I.2-1351 | 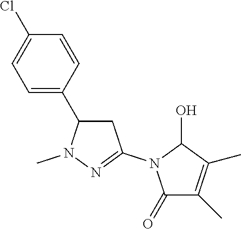 [SMILES: CC1=C(C)C(O)N(C2=NN(C)C(c3ccc(Cl)cc3)C2)C1=O] | MS: 319.11 | ¹H-NMR (400 MHz, CDCl₃ δ, ppm) 7.33-7.39 (4H), 5.71-5.73 (1H), 3.80 -3.94 (2H), 3.01-3.12 (1H), 2.63 (3H), 2.03 (3H), 1.81 (3H) |
| I.23-1228 | 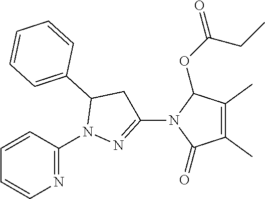 [SMILES: CCC(=O)OC1C(C)=C(C)C(=O)N1C1=NN(c2ccccn2)C(c2ccccc2)C1] | MS: 404.18 | ¹H-NMR (400 MHz, CDCl₃ δ, ppm) 7.95-7.97 (1H), 7.08-7.52 (1H), 6.53-6.56 (1H), 5.72 (1H), 3.90-4.07 (1H), 3.41-3.61 (1H), 2.40-2.51 (2H), 2.01 (3H), 1.83 (3H), 1.11-1.26 (3H) |
| I.33-1228 | 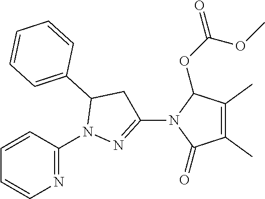 [SMILES: COC(=O)OC1C(C)=C(C)C(=O)N1C1=NN(c2ccccn2)C(c2ccccc2)C1] | MS: 406.16 | ¹H-NMR (400 MHz, CDCl₃ δ, ppm) 7.96-7.98 (1H), 7.41-7.46 (1H), 7.10-7.32 (5H), 6.95-6.98 (1H), 6.53-6.57 (1H), 5.70-5.74 (1H), 3.90-3.98 (2H), 3.40-3.59 (1H), 2.02 (3H), 1.83 (3H) |
| I.23-432 | 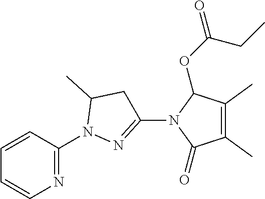 [SMILES: CCC(=O)OC1C(C)=C(C)C(=O)N1C1=NN(c2ccccn2)C(C)C1] | MS: 342.17 | ¹H-NMR (400 MHz, CDCl₃ δ, ppm) 8.10-8.12 (1H), 7.48-7.52 (1H), 7.00-7.12 (2H), 6.60-6.63 (1H), 4.76-4.83 (1H), 3.57-3.73 (1H), 3.24-3.39 (1H), 2.36-2.43 (2H), 1.96 (3H), 1.86 (3H), 1.41- 1.43 (3H), 1.14-1.19 (3H) |
| I.33-432 | 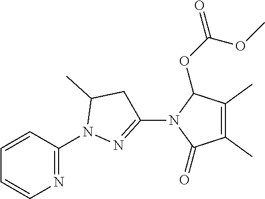 [SMILES: COC(=O)OC1C(C)=C(C)C(=O)N1C1=NN(c2ccccn2)C(C)C1] | MS: 344.15 | ¹H-NMR (400 MHz, CDCl₃ δ, ppm) 8.11-8.13 (1H), 7.41-7.45 (1H), 7.00-7.04 (1H), 6.89-6.92 (1H), 6.58-6.61 (1H), 4.69-4.77 (1H), 3.87-3.89 (3H), 3.57-3.67 (1H), 3.16-3.33 (1H), 2.01 (3H), 1.86 (3H), 1.42-1.44 (3H)) |
| I.23-1386 | 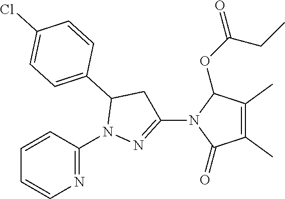 [SMILES: CCC(=O)OC1C(C)=C(C)C(=O)N1C1=NN(c2ccccn2)C(c2ccc(Cl)cc2)C1] | MS: 438.15 | ¹H-NMR (400 MHz, CDCl₃ δ, ppm) 7.95-7.96 (1H), 7.46-7.52 (1H), 7.08-7.31 (6H), 6.56-6.59 (1H), 5.67 (1H), 3.90-4.06 (1H), 3.36-3.57 (1H), 2.28-2.52 (2H), 1.97 (3H), 1.84 (3H), 1.15-1.28 (3H) |
| I.33-1386 | 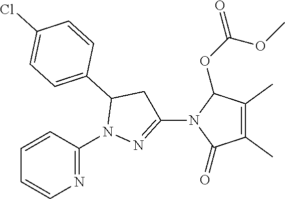 [SMILES: COC(=O)OC1C(C)=C(C)C(=O)N1C1=NN(c2ccccn2)C(c2ccc(Cl)cc2)C1] | MS: 440.13 | ¹H-NMR (400 MHz, CDCl₃ δ, ppm) 7.96-7.98 (1H), 7.42-7.47 (1H), 7.19-7.31 (4H), 7.09-7.12 (1H), 6.96-7.11 (1H), 6.56-6.59 (1H), 5.64-5.69 (1H), 3.90-3.97 (4H), 3.48-3.54 (1H), 2.02 (3H), 1.84 (3H), |
| I.5-432 | 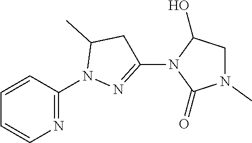 [SMILES: CC1CC(N2C(=O)N(C)CC2O)=NN1c1ccccn1] | MS: 275.14 | ¹H-NMR (400 MHz, CDCl₃ δ, ppm) 8.12-8.14 (1H), 7.41-7.45 (1H), 6.98-7.00 (1H), 6.55-6.59 (1H), 4.66 (1H), 4.46-4.48 (1H), 3.56-3.63 (1H), 3.49 (3H), 3.41-3.42 (1H), 1.36-1.40 (3H) |
| I.5-1228 | 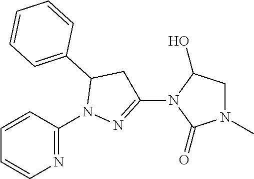 [SMILES: CN1CC(O)N(C2=NN(c3ccccn3)C(c3ccccc3)C2)C1=O] | MS: 337.15 | ¹H-NMR (400 MHz, CDCl₃ δ, ppm) 8.05-8.16 (1H), 7.36-7.42 (2H), 7.19-7.33 (4H), 6.85-6.91 (1H), 6.53-6.62 (1H), 5.95-5.98 (1H), 5.41-5.47 (1H), 4.09-4.12 (1H), 3.60-3.73 (1H), 3.33-3.50 (2H), 2.87-2.90 (3H) |

### FORMULATION EXAMPLES

a) A dust is obtained by mixing 10 parts by weight of a compound of the formula (I) and 90 parts by weight of talc as inert substance and comminuting the mixture in a hammer mill.
b) A wettable powder which is readily dispersible in water is obtained by mixing 25 parts by weight of a compound of the formula (I), 64 parts by weight of kaolin-containing quartz as inert substance, 10 parts by weight of potassium lignosulfonate and 1 part by weight of sodium oleoylmethyltaurate as wetting agent and dispersant, and grinding the mixture in a pinned-disk mill.
c) A readily water-dispersible dispersion concentrate is obtained by mixing 20 parts by weight of a compound of the formula (I) with 6 parts by weight of alkylphenol polyglycol ether (®Triton X 207), 3 parts by weight of isotridecanol polyglycol ether (8 EO) and 71 parts by weight of paraffinic mineral oil (boiling range for example about 255 to about 277° C.) and grinding the mixture in a ball mill to a fineness of below 5 microns.
d) An emulsifiable concentrate is obtained from 15 parts by weight of a compound of the formula (I), 75 parts by weight of cyclohexanone as solvent and 10 parts by weight of oxyethylated nonylphenol as emulsifier.
e) Water-dispersible granules are obtained by mixing

| | | |
|---|---|---|
| 75 | parts by weight | of a compound of the formula (I), |
| 10 | parts by weight | of calcium lignosulfonate, |
| 5 | parts by weight | of sodium lauryl sulfate, |
| 3 | parts by weight | of polyvinyl alcohol and |
| 7 | parts by weight | of kaolin, |

grinding the mixture in a pinned-disk mill, and granulating the powder in a fluidized bed by spray application of water as a granulating liquid.
f) Water-dispersible granules are also obtained by homogenizing and precomminuting, in a colloid mill,

| | | |
|---|---|---|
| 25 | parts by weight | of a compound of the formula (I), |
| 5 | parts by weight | of sodium 2,2′-dinaphthylmethane-6,6′-disulfonate, |
| 2 | parts by weight | of sodium oleoylmethyltaurinate, |
| 1 | part by weight | of polyvinyl alcohol, |
| 17 | parts by weight | of calcium carbonate and |
| 50 | parts by weight | of water, |

then grinding the mixture in a bead mill and atomizing and drying the resulting suspension in a spray tower by means of a one-phase nozzle.

### BIOLOGICAL EXAMPLES

Measurement of PS II Activity in Thylakoid Membranes

1. Preparation of the Thylakoid Membranes

Cooled fresh spinach leaves were comminuted and homogenized in 50 mM phosphate buffer, pH 6.8, 10 mM KCl, 0.34 M sucrose (sucrose buffer) (mixer, 1 g of plant material/ml). The homogenate was subsequently filtered through 4 layers of Miracloth and the chloroplasts were obtained by centrifugation:
10 minutes (min) of centrifugation at 4400×g (4° C.).
The sediment was suspended in 25 ml of sucrose buffer and centrifuged for another 10 min at 4400×g (4° C.).
The sediment was then suspended in 40 ml of 50 mM phosphate buffer, pH 6.8, 10 mM KCl, without sucrose.
In this step, the chloroplasts were osmotically ruptured and the thylakoid membranes were then obtained by centrifugation (10 min, 4400×g, 4° C.). The membrane sediment was finally suspended in about 20 ml of 50 mM phosphate buffer, pH 6.8, 10 mM KCl.
After protein determination and activity determination, the membrane suspension was aliquoted and frozen in liquid nitrogen. The aliquots were stored at −80° C. Under these conditions, the photosystem II preparation is storage-stable for at least three months.

2. Activity Determination of the Photosystem II (PS II)

Test Principle:

The electron transfer from PS II to an artificial electronic captor, 2,6-dichlorophenol-indophenol (DCPIP), is measured under the action of light. The concentration of the blue oxidized form of DCPIP can be determined spectraphotometrically at the wavelength l=595 nm. The enzyme-catalyzed reduction of DCPIP results in a colourless leuco form and thus to a decrease of absorption at 595 nm in the reaction batch, which is measured as a function of time.

Practice:

The activity determination was carried out in microtiter plates (96 wells) in a reaction volume of 200 μl. 155 μl of dilute membrane suspension in 50 mM phosphate buffer, pH 6.8, 10 mM KCl were initially charged. Depending on the activity of the PS II preparation, the dilution was adjusted such that measurement of the decrease in absorption (λ=595 nm) was linear for at least 10 min.

In each case, 5 μl of solutions of the test compounds of variable concentration in DMSO were added to the enzyme suspension; controls contained 5 μl of DMSO; the final concentration of DMSO in the reaction batch was thus 2.5% (v/v); this concentration does not adversely affect the enzymatic activity.

Typical dilution series of the test compounds comprised 10 concentration steps in the range between 10⁻⁹and 10⁻⁵ M.

On each microtiter plate, a known PS II inhibitor, e.g. metribuzin, was used as standard for assessing the quality of the PS II test.

The reaction was initiated by addition of 40 μl DCPIP solution (600 μM in distilled water); the final concentration of DCPIP was 120 μM.

Absorption was measured over a period of 10 min at 22° C. and with illumination. For calculating the active compound concentration at which 50% of the enzyme activity is inhibited (IC₅₀), the relative enzymatic activity (in %) was plotted as a function of the logarithm of the active compound concentration.

The table below shows the results of this activity determination of photosystem II for some compounds of the general formula (I) according to the invention, where the numeration of Tables I.1 to I.77 also applies here.

| No. | pI50 |
|---|---|
| I.1-21 | 4.8 |
| I.13-732 | 4.0 |
| I.2-1126 | 4.2 |
| I.2-1228 | 4.1 |
| I.2-132 | 4.4 |
| I.2-1351 | 4.8 |
| I.2-1386 | 4.2 |
| I.2-1413 | 5.3 |
| I.2-1451 | 4.5 |
| I.2-1486 | 4.2 |
| I.2-217 | 5.4 |
| I.2-321 | 5.1 |
| I.2-432 | 4.1 |
| I.2-621 | 4.9 |
| I.2-700 | 4.4 |
| I.2-732 | 4.0 |
| I.23-1126 | 4.1 |
| I.23-1228 | 4.2 |
| I.23-132 | 4.1 |
| I.23-1351 | 4.2 |
| I.23-1386 | 4.2 |
| I.23-1413 | 4.7 |
| I.23-1451 | 4.2 |
| I.23-1486 | 4.2 |
| I.23-321 | 4.1 |
| I.23-432 | 4.1 |
| I.23-621 | 4.1 |
| I.23-732 | 4.1 |
| I.24-1126 | 4.2 |
| I.24-1413 | 4.2 |
| I.24-432 | 4.8 |
| I.24-621 | 4.1 |
| I.26-1413 | 4.3 |
| I.3-1126 | 4.4 |
| I.3-1351 | 5.5 |
| I.3-1386 | 4.2 |
| I.3-1413 | 4.1 |
| I.3-1451 | 4.9 |
| I.3-21 | 4.7 |
| I.3-321 | 5.4 |
| I.3-432 | 4.1 |
| I.3-621 | 4.2 |
| I.33-1126 | 4.1 |
| I.33-1228 | 4.2 |
| I.33-1351 | 4.2 |
| I.33-1386 | 4.2 |
| I.33-1451 | 4.2 |
| I.33-1486 | 4.2 |
| I.33-432 | 4.1 |
| I.33-732 | 4.1 |
| I.34-1126 | 4.2 |
| I.5-1126 | 4.0 |
| I.5-1228 | 4.1 |
| I.5-1351 | 4.1 |
| I.5-1386 | 4.2 |
| I.5-1451 | 4.2 |
| I.5-432 | 4.0 |
| I.5-732 | 4.0 |
| I.72-21 | 4.1 |
| I.72-321 | 4.1 |
| I.73-1126 | 4.0 |
| I.73-1139 | 4.1 |
| I.73-1228 | 4.1 |
| I.73-132 | 4.1 |
| I.73-1351 | 4.7 |
| I.73-1386 | 4.2 |
| I.73-1413 | 4.1 |
| I.73-1451 | 4.1 |
| I.73-1486 | 4.2 |
| I.73-303 | 4.0 |
| I.73-321 | 4.0 |
| I.73-432 | 4.0 |
| I.73-621 | 4.0 |
| I.73-732 | 4.0 |
| I.74-1126 | 4.1 |
| I.74-1351 | 4.3 |
| I.74-1386 | 4.2 |
| I.74-1413 | 4.1 |
| I.74-21 | 4.1 |
| I.74-321 | 4.1 |
| I.74-432 | 4.1 |
| I.74-621 | 4.1 |
| I.8-1126 | 5.1 |
| I.8-1139 | 4.8 |
| I.8-1351 | 5.7 |
| I.8-1413 | 4.3 |
| I.8-432 | 4.5 |
| I.8-621 | 5.3 |

Pre-Emergence Herbicidal Action

Seeds of mono- and dicotyledonous weed plants or crop plants were placed in sandy loam in wood fibre pots and covered with soil. The formulated compounds of the general formula (I) according to the invention were then applied to the surface of the covering soil in the form of an aqueous suspension or emulsion at an application rate of 600 l of water/ha (converted) with addition of 0.2% wetting agent.

After the treatment, the pots were placed in a greenhouse and kept under good growth conditions for the test plants. After about 3 weeks, the effect was scored visually in percent in comparison to untreated controls. For example, 100% activity=the plants have died, 50% herbicidal activity or damage=plants reduced to 50% or plant material reduced by 50%, 0% activity=like control plants.

Post-Emergence Herbicidal Activity

Seeds of mono- and dicotyledonous weed plants and crop plants were placed in sandy loam in wood fibre pots, covered with soil and then cultivated in a greenhouse under good growth conditions. Two to 3 weeks after sowing, the test plants were treated at the one-leaf stage, with the formulated compounds of the general formula (I) according to the invention being sprayed as an aqueous suspension or emulsion at an application rate of 600 l of water/ha (converted) with addition of 0.2% wetting agent, onto the green parts of the plants. After the test plants had been left to stand in the greenhouse for about 3 weeks under optimum growth conditions, the activity of the preparations was scored visually in percent in comparison to untreated controls. For example, 100% activity=the plants have died, 50% herbicidal activity or damage=plants reduced to 50% or plant material reduced by 50%, 0% activity=like control plants.

Here, in the biological tests, the compounds of the general formula (I) according to the invention were in each case employed in a formulation, with the numerations of Tables I.1 to I.77 also applying here.

Here, the herbicidal action of the compounds of the general formula (I) according to the invention was examined for the following harmful plants:

ALOMY=Alopecurus myosuroides

ABUTH=Abutilon theophrasti

AMARE=Amaranthus retroflexus

HORMU=Hordeum murinum

MATIN=Matricaria inodora (=Tripleurospermum maritimum subsp. inodorum)

PHBPU=Ipomoea purpurea

POLCO=Polygonum convolvulus (=Fallopia convolvulus)

SETVI=Setaria viridis

STEME=Stellaria media

VIOTR=Viola tricolor

VERPE=Veronica persica

The respective herbicidal action was in each case determined at the same point in time after application of the formulation in question, i.e. damage of the harmful plant in question in %.

Pre-emergence herbicidal action of the compounds of the general formula (I) according to the invention:

| | Application rate | | | | | | | | | | | |
|---|---|---|---|---|---|---|---|---|---|---|---|---|
| No. | g/ha | ALOMY | SETVI | ABUTH | AMARE | MATIN | PHBPU | POLCO | STEME | VIOTR | VERPE | HORMU |
| I.2-1413 | 1280 | 70 | 70 | 0 | 60 | 100 | 0 | 100 | 100 | 100 | — | 60 |
| I.23-1413 | 1280 | 0 | 40 | 0 | 50 | 70 | 0 | 80 | 80 | 100 | 60 | 30 |
| I.24-1126 | 1280 | 10 | 0 | 0 | 0 | 90 | 100 | 0 | 20 | 10 | 0 | 20 |
| I.2-132 | 1280 | 70 | 40 | 70 | 60 | 0 | 0 | 40 | 100 | 80 | 30 | 0 |
| I.23-621 | 1280 | 0 | 50 | 0 | 40 | 50 | 0 | 100 | 0 | 70 | 0 | 0 |
| I.23-132 | 1280 | 60 | 0 | 0 | 70 | 40 | 30 | 0 | 100 | 70 | 60 | 20 |

Post-emergence herbicidal action of the compounds of the general formula (I) according to the invention:

| | Application rate | | | | |
|---|---|---|---|---|---|
| No. | g/ha | SETVI | PHBPU | POLCO | VIOTR |
| I.8-621 | 1280 | 60 | 90 | 90 | 80 |
| I.2-432 | 1280 | 80 | 0 | 0 | 80 |

1. Compound of formula (I) and/or a salt thereof, 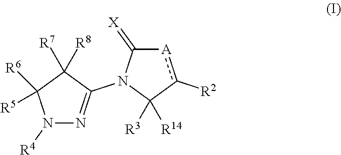
in which
X represents oxygen or sulfur,
A represents the grouping C—R¹ or the grouping N—R⁹, where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents hydrogen, halogen, hydroxy, (C₁-C₈)-alkyl, (C₁-C₈)-haloalkyl, (C₁-C₈)-hydroxyalkyl, (C₁-C₈)-alkoxy, (C₁-C₈)-alkoxyalkyl, (C₁-C₈)-alkoxy-(C₁-C₈)-alkyloxy, (C₁-C₈)-alkylthio, (C₁-C₈)-haloalkoxy, (C₁-C₈)-haloalkylthio, (C₃-C₁₀)-cycloalkyl, aryl, heteroaryl, (C₃-C₈) cycloalkyl-(C₁-C₈)-alkyl, heterocyclyl, (C₂-C₈)-alkenyl, (C₂-C₈)-alkenyloxy, (C₂-C₈)-alkynyl, (C₂-C₈)-alkynyloxy, NR¹⁰R¹¹, aryl-(C₁-C₈)-alkyl, heteroaryl-(C₁-C₈)-alkyl or heterocyclyl-(C₁-C₈)-alkyl,
R² represents hydrogen, halogen, hydroxy, (C₁-C₈)-alkyl, (C₁-C₈)-haloalkyl, (C₁-C₈)-hydroxyalkyl, hydroxycarbonyl-(C₁-C₈)-alkyl, (C₁-C₈)-alkoxy, (C₁-C₈)-alkoxyalkyl, (C₁-C₈)-alkoxy-(C₁-C₈)-alkyloxy, (C₁-C₈)-alkylthio, (C₁-C₈)-haloalkoxy, (C₁-C₈)-haloalkylthio, (C₃-C₁₀)-cycloalkyl, aryl, heteroaryl, (C₃-C₈)-cycloalkyl-(C₁-C₈)-alkyl, heterocyclyl, (C₂-C₈)-alkenyl, (C₂-C₈)-alkenyloxy, (C₂-C₈)-alkynyl, (C₂-C₈)-alkynyloxy, NR¹⁰R¹¹, aryl-(C₁-C₈)-alkyl, heteroaryl-(C₁-C₈)-alkyl, heterocyclyl-(C₁-C₈)-alkyl, (C₁-C₈)-cyanoalkyl, C(O)R¹², C(O)OR¹², C(O)NR¹⁰R¹¹, SO₂R¹³, (C₁-C₈)-alkoxycarbonyl-(C₁-C₈)-alkyl, (C₂-C₈)-alkenyloxycarbonyl-(C₁-C₈)-alkyl, aryl-(C₁-C₈)-alkoxycarbonyl-(C₁-C₈)-alkyl, heteroaryl-(C₁-C₈)-alkoxycarbonyl-(C₁-C₈)-alkyl, aryloxycarbonyl-(C₁-C₈)-alkyl, arylcarbonyl-(C₁-C₈)-alkyl, (C₁-C₈)-alkylcarbonyl-(C₁-C₈)-alkyl, heteroarylcarbonyl-(C₁-C₈)-alkyl or heterocyclylcarbonyl-(C₁-C₈)-alkyl,
or where R¹ and R² together with the two carbon atoms to which they are attached form a fully saturated or partially saturated ring which has 3 to 7 members in total, which is optionally interrupted by one to three heteroatoms from the group consisting of N, O and S and which is optionally substituted further,
R³ represents hydroxy, hydrothio, halogen, NR¹⁰R¹¹, (C₁-C₈)-alkoxy, (C₃-C₁₀)-cycloalkyl-(C₁-C₈)-alkoxy, aryl-(C₁-C₈)-alkoxy, (C₁-C₈)-alkoxy-(C₁-C₈)-alkoxy, arylcarbonyloxy, (C₁-C₈)-alkylcarbonyloxy, aryl-(C₁-C₈)-alkylcarbonyloxy, heteroarylcarbonyloxy, (C₃-C₁₀)-cycloalkylcarbonyloxy, heterocyclylcarbonyloxy, (C₁-C₈)-haloalkyl-carbonyloxy, (C₂-C₈)-alkenylcarbonyloxy, OC(O)OR¹², OC(O)SR¹², OC(S)OR¹², OC(S)SR¹², OC(O)NR¹⁰R¹¹, OC(S)NR¹⁰R¹¹, OSO₂R¹³, OSO₂OR¹² or OCHO,
R⁴ represents hydrogen, (C₁-C₈)-alkyl, (C₁-C₈)-haloalkyl, (C₃-C₁₀)-cycloalkyl, (C₃-C₁₀)-cycloalkyl-(C₁-C₈)-alkyl, aryl, heteroaryl, heterocyclyl, aryl-(C₁-C₈)-alkyl, heteroaryl-(C₁-C₈)-alkyl, heterocyclyl-(C₁-C₈)-alkyl, (C₂-C₈)-alkenyl, (C₂-C₈)-alkynyl, (C₂-C₈)-haloalkenyl, (C₂-C₈) haloalkynyl, (C₄-C₁₀)-cycloalkenyl, aryl-(C₂-C₈)-alkenyl, heteroaryl-(C₂-C₈)-alkenyl, heterocyclyl-(C₂-C₈)-alkenyl, arylcarbonyl-(C₁-C₈)-alkyl, (C₁-C₈)-alkylcarbonyl-(C₁-C₈)-alkyl, heteroarylcarbonyl-(C₁-C₈)-alkyl, (C₃-C₁₀)-cycloalkylcarbonyl-(C₁-C₈)-alkyl, aryl-(C₁-C₈)-alkoxycarbonyl-(C₁-C₈)-alkyl, (C₁-C₈)-alkoxycarbonyl-(C₁-C₈)-alkyl, (C₁-C₈)-alkoxy-(C₁-C₈)-alkyl, (C₁-C₈)-haloalkoxy-(C₁-C₈)-alkyl, C(O)R¹², SO₂R¹³, CHO, R¹⁰R¹¹N—(C₁-C₈)-alkyl or cyano-(C₁-C₈)-alkyl,
R⁵ represents hydrogen, (C₁-C₈)-alkyl, (C₁-C₈)-haloalkyl, (C₃-C₁₀)-cycloalkyl, (C₃-C₁₀)-cycloalkyl-(C₁-C₈)-alkyl, aryl, heteroaryl, heterocyclyl, aryl-(C₁-C₈)-alkyl, heteroaryl-(C₁-C₈)-alkyl, heterocyclyl-(C₁-C₈)-alkyl, cyano, C(O)OR¹², C(O)NR¹⁰R¹¹, R¹²O(O)C—(C₁-C₈)-alkyl, R¹²O—(C₁-C₈)-alkyl, (C₁-C₈)-alkylcarbonyloxy-(C₁-C₈)-alkyl, (C₃-C₁₀)-cycloalkylcarbonyloxy-(C₁-C₈)-alkyl, arylcarbonyloxy-(C₁-C₈)-alkyl, heteroarylcarbonyloxy-(C₁-C₈)-alkyl or heterocyclylcarbonyloxy-(C₁-C₈)-alkyl,
R⁶ represents hydrogen, (C₁-C₈)-alkyl, (C₁-C₈)-haloalkyl, (C₃-C₁₀)-cycloalkyl, (C₃-C₁₀)-cycloalkyl-(C₁-C₈)-alkyl, aryl, heteroaryl, heterocyclyl, aryl-(C₁-C₈)-alkyl, heteroaryl-(C₁-C₈)-alkyl or heterocyclyl-(C₁-C₈)-alkyl,
R⁷ and R⁸ are identical or different and independently of one another represent hydrogen, halogen or (C₁-C₈)-alkyl,
R⁹ represents hydrogen, hydroxy, (C₁-C₈)-alkyl, (C₁-C₈)-haloalkyl, (C₁-C₈)-hydroxyalkyl, hydroxycarbonyl-(C₁-C₈)-alkyl, (C₁-C₈)-alkoxy, (C₁-C₈)-alkoxy-(C₁-C₈)-alkyl, (C₁-C₈)-alkoxyalkyloxy, (C₁-C₈)-haloalkoxy, (C₃—C o)-cycloalkyl, aryl, heteroaryl, (C₃-C₁₀)-cycloalkyl-(C₁-C₈)-alkyl, heterocyclyl, (C₂-C₈)-alkenyl, (C₂-C₈)-alkenyloxy, (C₂-C₈)-alkynyl, (C₂-C₈)-alkynyloxy, NR¹⁰R¹¹, aryl-(C₁-C₈)-alkyl, heteroaryl-(C₁-C₈)-alkyl, heterocyclyl-(C₁-C₈)-alkyl, (C₁—C₈)-cyanoalkyl, C(O)R¹², C(O)OR¹², C(O)NR¹⁰R¹¹, SO₂R¹³, (C₁-C₈)-alkoxycarbonyl-(C₁-C₈)-alkyl, (C₂-C₈)-alkenyloxycarbonyl-(C₁-C₈)-alkyl, aryl-(C₁-C₈)-alkoxycarbonyl-(C₁-C₈)-alkyl, heteroaryl-(C₁-C₈)-alkoxycarbonyl-(C₁-C₈)-alkyl, aryloxycarbonyl-(C₁-C₈)-alkyl, arylcarbonyl-(C₁-C₈)-alkyl, (C₁-C₈)-alkylcarbonyl-(C₁-C₈)-alkyl, heteroarylcarbonyl-(C₁-C₈)-alkyl or heterocyclylcarbonyl-(C₁-C₈)-alkyl,
or where R² and R⁹ together with the nitrogen atom or carbon atom to which they are respectively attached form a fully saturated or partially saturated ring which has 3 to 7 members in total, which is optionally interrupted by one to three heteroatoms from the group consisting of N, O and S and which is optionally substituted further,
R¹⁰ and R¹¹ are identical or different and independently of one another represent hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₂-C₈)-haloalkynyl, (C₃-C₁₀)-cycloalkyl, (C₄-C₁₀)-cycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₁₀)-cycloalkyl-(C₁-C₈)-alkyl, (C₄-C₁₀)-cycloalkenyl-(C₁-C₈)-alkyl, C(O)R¹², SO₂R¹³, heterocyclyl, (C₁-C₈)-alkoxycarbonyl-(C₁-C₈)-alkyl, (C₁-C₈)-alkoxycarbonyl, aryl-(C₁-C₈)-alkoxycarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl or heterocyclyl-(C₁-C₈)-alkyl,
R¹² represents (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₂-C₈)-haloalkynyl, (C₃-C₁₀)-cycloalkyl, (C₄-C₁₀)-cycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₁₀)-cycloalkyl-(C₁-C₈)-alkyl, (C₄-C₁₀)-cycloalkenyl-(C₁-C₈)-alkyl, (C₁-C₈)-alkoxycarbonyl-(C₁-C₈)-alkyl, (C₂-C₈)-alkenyloxycarbonyl-(C₁-C₈)-alkyl, aryl-(C₁-C₈)-alkoxycarbonyl-(C₁-C₈)-alkyl, hydroxycarbonyl-(C₁-C₈)-alkyl, heterocyclyl or heterocyclyl-(C₁-C₈)-alkyl,
R¹³ represents (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₂-C₈)-haloalkynyl, (C₃-C₁₀)-cycloalkyl, (C₄-C₁₀)-cycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₁₀)-cycloalkyl-(C₁-C₈)-alkyl, (C₄-C₁₀)-cycloalkenyl-(C₁-C₈)-alkyl or NR¹⁰R¹¹,
R¹⁴ represents hydrogen or (C₁-C₈)-alkyl,
or where R³ and R¹⁴ together with the carbon atom to which they are attached form a carbonyl group,
where the cyclic structural elements of the radicals mentioned respectively in R¹, R², R³, R⁴, R⁵, R⁶, R⁹, R¹⁰, R¹¹, R¹² and R¹³ and the rings formed by R¹ and R² or R² and R⁹, having 3 to 7 members in total, are in each case unsubstituted or substituted by one or more radicals selected from the group consisting of halogen, nitro, hydroxy, cyano, NR¹⁰R¹¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxy-carbonyl, (C₁-C₄)-haloalkoxy-carbonyl, (C₁-C₄)-alkylcarboxy, (C₃-C₆)-cycloalkyl, (C₃-C₆)-cycloalkyl-(C₁-C₆)-alkyl, (C₁-C₄)-alkoxy-carbonyl-(C₁-C₄)-alkyl, hydroxycarbonyl, hydroxycarbonyl-(C₁-C₄)-alkyl, R¹⁰R¹¹N-carbonyl, and where the structural elements cycloalkyl, cycloalkenyl and heterocyclyl have n oxo groups, where n=0, 1 or 2.

2. Compound of formula (I) and/or salt thereof according to claim 1, wherein
X represents oxygen or sulfur,
A represents the grouping C—R¹ or the grouping N—R⁹, where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents hydrogen, halogen, hydroxy, (C₁-C₇)-alkyl, (C₁-C₇)-haloalkyl, (C₁-C₇)-hydroxyalkyl, (C₁-C₇)-alkoxy, (C₁-C₇)-alkoxyalkyl, (C₁-C₇)-alkoxy-(C₁-C₇)-alkyloxy, (C₁-C₇)-haloalkoxy, (C₃-C₁₀)-cycloalkyl, (C₂-C₇)-alkenyl, (C₂-C₇)-alkenyloxy, (C₂-C₇)-alkynyl or (C₂-C₇)-alkynyloxy,
R² represents hydrogen, halogen, hydroxy, (C₁-C₇)-alkyl, (C₁-C₇)-haloalkyl, (C₁-C₇)-hydroxyalkyl, (C₁-C₇)-alkoxy, (C₁-C₇)-alkoxyalkyl, (C₁-C₇)-alkoxy-(C₁-C₇)-alkyloxy, (C₁-C₇)-haloalkoxy, (C₃-C₁₀)-cycloalkyl, (C₂-C₇)-alkenyl, (C₂-C₇)-alkenyloxy, (C₂-C₇)-alkynyl, (C₂-C₇)-alkynyloxy or NR¹⁰R¹¹,
or where R¹ and R² together with the two carbon atoms to which they are attached form a fully saturated or partially saturated ring which has 3 to 7 members in total, which is optionally interrupted by one to three heteroatoms from the group consisting of N, O and S and which is optionally substituted further,
R³ represents hydroxy, hydrothio, halogen, NR¹⁰R¹¹, (C₁-C₇)-alkoxy, (C₃-C₁₀)-cycloalkyl-(C₁-C₇)-alkoxy, aryl-(C₁-C₇)-alkoxy, (C₁-C₇)-alkoxy-(C₁-C₇)-alkoxy, arylcarbonyloxy, (C₁-C₇)-alkylcarbonyloxy, aryl-(C₁-C₇)-alkylcarbonyloxy, heteroarylcarbonyloxy, (C₃-C₁₀)-cycloalkylcarbonyloxy, heterocyclylcarbonyloxy, (C₁-C₇)-haloalkylcarbonyloxy, (C₂-C₇)-alkenylcarbonyloxy, OC(O)OR¹², OC(O)SR¹², OC(S)OR¹², OC(S)SR¹², OC(O)NR¹⁰R¹¹, OC(S)NR¹⁰R¹¹, OSO₂R¹³, OSO₂OR¹² or OCHO,
R⁴ represents hydrogen, (C₁-C₇)-alkyl, (C₁-C₇)-haloalkyl, (C₃-C₁₀)-cycloalkyl, (C₃-C₁₀)-cycloalkyl-(C₁-C₇)-alkyl, aryl, heteroaryl, heterocyclyl, aryl-(C₁-C₇)-alkyl, heteroaryl-(C₁-C₇)-alkyl, heterocyclyl-(C₁-C₇)-alkyl, (C₂-C₇)-alkenyl, (C₂-C₇)-alkynyl, (C₂-C₇)-haloalkenyl, (C₂-C₇)-haloalkynyl, (C₄-C₈)-cycloalkenyl, aryl-(C₂-C₇)-alkenyl, heteroaryl-(C₂-C₇)-alkenyl, heterocyclyl-(C₂-C₇)-alkenyl, arylcarbonyl-(C₁-C₇)-alkyl, (C₁-C₇)-alkylcarbonyl-(C₁-C₇)-alkyl, heteroarylcarbonyl-(C₁-C₇)-alkyl, (C₃-C₁₀)-cycloalkylcarbonyl-(C₁-C₇)-alkyl, aryl-(C₁-C₇)-alkoxycarbonyl-(C₁-C₇)-alkyl, (C₁-C₇)-alkoxycarbonyl-(C₁-C₇)-alkyl, (C₁-C₇)-alkoxy-(C₁-C₇)-alkyl, (C₁-C₇)-haloalkoxy-(C₁-C₇)-alkyl, C(O)R¹², SO₂R¹³, CHO, R¹⁰R¹¹N—(C₁-C₈)-alkyl or cyano-(C₁-C₈)-alkyl,
R⁵ represents hydrogen, (C₁-C₇)-alkyl, (C₁-C₇)-haloalkyl, (C₃-C₁₀)-cycloalkyl, (C₃-C₁₀)-cycloalkyl-(C₁-C₇)-alkyl, aryl, heteroaryl, heterocyclyl, aryl-(C₁-C₇)-alkyl, heteroaryl-(C₁-C₇)-alkyl, heterocyclyl-(C₁-C₇)-alkyl, cyano, C(O)OR¹², C(O)NR¹⁰R¹¹, R¹²O(O)C—(C₁-C₇)-alkyl, R¹²O—(C₁-C₇)-alkyl, (C₁-C₇)-alkylcarbonyloxy-(C₁-C₇)-alkyl, (C₃-C₁₀)-cycloalkylcarbonyloxy-(C₁-C₇)-alkyl, arylcarbonyloxy-(C₁-C₇)-alkyl, heteroarylcarbonyloxy-(C₁-C₇)-alkyl or heterocyclylcarbonyloxy-(C₁-C₇)-alkyl,
R⁶ represents hydrogen, (C₁-C₇)-alkyl, (C₁-C₇)-haloalkyl, (C₃-C₁₀)-cycloalkyl, (C₃-C₁₀)-cycloalkyl-(C₁-C₇)-alkyl, aryl, heteroaryl, heterocyclyl, aryl-(C₁-C₇)-alkyl, heteroaryl-(C₁-C₇)-alkyl or heterocyclyl-(C₁-C₇)-alkyl,
R⁷ and R⁸ are identical or different and independently of one another represent hydrogen, halogen or (C₁-C₇)-alkyl,
R⁹ represents hydrogen, hydroxy, (C₁-C₇)-alkyl, (C₁-C₇)-haloalkyl, (C₁-C₇)-alkoxy, (C₁-C₇)-alkoxy-(C₁-C₇)-alkyl, (C₃-C₁₀)-cycloalkyl, (C₂-C₇)-alkenyl, (C₂-C₇)-alkynyl or (C₁-C₇)-cyanoalkyl,
or where R² and R⁹ together with the nitrogen atom or carbon atom to which they are respectively attached form a fully saturated or partially saturated ring which has 3 to 7 members in total, which is optionally interrupted by one to three heteroatoms from the group consisting of N, O and S and which is optionally substituted further,
R¹⁰ and R¹¹ are identical or different and independently of one another represent hydrogen, (C₁-C₇)-alkyl, (C₂-C₇)-alkenyl, (C₂-C₇)-alkynyl, (C₁-C₇)-cyanoalkyl, (C₁-C₈)-haloalkyl, (C₂-C₇)-haloalkenyl, (C₂-C₇)-haloalkynyl, (C₃-C₁₀)-cycloalkyl, (C₄-C₈)-cycloalkenyl, (C₁-C₇)-alkoxy-(C₁-C₇)-alkyl, (C₁-C₇)-haloalkoxy-(C₁-C₇)-alkyl, (C₁-C₇)-alkylthio-(C₁-C₇)-alkyl, (C₁-C₇)-haloalkylthio-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, (C₃-C₁₀)-cycloalkyl-(C₁-C₇)-alkyl, (C₄-C₈)-cycloalkenyl-(C₁-C₇)-alkyl, C(O)R¹², SO₂R¹³, heterocyclyl, (C₁-C₇)-alkoxycarbonyl-(C₁-C₇)-alkyl, (C₁-C₇)-alkoxycarbonyl, aryl-(C₁-C₇)-alkoxycarbonyl-(C₁-C₇)-alkyl, aryl-(C₁-C₇)-alkoxycarbonyl, heteroaryl-(C₁-C₇)-alkoxycarbonyl, (C₂-C₇)-alkenyloxycarbonyl, (C₂-C₇)-alkynyloxycarbonyl or heterocyclyl-(C₁-C₇)-alkyl,
R¹² represents (C₁-C₇)-alkyl, (C₂-C₇)-alkenyl, (C₂-C₇)-alkynyl, (C₁-C₇)-cyanoalkyl, (C₁-C₈)-haloalkyl, (C₂-C₇)-haloalkenyl, (C₂-C₇)-haloalkynyl, (C₃-C₁₀)-cycloalkyl, (C₄-C₈)-cycloalkenyl, (C₁-C₇)-alkoxy-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, (C₃-C₁₀)-cycloalkyl-(C₁-C₇)-alkyl, (C₄-C₈)-cycloalkenyl-(C₁-C₇)-alkyl, (C₁-C₇)-alkoxycarbonyl-(C₁-C₇)-alkyl, (C₂-C₇)-alkenyloxycarbonyl-(C₁-C₇)-alkyl, aryl-(C₁-C₇)-alkoxycarbonyl-(C₁-C₇)-alkyl, hydroxycarbonyl-(C₁-C₇)-alkyl, heterocyclyl or heterocyclyl-(C₁-C₇)-alkyl,
R¹³ represents (C₁-C₇)-alkyl, (C₂-C₇)-alkenyl, (C₂-C₇)-alkynyl, (C₁-C₇)-cyanoalkyl, (C₁-C₈)-haloalkyl, (C₂-C₇)-haloalkenyl, (C₂-C₇)-haloalkynyl, (C₃-C₁₀)-cycloalkyl, (C₄-C₈)-cycloalkenyl, (C₁-C₇)-alkoxy-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, heterocyclyl-(C₁-C₇)-alkyl, (C₃-C₁₀)-cycloalkyl-(C₁-C₇)-alkyl, (C₄-C₈)-cycloalkenyl-(C₁-C₇)-alkyl or NR¹⁰R¹¹,
R¹⁴ represents hydrogen or (C₁-C₄)-alkyl,
or where R³ and R¹⁴ together with the carbon atom to which they are attached form a carbonyl group,
where the cyclic structural elements of the radicals mentioned respectively in R¹, R², R³, R⁴, R⁵, R⁶, R⁹, R¹⁰, R¹¹, R¹² and R¹³ and the rings formed by R¹ and R² or R² and R⁹, having 3 to 7 members in total, are in each case unsubstituted or substituted by one or more radicals selected from the group consisting of halogen, nitro, hydroxy, cyano, NR¹⁰R¹¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxy-carbonyl, (C₁-C₄)-haloalkoxy-carbonyl, (C₁-C₄)-alkylcarboxy, (C₃-C₆)-cycloalkyl, (C₃-C₆)-cycloalkyl-(C₁-C₆)-alkyl, (C₁-C₄)-alkoxy-carbonyl-(C₁-C₄)-alkyl, hydroxycarbonyl, hydroxycarbonyl-(C₁-C₄)-alkyl, R¹⁰R¹¹N-carbonyl, and where the structural elements cycloalkyl, cycloalkenyl and heterocyclyl have n oxo groups, where n=0, 1 or 2.

3. Compound of formula (I) and/or salt thereof according to claim 1, wherein
X represents oxygen,
A represents the grouping C—R¹ or the grouping N—R⁹, where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents hydrogen, halogen, hydroxy, (C₁-C₆)-alkyl, (C₁-C₆)-haloalkyl, (C₁-C₆)-hydroxyalkyl, (C₁-C₆)-alkoxy, (C₁-C₆)-alkoxyalkyl, (C₁-C₆)-alkoxy-(C₁-C₆)-alkyloxy, (C₁-C₆)-haloalkoxy, (C₃-C₁₀)-cycloalkyl, (C₂-C₆)-alkenyl, (C₂-C₆)-alkenyloxy, (C₂-C₆)-alkynyl or (C₂-C₆)-alkynyloxy,
R² represents hydrogen, halogen, hydroxy, (C₁-C₆)-alkyl, (C₁-C₆)-haloalkyl, (C₁-C₆)-hydroxyalkyl, (C₁-C₆)-alkoxy, (C₁-C₆)-alkoxyalkyl, (C₁-C₆)-alkoxy-(C₁-C₆)-alkyloxy, (C₁-C₆)-haloalkoxy, (C₃-C₁₀)-cycloalkyl, (C₂-C₆)-alkenyl, (C₂-C₆)-alkenyloxy, (C₂-C₆)-alkynyl, (C₂-C₆)-alkynyloxy or NR¹⁰R¹¹,
or where R¹ and R² together with the two carbon atoms to which they are attached form a fully saturated or partially saturated ring which has 3 to 7 members in total, which is optionally interrupted by one to three heteroatoms from the group consisting of N, O and S and which is optionally substituted further,
R³ represents hydroxy, hydrothio, halogen, (C₁-C₆)-alkoxy, (C₃-C₁₀)-cycloalkyl-(C₁-C₆)-alkoxy, aryl-(C₁-C₆)-alkoxy, (C₁-C₆)-alkoxy-(C₁-C₆)-alkoxy, arylcarbonyloxy, (C₁-C₆)-alkylcarbonyloxy, aryl-(C₁-C₆)-alkylcarbonyloxy, heteroarylcarbonyloxy, (C₃-C₁₀)-cycloalkylcarbonyloxy, heterocyclylcarbonyloxy, (C₁-C₆)-haloalkyl-carbonyloxy, (C₂-C₆)-alkenylcarbonyloxy, OC(O)OR¹² or OSO₂R¹³,
R⁴ represents hydrogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₆)-alkyl, heteroaryl-(C₁-C₆)-alkyl, heterocyclyl-(C₁-C₆)-alkyl, (C₂-C₆)-alkenyl, (C₂-C₆)-alkynyl, (C₂-C₆)-haloalkenyl, (C₂-C₆)-haloalkynyl, (C₄-C₈)-cycloalkenyl, aryl-(C₂-C₆)-alkenyl, heteroaryl-(C₂-C₆)-alkenyl, heterocyclyl-(C₂-C₆)-alkenyl, arylcarbonyl-(C₁-C₆)-alkyl, (C₁-C₆)-alkylcarbonyl-(C₁-C₆)-alkyl, heteroarylcarbonyl-(C₁-C₆)-alkyl, (C₃-C₁₀)-cycloalkylcarbonyl-(C₁-C₆)-alkyl, aryl-(C₁-C₆)-alkoxycarbonyl-(C₁-C₆)-alkyl, (C₁-C₆)-alkoxycarbonyl-(C₁-C₆)-alkyl, (C₁-C₆)-alkoxy-(C₁-C₆)-alkyl, (C₁-C₆)-haloalkoxy-(C₁-C₆)-alkyl, C(O)R¹², SO₂R¹³, CHO, R¹⁰R¹¹N—(C₁-C₈)-alkyl or cyano-(C₁-C₈)-alkyl,
R⁵ represents hydrogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₆)-alkyl, heteroaryl-(C₁-C₆)-alkyl, heterocyclyl-(C₁-C₆)-alkyl, cyano, C(O)OR¹², C(O)NR¹⁰R¹¹, R¹²O(O)C—(C₁-C₆)-alkyl, R¹²O—(C₁-C₆)-alkyl, (C₁-C₆)-alkylcarbonyloxy-(C₁-C₆)-alkyl, (C₃-C₁₀)-cycloalkylcarbonyloxy-(C₁-C₆)-alkyl, arylcarbonyloxy-(C₁-C₆)-alkyl, heteroarylcarbonyloxy-(C₁-C₆)-alkyl or heterocyclylcarbonyloxy-(C₁-C₆)-alkyl,
R⁶ represents hydrogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₆)-alkyl, heteroaryl-(C₁-C₆)-alkyl or heterocyclyl-(C₁-C₆)-alkyl,
R⁷ and R⁸ are identical or different and independently of one another represent hydrogen, halogen or (C₁-C₆)-alkyl,
R⁹ represents hydrogen, hydroxy, (C₁-C₆)-alkyl, (C₁-C₆)-haloalkyl, (C₁-C₆)-alkoxy, (C₁-C₆)-alkoxy-(C₁-C₆)-alkyl, (C₃-C₁₀)-cycloalkyl, (C₂-C₆)-alkenyl, (C₂-C₆)-alkynyl or (C₁-C₆)-cyanoalkyl,
or where R² and R⁹ together with the nitrogen atom or carbon atom to which they are respectively attached form a fully saturated or partially saturated ring which has 3 to 7 members in total, which is optionally interrupted by one to three heteroatoms from the group consisting of N, O and S and which is optionally substituted further,
R¹⁰ and R¹¹ are identical or different and independently of one another represent hydrogen, (C₁-C₆)-alkyl, (C₂-C₆)-alkenyl, (C₂-C₆)-alkynyl, (C₁-C₆)-cyanoalkyl, (C₁-C₇)-haloalkyl, (C₂-C₆)-haloalkenyl, (C₂-C₆)-haloalkynyl, (C₃-C₁₀)-cycloalkyl, (C₄-C₇)-cycloalkenyl, (C₁-C₆)-alkoxy-(C₁-C₆)-alkyl, (C₁-C₆)-haloalkoxy-(C₁-C₆)-alkyl, (C₁-C₆)-alkylthio-(C₁-C₆)-alkyl, (C₁-C₆)-haloalkylthio-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, (C₃-C₁₀)-cycloalkyl-(C₁-C₆)-alkyl, (C₄-C₇)-cycloalkenyl-(C₁-C₆)-alkyl, C(O)R¹², SO₂R¹³, heterocyclyl, (C₁-C₆)-alkoxycarbonyl-(C₁-C₆)-alkyl, (C₁-C₆)-alkoxycarbonyl, aryl-(C₁-C₆)-alkoxycarbonyl-(C₁-C₆)-alkyl, aryl-(C₁-C₆)-alkoxycarbonyl, heteroaryl-(C₁-C₆)-alkoxycarbonyl, (C₂-C₆)-alkenyloxycarbonyl, (C₂-C₆)-alkynyloxycarbonyl or heterocyclyl-(C₁-C₆)-alkyl,
R¹² represents (C₁-C₆)-alkyl, (C₂-C₆)-alkenyl, (C₂-C₆)-alkynyl, (C₁-C₆)-cyanoalkyl, (C₁-C₇)-haloalkyl, (C₂-C₆)-haloalkenyl, (C₂-C₆)-haloalkynyl, (C₃-C₁₀)-cycloalkyl, (C₄-C₇)-cycloalkenyl, (C₁-C₆)-alkoxy-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, (C₃-C₁₀)-cycloalkyl-(C₁-C₆)-alkyl, (C₄-C₇)-cycloalkenyl-(C₁-C₆)-alkyl, (C₁-C₆)-alkoxycarbonyl-(C₁-C₆)-alkyl, (C₂-C₆)-alkenyloxycarbonyl-(C₁-C₆)-alkyl, aryl-(C₁-C₆)-alkoxycarbonyl-(C₁-C₆)-alkyl, hydroxycarbonyl-(C₁-C₆)-alkyl, heterocyclyl or heterocyclyl-(C₁-C₆)-alkyl,
R¹³ represents (C₁-C₆)-alkyl, (C₂-C₆)-alkenyl, (C₂-C₆)-alkynyl, (C₁-C₆)-cyanoalkyl, (C₁-C₇)-haloalkyl, (C₂-C₆)-haloalkenyl, (C₂-C₆)-haloalkynyl, (C₃-C₁₀)-cycloalkyl, (C₄-C₇)-cycloalkenyl, (C₁-C₆)-alkoxy-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, heterocyclyl-(C₁-C₆)-alkyl, (C₃-C₁₀)-cycloalkyl-(C₁-C₆)-alkyl, (C₄-C₇)-cycloalkenyl-(C₁-C₆)-alkyl or NR¹⁰R¹¹,
R¹⁴ represents hydrogen,
or where R³ and R¹⁴ together with the carbon atom to which they are attached form a carbonyl group,
where the cyclic structural elements of the radicals mentioned respectively in R¹, R², R³, R⁴, R⁵, R⁶, R⁹, R¹⁰, R¹¹, R¹² and R¹³ and the rings formed by R¹ and R² or R² and R⁹, having 3 to 7 members in total, are in each case unsubstituted or substituted by one or more radicals selected from the group consisting of halogen, nitro, hydroxy, cyano, NR¹⁰R¹¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxy-carbonyl, (C₁-C₄)-haloalkoxy-carbonyl, (C₁-C₄)-alkylcarboxy, (C₃-C₆)-cycloalkyl, (C₃-C₆)-cycloalkyl-(C₁-C₆)-alkyl, (C₁-C₄)-alkoxy-carbonyl-(C₁-C₄)-alkyl, hydroxycarbonyl, hydroxycarbonyl-(C₁-C₄)-alkyl, R¹⁰R¹¹N-carbonyl, and where the structural elements cycloalkyl, cycloalkenyl and heterocyclyl have n oxo groups, where n=0, 1 or 2.

4. Compound of formula (I) and/or salt thereof according to claim 1, wherein
X represents oxygen,
A represents the grouping C—R¹ or the grouping N—R⁹, where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents hydrogen, halogen, (C₁-C₄)-alkyl, (C₁-C₄)-haloalkyl, (C₁-C₄)-hydroxyalkyl, (C₁-C₄)-alkoxy, (C₁-C₄)-haloalkoxy or (C₃-C₈)-cycloalkyl,
R² represents hydrogen, halogen, hydroxy, (C₁-C₄)-alkyl, (C₁-C₄)-haloalkyl, (C₁-C₄)-hydroxyalkyl, (C₁-C₄)-alkoxy, (C₁-C₄)-haloalkoxy or (C₃-C₈)-cycloalkyl, (C₂-C₄)-alkenyl, (C₂-C₄)-alkynyl, (C₂-C₄)-alkynyloxy or NR¹⁰R¹¹,
or where R¹ and R² together with the two carbon atoms to which they are attached form a fully saturated or partially saturated ring which has 3 to 7 members in total, which is optionally interrupted by one to three heteroatoms from the group consisting of N, O and S and which is optionally substituted further,
R³ represents hydroxy, halogen, (C₁-C₄)-alkoxy, (C₃-C₁₀)-cycloalkyl-(C₁-C₄)-alkoxy, aryl-(C₁-C₄)-alkoxy, arylcarbonyloxy, (C₁-C₄)-alkylcarbonyloxy, aryl-(C₁-C₄)-alkylcarbonyloxy, heteroarylcarbonyloxy, (C₃-C₈)-cycloalkylcarbonyloxy, heterocyclylcarbonyloxy, (C₁-C₄)-haloalkyl-carbonyloxy, (C₂-C₄)-alkenylcarbonyloxy, OC(O)OR¹² or OSO₂R¹³,
R⁴ represents hydrogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₆)-alkyl, heteroaryl-(C₁-C₆)-alkyl, heterocyclyl-(C₁-C₆)-alkyl, (C₂-C₆)-alkenyl, (C₂-C₆)-alkynyl, (C₂-C₆)-haloalkenyl, (C₂-C₆)-haloalkynyl, (C₄-C₈)-cycloalkenyl, aryl-(C₂-C₆)-alkenyl, heteroaryl-(C₂-C₆)-alkenyl, heterocyclyl-(C₂-C₆)-alkenyl, arylcarbonyl-(C₁-C₆)-alkyl, (C₁-C₆)-alkylcarbonyl-(C₁-C₆)-alkyl, heteroarylcarbonyl-(C₁-C₆)-alkyl, (C₃-C₁₀)-cycloalkylcarbonyl-(C₁-C₆)-alkyl, aryl-(C₁-C₆)-alkoxycarbonyl-(C₁-C₆)-alkyl, (C₁-C₆)-alkoxycarbonyl-(C₁-C₆)-alkyl, (C₁-C₆)-alkoxy-(C₁-C₆)-alkyl, (C₁-C₆)-haloalkoxy-(C₁-C₆)-alkyl, C(O)R¹², SO₂R¹³, CHO, R¹⁰R¹¹N—(C₁-C₈)-alkyl or cyano-(C₁-C₈)-alkyl,
R⁵ represents hydrogen, (C₁-C₄)-alkyl, (C₁-C₄)-haloalkyl, (C₃-C₈)-cycloalkyl, (C₃-C₈)-cycloalkyl-(C₁-C₄)-alkyl, aryl, heteroaryl, heterocyclyl, aryl-(C₁-C₄)-alkyl, heteroaryl-(C₁-C₄)-alkyl, heterocyclyl-(C₁-C₄)-alkyl, cyano, C(O)OR¹², C(O)NR¹⁰R¹¹, R¹²O(O)C—(C₁-C₄)-alkyl, R¹²O—(C₁-C₄)-alkyl, (C₁-C₄)-alkylcarbonyloxy-(C₁-C₄)-alkyl, (C₃-C₈)-cycloalkylcarbonyloxy-(C₁-C₄)-alkyl, arylcarbonyloxy-(C₁-C₄)-alkyl, heteroarylcarbonyloxy-(C₁-C₄)-alkyl or heterocyclylcarbonyloxy-(C₁-C₄)-alkyl,
R⁶ represents hydrogen, (C₁-C₄)-alkyl, (C₁-C₄)-haloalkyl, (C₃-C₈)-cycloalkyl or (C₃-C₈)-cycloalkyl-(C₁-C₄)-alkyl,
R⁷ and R⁸ are identical or different and independently of one another represent hydrogen, halogen or (C₁-C₄)-alkyl,
R⁹ represents hydrogen, hydroxy, (C₁-C₄)-alkyl, (C₁-C₄)-haloalkyl, (C₁-C₄)-alkoxy, (C₁-C₄)-alkoxy-(C₁-C₄)-alkyl, (C₃-C₈)-cycloalkyl, (C₂-C₄)-alkenyl or (C₂-C₄)-alkynyl,
or where R² and R⁹ together with the nitrogen atom or carbon atom to which they are respectively attached form a fully saturated ring which has 3 to 7 members in total, which is optionally interrupted by one heteroatom from the group consisting of N, O and S and which is optionally substituted further,
R¹⁰ and R¹¹ are identical or different and independently of one another represent hydrogen, (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-haloalkoxy-(C₁-C₄)-alkyl, (C₁-C₄)-alkylthio-(C₁-C₄)-alkyl, (C₁-C₄)-haloalkylthio-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₈)-cycloalkyl-(C₁-C₄)-alkyl, (C₄-C₇)-cycloalkenyl-(C₁-C₄)-alkyl, C(O)R¹², SO₂R¹³, heterocyclyl, (C₁-C₄)-alkoxycarbonyl-(C₁-C₄)-alkyl, (C₁-C₄)-alkoxycarbonyl, aryl-(C₁-C₄)-alkoxycarbonyl-(C₁-C₄)-alkyl, aryl-(C₁-C₄)-alkoxycarbonyl, heteroaryl-(C₁-C₄)-alkoxycarbonyl, (C₂-C₄)-alkenyloxycarbonyl, (C₂-C₄)-alkynyloxycarbonyl or heterocyclyl-(C₁-C₄)-alkyl,
R¹² represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₈)-cycloalkyl-(C₁-C₄)-alkyl, (C₄-C₇)-cycloalkenyl-(C₁-C₄)-alkyl, (C₁-C₄)-alkoxycarbonyl-(C₁-C₄)-alkyl, (C₂-C₄)-alkenyloxycarbonyl-(C₁-C₄)-alkyl, aryl-(C₁-C₄)-alkoxycarbonyl-(C₁-C₄)-alkyl, hydroxycarbonyl-(C₁-C₄)-alkyl, heterocyclyl or heterocyclyl-(C₁-C₄)-alkyl,
R¹³ represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, heterocyclyl-(C₁-C₄)-alkyl, (C₃-C₈)-cycloalkyl-(C₁-C₄)-alkyl, (C₄-C₇)-cycloalkenyl-(C₁-C₄)-alkyl or NR¹⁰R¹¹,
R¹⁴ represents hydrogen,
or where R³ and R¹⁴ together with the carbon atom to which they are attached form a carbonyl group,
where the cyclic structural elements of the radicals mentioned respectively in R¹, R², R³, R⁴, R⁵, R⁶, R⁹, R¹⁰, R¹¹, R¹² and R¹³ and the rings formed by R¹ and R² or R² and R⁹, having 3 to 7 members in total, are in each case unsubstituted or substituted by one or more radicals selected from the group consisting of halogen, nitro, hydroxy, cyano, NR¹⁰R¹¹, (C₁-C₄)-alkyl, (C₁—C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxy-carbonyl, (C₁-C₄)-haloalkoxy-carbonyl, (C₁-C₄)-alkylcarboxy, (C₃-C₆)-cycloalkyl, (C₃-C₆)-cycloalkyl-(C₁-C₆)-alkyl, (C₁-C₄)-alkoxy-carbonyl-(C₁-C₄)-alkyl, hydroxycarbonyl, hydroxycarbonyl-(C₁-C₄)-alkyl, R¹⁰R¹¹N-carbonyl, and where the structural elements cycloalkyl, cycloalkenyl and heterocyclyl have n oxo groups, where n=0, 1 or 2.

5. Compound of formula (I) and/or salt thereof according to claim 1, wherein
X represents oxygen,
A represents the grouping C—R¹ or the grouping N—R⁹, where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents hydrogen, halogen, (C₁-C₄)-alkyl, (C₁-C₄)-haloalkyl, (C₁-C₄)-alkoxy, (C₁-C₄)-haloalkoxy or (C₅-C₇)-cycloalkyl,
R² represents hydrogen, halogen, (C₁-C₄)-alkyl, (C₁-C₄)-haloalkyl, (C₁-C₄)-alkoxy, (C₁-C₄)-haloalkoxy or (C₅-C₇)-cycloalkyl,
R³ represents hydroxy, arylcarbonyloxy, (C₁-C₄)-alkylcarbonyloxy, aryl-(C₁-C₄)-alkylcarbonyloxy, (C₁-C₄)-haloalkyl-carbonyloxy, OC(O)OR¹² or OSO₂R¹³,
R⁴ represents hydrogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₆)-alkyl, heteroaryl-(C₁-C₆)-alkyl, heterocyclyl-(C₁-C₆)-alkyl, (C₂-C₆)-alkenyl, (C₂-C₆)-alkynyl, (C₂-C₆)-haloalkenyl, (C₂-C₆) haloalkynyl, (C₄-C₈)-cycloalkenyl, aryl-(C₂-C₆)-alkenyl, heteroaryl-(C₂-C₆)-alkenyl, heterocyclyl-(C₂-C₆)-alkenyl, arylcarbonyl-(C₁-C₆)-alkyl, (C₁-C₆)-alkylcarbonyl-(C₁-C₆)-alkyl, heteroarylcarbonyl-(C₁-C₆)-alkyl, (C₃-C₁₀)-cycloalkylcarbonyl-(C₁-C₆)-alkyl, aryl-(C₁-C₆)-alkoxycarbonyl-(C₁-C₆)-alkyl, (C₁-C₆)-alkoxycarbonyl-(C₁-C₆)-alkyl, (C₁-C₆)-alkoxy-(C₁-C₆)-alkyl, (C₁-C₆)-haloalkoxy-(C₁-C₆)-alkyl, C(O)R¹², SO₂R¹³, CHO, R¹⁰R¹¹N—(C₁-C₈)-alkyl or cyano-(C₁-C₈)-alkyl,
R⁵ represents hydrogen, (C₁-C₄)-alkyl, (C₁-C₄)-haloalkyl, (C₅-C₈)-cycloalkyl, aryl, heteroaryl, heterocyclyl, C(O)OR¹², C(O)NR¹⁰R¹¹ or R¹²O(O)C—(C₁-C₄)-alkyl,
R⁶ represents hydrogen, (C₁-C₄)-alkyl or (C₁-C₄)-haloalkyl,
R⁷ and R⁸ are identical or different and independently of one another represent hydrogen, halogen or (C₁-C₄)-alkyl,
R⁹ represents hydrogen, hydroxy, (C₁-C₄)-alkyl, (C₁-C₄)-haloalkyl, (C₁-C₄)-alkoxy or (C₃-C₆)-cycloalkyl,
or where R² and R⁹ together with the nitrogen atom or carbon atom to which they are respectively attached form a preferably fully saturated ring which has 3 to 7 members in total, which is optionally interrupted by one heteroatom from the group consisting of N and O and which is optionally substituted further,
R¹⁰ and R¹¹ are identical or different and independently of one another represent hydrogen, (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-haloalkoxy-(C₁-C₄)-alkyl, (C₁-C₄)-alkylthio-(C₁-C₄)-alkyl, (C₁-C₄)-haloalkylthio-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₈)-cycloalkyl-(C₁-C₄)-alkyl, (C₄-C₇)-cycloalkenyl-(C₁-C₄)-alkyl, C(O)R¹², SO₂R¹³, heterocyclyl, (C₁-C₄)-alkoxycarbonyl-(C₁-C₄)-alkyl, (C₁-C₄)-alkoxycarbonyl, aryl-(C₁-C₄)-alkoxycarbonyl-(C₁-C₄)-alkyl, aryl-(C₁-C₄)-alkoxycarbonyl, heteroaryl-(C₁-C₄)-alkoxycarbonyl, (C₂-C₄)-alkenyloxycarbonyl, (C₂-C₄)-alkynyloxycarbonyl or heterocyclyl-(C₁-C₄)-alkyl,
R¹² represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₈)-cycloalkyl-(C₁-C₄)-alkyl, (C₄-C₇)-cycloalkenyl-(C₁-C₄)-alkyl, (C₁-C₄)-alkoxycarbonyl-(C₁-C₄)-alkyl, (C₂-C₄)-alkenyloxycarbonyl-(C₁-C₄)-alkyl, aryl-(C₁-C₄)-alkoxycarbonyl-(C₁-C₄)-alkyl, hydroxycarbonyl-(C₁-C₄)-alkyl, heterocyclyl or heterocyclyl-(C₁-C₄)-alkyl,
R¹³ represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, heterocyclyl-(C₁-C₄)-alkyl, (C₃-C₈)-cycloalkyl-(C₁-C₄)-alkyl, (C₄-C₇) cycloalkenyl-(C₁-C₄)-alkyl or NR¹⁰R¹¹,
R¹⁴ represents hydrogen,
or where R³ and R¹⁴ together with the carbon atom to which they are attached form a carbonyl group,
where the cyclic structural elements of the radicals mentioned respectively in R¹, R², R³, R⁴, R⁵, R⁶, R⁹, R¹⁰, R¹¹, R¹² and R¹³ and the ring formed by R² and R⁹, having 3 to 7 members in total, are in each case unsubstituted or substituted by one or more radicals selected from the group consisting of halogen, nitro, hydroxy, cyano, NR¹⁰R¹¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxy-carbonyl, (C₁-C₄)-haloalkoxy-carbonyl, (C₁-C₄)-alkylcarboxy, (C₃-C₆)-cycloalkyl, (C₃-C₆)-cycloalkyl-(C₁-C₆)-alkyl, (C₁-C₄)-alkoxy-carbonyl-(C₁-C₄)-alkyl, hydroxycarbonyl, hydroxycarbonyl-(C₁-C₄)-alkyl,
R¹⁰R¹¹N-carbonyl, and where the structural elements cycloalkyl, cycloalkenyl and heterocyclyl have n oxo groups, where n=0, 1 or 2.

6. Compound of formula (I) and/or salt thereof according to claim 1, wherein
X represents oxygen,
A represents the grouping C—R¹ or the grouping N—R⁹, where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents hydrogen, halogen, (C₁-C₄)-alkyl, (C₁-C₄)-alkoxy or (C₅-C₇)-cycloalkyl,
R² represents hydrogen, halogen, (C₁-C₄)-alkyl, (C₁-C₄)-alkoxy or (C₅-C₇)-cycloalkyl,
R³ represents hydroxy, arylcarbonyloxy, (C₁-C₄)-alkylcarbonyloxy, (C₁-C₄)-haloalkyl-carbonyloxy, OC(O)O—(C₁-C₄)-alkyl, OSO₂—(C₁-C₄)-alkyl or OSO₂-aryl,
R⁴ represents hydrogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₆)-alkyl, heteroaryl-(C₁-C₆)-alkyl, heterocyclyl-(C₁-C₆)-alkyl, (C₂-C₆)-alkenyl, (C₂-C₆)-alkynyl, (C₂-C₆)-haloalkenyl, (C₂-C₆) haloalkynyl, (C₄-C₈)-cycloalkenyl, aryl-(C₂-C₆)-alkenyl, heteroaryl-(C₂-C₆)-alkenyl, heterocyclyl-(C₂-C₆)-alkenyl, arylcarbonyl-(C₁-C₆)-alkyl, (C₁-C₆)-alkylcarbonyl-(C₁-C₆)-alkyl, heteroarylcarbonyl-(C₁-C₆)-alkyl, (C₃-C₁₀)-cycloalkylcarbonyl-(C₁-C₆)-alkyl, aryl-(C₁-C₆)-alkoxycarbonyl-(C₁-C₆)-alkyl, (C₁-C₆)-alkoxycarbonyl-(C₁-C₆)-alkyl, (C₁-C₆)-alkoxy-(C₁-C₆)-alkyl, (C₁-C₆)-haloalkoxy-(C₁-C₆)-alkyl, C(O)R¹², SO₂R¹³, CHO, R¹⁰R¹¹N—(C₁-C₈)-alkyl or cyano-(C₁-C₈)-alkyl,
R⁵ represents hydrogen, (C₁-C₃)-alkyl, (C₁-C₃)-haloalkyl, aryl, heteroaryl, C(O)O—(C₁—C₄)-alkyl or (C₁-C₄)-alkyl-O(O)C—(C₁-C₄)-alkyl,
R⁶ represents hydrogen or (C₁-C₄)-alkyl,
R⁷ and R⁸ are identical or different and independently of one another represent hydrogen, bromine, chlorine or (C₁-C₃)-alkyl,
R⁹ represents hydrogen, hydroxy, (C₁-C₄)-alkyl, (C₁-C₄)-haloalkyl, (C₁-C₄)-alkoxy or (C₃-C₆)-cycloalkyl,
or where R² and R⁹ together with the nitrogen atom or carbon atom to which they are respectively attached form a fully saturated ring which has 4 to 6 members in total, which is optionally interrupted by one heteroatom from the group consisting of N and O and which is optionally substituted further,
R¹⁰ and R¹¹ are identical or different and independently of one another represent hydrogen, (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-haloalkoxy-(C₁-C₄)-alkyl, (C₁-C₄)-alkylthio-(C₁-C₄)-alkyl, (C₁-C₄)-haloalkylthio-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₈)-cycloalkyl-(C₁-C₄)-alkyl, (C₄-C₇)-cycloalkenyl-(C₁-C₄)-alkyl, C(O)R¹², SO₂R¹³, heterocyclyl, (C₁-C₄)-alkoxycarbonyl-(C₁-C₄)-alkyl, (C₁-C₄)-alkoxycarbonyl, aryl-(C₁-C₄)-alkoxycarbonyl-(C₁-C₄)-alkyl, aryl-(C₁-C₄)-alkoxycarbonyl, heteroaryl-(C₁-C₄)-alkoxycarbonyl, (C₂-C₄)-alkenyloxycarbonyl, (C₂-C₄)-alkynyloxycarbonyl or heterocyclyl-(C₁-C₄)-alkyl,
R¹² represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₈)-cycloalkyl-(C₁-C₄)-alkyl, (C₄-C₇)-cycloalkenyl-(C₁-C₄)-alkyl, (C₁-C₄)-alkoxycarbonyl-(C₁-C₄)-alkyl, (C₂-C₄)-alkenyloxycarbonyl-(C₁-C₄)-alkyl, aryl-(C₁-C₄)-alkoxycarbonyl-(C₁-C₄)-alkyl, hydroxycarbonyl-(C₁-C₄)-alkyl, heterocyclyl or heterocyclyl-(C₁-C₄)-alkyl,
R¹³ represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, heterocyclyl-(C₁-C₄)-alkyl, (C₃-C₈)-cycloalkyl-(C₁-C₄)-alkyl, (C₄-C₇) cycloalkenyl-(C₁-C₄)-alkyl or NR¹⁰R¹¹,
R¹⁴ represents hydrogen,
or where R³ and R¹⁴ together with the carbon atom to which they are attached form a carbonyl group,
where the cyclic structural elements of the radicals mentioned respectively in R¹, R², R³, R⁴, R⁵, R⁹, R¹⁰, R¹¹, R¹² and R¹³ and the ring formed by R² and R⁹, having 4 to 6 members in total, are in each case unsubstituted or substituted by one or more radicals selected from the group consisting of halogen, nitro, hydroxy, cyano, NR¹⁰R¹¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxy-carbonyl, (C₁-C₄)-haloalkoxy-carbonyl, (C₁-C₄)-alkylcarboxy, (C₃-C₆)-cycloalkyl, (C₃-C₆)-cycloalkyl-(C₁-C₆)-alkyl, (C₁-C₄)-alkoxy-carbonyl-(C₁-C₄)-alkyl, hydroxycarbonyl, hydroxycarbonyl-(C₁-C₄)-alkyl, R¹⁰R¹¹N-carbonyl, and where the structural elements cycloalkyl, cycloalkenyl and heterocyclyl have n oxo groups, where n=0, 1 or 2.

7. Compound of formula (I) and/or salt thereof according to claim 1, wherein
X represents oxygen,
A represents the grouping C—R¹ or the grouping N—R⁹, where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents hydrogen, halogen, (C₁-C₃)-alkyl, (C₁-C₃)-alkoxy or (C₅-C₇)-cycloalkyl,
R² represents hydrogen, halogen, (C₁-C₃)-alkyl, (C₁-C₃)-alkoxy or (C₅-C₇)-cycloalkyl,
R³ represents hydroxy, arylcarbonyloxy, (C₁-C₄)-alkylcarbonyloxy, OC(O)O—(C₁-C₄)-alkyl or OSO₂—(C₁-C₄)-alkyl,
R⁴ represents hydrogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₆)-alkyl, heteroaryl-(C₁-C₆)-alkyl, heterocyclyl-(C₁-C₆)-alkyl, (C₂-C₆)-alkenyl, (C₂-C₆)-alkynyl, (C₂-C₆)-haloalkenyl, (C₂-C₆) haloalkynyl, (C₄-C₈)-cycloalkenyl, aryl-(C₂-C₆)-alkenyl, heteroaryl-(C₂-C₆)-alkenyl, heterocyclyl-(C₂-C₆)-alkenyl, arylcarbonyl-(C₁-C₆)-alkyl, (C₁-C₆)-alkylcarbonyl-(C₁-C₆)-alkyl, heteroarylcarbonyl-(C₁-C₆)-alkyl, (C₃-C₁₀)-cycloalkylcarbonyl-(C₁-C₆)-alkyl, aryl-(C₁-C₆)-alkoxycarbonyl-(C₁-C₆)-alkyl, (C₁-C₆)-alkoxycarbonyl-(C₁-C₆)-alkyl, (C₁-C₆)-alkoxy-(C₁-C₆)-alkyl, (C₁-C₆)-haloalkoxy-(C₁-C₆)-alkyl, C(O)R¹², SO₂R¹³, CHO, R¹⁰R¹¹N—(C₁-C₈)-alkyl or cyano-(C₁—C₈)-alkyl,
R⁵ represents hydrogen, (C₁-C₃)-alkyl, (C₁-C₃)-haloalkyl, phenyl, N-heteroaryl, C(O)O—(C₁-C₄)-alkyl or (C₁-C₄)-alkyl-O(O)C—(C₁-C₄)-alkyl,
R⁶ represents hydrogen or (C₁-C₃)-alkyl,
R⁷ represents hydrogen, bromine, chlorine or (C₁-C₃)-alkyl,
R⁸ represents hydrogen, bromine or chlorine,
R⁹ represents (C₁-C₄)-alkyl or (C₁-C₄)-alkoxy,
or where R² and R⁹ together with the nitrogen atom or carbon atom to which they are respectively attached form a fully saturated ring which has 5 or 6 members in total, which is optionally interrupted by one heteroatom from the group consisting of N and O and which is optionally substituted further,
R¹⁰ and R¹¹ are identical or different and independently of one another represent hydrogen, (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-haloalkoxy-(C₁-C₄)-alkyl, (C₁-C₄)-alkylthio-(C₁-C₄)-alkyl, (C₁-C₄)-haloalkylthio-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₈)-cycloalkyl-(C₁-C₄)-alkyl, (C₄-C₇)-cycloalkenyl-(C₁-C₄)-alkyl, C(O)R¹², SO₂R¹³, heterocyclyl, (C₁-C₄)-alkoxycarbonyl-(C₁-C₄)-alkyl, (C₁-C₄)-alkoxycarbonyl, aryl-(C₁-C₄)-alkoxycarbonyl-(C₁-C₄)-alkyl, aryl-(C₁-C₄)-alkoxycarbonyl, heteroaryl-(C₁-C₄)-alkoxycarbonyl, (C₂-C₄)-alkenyloxycarbonyl, (C₂-C₄)-alkynyloxycarbonyl or heterocyclyl-(C₁-C₄)-alkyl,
R¹² represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₈)-cycloalkyl-(C₁-C₄)-alkyl, (C₄-C₇)-cycloalkenyl-(C₁-C₄)-alkyl, (C₁-C₄)-alkoxycarbonyl-(C₁-C₄)-alkyl, (C₂-C₄)-alkenyloxycarbonyl-(C₁-C₄)-alkyl, aryl-(C₁-C₄)-alkoxycarbonyl-(C₁-C₄)-alkyl, hydroxycarbonyl-(C₁-C₄)-alkyl, heterocyclyl or heterocyclyl-(C₁-C₄)-alkyl,
R¹³ represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, heterocyclyl-(C₁-C₄)-alkyl, (C₃-C₈)-cycloalkyl-(C₁-C₄)-alkyl, (C₄-C₇)-cycloalkenyl-(C₁-C₄)-alkyl or NR¹⁰R¹¹,
R¹⁴ represents hydrogen,
or where R³ and R¹⁴ together with the carbon atom to which they are attached form a carbonyl group,
where the cyclic structural elements of the radicals mentioned respectively in R⁴, R⁵, R¹⁰, R¹, R¹² and R¹³ and the ring formed by R² and R⁹, having 5 or 6 members in total, are in each case unsubstituted or substituted by one or more radicals selected from the group consisting of halogen, nitro, hydroxy, cyano, NR¹⁰R¹¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxy-carbonyl, (C₁-C₄)-haloalkoxy-carbonyl, (C₁-C₄)-alkylcarboxy, (C₃-C₆)-cycloalkyl, (C₃-C₆)-cycloalkyl-(C₁-C₆)-alkyl, (C₁-C₄)-alkoxy-carbonyl-(C₁-C₄)-alkyl, hydroxycarbonyl, hydroxycarbonyl-(C₁-C₄)-alkyl, R¹⁰R¹¹N-carbonyl, and where the structural elements cycloalkyl, cycloalkenyl and heterocyclyl have n oxo groups, where n=0, 1 or 2.

8. Compound of formula (I) and/or salt thereof according to claim 1, wherein
X represents oxygen,
A represents the grouping C—R¹ or the grouping N—R⁹, where R¹ in the grouping C—R¹ and R⁹ in the grouping N—R⁹ each have the meanings according to the definition below and where, if A represents the grouping C—R¹, the adjacent grouping C—R² is attached via a double bond and where, if A represents the grouping N—R⁹, the adjacent grouping CHR² is attached via a single bond,
R¹ represents hydrogen, methyl, chlorine, bromine, methoxy or cyclohexyl,
R² represents hydrogen, methyl, chlorine, bromine, methoxy or cyclohexyl,
R³ represents hydroxy, OC(O)CH₃ (acetoxy), OC(O)CH₂CH₃ (propionyloxy), OC(O)OCH₃ (methylcarbonato), or OSO₂CH₃ (mesylate) and simultaneously R¹⁴ represents hydrogen,
or R³ and R¹⁴ together with the carbon atom to which they are attached form a carbonyl group,
R⁴ represents hydrogen, (C₁-C₆)-alkyl, (C₁-C₆)-haloalkyl, (C₃-C₁₀)-cycloalkyl, (C₃-C₁₀)-cycloalkyl-(C₁-C₆)-alkyl, aryl, heteroaryl, heterocyclyl, aryl-(C₁-C₆)-alkyl, heteroaryl-(C₁-C₆)-alkyl, heterocyclyl-(C₁-C₆)-alkyl, (C₂-C₆)-alkenyl, (C₂-C₆)-alkynyl, (C₂-C₆)-haloalkenyl, (C₂-C₆)-haloalkynyl, (C₄-C₈)-cycloalkenyl, aryl-(C₂-C₆)-alkenyl, heteroaryl-(C₂-C₆)-alkenyl, heterocyclyl-(C₂-C₆)-alkenyl, arylcarbonyl-(C₁-C₆)-alkyl, (C₁-C₆)-alkylcarbonyl-(C₁-C₆)-alkyl, heteroarylcarbonyl-(C₁-C₆)-alkyl, (C₃-C₁₀)-cycloalkylcarbonyl-(C₁-C₆)-alkyl, aryl-(C₁-C₆)-alkoxycarbonyl-(C₁-C₆)-alkyl, (C₁-C₆)-alkoxycarbonyl-(C₁-C₆)-alkyl, (C₁-C₆)-alkoxy-(C₁-C₆)-alkyl, (C₁-C₆)-haloalkoxy-(C₁-C₆)-alkyl, C(O)R¹², SO₂R¹³, CHO, R¹⁰R¹¹N—(C₁-C₈)-alkyl or cyano-(C₁-C₈)-alkyl,
R⁵ represents hydrogen, methyl, trifluoromethyl, CO₂CH₃, CH₂CO₂CH₃, CH₂OC(O)CH₃, optionally substituted phenyl (and here preferably chlorophenyl or methoxyphenyl) or pyridinyl (and here preferably pyridin-2-yl),
R⁶ represents hydrogen or methyl,
R⁷ represents hydrogen, bromine, chlorine or methyl,
R⁸ represents hydrogen, bromine or chlorine,
R⁹ represents methyl, ethyl, isopropyl or methoxy, where R² preferably simultaneously represents hydrogen or methyl,
or R² and R⁹ together with the nitrogen atom or carbon atom to which they are attached form a fully saturated ring which has a total of 5 members and which is optionally interrupted by a further heteroatom from the group consisting of N and O, preferably by O,
R¹⁰ and R¹¹ are identical or different and independently of one another represent hydrogen, (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-haloalkoxy-(C₁-C₄)-alkyl, (C₁-C₄)-alkylthio-(C₁-C₄)-alkyl, (C₁-C₄)-haloalkylthio-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₈)-cycloalkyl-(C₁-C₄)-alkyl, (C₄-C₇)-cycloalkenyl-(C₁-C₄)-alkyl, C(O)R¹², SO₂R¹³, heterocyclyl, (C₁-C₄)-alkoxycarbonyl-(C₁-C₄)-alkyl, (C₁-C₄)-alkoxycarbonyl, aryl-(C₁-C₄)-alkoxycarbonyl-(C₁-C₄)-alkyl, aryl-(C₁-C₄)-alkoxycarbonyl, heteroaryl-(C₁-C₄)-alkoxycarbonyl, (C₂-C₄)-alkenyloxycarbonyl, (C₂-C₄)-alkynyloxycarbonyl or heterocyclyl-(C₁-C₄)-alkyl,
R¹² represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, (C₃-C₈)-cycloalkyl-(C₁-C₄)-alkyl, (C₄-C₇)-cycloalkenyl-(C₁-C₄)-alkyl, (C₁-C₄)-alkoxycarbonyl-(C₁-C₄)-alkyl, (C₂-C₄)-alkenyloxycarbonyl-(C₁-C₄)-alkyl, aryl-(C₁-C₄)-alkoxycarbonyl-(C₁-C₄)-alkyl, hydroxycarbonyl-(C₁-C₄)-alkyl, heterocyclyl or heterocyclyl-(C₁-C₄)-alkyl,
R¹³ represents (C₁-C₄)-alkyl, (C₂-C₄)-alkenyl, (C₂-C₄)-alkynyl, (C₁-C₄)-cyanoalkyl, (C₁-C₄)-haloalkyl, (C₂-C₄)-haloalkenyl, (C₂-C₄)-haloalkynyl, (C₃-C₈)-cycloalkyl, (C₄-C₇)-cycloalkenyl, (C₁-C₄)-alkoxy-(C₁-C₄)-alkyl, (C₁-C₄)-alkoxy-(C₁-C₄)-haloalkyl, aryl, aryl-(C₁-C₄)-alkyl, heteroaryl, heteroaryl-(C₁-C₄)-alkyl, heterocyclyl-(C₁-C₄)-alkyl, (C₃-C₈)-cycloalkyl-(C₁-C₄)-alkyl, (C₄-C₇)-cycloalkenyl-(C₁-C₄)-alkyl or NR¹⁰R¹¹,
where the cyclic structural elements of the radicals mentioned respectively in R⁴, R⁵, R¹⁰, R¹¹, R¹² and R¹³ and the ring formed by R² and R⁹, having 5 members in total, are in each case unsubstituted or substituted by one or more radicals selected from the group consisting of halogen, nitro, hydroxy, cyano, NR¹⁰R¹¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-haloalkylthio, (C₁-C₄)-haloalkylsulfoxy, (C₁-C₄)-haloalkylsulfone, (C₁-C₄)-alkoxy-carbonyl, (C₁-C₄)-haloalkoxy-carbonyl, (C₁-C₄)-alkylcarboxy, (C₃-C₆)-cycloalkyl, (C₃-C₆)-cycloalkyl-(C₁-C₆)-alkyl, (C₁-C₄)-alkoxy-carbonyl-(C₁-C₄)-alkyl, hydroxycarbonyl, hydroxycarbonyl-(C₁-C₄)-alkyl, R¹⁰R¹¹N-carbonyl, and where the structural elements cycloalkyl, cycloalkenyl and heterocyclyl have n oxo groups, where n=0, 1 or 2.

9. One or more compound of formula (I) and/or salts thereof, as defined in claim 1,
as a herbicide and/or plant growth regulator, preferably in crops of useful plants and/or ornamental plants.

10. Herbicidal and/or plant growth-regulating composition, wherein the composition comprises one or more compounds of the formula (I) and/or salts thereof as defined in claim 1,
and one or more further substances selected from groups (i) and/or (ii):
(i) one or more further agrochemically active substances, optionally selected from the group consisting of insecticides, acaricides, nematicides, further herbicides, fungicides, safeners, fertilizers and/or further growth regulators,
(ii) one or more formulation auxiliaries customary in crop protection.

11. Method for controlling one or more harmful plants and/or for regulating the growth of one or more plants, comprising applying an effective amount
of one or more compounds of the formula (I) and/or salts thereof,
or a composition thereof,
to the plants, seeds of plants, the soil in which or on which the plants can grow or an area under cultivation.

12. Process for preparing a compound of the formula (I) as defined in claim 1 or a salt thereof, said process comprising converting 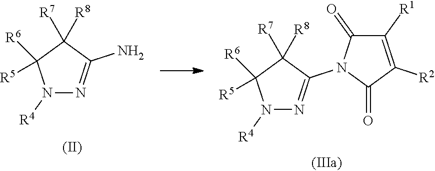
a compound of formula (II) into a compound of formula (IIIa),
or
converting a compound of formula (II) into a compound of formula (XX)

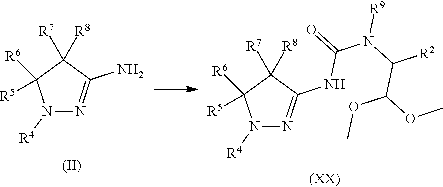

13. Process for preparing a compound of formula (I) or a salt thereof according to claim 12, said process comprising
Converting a compound of formula (II) into a compound of formula (IIIa), and a reaction is carried out with a compound of the formula (XVII) in the presence of a suitable solvent and using a suitable acid or base, or
via a two-step synthesis where initially the compound of the formula (XVIII) is formed and subsequently cyclization takes place, 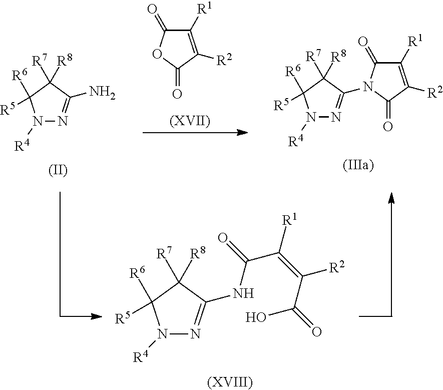
or
a compound of formula (II) is converted into a compound of formula (XX) which is then converted further in a cyclization reaction into a compound of formula (Ic), [chemical structure]
